SEQUENCE LISTING (236 sequences)

SEQ ID NO: 1 - DNA, 385 bp, Artificial Sequence
  FNI1 VH (wt-nt)
caagttcagctggtgcagtctggggctgaggtgaagaggcctgggtcctcggtgaggatctcctgcaaggcctctggtgacaccttcaacaactatgttctcagctgggtgcgacaggcccctggacaagggcttgagtggatggggggaatcatccctatctctggtatcccacattacgcacagaagttccagggcagagtcgcaattatcgcggacgaatccgcgagcacagtctacatggagttgagcagcctacgatctgaggactcggccgtatattactgtgcgagagcggtttccgattattttaatcgagacctcggctgggatgattactactttcctttgtggggccagggcaccctggtcaccgtctcctcag

SEQ ID NO: 2 - protein, 128 aa, Artificial Sequence
  FNI1 VH (aa)
QVQLVQSGAEVKRPGSSVRISCKASGDTFNNYVLSWVRQAPGQGLEWMGGIIPISGIPHYAQKFQGRVAIIADESASTVYMELSSLRSEDSAVYYCARAVSDYFNRDLGWDDYYFPLWGQGTLVTVSS

SEQ ID NO: 3 - protein, 8 aa, Artificial Sequence
  FNI1 CDRH1 (aa)
GDTFNNYV

SEQ ID NO: 4 - protein, 8 aa, Artificial Sequence
  FNI1 CDRH2 (aa)
IIPISGIP

SEQ ID NO: 5 - protein, 21 aa, Artificial Sequence
  FNI1 CDRH3 (aa)
ARAVSDYFNRDLGWDDYYFPL

SEQ ID NO: 6 - DNA, 384 bp, Artificial Sequence
  FNI1 VH (co-nt)
caggtgcagctggtgcagtctggagctgaggtgaagaggccaggatccagcgtgcggatcagctgcaaggcttctggcgacaccttcaacaattacgtgctgtcctgggtgaggcaggctccaggacagggactggagtggatgggcggcatcatccccatcagcggcatccctcactacgcccagaagtttcagggcagggtggccatcatcgctgacgagtccgctagcacagtgtatatggagctgtcttccctgagatctgaggattccgccgtgtactattgtgccagagccgtgtccgactatttcaaccgcgatctgggctgggacgattactattttccactgtggggacagggcaccctggtgacagtgagctct

SEQ ID NO: 7 - DNA, 325 bp, Artificial Sequence
  FNI1 Vk (wt-nt)
gaaatagtgatgacgcagtctccagccaccctgtctgtgtctccaggggaaagagccaccctcttctgcagggccagtcggagtgttagtgacaacttagcctggtaccagcagaaacctggccaggctcccaggctcctcatctttggtgcctccaccagggccactggtgtcccagccaggttcggtggcagtgggtctgggacacagttcactctcaccatcagcagcctgcagtctgaagattttgcagtttattactgtcagcattataatacctggcctccgtggaccttcggccaagggaccaaggtggaaatcaaac

SEQ ID NO: 8 - protein, 108 aa, Artificial Sequence
  FNI1 VK (aa)
EIVMTQSPATLSVSPGERATLFCRASRSVSDNLAWYQQKPGQAPRLLIFGASTRATGVPARFGGSGSGTQFTLTISSLQSEDFAVYYCQHYNTWPPWTFGQGTKVEIK

SEQ ID NO: 9 - protein, 6 aa, Artificial Sequence
  FNI1 CDRL1(aa)
RSVSDN

SEQ ID NO: 10 - protein, 3 aa, Artificial Sequence
  FNI1 CDRL2(aa)
GAS

SEQ ID NO: 11 - protein, 10 aa, Artificial Sequence
  FNI1 CDRL3(aa)
QHYNTWPPWT

SEQ ID NO: 12 - DNA, 324 bp, Artificial Sequence
  FNI1 Vk (co-nt)
gagatcgtgatgacccagtctcctgccacactgtccgtgtccccaggcgagagggccacactgttctgcagggctagcaggtccgtgtccgacaacctggcctggtaccagcagaagccaggccaggctcccagactgctgatctttggagcttccaccagagctacaggcgtgccagctaggttcggaggaagcggatctggcacccagtttaccctgacaatctccagcctgcagagcgaggatttcgccgtgtactattgtcagcactataatacctggcccccttggacatttggccagggcaccaaggtggagatcaag

SEQ ID NO: 13 - DNA, 385 bp, Artificial Sequence
  FNI2 VH (wt-nt)
caggttcagctggtgcagtctggggctgaggtgaagaggcctgggtcctcggtgagggtctcctgcaaggcttctggagccaccttcaataaccatgttctcacctgggtgcgacaggcccctggacaagggcttgagtggatgggagggatcatccctgtctctggaaaaacaacctacgcacagaagttccagggcagagtcgcgataagcacggacgaatccgcgagcacagcctatatggagttgagcagcctgagatctgaggactcggccatatattactgtgcgagagcggtttccgattactttaatcgagacctcggctgggaagattattactttccgatctggggccagggcaccctggtcaccgtctcttcag

SEQ ID NO: 14 - protein, 128 aa, Artificial Sequence
  FNI2 VH (aa)
QVQLVQSGAEVKRPGSSVRVSCKASGATFNNHVLTWVRQAPGQGLEWMGGIIPVSGKTTYAQKFQGRVAISTDESASTAYMELSSLRSEDSAIYYCARAVSDYFNRDLGWEDYYFPIWGQGTLVTVSS

SEQ ID NO: 15 - protein, 8 aa, Artificial Sequence
  FNI2 CDRH1 (aa)
GATFNNHV

SEQ ID NO: 16 - protein, 8 aa, Artificial Sequence
  FNI2 CDRH2 (aa)
IIPVSGKT

SEQ ID NO: 17 - protein, 21 aa, Artificial Sequence
  FNI2 CDRH3 (aa)
ARAVSDYFNRDLGWEDYYFPI

SEQ ID NO: 18 - DNA, 384 bp, Artificial Sequence
  FNI2 VH (co-nt)
caggtgcagctggtgcagtctggagctgaggtgaagaggccaggatccagcgtgcgggtgagctgcaaggcttctggagctaccttcaacaatcacgtgctgacatgggtgaggcaggctccaggacagggactggagtggatgggcggcatcatccccgtgtccggcaagaccacatacgcccagaagtttcagggcagggtggctatcagcaccgatgagtccgccagcacagcttatatggagctgtcttccctgagatctgaggactccgccatctactattgtgccagagccgtgtccgactacttcaaccgcgatctgggctgggaggactactattttcccatctggggccagggcaccctggtgacagtgagctct

SEQ ID NO: 19 - DNA, 325 bp, Artificial Sequence
  FNI2 Vk (wt-nt)
gacgtagtgatgacgcagtctccagccaccctgtctgtgtctccaggggaaagagccaccctctcctgcagggccagtcagagtgttagtagcaacttggcctggtaccagcagaaacctggccaggctcccaggctcctcatctatggtgcatccaccagggccactggtgtcccagccaggttcagtggcagtgggtctgggacacagttcactctcaccatcagcagcctgcagtctgaagattttgcagtttattactgtcagcactataataactggcctccgtggacgttcggccaagggaccaagttggaaatcaaac

SEQ ID NO: 20 - protein, 108 aa, Artificial Sequence
  FNI2 VK (aa)
DVVMTQSPATLSVSPGERATLSCRASQSVSSNLAWYQQKPGQAPRLLIYGASTRATGVPARFSGSGSGTQFTLTISSLQSEDFAVYYCQHYNNWPPWTFGQGTKLEIK

SEQ ID NO: 21 - protein, 6 aa, Artificial Sequence
  FNI2 CDRL1(aa)
QSVSSN

SEQ ID NO: 22 - protein, 3 aa, Artificial Sequence
  FNI2 CDRL2(aa)
GAS

SEQ ID NO: 23 - protein, 10 aa, Artificial Sequence
  FNI2 CDRL3(aa)
QHYNNWPPWT

SEQ ID NO: 24 - DNA, 324 bp, Artificial Sequence
  FNI2 Vk (co-nt)
gacgtggtcatgacccagtctcctgccacactgagcgtgtctccaggagagagggccaccctgtcctgcagggcttcccagagcgtgtccagcaacctggcctggtaccagcagaagccaggccaggctcccaggctgctgatctatggagctagcaccagagctacaggcgtgccagctcgcttctctggatccggaagcggcacacagtttaccctgacaatctcttccctgcagtctgaggatttcgccgtgtactattgtcagcactacaacaattggcccccttggacctttggccagggcacaaagctggagatcaag

SEQ ID NO: 25 - DNA, 385 bp, Artificial Sequence
  FNI3 VH (wt-nt)
caggttcagctggtgcagtcgggggctgaggtgaagaggcctgggtcctcggtgaaggtctcctgcaaggcttctggagccaccttcagcaacaatgttatagcctgggtgcgacaggcccctggacaagggcttgagtggatgggggggatccaccctatctctgctacagcaacctacgcacagaagttccagggcagagtcgcgattgccgcggacgaattaacgagcacagcctacatggagttgaatggcctgagatctgaggactcggccgtgtattactgtgcgagagcggggtccgattactttaatagagacctcggctgggaaaattactactttgactcctggggccagggaaccctggtcaccgtctcgtcag

SEQ ID NO: 26 - protein, 128 aa, Artificial Sequence
  FNI3 VH (aa)
QVQLVQSGAEVKRPGSSVKVSCKASGATFSNNVIAWVRQAPGQGLEWMGGIHPISATATYAQKFQGRVAIAADELTSTAYMELNGLRSEDSAVYYCARAGSDYFNRDLGWENYYFDSWGQGTLVTVSS

SEQ ID NO: 27 - protein, 8 aa, Artificial Sequence
  FNI3 CDRH1 (aa)
GATFSNNV

SEQ ID NO: 28 - protein, 8 aa, Artificial Sequence
  FNI3 CDRH2 (aa)
IHPISATA

SEQ ID NO: 29 - protein, 21 aa, Artificial Sequence
  FNI3 CDRH3 (aa)
ARAGSDYFNRDLGWENYYFDS

SEQ ID NO: 30 - DNA, 384 bp, Artificial Sequence
  FNI3 VH (co-nt)
caggtgcagctggtgcagtccggagctgaggtgaagaggccaggatccagcgtgaaggtgtcctgcaaggccagcggcgctaccttcagcaacaatgtgatcgcttgggtgagacaggctccaggacagggactggagtggatgggaggaatccaccctatcagcgccaccgctacatacgcccagaagtttcagggcagagtggctatcgccgctgacgagctgacctctacagcctatatggagctgaacggcctgcgcagcgaggattccgccgtgtactattgtgccagggctggctctgactacttcaaccgggatctgggctgggagaattactattttgactcctggggccagggcaccctggtgacagtgtcttcc

SEQ ID NO: 31 - DNA, 325 bp, Artificial Sequence
  FNI3 Vk (wt-nt)
gaaatattgatgacgcagtctccagccaccctgtctgtgtctccaggggaaagagccaccctctcctgcagggccagtcaggatgttagcggcaacttagcctggtaccagcagagacctggccaggctcccaggctccttatctatggtgcatccacgagggccactggtgtcccagccaggttcactggcgctgggtctgggacagagttcactctcaccatcagcagcctgcagtctgaggattttgcactttattactgtcagcactataataactggcctccgtggaccttcggccaagggaccaaggtggaaatcaaac

SEQ ID NO: 32 - protein, 108 aa, Artificial Sequence
  FNI3 Vk (aa)
EILMTQSPATLSVSPGERATLSCRASQDVSGNLAWYQQRPGQAPRLLIYGASTRATGVPARFTGAGSGTEFTLTISSLQSEDFALYYCQHYNNWPPWTFGQGTKVEIK

SEQ ID NO: 33 - protein, 6 aa, Artificial Sequence
  FNI3 CDRL1(aa)
QDVSGN

SEQ ID NO: 34 - protein, 3 aa, Artificial Sequence
  FNI3 CDRL2(aa)
GAS

SEQ ID NO: 35 - protein, 10 aa, Artificial Sequence
  FNI3 CDRL3(aa)
QHYNNWPPWT

SEQ ID NO: 36 - DNA, 324 bp, Artificial Sequence
  FNI3 Vk (co-nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattggcccccttggacctttggccagggcacaaaggtggagatcaag

SEQ ID NO: 37 - DNA, 385 bp, Artificial Sequence
  FNI4 VH (wt-nt)
caggagcagctggtacagtctggggctgaggtgaagaagccggggtcctcggtgagggtctcctgcaaggcctctggagacaccttcagcagatatactatcagctgggttcgacaggcccccggacaaggacttgagtggatgggagggatcatcgctctctctcgaagagcgacatacgcacagaagttccagggcagagttaccattaccgcggacgaatccgcgaccacagcctacatacaactgagcggcctgacatctgacgacacggccgtatattactgtgcgagagcacactccgattactttaatagagacctcggctgggaagattactactttgactactggggccagggaaccctggtcaccgtctcctcag

SEQ ID NO: 38 - protein, 128 aa, Artificial Sequence
  FNI4 VH (aa)
QEQLVQSGAEVKKPGSSVRVSCKASGDTFSRYTISWVRQAPGQGLEWMGGIIALSRRATYAQKFQGRVTITADESATTAYIQLSGLTSDDTAVYYCARAHSDYFNRDLGWEDYYFDYWGQGTLVTVSS

SEQ ID NO: 39 - protein, 8 aa, Artificial Sequence
  FNI4 CDRH1 (aa)
GDTFSRYT

SEQ ID NO: 40 - protein, 8 aa, Artificial Sequence
  FNI4 CDRH2 (aa)
IIALSRRA

SEQ ID NO: 41 - protein, 21 aa, Artificial Sequence
  FNI4 CDRH3 (aa)
ARAHSDYFNRDLGWEDYYFDY

SEQ ID NO: 42 - DNA, 384 bp, Artificial Sequence
  FNI4 VH (co-nt)
caggagcagctggtgcagtccggagctgaggtgaagaagccaggatccagcgtgagagtgagctgcaaggcttctggcgacaccttctctagatacacaatctcctgggtgcgccaggctcctggacagggactggagtggatgggaggaatcatcgctctgagcaggcgggccacctacgctcagaagtttcagggccgcgtgaccatcacagccgatgagtctgccaccacagcttatatccagctgtccggcctgaccagcgacgatacagccgtgtactattgtgccagggctcacagcgactacttcaaccgggatctgggctgggaggactactattttgattattggggccagggcaccctggtgacagtgtcttcc

SEQ ID NO: 43 - DNA, 325 bp, Artificial Sequence
  FNI4 Vk (wt-nt)
gaagtagtgctgacgcagtctccagccaccctgtctgtgtctctaggggaaagagccatcctctcctgcagggccagtcagagtgttagcaccaacttagcctggtaccagcagagacctggccaggctcccaggctcctcatctctggtgcatccaccagggccacgggtatcccagccaggttcagtggcagtgggtctgggacagagttcacgctcaccatcagcagcctgcagtctgaagattttgcagtttattactgtcagcagtataataactggcctccgtggacgttcggccaagggaccaaggtggaaatcagac

SEQ ID NO: 44 - protein, 108 aa, Artificial Sequence
  FNI4 VK (aa)
EVVLTQSPATLSVSLGERAILSCRASQSVSTNLAWYQQRPGQAPRLLISGASTRATGIPARFSGSGSGTEFTLTISSLQSEDFAVYYCQQYNNWPPWTFGQGTKVEIR

SEQ ID NO: 45 - protein, 6 aa, Artificial Sequence
  FNI4 CDRL1 (aa)
QSVSTN

SEQ ID NO: 46 - protein, 3 aa, Artificial Sequence
  FNI4 CDRL2 (aa)
GAS

SEQ ID NO: 47 - protein, 10 aa, Artificial Sequence [sequence omitted]

SEQ ID NO: 48 - DNA, 324 bp, Artificial Sequence
  FNI4 Vk (co-nt)
gaggtggtgctgacccagtcccctgccacactgtccgtgtccctgggagagagggctatcctgagctgcagggctagccagtccgtgtccaccaacctggcctggtaccagcagagaccaggacaggctccaaggctgctgatcagcggagcttctaccagggctacaggcatcccagccagattcagcggctctggctccggcacagagtttaccctgacaatctccagcctgcagtctgaggacttcgccgtgtactattgtcagcagtataacaattggcccccttggacctttggccagggcacaaaggtggagatcagg

SEQ ID NO: 49 - DNA, 385 bp, Artificial Sequence
  FNI5 VH (wt-nt)
caggtgcagctgatacaatctgaggctgaggtgaagaagcctgggtcctcggtgagggtctcctgcaaggcttctggagacaccttcagcaaatatactatcggctgggtgcgacaggcccccggacaagggcttgagtggatgggagggatcatccctctctctcgaacagcgacctacgcacagaagttccagggcagagtcacgattaccgcggacgaatccacgaccacagtttacatgcaactgagcggcctgagatctgacgacacggccgcatattactgtgcgagagcacgctcggattactttaatagagacctcggctgggacgattactactttgattactggggccagggaaccctggtcaccgtctcctcag

SEQ ID NO: 50 - protein, 128 aa, Artificial Sequence
  FNI5 VH (aa)
QVQLIQSEAEVKKPGSSVRVSCKASGDTFSKYTIGWVRQAPGQGLEWMGGIIPLSRTATYAQKFQGRVTITADESTTTVYMQLSGLRSDDTAAYYCARARSDYFNRDLGWDDYYFDYWGQGTLVTVSS

SEQ ID NO: 51 - protein, 8 aa, Artificial Sequence
  FNI5 CDRH1 (aa)
GDTFSKYT

SEQ ID NO: 52 - protein, 8 aa, Artificial Sequence
  FNI5 CDRH2 (aa)
IIPLSRTA

SEQ ID NO: 53 - protein, 21 aa, Artificial Sequence
  FNI5 CDRH3 (aa)
ARARSDYFNRDLGWDDYYFDY

SEQ ID NO: 54 - DNA, 384 bp, Artificial Sequence
  FNI5 VH (co-nt)
caggtgcagctgatccagagcgaggccgaggtgaagaagccaggctccagcgtgagggtgagctgcaaggcttctggcgacacattctctaagtacaccatcggatgggtgcggcaggctccaggacagggcctggagtggatgggcggcatcatccctctgtctagaacagccacctacgctcagaagtttcagggccgcgtgacaatcaccgctgacgagtccaccacaaccgtgtatatgcagctgtccggcctgagaagcgacgatacagccgcttactattgtgccagggctcggtccgactacttcaaccgcgatctgggctgggacgattactattttgattattggggccagggcacactggtgaccgtgtcttcc

SEQ ID NO: 55 - DNA, 325 bp, Artificial Sequence
  FNI5 Vk (wt-nt)
gaaatagtgatgacgcagtctccagccaacctgtctgtgtctccaggggaaagagccaccctctcctgcagggccagtcagactgttagcaccaacttagcctggtaccagcagaagcctggccaggctcccaggctcctcatctctggtgcatccaccagggccactggtatcccagccaggttcagtggcagtgggtctgggacagagttcacgctcaccatcagcagcctgcagtctgaagattttgcagtttattactgtcagcagtataataattggcctccgtggacgttcggccaagggaccaaggtggaaatcagac

SEQ ID NO: 56 - protein, 108 aa, Artificial Sequence
  FNI5 VK (aa)
EIVMTQSPANLSVSPGERATLSCRASQTVSTNLAWYQQKPGQAPRLLISGASTRATGIPARFSGSGSGTEFTLTISSLQSEDFAVYYCQQYNNWPPWTFGQGTKVEIR

SEQ ID NO: 57 - protein, 6 aa, Artificial Sequence
  FNI5 CDRL1(aa)
QTVSTN

SEQ ID NO: 58 - protein, 3 aa, Artificial Sequence
  FNI5 CDRL2(aa)
GAS

SEQ ID NO: 59 - protein, 10 aa, Artificial Sequence [sequence omitted]

SEQ ID NO: 60 - DNA, 324 bp, Artificial Sequence
  FNI5 Vk (co-nt)
gagatcgtgatgacccagtcccctgctaacctgtccgtgtccccaggagagagggccacactgtcctgccgggctagccagaccgtgtctacaaatctggcctggtaccagcagaagccaggacaggctccaaggctgctgatcagcggagcttctaccagagctacaggcatcccagctcgcttcagcggatctggatccggcaccgagtttaccctgacaatctccagcctgcagagcgaggacttcgccgtgtactattgtcagcagtataacaattggcccccttggacctttggccagggcacaaaggtggagatcaga

SEQ ID NO: 61 - DNA, 385 bp, Artificial Sequence
  FNI6 VH (wt-nt)
caggtgcagctggtgcagtctggggctgaggtgaagaagcctgggtcctcggtgaaggtctcctgcaaggcctctggaggcaccttcagtagtcaagttatcagctgggtgcgagaggccccaggacaagggcttgagtggatgggagggatcattcctatcactggaatagcgaacaacgcacagaagttccagggcagagtcacgattaccgcggacggatccacgggcacagtctacatggagttgagcagcctgagatctggggacacggccgtctattactgtgcgagagcgggttcggattattttaatagagacctcggctgggaaaattactactttgaatattggggccagggaaccctggtcaccgtctcctcag

SEQ ID NO: 62 - protein, 128 aa, Artificial Sequence
  FNI6 VH (aa)
QVQLVQSGAEVKKPGSSVKVSCKASGGTFSSQVISWVREAPGQGLEWMGGIIPITGIANNAQKFQGRVTITADGSTGTVYMELSSLRSGDTAVYYCARAGSDYFNRDLGWENYYFEYWGQGTLVTVSS

SEQ ID NO: 63 - protein, 8 aa, Artificial Sequence
  FNI6 CDRH1 (aa)
GGTFSSQV

SEQ ID NO: 64 - protein, 8 aa, Artificial Sequence
  FNI6 CDRH2 (aa)
IIPITGIA

SEQ ID NO: 65 - protein, 21 aa, Artificial Sequence
  FNI6 CDRH3 (aa)
ARAGSDYFNRDLGWENYYFEY

SEQ ID NO: 66 - DNA, 384 bp, Artificial Sequence
  FNI6 VH (co-nt)
caggtgcagctggtgcagagcggagctgaggtgaagaagccaggctccagcgtgaaggtgtcttgcaaggcttccggcggcaccttctcttcccaggtcatctcttgggtgagggaggctccaggacagggactggagtggatgggcggcatcatccctatcacaggcatcgccaacaatgctcagaagtttcagggcagagtgaccatcacagccgacggcagcaccggcacagtgtacatggagctgagctctctgcgctctggcgataccgccgtgtactattgtgccagggctggctccgactacttcaaccgggatctgggctgggagaattactattttgagtattggggccagggcaccctggtgacagtgtccagc

SEQ ID NO: 67 - DNA, 325 bp, Artificial Sequence
  FNI6 Vk (wt-nt)
gaaatcgtgatgacacagtctccagccaccctgtctgtatctccaggggaaagagccatcctctcctgcagggccagtcagagtgttagcacccacttagcctggtaccagcagaaacctggccaggctcccagactcctcgtttttgatgcatccaccagggccactggtgtcccagccagattcggtggcagtgggtctgggacagagttcactctcaccatcagcagcctgcagtctgaagattctgctgtttattactgtcaacactataataactggcctccgtggacgttcggccaagggaccaacgtggaaatcagac

SEQ ID NO: 68 - protein, 108 aa, Artificial Sequence
  FNI6 VK (aa)
EIVMTQSPATLSVSPGERAILSCRASQSVSTHLAWYQQKPGQAPRLLVFDASTRATGVPARFGGSGSGTEFTLTISSLQSEDSAVYYCQHYNNWPPWTFGQGTNVEIR

SEQ ID NO: 69 - protein, 6 aa, Artificial Sequence
  FNI6 CDRL1(aa)
QSVSTH

SEQ ID NO: 70 - protein, 3 aa, Artificial Sequence
  FNI6 CDRL2(aa)
DAS

SEQ ID NO: 71 - protein, 10 aa, Artificial Sequence
  FNI6 CDRL3(aa)
QHYNNWPPWT

SEQ ID NO: 72 - DNA, 324 bp, Artificial Sequence
  FNI6 Vk (co-nt)
gagatcgtgatgacccagtctcctgccacactgtccgtgtccccaggagagagggctatcctgtcctgcagggctagccagtccgtgtccacccacctggcctggtaccagcagaagccaggccaggctcccaggctgctggtgttcgacgctagcaccagagctacaggcgtgccagctaggttcggaggaagcggatctggcacagagtttaccctgacaatctccagcctgcagtccgaggattccgccgtgtactattgtcagcattataacaattggcccccttggacctttggccagggcacaaacgtggagatcaga

SEQ ID NO: 73 - DNA, 385 bp, Artificial Sequence
  FNI7 VH (wt-nt)
caagtgcagctggtgcagtctggggctgaggtgaagaagcctgggtcctcggtgaaagtctcctgtaagacttctggaggcaccttcaataggcaagttatcagctgggtgcgacaggccccaggacaaggacttgagtggatgggagggatcctccctcttactggtagaggggacgaggcagagaggtttcagggcagagtcaccattaccgcggacgaatctgagagtacagtctacatggacttgagcagcctgagatctggggacacggccgtctattactgtgcgagagcgcgttcggattactttaatagagacctcggctgggaaaattactactttgaatcttggggccagggaaccctggtcaccgtctcctcag

SEQ ID NO: 74 - protein, 128 aa, Artificial Sequence
  FNI7 VH (aa)
QVQLVQSGAEVKKPGSSVKVSCKTSGGTFNRQVISWVRQAPGQGLEWMGGILPLTGRGDEAERFQGRVTITADESESTVYMDLSSLRSGDTAVYYCARARSDYFNRDLGWENYYFESWGQGTLVTVSS

SEQ ID NO: 75 - protein, 8 aa, Artificial Sequence
  FNI7 CDRH1 (aa)
GGTFNRQV

SEQ ID NO: 76 - protein, 8 aa, Artificial Sequence
  FNI7 CDRH2 (aa)
ILPLTGRG

SEQ ID NO: 77 - protein, 21 aa, Artificial Sequence
  FNI7 CDRH3 (aa)
ARARSDYFNRDLGWENYYFES

SEQ ID NO: 78 - DNA, 384 bp, Artificial Sequence
  FNI7 VH (co-nt)
caggtgcagctggtgcagtccggagctgaggtgaagaagccaggctccagcgtgaaggtgtcttgcaagacctccggcggcacattcaacaggcaggtcatcagctgggtgcggcaggctccaggacagggactggagtggatgggaggaatcctgcctctgaccggcaggggcgacgaggccgagagatttcagggccgcgtgaccatcacagctgatgagtccgagagcaccgtgtacatggacctgtcttccctgagaagcggcgatacagccgtgtactattgtgccagggctcggtctgactatttcaaccgcgatctgggctgggagaattactattttgagtcttggggccagggcaccctggtgacagtgagctct

SEQ ID NO: 79 - DNA, 325 bp, Artificial Sequence
  FNI7 Vk (wt-nt)
gaaatcgtgatgacgcagtctccagccaccctgtctgtatctccaggggaaagagccaccctctcctgcagggccagtcagagtgttagtaccgacttagtctggtaccagcagaaacctggccaggctccccggctcctcatttatgatgcatccactagggccactggtatcccagccaggttcggtggcagggggtctgggacagagttcactctcaccatcagcagcctgcagtctgaagattctgctgtttattactgtcagcactattcttactggcctccgtggacattcggccaagggaccaaagtggaaatcaatc

SEQ ID NO: 80 - protein, 108 aa, Artificial Sequence
  FNI7 VK (aa)
EIVMTQSPATLSVSPGERATLSCRASQSVSTDLVWYQQKPGQAPRLLIYDASTRATGIPARFGGRGSGTEFTLTISSLQSEDSAVYYCQHYSYWPPWTFGQGTKVEIN

SEQ ID NO: 81 - protein, 6 aa, Artificial Sequence
  FNI7 CDRL1(aa)
QSVSTD

SEQ ID NO: 82 - protein, 3 aa, Artificial Sequence
  FNI7 CDRL2(aa)
DAS

SEQ ID NO: 83 - protein, 10 aa, Artificial Sequence
  FNI7 CDRL3(aa)
QHYSYWPPWT

SEQ ID NO: 84 - DNA, 324 bp, Artificial Sequence
  FNI7 Vk (co-nt)
gagatcgtgatgacccagtcccctgccacactgtccgtgtccccaggagagagagccaccctgagctgcagggctagccagtccgtgtccacagacctggtgtggtaccagcagaagccaggacaggctccaaggctgctgatctatgatgcctctaccagagctacaggcatcccagctaggttcggaggaaggggatccggcaccgagtttaccctgacaatctccagcctgcagagcgaggactccgccgtgtactattgtcagcactacagctattggcccccttggaccttcggccagggcacaaaggtggagatcaac

SEQ ID NO: 85 - DNA, 385 bp, Artificial Sequence
  FNI9 VH (wt-nt)
caggtccacctggtgcagtctggggctgaggtgaaggagcctgggtcctcggtgacggtctcctgcaaggcatctggaggcagcttcaacaaccaggctattagctgggtgcgacaggccccaggacaaggccttgagtggatgggagggatcttccctatctctggcacaccgaccagcgcacagaggttccagggcagagtcacatttaccgcggacgagtccacgaccacagtctacatggatctgagcagcctgagatctgacgacacggccgtctactactgtgcgagagcgggttcggattactttaatagagacctcggctgggaaaactactactttgcgtcctggggccagggaaccctggtcaccgtctcctcag

SEQ ID NO: 86 - protein, 128 aa, Artificial Sequence [sequence omitted]

SEQ ID NO: 87 - protein, 8 aa, Artificial Sequence
  FNI9 CDRH1 (aa)
GGSFNNQA

SEQ ID NO: 88 - protein, 8 aa, Artificial Sequence
  FNI9 CDRH2 (aa)
IFPISGTP

SEQ ID NO: 89 - protein, 21 aa, Artificial Sequence
  FNI9 CDRH3 (aa)
ARAGSDYFNRDLGWENYYFAS

SEQ ID NO: 90 - DNA, 384 bp, Artificial Sequence
  FNI9 VH (co-nt)
caggtgcacctggtgcagagcggagctgaggtgaaggagccaggatccagcgtgacagtgtcttgcaaggcttccggcggcagcttcaacaatcaggctatctcctgggtgaggcaggctccaggacagggactggagtggatgggcggcatctttcccatctctggcacacctacctccgcccagaggttccagggaagggtgaccttcaccgctgacgagagcaccacaaccgtgtacatggatctgtcttccctgagatctgacgataccgccgtgtactattgtgccagagctggctccgactatttcaaccgcgatctgggctgggagaattactattttgcttcctggggccagggcacactggtgaccgtgagctct

SEQ ID NO: 91 - DNA, 325 bp, Artificial Sequence
  FNI9 Vk (wt-nt)
gaaatcgtgatgacgcagtctccagccaccctgtctctatcttcaggggaaagagccaccctctcctgcagggccagtcggagtgttagtagcaacttagcctggtaccagcagaaacctggccaggctcccaggctcctcatttatgatgcatccaccagggccactggtttttcagccaggttcgctggcagtgggtctgggacagagttcactctcaccatcagcagcctgcagtctgaagattctgctatttattactgtcagcagtataataactggcctccgtggacgttcggccaagggaccaaggtggaaatcaaac

SEQ ID NO: 92 - protein, 108 aa, Artificial Sequence
  FNI9 VK (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNWPPWTFGQGTKVEIK

SEQ ID NO: 93 - protein, 6 aa, Artificial Sequence
  FNI9 CDRL1(aa)
RSVSSN

SEQ ID NO: 94 - protein, 3 aa, Artificial Sequence
  FNI9 CDRL2(aa)
DAS

SEQ ID NO: 95 - protein, 10 aa, Artificial Sequence [sequence omitted]

SEQ ID NO: 96 - DNA, 324 bp, Artificial Sequence
  FNI9 Vk (co-nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattggcccccttggacctttggccagggcacaaaggtggagatcaag

SEQ ID NO: 97 - DNA, 385 bp, Artificial Sequence
  FNI10 VH (wt-nt)
caggtgcagctggtgcagtctggggctgaggtgaagaagcctgggtcctcggtgaaagtctcctgcaaggcttctggaggcaccttgagtagtcaagttattagctgggtgcgacaggccccaggacaaggactggagtggatcggagggatcatccccaccactggtacagggggcgcggcagaggggttccagggcagagtctccatttccgcggacgaatccaggagcacagtctacatggaactgaccagcctgacttctggggacacggccgtctattattgtgcgagagcggtttcggattactttaatagagacctcggctgggaaaattactactttgaatcttggggccagggaaccctggtcaccgtctcctcag

SEQ ID NO: 98 - protein, 128 aa, Artificial Sequence
  FNI10 VH (aa)
QVQLVQSGAEVKKPGSSVKVSCKASGGTLSSQVISWVRQAPGQGLEWIGGIIPTTGTGGAAEGFQGRVSISADESRSTVYMELTSLTSGDTAVYYCARAVSDYFNRDLGWENYYFESWGQGTLVTVSS

SEQ ID NO: 99 - protein, 8 aa, Artificial Sequence
  FNI10 CDRH1 (aa)
GGTLSSQV

SEQ ID NO: 100 - protein, 8 aa, Artificial Sequence
  FNI10 CDRH2 (aa)
IIPTTGTG

SEQ ID NO: 101 - protein, 21 aa, Artificial Sequence
  FNI10 CDRH3 (aa)
ARAVSDYFNRDLGWENYYFES

SEQ ID NO: 102 - DNA, 384 bp, Artificial Sequence
  FNI10 VH (co-nt)
caggtgcagctggtgcagagcggagctgaggtgaagaagccaggctccagcgtgaaggtgtcctgcaaggctagcggcggcaccctgtcttcccaggtcatctcttgggtgaggcaggctccaggacagggactggagtggatcggcggcatcatccctaccacaggcacaggcggagctgctgagggattccagggcagagtgtccatcagcgccgacgagtctcgctccaccgtgtacatggagctgaccagcctgacatctggcgatacagccgtgtactattgtgccagggccgtgtccgactatttcaaccgggatctgggctgggagaattactattttgagtcctggggccagggcaccctggtgacagtgagctct

SEQ ID NO: 103 - DNA, 325 bp, Artificial Sequence
  FNI10 Vk (wt-nt)
gaaatcgtgatgacgcagtctccagccaccctgtctgtgtctccaggggaaagagccaccctctcttgcagggccagtcggagtgttagtatcaacttagcctggtaccaacagaaacctggccaggctccccggctcctcatttatgatgcatctacgagggccactggcatcccagccaggttcggtggcagggggtctggaacagagttcactctcaccatcagcagcctgcagtctgaagattctgctgtttattactgtcagcactataataactggcctccgtggacattcggccaagggaccagagtggaaatcaaac

SEQ ID NO: 104 - protein, 108 aa, Artificial Sequence
  FNI10 VK (aa)
EIVMTQSPATLSVSPGERATLSCRASRSVSINLAWYQQKPGQAPRLLIYDASTRATGIPARFGGRGSGTEFTLTISSLQSEDSAVYYCQHYNNWPPWTFGQGTRVEIK

SEQ ID NO: 105 - protein, 6 aa, Artificial Sequence
  FNI10 CDRL1(aa)
RSVSIN

SEQ ID NO: 106 - protein, 3 aa, Artificial Sequence
  FNI10 CDRL2(aa)
DAS

SEQ ID NO: 107 - protein, 10 aa, Artificial Sequence
  FNI10 CDRL3(aa)
QHYNNWPPWT

SEQ ID NO: 108 - DNA, 324 bp, Artificial Sequence
  FNI10 Vk (co-nt)
gagatcgtgatgacccagtcccctgccacactgtccgtgtccccaggagagagagccaccctgagctgcagggctagcaggtccgtgtccatcaacctggcctggtaccagcagaagccaggccaggctcccaggctgctgatctatgacgcttctaccagggctacaggcatcccagctagattcggaggaaggggatccggaacagagtttaccctgacaatctccagcctgcagagcgaggattccgccgtgtactattgtcagcactacaacaattggccaccttggaccttcggccagggaacacgcgtggagatcaag

SEQ ID NO: 109 - DNA, 385 bp, Artificial Sequence
  FNI12 VH (wt-nt)
caggtgcacctggtacagtctggggctgaggtgaagaagcctgggtcctcggtgagggtctcctgcaaggcttctggagactccttcaacaaatatgaagtcagctgggtgcgacaggcccccggacatggacttgagtggatgggagggatcatccctctctctcctatagcgaggtacgcagagaaatttcagggcagagtcacgattaccgcggacgaattcacgagcacggtctatatacaactgaccagcctgagatctgacgacacggccgtatactactgtgcgacaacacgttcggattactttaatagagacctcggctgggaagattacttctttgaccactggggccagggaaccctggtcaccgtctcctcag

SEQ ID NO: 110 - protein, 128 aa, Artificial Sequence
  FNI12 VH (aa)
QVHLVQSGAEVKKPGSSVRVSCKASGDSFNKYEVSWVRQAPGHGLEWMGGIIPLSPIARYAEKFQGRVTITADEFTSTVYIQLTSLRSDDTAVYYCATTRSDYFNRDLGWEDYFFDHWGQGTLVTVSS

SEQ ID NO: 111 - protein, 8 aa, Artificial Sequence
  FNI12 CDRH1 (aa)
GDSFNKYE

SEQ ID NO: 112 - protein, 8 aa, Artificial Sequence
  FNI12 CDRH2 (aa)
IIPLSPIA

SEQ ID NO: 113 - protein, 21 aa, Artificial Sequence
  FNI12 CDRH3 (aa)
ATTRSDYFNRDLGWEDYFFDH

SEQ ID NO: 114 - DNA, 384 bp, Artificial Sequence
  FNI12 VH (co-nt)
caggtgcacctggtgcagtctggcgccgaggtgaagaagccaggctccagcgtgagggtgtcctgcaaggctagcggcgactctttcaacaagtacgaggtgagctgggtgagacaggctccaggacatggactggagtggatgggcggcatcatccccctgtctcctatcgccagatacgctgagaagttccagggccgcgtgaccatcacagctgatgagtttacctccacagtgtatatccagctgacctccctgaggagcgacgatacagccgtgtactattgtgctaccacaaggagcgactactttaatcgggatctgggctgggaggactatttctttgatcactggggccagggcaccctggtgacagtgtcttcc

SEQ ID NO: 115 - DNA, 325 bp, Artificial Sequence
  FNI12 Vk (wt-nt)
gaaatagtgatgacgcagtctccagccaccctgtctgtgtctccaggggaaagagccaccctctcctgcagggccagtcagagtattagcaccaacttagcctggtaccagcagaaacctggccaggctcccaggctcctcatctctggtgcatccaccagggccactggtatcccagccaggttcagtggcagtgggtctgggacagagttcactctcaccatcagcagcctgcagtctgaagattttggagtttattactgtcagcactataataactggcctccgtggacgttcggccaagggaccaaggtggaaatcaaac

SEQ ID NO: 116 - protein, 108 aa, Artificial Sequence
  FNI12 VK (aa)
EIVMTQSPATLSVSPGERATLSCRASQSISTNLAWYQQKPGQAPRLLISGASTRATGIPARFSGSGSGTEFTLTISSLQSEDFGVYYCQHYNNWPPWTFGQGTKVEIK

SEQ ID NO: 117 - protein, 6 aa, Artificial Sequence
  FNI12 CDRL1(aa)
QSISTN

SEQ ID NO: 118 - protein, 3 aa, Artificial Sequence
  FNI12 CDRL2(aa)
GAS

SEQ ID NO: 119 - protein, 10 aa, Artificial Sequence
  FNI12 CDRL3(aa)
QHYNNWPPWT

SEQ ID NO: 120 - DNA, 324 bp, Artificial Sequence
  FNI12 Vk (co-nt)
gagatcgtgatgacccagtcccctgccacactgtccgtgtccccaggagagagggccaccctgagctgccgggctagccagtctatctccacaaacctggcctggtaccagcagaagccaggacaggctccaaggctgctgatcagcggagcttctaccagagctacaggcatcccagctcgcttcagcggatctggatccggaaccgagtttaccctgacaatctccagcctgcagtctgaggacttcggcgtgtactattgtcagcactataacaattggcccccttggacctttggccagggcacaaaggtggagatcaag

SEQ ID NO: 121 - DNA, 385 bp, Artificial Sequence
  FNI13 VH (wt-nt)
caggttcagctggtgcaatctggggctgaggtgaagaggcctgggtcctcggtgagggtctcctgcaagggttctggagacaccttcaacaactatgttatcagttgggtgcgacaggcccctggccaagggcttgagtggatgggggggatcatccctatctttcaaacaccaaactacgcagagaagttccagggcagagtcgcgattaccgcggacgaatccacgagcacggcctacatggagttgagcagcctgagatctgaggactcggccatttattactgtgcgagagcgaattccgattactttaatagagacctcggctgggaaaattactactttgaagactggggccagggaaccctggtcaccgtctcctcag

SEQ ID NO: 122 - protein, 128 aa, Artificial Sequence
  FNI13 VH (aa)
QVQLVQSGAEVKRPGSSVRVSCKGSGDTFNNYVISWVRQAPGQGLEWMGGIIPIFQTPNYAEKFQGRVAITADESTSTAYMELSSLRSEDSAIYYCARANSDYFNRDLGWENYYFEDWGQGTLVTVSS

SEQ ID NO: 123 - protein, 8 aa, Artificial Sequence
  FNI13 CDRH1 (aa)
GDTFNNYV

SEQ ID NO: 124 - protein, 8 aa, Artificial Sequence
  FNI13 CDRH2 (aa)
IIPIFQTP

SEQ ID NO: 125 - protein, 21 aa, Artificial Sequence
  FNI13 CDRH3 (aa)
ARANSDYFNRDLGWENYYFED

SEQ ID NO: 126 - DNA, 384 bp, Artificial Sequence
  FNI13 VH (co-nt)
caggtgcagctggtgcagtccggagctgaggtgaagaggccaggatccagcgtgcgggtgagctgcaagggatctggcgacaccttcaacaattacgtgatcagctgggtgaggcaggctccaggacagggactggagtggatgggcggcatcatccccatcttccagacccctaactacgctgagaagtttcagggcagggtggccatcacagctgacgagtccaccagcacagcctatatggagctgtcttccctgagatctgaggattccgctatctactattgtgccagagctaactctgactatttcaatcgcgatctgggctgggagaattactattttgaggattggggccagggcaccctggtgacagtgagctct

SEQ ID NO: 127 - DNA, 325 bp, Artificial Sequence
  FNI13 Vk (wt-nt)
gaaagagtgatgacgcagtctccagccaccctttctgtgtctccagggggaagagccaccctctcctgcagggccagtcagagtgttggtagcaacttagcctggtaccagcagaaacctggccaggctcccaggctcctcatctatgatgcttctgccagggccactggtgtcccagccaggttcagtggcagtgggtctgggacagagttctctctctccatcaacagcctgcagtctgaagattctgcagtttattactgtcagcactataatatctggccgccgtggacgttcggccaagggaccaaggtggaaatcaaac

SEQ ID NO: 128 - protein, 108 aa, Artificial Sequence
  FNI13 VK (aa)
ERVMTQSPATLSVSPGGRATLSCRASQSVGSNLAWYQQKPGQAPRLLIYDASARATGVPARFSGSGSGTEFSLSINSLQSEDSAVYYCQHYNIWPPWTFGQGTKVEIK

SEQ ID NO: 129 - protein, 6 aa, Artificial Sequence
  FNI13 CDRL1(aa)
QSVGSN

SEQ ID NO: 130 - protein, 3 aa, Artificial Sequence
  FNI13 CDRL2(aa)
DAS

SEQ ID NO: 131 - protein, 10 aa, Artificial Sequence
  FNI13 CDRL3(aa)
QHYNIWPPWT

SEQ ID NO: 132 - DNA, 324 bp, Artificial Sequence
  FNI13 Vk (co-nt)
gagagagtgatgacccagtctcctgctacactgtccgtgagcccaggaggaagggctaccctgtcctgcagggcttctcagtccgtgggaagcaacctggcttggtaccagcagaagccaggccaggcccccagactgctgatctatgacgcttccgctagagctaccggcgtgccagctcgcttcagcggatctggctccggcacagagtttagcctgtctatcaactccctgcagagcgaggattctgccgtgtactattgtcagcactacaatatctggccaccttggaccttcggccagggaacaaaggtggagatcaag

SEQ ID NO: 133 - DNA, 385 bp, Artificial Sequence
  FNI14 VH (wt-nt)
caagttcagttggtgcagtctggggctgagctgaagcggcctgggtcctcggtgaggatctcctgcaaggcctctggtgtcaccttcaacaagtatgttctcagctgggtgcgactggcccctggacaagggcttgagtggatgggaggaatcatccctatttctggtataccacattacgcagagaagttccagggcagagtcgcgattaccgcggacgaatccacgagcacagtctacatggagttgagcagcctacgatctgaggactcggccctatattactgtgcgagagcggtctccgattattttaatcgggacctcggctgggatgattactactttcctttgtggggccacggcaccctggtcaccgtctcctcag

SEQ ID NO: 134 - protein, 128 aa, Artificial Sequence
  FNI14 VH (aa)
QVQLVQSGAELKRPGSSVRISCKASGVTFNKYVLSWVRLAPGQGLEWMGGIIPISGIPHYAEKFQGRVAITADESTSTVYMELSSLRSEDSALYYCARAVSDYFNRDLGWDDYYFPLWGHGTLVTVSS

SEQ ID NO: 135 - protein, 8 aa, Artificial Sequence
  FNI14 CDRH1 (aa)
GVTFNKYV

SEQ ID NO: 136 - protein, 8 aa, Artificial Sequence
  FNI14 CDRH2 (aa)
IIPISGIP

SEQ ID NO: 137 - protein, 21 aa, Artificial Sequence
  FNI14 CDRH3 (aa)
ARAVSDYFNRDLGWDDYYFPL

SEQ ID NO: 138 - DNA, 384 bp, Artificial Sequence
  FNI14 VH (co-nt)
caggtgcagctggtgcagtctggagctgagctgaagaggccaggatccagcgtgcggatcagctgcaaggcttctggcgtgaccttcaacaagtacgtgctgtcctgggtgaggctggctccaggacagggactggagtggatgggcggcatcatccccatcagcggcatccctcactacgctgagaagtttcagggcagggtggccatcacagctgacgagtccaccagcacagtgtatatggagctgtcttccctgagatctgaggattccgccctgtactattgtgccagagccgtgtccgactatttcaatcgcgatctgggctgggacgattactattttcccctgtggggccatggcaccctggtgacagtgagctct

SEQ ID NO: 139 - DNA, 325 bp, Artificial Sequence
  FNI14 Vk (wt-nt)
gaaatagtgatgacgcagtctccagccaccctgtctgtgtctccaggggaaagcgccaccctcttctgcagggccagtcggagtgttagtgacaacttagcctggtaccagcagaaacctggccaggctcccaggctcctcatctttggtgcttccaccagggccactggtgtcccagccaggttcggtggcagtgggtctgggacacagttcactctcaccatcagcagcctgcagtctgaagattttgcagtttattactgtcagcattataataactggcctccgtggacgttcggccaagggaccaaggtggagatcaaac

SEQ ID NO: 140 - protein, 108 aa, Artificial Sequence
  FNI14 VK (aa)
EIVMTQSPATLSVSPGESATLFCRASRSVSDNLAWYQQKPGQAPRLLIFGASTRATGVPARFGGSGSGTQFTLTISSLQSEDFAVYYCQHYNNWPPWTFGQGTKVEIK

SEQ ID NO: 141 - protein, 6 aa, Artificial Sequence
  FNI14 CDRL1(aa)
RSVSDN

SEQ ID NO: 142 - protein, 3 aa, Artificial Sequence
  FNI14 CDRL2(aa)
GAS

SEQ ID NO: 143 - protein, 10 aa, Artificial Sequence
  FNI14 CDRL3(aa)
QHYNNWPPWT

SEQ ID NO: 144 - DNA, 324 bp, Artificial Sequence
  FNI14 Vk (co-nt)
gagatcgtgatgacccagtcccctgccacactgtccgtgtccccaggagagagcgccaccctgttctgcagggctagcaggtccgtgtccgacaacctggcctggtaccagcagaagccaggccaggctcccaggctgctgatctttggcgcctctaccagagctacaggcgtgccagctaggttcggaggaagcggatctggcacacagtttaccctgacaatctccagcctgcagtccgaggatttcgccgtgtactattgtcagcactataacaattggcccccttggacctttggccagggcacaaaggtggagatcaag

SEQ ID NO: 145 - DNA, 385 bp, Artificial Sequence
  FNI17 VH (wt-nt)
caggttcaactggtgcagtctggggctgaggtgaagaggcctgggtcctcggtgaaggtctcctgcaagccttccggaggcaccttcagcaacaatgttatcagctgggtgcgacaggcccctggacaagggcttgagtggatgggagggatcatccccacctctggtatagcaaactacgcgcagaagttccagggcagagtcgcgattattgcggacaaatctacgagcacagtctacatggcgttgagcagcctgagatctgaggactcggccgtgtatttctgtgccagagcgcggtccgactacttcaatagagacctcggctgggaagattactactttgagaactggggccagggaaccctggtcaccgtctcctcag

SEQ ID NO: 146 - protein, 128 aa, Artificial Sequence
  FNI17 VH (aa)
QVQLVQSGAEVKRPGSSVKVSCKPSGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSS

SEQ ID NO: 147 - protein, 8 aa, Artificial Sequence
  FNI17 CDRH1 (aa)
GGTFSNNV

SEQ ID NO: 148 - protein, 8 aa, Artificial Sequence
  FNI17 CDRH2 (aa)
IIPTSGIA

SEQ ID NO: 149 - protein, 21 aa, Artificial Sequence
  FNI17 CDRH3 (aa)
ARARSDYFNRDLGWEDYYFEN

SEQ ID NO: 150 - DNA, 384 bp, Artificial Sequence
  FNI17 VH (co-nt)
caggtgcagctggtgcagtccggagctgaggtgaagaggccaggctccagcgtgaaggtgagctgcaagccttctggcggcaccttctccaacaatgtgatcagctgggtgagacaggctccaggacagggactggagtggatgggaggaatcatccccacatctggcatcgccaactacgctcagaagtttcagggcagggtggccatcatcgctgataagtccaccagcacagtgtatatggccctgtcttccctgagatctgaggactccgccgtgtacttctgtgccagggctcggtccgactacttcaaccgcgatctgggctgggaggactactatttcgagaattggggccagggcaccctggtgacagtgagctct

SEQ ID NO: 151 - DNA, 325 bp, Artificial Sequence
  FNI17 Vk (wt-nt)
gaaatagtgatgacgcagtctccagccaccctgtctgtgtctccaggggaaagagccaccctctcctgcagggccagtcagagtgttggcagcagcttagtctggtaccagcagaaacctggccaggctcccaggctcctcatctatggtgcatccaccagggccactggtgtcccagccaggttcagtggcagtgggtctgggacagagttcactctcaccatcagcagcctgcagtctgaagattttgcagtttattactgtcagcactataataactggcctccgtggacgttcggccaagggaccaaggtggaaatcaaac

SEQ ID NO: 152 - protein, 108 aa, Artificial Sequence
  FNI17 VK (aa)
EIVMTQSPATLSVSPGERATLSCRASQSVGSSLVWYQQKPGQAPRLLIYGASTRATGVPARFSGSGSGTEFTLTISSLQSEDFAVYYCQHYNNWPPWTFGQGTKVEIK

SEQ ID NO: 153 - protein, 6 aa, Artificial Sequence
  FNI17 CDRL1(aa)
QSVGSS

SEQ ID NO: 154 - protein, 3 aa, Artificial Sequence
  FNI17 CDRL2(aa)
GAS

SEQ ID NO: 155 - protein, 10 aa, Artificial Sequence
  FNI17 CDRL3(aa)
QHYNNWPPWT

SEQ ID NO: 156 - DNA, 324 bp, Artificial Sequence
  FNI17 Vk (co-nt)
gagatcgtgatgacccagtctcctgccacactgagcgtgtctccaggagagagggccaccctgtcctgcagggcttcccagagcgtgggatccagcctggtgtggtaccagcagaagccaggacaggctccaaggctgctgatctatggagctagcaccagagctacaggcgtgccagctcgcttctctggatccggaagcggcacagagtttaccctgacaatctcttccctgcagtctgaggacttcgccgtgtactattgtcagcactacaacaattggcccccttggacctttggccagggcacaaaggtggagatcaag

SEQ ID NO: 157 - DNA, 385 bp, Artificial Sequence
  FNI19 VH (wt-nt)
caagttcagctggtgcagtctggggctgaggtgaagaggcctgggtcctcggtgagggtctcctgcaaggcttctgaaggcaccttcaacaagtatactctcacctgggtgcgacaggcccctggacagggacttgagtggatgggaggaatcatccctatctccggtatagcaaactacgcacagaagttccagggcagagtcgcgattaccgcggacgaatccacgaccacagcctacatggaattgagcagcctaagatctgaagactcggccgtatattactgtgcgacagcggtctccgattattttaatcgagacctcggctgggaagattactactttccgttctggggccagggcaccctggtcaccgtcgcctcag

SEQ ID NO: 158 - protein, 128 aa, Artificial Sequence
  FNI19 VH (aa)
QVQLVQSGAEVKRPGSSVRVSCKASEGTFNKYTLTWVRQAPGQGLEWMGGIIPISGIANYAQKFQGRVAITADESTTTAYMELSSLRSEDSAVYYCATAVSDYFNRDLGWEDYYFPFWGQGTLVTVAS

SEQ ID NO: 159 - protein, 8 aa, Artificial Sequence
  FNI19 CDRH1 (aa)
EGTFNKYT

SEQ ID NO: 160 - protein, 8 aa, Artificial Sequence
  FNI19 CDRH2 (aa)
IIPISGIA

SEQ ID NO: 161 - protein, 21 aa, Artificial Sequence
  FNI19 CDRH3 (aa)
ATAVSDYFNRDLGWEDYYFPF

SEQ ID NO: 162 - DNA, 384 bp, Artificial Sequence
  FNI19 VH (co-nt)
caggtgcagctggtgcagtccggagctgaggtgaagaggccaggatccagcgtgcgggtgtcctgcaaggctagcgagggcacattcaacaagtacacactgacctgggtgaggcaggctccaggacagggactggagtggatgggcggcatcatccctatctctggcatcgccaattacgctcagaagtttcagggcagagtggccatcacagctgatgagtccaccacaaccgcctatatggagctgtcttccctgagaagcgaggactccgccgtgtactattgtgccaccgctgtgagcgactatttcaaccgcgatctgggctgggaggactactatttccccttttggggccagggcacactggtgaccgtggcttct

SEQ ID NO: 163 - DNA, 325 bp, Artificial Sequence
  FNI19 Vk (wt-nt)
gaaatagtgatgacgcagtctccagccaccctgtctgtgtctccgggggccagagccaccctcttctgcagggccagtcggagtgttagtgacaacttagcctggtaccagcagaaacctggccaggctcccaggctcctcatctttggtgcatccaccagggccactggtgtcccagccaggttcagtggaagtgggtctgggacacagttcactctcaccatcagcagcctgcagtccgaagattttgcagtttattactgtcagcattataatatttggcctccgtggacgttcggccaagggaccaaggtggagatcaaac

SEQ ID NO: 164 - protein, 108 aa, Artificial Sequence
  FNI19 VK (aa)
EIVMTQSPATLSVSPGARATLFCRASRSVSDNLAWYQQKPGQAPRLLIFGASTRATGVPARFSGSGSGTQFTLTISSLQSEDFAVYYCQHYNIWPPWTFGQGTKVEIK

SEQ ID NO: 165 - protein, 6 aa, Artificial Sequence
  FNI19 CDRL1(aa)
RSVSDN

SEQ ID NO: 166 - protein, 3 aa, Artificial Sequence
  FNI19 CDRL2(aa)
GAS

SEQ ID NO: 167 - protein, 10 aa, Artificial Sequence
  FNI19 CDRL3(aa)
QHYNIWPPWT

SEQ ID NO: 168 - DNA, 324 bp, Artificial Sequence
  FNI19 Vk (co-nt)
gagatcgtgatgacccagtcccctgctacactgtccgtgtccccaggagctagggctaccctgttctgcagggctagcaggtccgtgtccgacaacctggcttggtaccagcagaagccaggccaggcccccagactgctgatctttggagctagcaccagagctacaggcgtgccagctcgcttcagcggatctggatccggcacacagtttaccctgacaatctccagcctgcagtctgaggatttcgccgtgtactattgtcagcactataatatctggcccccttggacctttggccagggcacaaaggtggagatcaag

SEQ ID NO: 169 - skipped

SEQ ID NO: 170 - DNA, 384 bp, Artificial Sequence
  FNI3-VH-W110F (nt)
caggtgcagctggtgcagtccggagctgaggtgaagaggccaggatccagcgtgaaggtgtcctgcaaggccagcggcgctaccttcagcaacaatgtgatcgcttgggtgagacaggctccaggacagggactggagtggatgggaggaatccaccctatcagcgccaccgctacatacgcccagaagtttcagggcagagtggctatcgccgctgacgagctgacctctacagcctatatggagctgaacggcctgcgcagcgaggattccgccgtgtactattgtgccagggctggctctgactacttcaaccgggatctgggcttcgagaattactattttgactcctggggccagggcaccctggtgacagtgtcttcc

SEQ ID NO: 171 - protein, 128 aa, Artificial Sequence
  FNI3-VH-W110F (aa)
QVQLVQSGAEVKRPGSSVKVSCKASGATFSNNVIAWVRQAPGQGLEWMGGIHPISATATYAQKFQGRVAIAADELTSTAYMELNGLRSEDSAVYYCARAGSDYFNRDLGFENYYFDSWGQGTLVTVSS

SEQ ID NO: 172 - protein, 21 aa, Artificial Sequence
  FNI3-VH-W110F CDRH3 (aa)
ARAGSDYFNRDLGFENYYFDS

SEQ ID NO: 173 - DNA, 324 bp, Artificial Sequence
  FNI3-VK-W94F (nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattttcccccttggacctttggccagggcacaaaggtggagatcaag

SEQ ID NO: 174 - protein, 108 aa, Artificial Sequence
  FNI3-VK-W94F (aa)
EILMTQSPATLSVSPGERATLSCRASQDVSGNLAWYQQRPGQAPRLLIYGASTRATGVPARFTGAGSGTEFTLTISSLQSEDFALYYCQHYNNFPPWTFGQGTKVEIK

SEQ ID NO: 175 - protein, 10 aa, Artificial Sequence
  FNI3-VK-W94F CDRL3 (aa)
QHYNNFPPWT

SEQ ID NO: 176 - DNA, 324 bp, Artificial Sequence
  FNI3-VK-W97F (nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattggccccctttcacctttggccagggcacaaaggtggagatcaag

SEQ ID NO: 177 - protein, 108 aa, Artificial Sequence
  FNI3-VK-W97F (aa)
EILMTQSPATLSVSPGERATLSCRASQDVSGNLAWYQQRPGQAPRLLIYGASTRATGVPARFTGAGSGTEFTLTISSLQSEDFALYYCQHYNNWPPFTFGQGTKVEIK

SEQ ID NO: 178 - protein, 10 aa, Artificial Sequence
  FNI3-VK-W97F CDRL3 (aa)
QHYNNWPPFT

SEQ ID NO: 179 - DNA, 324 bp, Artificial Sequence
  FNI3-VK-W94F-W97F (nt)
gagatcctgatgacccagtcccctgccacactgtccgtgtccccaggagagagggccaccctgagctgcagggcttctcaggacgtgtccggcaacctggcctggtaccagcagagaccaggacaggctccaaggctgctgatctatggagcttccaccagggctacaggcgtgccagctagattcaccggcgctggaagcggcacagagtttaccctgacaatctccagcctgcagtctgaggatttcgctctgtactattgtcagcactacaacaattttccccctttcacctttggccagggcacaaaggtggagatcaag

SEQ ID NO: 180 - protein, 108 aa, Artificial Sequence
  FNI3-VK-W94F-W97F (aa)
EILMTQSPATLSVSPGERATLSCRASQDVSGNLAWYQQRPGQAPRLLIYGASTRATGVPARFTGAGSGTEFTLTISSLQSEDFALYYCQHYNNFPPFTFGQGTKVEIK

SEQ ID NO: 181 - protein, 10 aa, Artificial Sequence
  FNI3-VK-W94F-W97F CDRL3 (aa)
QHYNNFPPFT

SEQ ID NO: 182 - DNA, 384 bp, Artificial Sequence
  FNI9-VH-W110F (nt)
caggtgcacctggtgcagagcggagctgaggtgaaggagccaggatccagcgtgacagtgtcttgcaaggcttccggcggcagcttcaacaatcaggctatctcctgggtgaggcaggctccaggacagggactggagtggatgggcggcatctttcccatctctggcacacctacctccgcccagaggttccagggaagggtgaccttcaccgctgacgagagcaccacaaccgtgtacatggatctgtcttccctgagatctgacgataccgccgtgtactattgtgccagagctggctccgactatttcaaccgcgatctgggcttcgagaattactattttgcttcctggggccagggcacactggtgaccgtgagctct

SEQ ID NO: 183 - protein, 128 aa, Artificial Sequence
  FNI9-VH-W110F (aa)
QVHLVQSGAEVKEPGSSVTVSCKASGGSFNNQAISWVRQAPGQGLEWMGGIFPISGTPTSAQRFQGRVTFTADESTTTVYMDLSSLRSDDTAVYYCARAGSDYFNRDLGFENYYFASWGQGTLVTVSS

SEQ ID NO: 184 - protein, 21 aa, Artificial Sequence
  FNI9-VH-W110F CDRH3 (aa)
ARAGSDYFNRDLGFENYYFAS

SEQ ID NO: 185 - DNA, 324 bp, Artificial Sequence
  FNI9-VK-W94F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaatttccccccttggacctttggccagggcacaaaggtggagatcaag

SEQ ID NO: 186 - protein, 108 aa, Artificial Sequence
  FNI9-VK-W94F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNFPPWTFGQGTKVEIK

SEQ ID NO: 187 - protein, 10 aa, Artificial Sequence
  FNI9-VK-W94F CDRL3 (aa)
QQYNNFPPWT

SEQ ID NO: 188 - DNA, 324 bp, Artificial Sequence
  FNI9-VK-W97F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattggccccctttcacctttggccagggcacaaaggtggagatcaag

SEQ ID NO: 189 - protein, 108 aa, Artificial Sequence
  FNI9-VK-W97F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNWPPFTFGQGTKVEIK

SEQ ID NO: 190 - protein, 10 aa, Artificial Sequence
  FNI9-VK-W97F CDRL3 (aa)
QQYNNWPPFT

SEQ ID NO: 191 - DNA, 324 bp, Artificial Sequence
  FNI9-VK-W94F-W97F (nt)
gagatcgtgatgacccagtccccagccacactgagcctgtccagcggagagagggccaccctgtcctgcagggcttcccggagcgtgtcttccaacctggcctggtaccagcagaagccaggccaggctcccagactgctgatctatgacgcctctaccagagctacaggcttctccgccaggtttgctggatctggatccggcacagagttcaccctgacaatcagctctctgcagagcgaggattctgctatctactattgtcagcagtacaacaattttccccctttcacctttggccagggcacaaaggtggagatcaag

SEQ ID NO: 192 - protein, 108 aa, Artificial Sequence
  FNI9-VK-W94F-W97F (aa)
EIVMTQSPATLSLSSGERATLSCRASRSVSSNLAWYQQKPGQAPRLLIYDASTRATGFSARFAGSGSGTEFTLTISSLQSEDSAIYYCQQYNNFPPFTFGQGTKVEIK

SEQ ID NO: 193 - protein, 10 aa, Artificial Sequence
  FNI9-VK-W94F-W97F CDRL3 (aa)
QQYNNFPPFT

SEQ ID NO: 194 - protein, 128 aa, Artificial Sequence
  FM08 VH
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 195 - protein, 103 aa, Artificial Sequence
  Synthetic sequence FM08 VL
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 196 - protein, 217 aa, Artificial Sequence
  WT hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 19 aa, Artificial Sequence
  Chimeric hinge sequence
ESKYGPPCPPCPAPPVAGP

SEQ ID NO: 198 - DNA, 386 bp, Artificial Sequence
  FNI17-v19-VH (co-nt)
caggtccagctggtccagagtggggcagaggtcaaagagccagggtcttcagtcacagtctcatgcaaagcaagcggaggaacattttccaacaatgtgatcagctgggtgaggcaggctccaggacagggactggagtggatgggcggcatcatccctacctctggcatcgccaactacgctcagaagttccagggcagagtggccatcatcgctgacaagtctacctccacagtgtatatggccctgtccagcctgagaagcgaggattccgccgtgtacttctgcgccagggctcggtccgactacttcaaccgcgatctgggttgggaggactattactttgaaaactgggggcagggcacactggtcactgtctcatcagc

SEQ ID NO: 199 - protein, 128 aa, Artificial Sequence
  FNI17-v19-VH (aa)
QVQLVQSGAEVKEPGSSVTVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSS

SEQ ID NO: 200 - DNA, 324 bp, Artificial Sequence
  FNI17-v19-VK (co-nt)
gaaattgtgatgacccagtctccagccactctgtcagtctctccaggggaacgagccactctgtcatgtcgggcctctcagtccgtcggctccagcctggcttggtaccagcagaagccaggacaggctcctaggctgctgatctatggagctagcaccagggctacaggcgtgccagctcggttcagcggatctggatccggcaccgagtttaccctgacaatctcttccctgcagtctgaggacttcgccgtgtactattgccagcactacaataactggcctccttggacattcgggcaggggacaaaagtcgagattaag

SEQ ID NO: 201 - protein, 108 aa, Artificial Sequence
  FNI17-v19-VK (aa)
EIVMTQSPATLSVSPGERATLSCRASQSVGSSLAWYQQKPGQAPRLLIYGASTRATGVPARFSGSGSGTEFTLTISSLQSEDFAVYYCQHYNNWPPWTFGQGTKVEIK

SEQ ID NO: 202 - DNA, 384 bp, Artificial Sequence
  FNI19-v3-VH (co-nt)
caggtccagctggtgcagagtggtgccgaggtcaaaaagccagggtcaagtgtcaaagtcagttgtaaagcatcagagggaacattcaacaagtacacaatcagctgggtgagacaggctccaggacagggactggagtggatgggcggcatcatccctatctctggcatcgccaattacgctcagaagttccagggccgcgtggccatcacagctgacgagtccaccacaaccgcctatatggagctgtccagcctgaggtctgaggattccgccgtgtactattgcgccaccgctgtgagcgactacttcaaccgggatctgggctgggaggactattattttccattctggggtcaggggacactggtcaccgtctcttcc

SEQ ID NO: 203 - protein, 128 aa, Artificial Sequence
  FNI19-v3-VH (aa)
QVQLVQSGAEVKKPGSSVKVSCKASEGTFNKYTISWVRQAPGQGLEWMGGIIPISGIANYAQKFQGRVAITADESTTTAYMELSSLRSEDSAVYYCATAVSDYFNRDLGWEDYYFPFWGQGTLVTVSS

SEQ ID NO: 204 - DNA, 324 bp, Artificial Sequence; identical to SEQ ID NO: 168
  FNI19-v3-VK (co-nt)

SEQ ID NO: 205 - protein, 108 aa, Artificial Sequence; identical to SEQ ID NO: 164
  FNI19-v3-VK (aa)

SEQ ID NO: 206 - DNA, 384 bp, Artificial Sequence; identical to SEQ ID NO: 90
  FNI9-v5-VH (co-nt)

SEQ ID NO: 207 - protein, 128 aa, Artificial Sequence [sequence omitted]

SEQ ID NO: 208 - DNA, 324 bp, Artificial Sequence
  FNI9-v5-VK (co-nt)
gagattgtgatgacccagtcccctgctaccctgagcgtgtcccccggagagagagctaccctgagttgccgcgccagccgcagtgtctctgacaacctggcttggtaccagcagaagccaggacaggctcctaggctgctgatctatggcgcctccaccagggctacaggcatcccagctcggttctctggatccggaagcggcaccgagtttaccctgacaatctccagcctgcagagcgaggatttcgccgtgtactattgccagcattacaacatctggcctccttggacattcggtcagggaactaaagtggaaattaag

SEQ ID NO: 209 - protein, 108 aa, Artificial Sequence
  FNI9-v5-VK (aa)
EIVMTQSPATLSVSPGERATLSCRASRSVSDNLAWYQQKPGQAPRLLIYGASTRATGIPARFSGSGSGTEFTLTISSLQSEDFAVYYCQHYNIWPPWTFGQGTKVEIK

SEQ ID NO: 210 - protein, 330 aa, Artificial Sequence
  IgHG1*01, G1m3 CH1-CH3 with M428L and N434S mutations and C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 211 - protein, 107 aa, Artificial Sequence [sequence omitted]

SEQ ID NO: 212 - protein, 458 aa, Artificial Sequence
  FNI17-v19 heavy chain with M428L and N434S mutations in CH3 and a C-terminal lysine
QVQLVQSGAEVKEPGSSVTVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 213 - protein, 457 aa, Artificial Sequence
  FNI17-v19 heavy chain with M428L and N434S mutations in CH3, without C-terminal lysine
QVQLVQSGAEVKEPGSSVTVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 214 - protein, 215 aa, Artificial Sequence
  FNI17-v19 light chain
EIVMTQSPATLSVSPGERATLSCRASQSVGSSLAWYQQKPGQAPRLLIYGASTRATGVPARFSGSGSGTEFTLTISSLQSEDFAVYYCQHYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 329 aa, Artificial Sequence
  IgHG1*01, G1m3 CH1-CH3 with M428L and N434S mutations, without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 216 - protein, 128 aa, Artificial Sequence
  Synthetic sequence FNI17-v13 VH
QVQLVQSGARVKEPGSSVKVSCKASGGTFSNNVISWVRQAPGQGLEWMGGIIPTSGIANYAQKFQGRVAIIADKSTSTVYMALSSLRSEDSAVYFCARARSDYFNRDLGWEDYYFENWGQGTLVTVSS

SEQ ID NO: 217 - protein, 108 aa, Artificial Sequence; identical to SEQ ID NO: 201
  Synthetic sequence FNI17-v13 VK

SEQ ID NO: 218 - DNA, 407 bp, Artificial Sequence
  Synthetic sequence Codon-optimized nucleotide sequence encoding FNI17-v19-VH with N-terminal amino acids T-G-V-H-S and C-terminal amino acids A-S
accggtgtacattctcaggtccagctggtccagagtggggcagaggtcaaagagccagggtcttcagtcacagtctcatgcaaagcaagcggaggaacattttccaacaatgtgatcagctgggtgaggcaggctccaggacagggactggagtggatgggcggcatcatccctacctctggcatcgccaactacgctcagaagttccagggcagagtggccatcatcgctgacaagtctacctccacagtgtatatggccctgtccagcctgagaagcgaggattccgccgtgtacttctgcgccagggctcggtccgactacttcaaccgcgatctgggttgggaggactattactttgaaaactgggggcagggcacactggtcactgtctcatcagcgtcgac

SEQ ID NO: 219 - protein, 21 aa, Artificial Sequence
  Synthetic sequence FNI antibody consensus CDRH3 amino acid sequence
  VARIANT 2: Xaa = R or T
  VARIANT 3: Xaa = A or T
  VARIANT 4: Xaa = V, G, H, R, or N
  VARIANT 14: Xaa = W or F
  VARIANT 15: Xaa = D or E
  VARIANT 16: Xaa = D or N
  VARIANT 18: Xaa = Y or F
  VARIANT 20: Xaa = P, D, E, or A
  VARIANT 21: Xaa = L, I, S, Y, H, D, N, or F
AXXXSDYFNRDLGXXXYXFXX

SEQ ID NO: 220 - protein, 8 aa, Artificial Sequence
  Synthetic sequence FNI antibody consensus CDRH1 amino acid sequence
  VARIANT 1: Xaa = G or E
  VARIANT 2: Xaa = D, A, G, or V
  VARIANT 3: Xaa = T or S
  VARIANT 4: Xaa = F or L
  VARIANT 5: Xaa = N or S
  VARIANT 6: Xaa = N, R, K, or S
  VARIANT 7: Xaa = Y, H, Q, or N
  VARIANT 8: Xaa = V, T, A, or E
XXXXXXXX

SEQ ID NO: 221 - protein, 8 aa, Artificial Sequence
  Synthetic sequence FNI antibody consensus CDRH2 amino acid sequence
  VARIANT 2: Xaa = I, H, L, or F
  VARIANT 3: Xaa = P or A
  VARIANT 4: Xaa = I, V, L, or T
  VARIANT 5: Xaa = S, T, or F
  VARIANT 6: Xaa = G, A, R, P, or Q
  VARIANT 7: Xaa = I, K, R, or T
  VARIANT 8: Xaa = P, T, A, or G
IXXXXXXX

SEQ ID NO: 222 - protein, 6 aa, Artificial Sequence
  Synthetic sequence FNI antibody consensus CDRL1 amino acid sequence
  VARIANT 1: Xaa = Q or R
  VARIANT 2: Xaa = T, S or D
  VARIANT 3: Xaa = V or I
  VARIANT 4: Xaa = S or G
  VARIANT 5: Xaa = S, G, I, T, or D
  VARIANT 6: Xaa = N, D, H, or S
XXXXXX

SEQ ID NO: 223 - protein, 3 aa, Artificial Sequence
  Synthetic sequence FNI antibody consensus CDRL2 amino acid sequence
  VARIANT 1: Xaa = G or D
XAS

SEQ ID NO: 224 - protein, 10 aa, Artificial Sequence
  Synthetic sequence FNI antibody consensus CDRL3 amino acid sequence
  VARIANT 2: Xaa = Q or H
  VARIANT 5: Xaa = N, T, or I
  VARIANT 6: Xaa = W or F
  VARIANT 9: Xaa = W or F;
QXYNXXPPXT

SEQ ID NO: 225-226 - skipped (2 entries)

SEQ ID NO: 227 - DNA, 384 bp, Artificial Sequence
  Synthetic sequence FNI-UCA-IGH
caggtgcagctggtgcagtctggcgccgaggtgaagaagccaggctccagcgtgaaggtgagctgcaaggcttctggcggcaccttctcttcctacgctatctcctgggtgaggcaggctccaggacagggactggagtggatgggcggcatcatccctatcttcggcacagccaactacgctcagaagtttcagggcagagtgaccatcacagccgacgagtctacctccacagcttatatggagctgagctctctgcgctccgaggataccgccgtgtactattgtgccagggctggcagcgactacttcaaccgggatctgggctgggagaattactattttgactattggggccagggcaccctggtgacagtgtccagc

SEQ ID NO: 228 - protein, 128 aa, Artificial Sequence
  Synthetic sequence FNI-UCA VH
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARAGSDYFNRDLGWENYYFDYWGQGTLVTVSS

SEQ ID NO: 229 - DNA, 324 bp, Artificial Sequence
  Synthetic sequence FNI-UCA-IGK
gagatcgtgatgacccagtctcctgccacactgagcgtgtctccaggagagagggccaccctgtcctgcagggcttcccagagcgtgtccagcaacctggcctggtaccagcagaagccaggccaggctcccaggctgctgatctatggcgccagcaccagagctacaggcatcccagctcgcttctctggatccggaagcggcacagagtttaccctgacaatctcttccctgcagtctgaggacttcgccgtgtactattgtcagcagtacaacaattggcccccttggacctttggccagggcacaaaggtggagatcaag

SEQ ID NO: 230 - protein, 108 aa, Artificial Sequence
  Synthetic sequence FNI-UCA VK
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 231 - protein, 8 aa, Artificial Sequence
  Synthetic sequence FNI-UCA CDRH1
GGTFSSYA

SEQ ID NO: 232 - protein, 8 aa, Artificial Sequence
  Synthetic sequence FNI-UCA CDRH2
IIPIFGTA

SEQ ID NO: 233 - protein, 21 aa, Artificial Sequence
  Synthetic sequence FNI-UCA CDRH3
ARAGSDYFNRDLGWENYYFDY

SEQ ID NO: 234 - protein, 6 aa, Artificial Sequence
  Synthetic sequence FNI-UCA CDRL1
QSVSSN

SEQ ID NO: 235 - protein, 3 aa, Artificial Sequence
  Synthetic sequence FNI-UCA CDRL2
GAS

SEQ ID NO: 236 - protein, 10 aa, Artificial Sequence [sequence omitted]

(19) United States
(12) Patent Application Publication
(10) Pub. No.: US 2024/0092876 A1
(43) Pub. Date: Mar. 21, 2024

(54) BROADLY NEUTRALIZING ANTIBODIES AGAINST INFLUENZA NEURAMINIDASE

(71) Applicants: Vir Biotechnology, Inc., San Francisco, CA (US); Humabs BioMed SA, Bellinzona (CH)

(72) Inventors: Davide CORTI, Bellinzona (CH); Matteo Samuele PIZZUTO, Bellinzona (CH); Andrea MINOLA, Bellinzona (CH); Elisabetta CAMERONI, Bellinzona (CH); Gyorgy SNELL, San Francisco, CA (US); Elena FERRI, San Francisco, CA (US)

(21) Appl. No.: 18/253,385

(22) PCT Filed: Nov. 19, 2021
(86) PCT No.: PCT/US2021/060155

§ 371 Date: May 17, 2023

Related U.S. Application Data
(60) Provisional application No. 63/117,448, filed on Nov. 23, 2020.
(60) Provisional application No. 63/123,424, filed on Dec. 9, 2020.
(60) Provisional application No. 63/197,160, filed on Jun. 4, 2021.
(60) Provisional application No. 63/261,463, filed on Sep. 21, 2021.

(51) Int. Cl. C07K 16/10; A61P 31/16

(52) U.S. Cl. CPC C07K 16/1018; A61P 31/16; A61K 2039/505

(57) ABSTRACT

The instant disclosure provides antibodies and antigen-binding fragments thereof that can bind to an influenza virus neuraminidase (NA) and can neutralize an influenza virus infection. Also provided are polynucleotides that encode an antibody, vectors that comprise such polynucleotides, host cells that can express the antibodies, related compositions, and methods of using the herein disclosed compositions to, for example, treat or prevent an influenza infection.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930585_414WO_SEQUENCE_LISTING.txt. The text file is 137 KB, was created on Nov. 16, 2021, and is being submitted electronically via EFS-Web.

### BACKGROUND

Influenza is an infectious disease which spreads around the world in yearly outbreaks, resulting per year in about three million to about five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat and a runny nose. The incubation period varies between one to four days, although usually symptoms begin about two days after exposure to the virus. Complications of influenza may include pneumonia, sinus infections, and worsening of previous health problems such as asthma or heart failure, sepsis or exacerbation of chronic underlying disease.

Influenza is caused by influenza virus, an antigenically and genetically diverse group of viruses of the family Orthonyxoviridae that contains a negative-sense, single-stranded, segmented RNA genome. Of the four types of influenza virus (A, B, C and D), three types (A, B and C) are known to affect humans. Influenza viruses can be categorized based on the different subtypes of major surface proteins present: Hemagglutinin (HA) and Neuraminidase (NA). There are at least 18 influenza A subtypes defined by their hemagglutinin (“HA”) proteins. The HAs can be classified into two groups. Group 1 includes H1, H2, H5, H6, H8, H9, H11, H12, H13, H16 and H17 subtypes, and group 2 includes H3, H4, H7, H10, H14 and H15 subtypes. There are at least 11 different neuraminidase subtypes (N1 through N11, respectively (cdc.gov/flu/about/viruses/types.htm)). Neuraminidases function in viral mobility and spread by catalyzing hydrolysis of sialic acid residues on virions prior to release from an infected host cell, and on target cell surface glycoproteins. Drugs designed to inhibit neuraminidase (NAIs) have been developed (e.g., oseltamivir, zanamivir, peramivir, laninamivir), though naturally acquired mutations of IAV subtypes have reduced susceptibility to current NAIs (Hussain et al., Infection and Drug Resistance 10:121-134 (2017).

New modalities for treating influenza virus infections are needed.

### BRIEF DESCRIPTION OF THE DRAWINGS

FIG. 1 shows a workflow for anti-“NA” (neuraminidase) monoclonal antibody discovery. Donors were selected by screening serum from tonsillar donor samples (n=50) for reactivity against neuraminidase subtype N1 and N2 antigens, and serum from PBMC (peripheral blood mononuclear cell) donor samples (n=124) for reactivity against neuraminidase subtype N4, N3, and N9. Neuraminidase antigens for screening were expressed in mammalian cells and binding was evaluated by flow cytometry. B memory cells from five donors were sorted by flow cytometry for input into the discovery workflow. Single sorted B cells (n=39,350) were co-cultured with mesenchymal stromal cells (MSC) in 50 μl cultures to stimulate antibody secretion. Secreted antibodies were evaluated by binding and NA inhibition assays. Inhibition of N1 sialidase activity was evaluated using ELLA (enzyme-linked lectin assay), and inhibition of N1, N2, and N9 sialidase activity was measured using a fluorescence-based assay that measures cleavage of the 2′-(4-Methylumbelliferyl)-α-D-N-acetylneuraminic acid (MUNANA). “N1 activity” refers to neuraminidase inhibition activity. Binding to NAs from group 1 IAV N1 A/Vietnam/1203/2004, and group 2 IAVs N2 A/Tanzania/205/2010 and N9 A/Hong Kong/56/2015 was evaluated by ELISA to determine breadth. Antibody sequences from selected B cells were cloned as cDNAs and sequenced.

FIG. 2A shows VH domain sequence alignments of monoclonal antibodies (with “FNI” prefix) against Influenza A Viruses (“IAV”) that were isolated from human donor PBMCs. FIG. 2B shows VH domain sequence alignments of “FNI3” (VH: SEQ ID NO.:26; VL: SEQ ID NO.: 32) and “FNI9” (VH: SEQ ID NO.:86; VL: SEQ ID NO.: 92) with the unmutated common ancestor, “UCA” (VH: SEQ ID NO.:228; VL: SEQ ID NO.:230).

FIGS. 3A-3C show binding of FNI3 and FNI9 to N1 (FIG. 3A), N2 (FIG. 3B), and N9 (FIG. 3C) NAs measured by enzyme-linked immunosorbent assay (ELISA), reported as OD versus concentration in ng/ml. Binding by a comparator antibody, 1G01-LS, and a negative control antibody against an irrelevant antigen, “K−” was also measured.

FIGS. 4A-4C show binding kinetics of FNI3 bearing M428L/N434S Fc mutations (“FNI3-LS” in the figures) and FNI9 bearing M428L/N434S Fc mutations (“FNI9-LS” in the figures) to N1 (FIG. 4A), N2 (FIG. 4B), and N9 (FIG. 4C) NAs, as measured by Bio-Layer Interferometry (BLI). Dissociation is reported as kdis (1/s), association is reported as kon (1/Ms), and KD was calculated from the ratio of kdis/kon. Binding by a comparator antibody, 1G01-LS, was also measured.

FIG. 5 summarizes results from flow cytometry assays testing binding by FNI3 and FNI9, as well as by comparator antibody 1G01, against a panel of group I IAV, group II IAV, and Influenza B Virus (IBV) NAs. Bold font indicates NAs from influenza viruses isolated from humans. Values on the scale at right show range of calculated EC50. Values were selected based on the lowest concentration at which binding was observed.

FIG. 6 shows phylogenetic relatedness of NAs from group 1 IAVs, group 2 IAVs, and IBVs.

FIGS. 7A-7C relate to activity of FNI3 and FNI9 against NAs that bear a glycosylation site. FIG. 7A shows glycosylation sites of group 2 IAV N2 subtype NAs at positions 245 (245Gly+) and 247 (247Gly+) in A/South Australia/34/2019, A/Switzerland/8060/2017, A/Singapore/INFIMH-16-0019/2016, and A/Switzerland/9715293/2013. FIG. 7B summarizes inhibition of sialidase activity (NAI) in A/Switzerland/8060/2017, A/Singapore/INFIMH-16-0019/2016, and A/Switzerland/9715293/2013 live virus stocks, reported as EC50 in μg/ml. FIG. 7C shows binding of FNI3 and FNI9 to NA in mammalian cells infected with A/South Australia/34/2019 (245Gly+) measured by flow cytometry. Mock staining is shown as a negative control.

FIG. 8 shows binding of FNI3 and FNI9 to NA expressed on mammalian cells infected with a H1N1 Swine Eurasian avian-like (EA) strain, A/Swine/Jiangsu/J004/2018, measured by flow cytometry. Mock staining is shown as a negative control.

FIG. 9 shows lack of polyreactivity of FNI3 and FNI9 binding using human epithelial type 2 (HEP-2) cells. Anti-HA antibody FI6v3 was used as a positive control, and anti-paramyxovirus antibody MPE8 was included as a negative control.

FIG. 10 summarizes inhibition of sialidase activity (“NAI”) by FNI3 and FNI9 against a panel of group I IAV, group II IAV, and Influenza B Virus (IBV) NAs, as measured by MUNANA assay. Bold font indicates NAs from influenza viruses isolated from humans.

FIG. 11 shows in vitro inhibition of sialidase activity (reported as IC50 in μg/ml) by FNI3 and FNI9 against group I (H1N1) IAV, group I (H3N2) IAV, and IBV NAs.

FIGS. 12A-12B show in vitro inhibition of sialidase activity (reported as IC50 in μg/ml) by FNI3 and FNI9 against group I (H1N1) IAV, group 11 (H3N2) IAV, and IBV NAs. FIG. 12A depicts inhibition activity against group I IAVs, group II IAVs, and IBVs within the same plot and FIG. 12B depicts against these IAVs in separate plots.

FIG. 13A shows a panel of IAV and IBV strains tested in an in vitro inhibition of sialidase activity assay.

FIG. 13B shows results from the assay (reported as IC50 in μg/ml) for FNI3, FNI9, FNI14 (VH: SEQ ID NO.:134; VL: SEQ ID NO.: 140), FNI17 (VH: SEQ ID NO.: 146: VL: SEQ ID NO.: 152), and FNI19 (VH: SEQ ID NO.: 158: VL: SEQ ID NO.: 164). Asterisk in figure key indicates a glycosylation site is present in position 245.

FIGS. 14A-14D show neutralization of antibodies FNI1 (VH: SEQ ID NO.:2; VL: SEQ ID NO.: 8), FNI3, FNI9, FNI14, FNI17, and FNI19 against H1N1 A/California/07/2009 (FIG. 14A), H3N2 A/Hong Kong/8/68 (FIG. 14B), B/Malaysia/2506/2004 (FIG. 14C), and B/Jiangsu/10/2003 (FIG. 14D) NAs (reported as IC50 in μg/ml).

FIGS. 15A and 15B show antibody activation of FcγRIIIa (FIG. 15A; F158 allele) and FcγRIIa (FIG. 15B; H131 allele). Activation was measured using an NFAT-mediated Luciferase reporter in engineered Jurkat cells. FNI3 and FNI9 were tested, along with a comparator antibody FM08 (“FM08_LS” in the figure; VH: SEQ ID NO.:194; VL: SEQ ID NO.: 195) and a negative control antibody (FY1-GRLR).

FIGS. 16A and 16B show frequency by year of NA antiviral-resistant mutations in (FIG. 16A) N1 (H1N1, swine H1N1, and avian H5N1) and (FIG. 16B) N2 (H3N2, H2N2) subtypes.

FIGS. 17A to 17E show neutralization of H1N1 A/California/07/2009 virus engineered with reverse genetics to harbor oseltamivir (OSE)-resistant mutations (H275Y, E119D and H275Y, or S247N and H275Y) by anti-flu antibodies or oseltamivir. Neutralization activity of FNI3 (FIG. 17A), FNI9 (FIG. 17B), and oseltamivir (FIG. 17C) were measured, along with comparator antibodies FM08 (FIG. 17D) and 1G01 (FIG. 17E).

FIGS. 18A and 18B show neutralization of group I (H1N1) IAV, group II (H3N2) IAV, IBV viruses, and IAV and IBV viruses engineered with reverse genetics to harbor OSE-resistant mutations (H275Y, E119D/H275Y, H275Y/S247N, I222V, or N294S), by anti-NA antibodies (reported as IC50 in μg/ml). Asterisks in FIG. 18A (x-axis) indicate viruses bearing OSE-resistant mutations. Neutralization activity of FNI3, FNI9, and a comparator antibody, 1G01, was measured. FIG. 18A depicts neutralization of individual viral strains and FIG. 18B depicts neutralization of viral strains grouped by neutralizing anti-NA antibody.

FIG. 19 shows data from crystal structure studies showing docking of the antigen-binding fragment (Fab) domain of the FNI3 antibody with NA.

FIGS. 20A and 20B show diagrams constructed from crystal structure studies of the heavy chain complementarity-determining region 3 (H-CDR3) of the FNI3 heavy chain when it is unbound (FIG. 20A) or bound to N2 NA (FIG. 20B). The unbound FNI3 H-CDR3 crystal structure (FIG. 20A) shows a beta sheet conformation and intact main chain hydrogen bonds between carboxylic acid groups (CO) and amino groups (NH) of residues E111 (CO)-D102 (NH), E111 (NH)-D102 (CO), G109 (CO)-F104 (NH), G109 (NH)-N105 (CO), and L108 (NH)-N105 (CO). The FNI3-N2 crystal structure (FIG. 20B) shows disruption of the H-CDR3 beta sheet conformation and one intact main chain hydrogen bond between G109 (CO)-F104 (NH).

FIGS. 21A and 21B show diagrams generated from crystal structure studies showing angle of docking of the antigen-binding fragment (Fab) domain of FNI3 and of comparator antibodies 1G01, 1G04, and 1E01, in complex with NA subtypes. Lines indicate angle of docking in all panels and Protein Data Bank (PDB) identification codes are shown for comparator antibodies 1G01, 1G04, and 1E01. FIG. 21A shows 1G01 in complex with N1 NA (upper panel) and 1G04 in complex with N9 NA (lower panel). FIG. 21B shows FNI3 in complex with N2 NA (upper panel) and 1E01 in complex with N2 NA (lower panel).

FIG. 22 shows conformation and interactions of FNI3 CDRs: H-CDR3, H-CDR2, and L-CDRs. To generate these data, proteins were “quick prepped” using MOE (Molecular Operating Environment).

FIG. 23 shows crystal structure of FNI3 in complex with N2 NA, including residues of light chain CDRs (L-1, L-2, L-3) and heavy chain CDRs (H-1, H-2, H-3). The interaction of H-CDR3 with N2 NA is shown in enhanced resolution in the right panel. Negative numbers are interaction energy in kcal/mol. Proteins were “quick prepped” using MOE (Molecular Operating Environment) software.

FIG. 24 shows a crystal structure representation of FNI3 in complex with oseltamivir-bound N2 NA. Oseltamivir is shown interacting with R292, R371, and R118 of N2 NA.

FIG. 25 shows an alternative view of the crystal structure showing FNI3 in complex with oseltamivir-bound N2 NA.

FIGS. 26A and 26B show analysis of FNI3 epitope conservation in N2 NA sequences from H3N2 (n=60, 597) isolated between the years 2000 and 2020. The table in FIG. 26A shows frequency of an amino acid at a particular position in the analyzed N2 NA sequences. Circled values indicate amino acids appearing at the lowest three frequencies, Glu221 (E221, 17.41%), Ser245 (S245, 33.69/6), and Ser247 (S247, 36.16%). Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine. FIG. 26B shows interaction of VH Y60 and Y94 from FNI3 with E221, S245, and S247 of N2 NA.

FIG. 27 shows a comparison of N2 NA FNI3 epitope conservation (top; as shown in FIGS. 26A and 26B) with FNI3 epitope conservation in N1 NA sequences from H1N1 (n=57,597) isolated between the year 2000 and 2020 (bottom). Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine. FIGS. 28A and 28B show the design of an in vivo study to evaluate prophylactic activity of FNI3 (“mAb-03” in FIG. 28A) and FNI9 (“mAb-09” in FIG. 28A) in BALB/c mice infected with IAV A/Puerto Rico/8/34 or A/Hong Kong/8/68. FIG. 28A shows the dosing and virus strains used in the study. FIG. 28B shows the timeline and endpoints of the study.

FIGS. 29A-29D show measurements of body weight over fifteen days in BALB/c mice that were infected with H1N1 A/Puerto Rico/8/34 following pre-treatment with FNI3. Antibody was administered at 6 mg/kg (FIG. 29A), 2 mg/kg (FIG. 29B), 0.6 mg/kg (FIG. 29C), or 0.2 mg/kg (FIG. 29D), one day prior to infection with a LD90 (90% lethal dose) of A/Puerto Rico/8/34. Body weight of mice administered a vehicle control was also measured (left graph in each figure).

FIGS. 30A-30D show measurements of body weight over fifteen days in BALB/c mice infected with H1N1 A/Puerto Rico/8/34 following pre-treatment with FNI9. Antibody was administered at 6 mg/kg (FIG. 30A), 2 mg/kg (FIG. 30B), 0.6 mg/kg (FIG. 30C), or 0.2 mg/kg (FIG. 30D), one day prior to infection with a LD90 (90% lethal dose) of A/Puerto Rico/8/34. Body weight of mice administered a vehicle control was also measured (left graph in each figure).

FIGS. 31A-31D show measurements of body weight over fifteen days in BALB/c mice infected with H3N2 A/Hong Kong/8/68 following pre-treatment with FNI3. Antibody was administered at 6 mg/kg (FIG. 31A), 2 mg/kg (FIG. 31B), 0.6 mg/kg (FIG. 31C), or 0.2 mg/kg (FIG. 31D), one day prior to infection with a LD90 (90% lethal dose) of A/Hong Kong/8/68. Body weight of mice administered a vehicle control was also measured (left graph in each figure).

FIGS. 32A-32D show measurements of body weight over fifteen days in BALB/c mice infected with H3N2 A/Hong Kong/8/68 following pre-treatment with FNI9. Antibody was administered at 6 mg/kg (FIG. 32A), 2 mg/kg (FIG. 32B), 0.6 mg/kg (FIG. 32C), or 0.2 mg/kg (FIG. 32D), one day prior to infection with a LD90 (90% lethal dose) of A/Hong Kong/8/68. Body weight of mice receiving a vehicle control was also measured (left graph in each figure).

FIGS. 33A and 33B show survival over fifteen days in BALB/c mice infected with A/Puerto Rico/8/34 (FIG. 33A) or A/Hong Kong/8/68 (FIG. 33B) following treatment with FNI3 or FNI9. Survival in mice pre-treated with a vehicle control was also measured.

FIGS. 34A and 34B show body weight loss from day 4 to 14 post-infection (reported as area-under-the-curve in BALB/c mice infected with A/Puerto Rico/8/34 (FIG. 34A) or A/Hong Kong/8/68 (FIG. 34B) following pre-treatment with FNI3 or FNI9. Body weight loss in mice pre-treated with a vehicle control was also measured.

FIGS. 35A and 35B show negative area-under-the-curve peak values compared with IgG in serum from area-under-the-curve analysis of body weight loss in BALB/c mice infected with A/Puerto Rico/8/34 (FIG. 35A) or A/Hong Kong/8/68 (FIG. 35B) following treatment with FNI3 or FNI9.

FIG. 36 shows in vivo pharmacokinetics of FNI3 (“FNI3-LS”), FNI9 (“FNI9-LS”) and comparator antibodies FM08 and 1G01 (“1G01-LS”), all bearing M428L/N434S mutations, in tg32 mice. Calculated half-life is highlighted by a rectangle.

FIG. 37 summarizes results from flow cytometry assays testing binding by FNI3, FNI9, FNI17, and FNI19 at the indicated concentrations (μg/mL) against a panel of group I IAV-, group II IAV-, and Influenza B Virus (IBV) NAs transiently expressed on mammalian cells. Bold font indicates NAs from influenza viruses isolated from humans. Values on the scale at right show range of calculated EC50. Values were selected based on the lowest concentration at which binding was observed.

FIG. 38 shows in vitro inhibition of sialidase activity (reported as IC50 in μg/ml) by FNI3, FNI9, FNI17, and FNI19 against group I (H1N1) and group II (H3N2) NAs from IAVs circulating in humans. Rectangles indicate group II (H3N2) NAs harboring glycosylation at position 245 and corresponding sialidase inhibition values (reported as IC50 in μg/ml).

FIG. 39 shows in vitro inhibition of sialidase activity (reported as IC50 in μg/ml) by FNI3, FNI9, FNI17, and FNI19 against a panel of human ancestral, Victoria-lineage, and Yamagata-lineage IBV NAs.

FIG. 40 shows in vitro neutralizing activity measured by nucleoprotein (NP) staining of FNI3, FNI9, FNI17, and FNI19 against group I (H1N1) IAV, group II (H3N2) IAV, and IBV NAs. Neutralizing activity of comparator anti-HA antibodies “FM08_LS” and “FHF11v9” was also measured.

FIG. 41 shows in vitro neutralizing activity, measured by nucleoprotein (NP) staining, by FNI3, FNI9, FNI17, FNI19, and oseltamivir (OSE) against group I (H1N1) IAV, group II (H3N2) IAV, and IBV NAs. 1 nM=150 μl.

FIGS. 42A and 42B show in vitro inhibition of sialidase activity (reported as IC50 in μg/ml) by FNI3 and FNI9 against NAs from OSE-resistant influenza viruses, as measured by MUNANA assay. OSE-resistant IAVs were engineered with reverse genetics to harbor Oseltamivir (OSE)-resistant mutations. FIG. 42A shows inhibition of sialidase activity against Cal/09 N1 and Cal/09 N1 OSE-resistant (H1N1). FIG. 42B shows inhibition of sialidase activity against Aichi/68 N2 and Aichi/68 N2 OSE-resistant NAs (H3N2).

FIG. 43 shows antibody activation of FcγRIIIa (F158 allele) and FcγRIIa (H131 allele). Activation was measured using an NFAT-mediated luciferase reporter in engineered Jurkat cells. Activation was assessed following incubation with A549 cells infected with H1N1 influenza strain A/Puerto Rico/8/34 at a multiplicity of infection (MOI) of 6. FNI3, FNI9, FNI17, and FNI19 were tested, along with a comparator antibody “FM08_MLNS” bearing M428L/N434S mutations, and a negative control antibody (FY1-GRLR).

FIGS. 44A and 44B show antibody activation of FcγRIIIa (V158 allele) following incubation with IAV (FIG. 44A) and IBV (FIG. 44B) NAs. Activation was measured using an NFAT-mediated luciferase reporter in engineered Jurkat cells following incubation with Expi-CHO cells transiently transfected with plasmids encoding different IAV and IBV NAs. FNI3, FNI9, FNI17, and FNI19 were tested, along with a negative control antibody (FY1-GRLR).

FIGS. 45A and 45B show antibody activation of FcγRIIa (H131 allele) following incubation with IAV (FIG. 45A) and IBV (FIG. 45B) NAs. Activation was measured using an NFAT-mediated luciferase reporter in engineered Jurkat cells following incubation with Expi-CHO cells transiently transfected with plasmids encoding different IAV and IBV NAs. FNI3, FNI9, FNI17, and FNI19 were tested, along with a negative control antibody (FY1-GRLR).

FIG. 46 shows negative area-under-the-curve peak values (reported as EC50 in μg/ml) compared with IgG in serum from area-under-the-curve analysis of body weight loss in BALB/c mice infected with A/Puerto Rico/8/34 (H1N1) or A/Hong Kong/8/68 (H3N2) following treatment with FNI3, FNI9, or FM08_LS. For fitting purposes the Area of Negative Peaks from the vehicle groups have been calculated at the IgG concentration of 10⁻¹ μg/ml.

FIGS. 47A and 47B show the design of an in vivo study to evaluate prophylactic activity of FNI3_MLNS (“mAb-03” in FIG. 47A) and FNI9_MLNS (“mAb-09” in FIG. 47A) in DBA/2J mice infected with IBVs B/Victoria/504/2000 (Yamagata) or B/Brisbane/60/2008 (Victoria). FIG. 47A shows the dosing and virus strains used in the study. FIG. 47B shows the timeline and endpoints of the study.

FIGS. 48A-48D show measurements of body weight over fifteen days in DBA/2 mice that were infected with IBV B/Victoria/504/2000 (Yamagata) following pre-treatment with FNI3 or FNI9. Antibody was administered at 6 mg/kg (FIG. 48A), 2 mg/kg (FIG. 48B), 0.6 mg/kg (FIG. 48C), or 0.2 mg/kg (FIG. 48D), one day prior to infection with a LD90 (90% lethal dose) of IBV B/Victoria/504/2000 (Yamagata). Body weight of mice administered a vehicle control was also measured (left graph in each figure).

FIGS. 49A-49D show measurements of body weight over fifteen days in DBA/2 mice that were infected with IBV B/Brisbane/60/2008 (Victoria) following pre-treatment with FNI3 or FNI9. Antibody was administered at 6 mg/kg (FIG. 49A), 2 mg/kg (FIG. 49B), 0.6 mg/kg (FIG. 49C), or 0.2 mg/kg (FIG. 49D), one day prior to infection with a LD90 (90% lethal dose) of IBV B/Brisbane/60/2008 (Victoria). Body weight of mice administered a vehicle control was also measured (left graph in each figure).

FIGS. 50A and 50B show body weight loss from day 4 to 14 post-infection (reported as change in weight area-under-the-curve) in DBA/2 mice infected with B/Victoria/504/2000 (Yamagata) (FIG. 50A) or B/Brisbane/60/2008 (Victoria) (FIG. 50B) following pre-treatment with FNI3 or FNI9. Body weight loss in mice pre-treated with a vehicle control was also measured.

FIGS. 51A and 51B show survival over fifteen days in DBA/2 mice infected with B/Victoria/504/2000 (Yamagata) (FIG. 51A) or B/Brisbane/60/2008 (Victoria) (FIG. 51B) following treatment with FNI3 or FNI9. Survival in mice pre-treated with a vehicle control was also measured.

FIGS. 52A and 52B show FNI3 epitope conservation in IAV and IBV NAs. FIG. 52A shows an analysis of N2 NA sequences from H1N2, H2N2, H3N2, and H5N2 IAVs (n=65,5262) (top) versus N1 NA sequences from H1N1 and H5N1 (N=58,954) (bottom). All sequences were isolated between the year 2000 and 2020. Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine. Residues surrounded by squares in FIG. 52A indicate certain amino acids described in the lower panel of FIG. 52B. The table in FIG. 52B shows important FNI3-interacting residues within N2 NA and counterpart FNI3 CDRH3 residues.

FIG. 53 shows FNI3 epitope conservation in IBV NAs. IBV NA sequences from B/Brisbane/60/2008 (“FluB Victoria” in the figure; N=7,814; top) versus IBV NA sequences from B/Victoria/504/2000 (“FluB Yamagata” in the figure; N=13,243; bottom) were analyzed. Acidic amino acids include: aspartic acid, glutamic acid; basic amino acids include: arginine, histidine, lysine; hydrophobic amino acids include: isoleucine, leucine, tryptophan, valine, alanine, proline; neutral amino acids include: asparagine, glutamine; and polar amino acids include: serine, threonine, glycine, tyrosine. Residues surrounded by squares indicate primary NA residues interacting with the FNI3 HCDR3 which are 100% conserved among IAV N1/N2 and IBVs.

FIGS. 54A and 54B show in vivo pharmacokinetics of FNI antibodies bearing MLNS Fc mutations (FNI3 (“FNI3-LS”), FNI9 (“FNI9-LS”), FNI17 (“FNI17-LS”), FNI19 (“FNI19-LS”)), and comparator antibody FM08_MLNS in SCID tg32 mice over 30 days post-administration. Concentration over time (reported as μg/ml) is shown in FIG. 54A. The table in FIG. 54B shows half-life (reported in days), AUC (reported in day*μg/ml), clearance (reported in μg/ml), and volume (reported in ml).

FIG. 55 shows lack of polyreactivity of FNI3, FNI9, FNI17, and FNI19 binding against human epithelial type 2 (HEP-2) cells.

FIGS. 56A-56C relate to FNI antibodies and crystal structure studies showing docking of the antigen-binding fragment (Fab) domain of FNI antibodies with NA. FIG. 56A shows FNI3 docking on N2 NA. FIG. 56B shows an overlay of FNI3, FNI17, and FNI19 antibodies docking with NA. FIG. 56C shows VH amino acid sequence alignments of FNI3, FNI9, FNI17, and FNI19 with unmutated common ancestor, “UCA”. CDRH3, which interacts with NA, is highlighted by a rectangle.

FIG. 57A shows crystal structure of FNI17 in complex with N2 NA, including residues of light chain CDRs (L-1, L-2, L-3) and heavy chain CDRs (H-1, H-2, H-3). The interaction of H-CDR3 with N2 NA is shown in enhanced resolution in the right panel. Percentages indicate each residue's contribution to calculated binding energy. FIG. 57B shows VH amino acid sequence alignments of FNI3, FNI9, FNI17, and FNI19 with unmutated common ancestor, “UCA”. VH residues D107 and R106, which interact with NA, are highlighted by a rectangle.

FIG. 58 shows conservation of the top five interacting residues within the FNI NA epitope in group I IAVs, group II IAVs, and IBVs from 2009 to 2019.

FIG. 59 shows in vitro neutralizing activity measured by nucleoprotein (NP) staining by FNI9, Oseltamivir (OSE), and a comparator antibody “FM08” against H3N2 A/Hong Kong/8/68 virus. Calculated IC50 (in nM), IC80 (in nM), and maximum inhibition (reported as a percentage) are shown below the graph.

FIG. 60 shows in vitro inhibition of sialidase activity by FNI17 variant FNI17-v19 (VH: SEQ ID NO.:199; VL: SEQ ID NO.: 201) and FNI19 variant FNI19-v3 (VH: SEQ ID NO.:203; VL: SEQ ID NO.: 205) of group I (H1N1) IAV, group II (H3N2) IAV, Victoria-lineage IBV, and Yamagata-lineage IBV NAs as measured by ViroSpot microneutralization assay. Rectangles indicate NAs harboring glycosylation at position 245. Neutralization by a comparator antibody, FM08_LS, was also measured. Neutralization is reported as IC50 (in μg/ml).

FIG. 61 shows antibody activation of FcγRIIIa (F158 allele) and FcγRIIa (H131 allele) by “GAALIE” Fc variant antibodies (comprising G236A/A330L/I332E mutations in the Fc). Activation was measured using an NFAT-mediated luciferase reporter in engineered Jurkat cells. Activation was assessed following incubation with A549 cells infected with H1N1 influenza strain A/Puerto Rico/8/34 at a multiplicity of infection (MOI) of 6. FNI3, FNI9, FNI17, and FNI19 were tested, along with FNI3, FNI9, FNI17, and FNI19 antibodies bearing GAALIE mutations (suffix “-GAALIE”). A comparator antibody “FM08_LS” and a negative control antibody (FY1-GRLR) were also tested.

FIG. 62 shows the design of an inter-experiment in vivo study to compare prophylactic activity of FM08_LS with FNI3_LS and FNI9_LS in BALB/c mice infected with IAV A/Puerto Rico/8/34 or A/Hong Kong/8/68. The table shows dosing and virus strains used in the study. The timeline and endpoints of the study are the same as those shown in FIG. 28B. Body weight data from Experiment A (“Exp-A”) are shown in FIGS. 29A-29D (FNI3-LS, A/Puerto Rico/8/34), FIGS. 30A-30D (FNI9-LS, A/Puerto Rico/8/34), FIGS. 31A-31D (FNI3-LS, A/Hong Kong/8/68), and FIGS. 32A-32D (FNI9-LS, A/Hong Kong/8/68). Body weight data from Experiment B (“Exp-B”) are shown in FIGS. 63A-63D (FM08_LS, A/Puerto Rico/8/34) and FIGS. 64A-64D (FM08_LS, A/Hong Kong/8/68).

FIGS. 63A-63D show measurements of body weight over fifteen days in BALB/c mice infected with H1N1 A/Puerto Rico/8/34 following pre-treatment with FM08_LS. Antibody was administered at 6 mg/kg (FIG. 63A), 2 mg/kg (FIG. 63B), 0.6 mg/kg (FIG. 63C), or 0.2 mg/kg (FIG. 63D), one day prior to infection with a LD90 (90% lethal dose) of A/Puerto Rico/8/34. Body weight of mice administered a vehicle control was also measured (left graph in each figure).

FIGS. 64A-64D show measurements of body weight over fifteen days in BALB/c mice infected with H3N2 A/Hong Kong/8/68 following pre-treatment with FM08_LS. Antibody was administered at 6 mg/kg (FIG. 64A), 2 mg/kg (FIG. 64B), 0.6 mg/kg (FIG. 64C), or 0.2 mg/kg (FIG. 64D), one day prior to infection with a LD90 (90% lethal dose) of A/Hong Kong/8/68. Body weight of mice receiving a vehicle control was also measured (left graph in each figure).

FIG. 65 shows dosing used in the design of an in vivo study to compare prophylactic activity of FNI17-LS and FM08_LS in BALB/c mice infected with IAV A/Puerto Rico/8/34.

FIGS. 66A-66D show measurements of body weight over twelve days in BALB/c mice infected with H1N1 A/Puerto Rico/8/34 following pre-treatment with FNI17-LS or FM08_LS. Antibody was administered at 1 mg/kg (FIG. 66A), 0.5 mg/kg (FIG. 66B), 0.25 mg/kg (FIG. 66C), or 0.125 mg/kg (FIG. 66D), one day prior to infection with a LD90 (90% lethal dose) of A/Puerto Rico/8/34.

FIG. 67 shows survival over twelve days in BALB/c mice infected with H1N1 A/Puerto Rico/8/34 following treatment with FNI17-LS or FM08_LS. Survival in mice pre-treated with a vehicle control was also measured.

FIG. 68 shows the design of an in vivo study to evaluate biological potency of oseltamivir (OSE) in female BALB/c mice infected with IAV A/Puerto Rico/8/34. The timeline shows time of infection, OSE dosing, and endpoints of the study. OSE was administered at 10 mg/kg by oral gavage on Day 0 beginning at two hours prior to infection with 10-fold LD50 (50% lethal dose) of A/Puerto Rico/8/34. OSE was administered at the same dose at 6 hours post-infection and then twice daily until day 6 post-infection.

FIG. 69 shows measurements of body weight over fourteen days in BALB/c mice infected with H1N1 A/Puerto Rico/8/34 following pre-treatment with oseltamivir (OSE). Weight loss in mice pre-treated with a vehicle control (H2O) was also measured.

FIG. 70 shows survival over fourteen days in BALB/c mice infected with H1N1 A/Puerto Rico/8/34 following treatment with oseltamivir (OSE). Survival in mice pre-treated with a vehicle control (H2O) was also measured.

FIG. 71 shows viral titer in lung homogenates from BALB/c mice treated with OSE and infected with H1N1 A/Puerto Rico/8/34. Lung tissue was collected at two and four days post-infection. Titer is reported as 50/6 tissue culture infectious dose per gram tissue (TCID50/g).

FIGS. 72A-72B show acid sequences of FNI3, FNI9, FNI17, and FNI19 VH (FIG. 72A) and VK (FIG. 72B) aligned to unmutated common ancestor, “UCA”.

FIGS. 73A-73E show in vitro inhibition of sialidase activity (reported as IC50 in μg/ml) by FNI3 and eleven FNI3 variants (FNI3-v8 through FNI3-v18; see Tables 1 and 2 for amino acid and nucleic acid sequences) against group I (H1N1) IAV NAs and IBV NAs. Neutralization activity of FNI3 and FNI3 variants is shown for group I (H1N1) IAV NAI from H5N1 A/Vietnam/1203/2004 (FIG. 73A), NA2 from H3N2 A/Tanzania/205/2010 (FIG. 73B), and NA9 from H7N9 A/Hong Kong/56/2015 (FIG. 73C). Neutralization activity of FNI3 and FNI3 variants is shown for BNA2 from B/Malaysia/2506/2004 (FIG. 73D) and BNA7 from B/Perth/211/2011 (FIG. 73E).

FIGS. 74A-74E show in vitro inhibition of sialidase activity (reported as IC50 in μg/ml) by FNI9 and five FNI9 variants (FNI9-v5 through FNI9-v9; see Tables 1 and 2 for amino acid and nucleic acid sequences) against IAV NAs and IBV NAs. Neutralization activity of FNI9 and FNI9 variants is shown for group I (H1N1) IAV NA1 from H5N1 A/Vietnam/1203/2004 (FIG. 74A), NA2 from H3N2 A/Tanzania/205/2010 (FIG. 74B), and NA9 from H7N9 A/Hong Kong/56/2015 (FIG. 74C). Neutralization activity of FNI9 and variants is shown for BNA2 from B/Malaysia/2506/2004 (FIG. 74D) and BNA7 from B/Perth/211/2011 (FIG. 74E).

FIGS. 75A-75E show in vitro inhibition of sialidase activity (reported as IC50 in μg/ml) by FNI17 and eleven FNI17 variants (FNI17-v6 through FNI17-v16; see Table 2 for amino acid and nucleic acid sequences) against IAV NAs and IBV NAs. Neutralization activity of FNI17 and FNI17 variants is shown for group I (H1N1) IAV NA1 from H5N1 A/Vietnam/1203/2004 (FIG. 75A), NA2 from H3N2 A/Tanzania/205/2010 (FIG. 75B), and NA9 from H7N9 A/Hong Kong/56/2015 (FIG. 75C). Neutralization activity of FNI17 and variants is shown for BNA2 from B/Malaysia/2506/2004 (FIG. 75D) and BNA7 from B/Perth/211/2011 (FIG. 75E).

FIGS. 76A-76E show in vitro inhibition of sialidase activity (reported as IC50 in μg/ml) by FNI19 and five FNI19 variants (FNI19-v1 through FNI19-v5; see Table 2 for amino acid and nucleic acid sequences) against IAV NAs and IBV NAs. Neutralization activity of FNI19 and FNI19 variants is shown for group I (H1N1) IAV NA1 from H5N1 A/Vietnam/1203/2004 (FIG. 76A), NA2 from H3N2 A/Tanzania/205/2010 (FIG. 76B), and NA9 from H7N9 A/Hong Kong/56/2015 (FIG. 76C). Neutralization activity of FNI19 and FNI19 variants is shown for BNA2 from B/Malaysia/2506/2004 (FIG. 76D) and BNA7 from B/Perth/211/2011 (FIG. 76E).

FIGS. 77A-77D show binding of FNI3, FNI9, FNI17, and FNI19 variants to IAV NAs and IBV NAs as measured by flow cytometry. FIG. 77A shows binding to N1 from A/Stockholm/18/2007, N1 from A/California/07/2009, and N1 from A/California/07/2009 I23R/H275Y. FIG. 77B shows binding to N2 from A/South Australia/34/2019, N2 from A/Leningrad/134/17/57, and N2 from A/Washington/01/2007. FIG. 77C shows binding to N3 from A/Canada/rv504/2004, N6 from A/swine/Ontario/01911/1/99, and N7 from A/Netherlands/078/03. FIG. 77D shows binding to B/Yamanashi/166/1998 (Yamagata), B/Malaysia/2506/2004 (Victoria), and B/Lee/10/1940 (Ancestral).

FIGS. 78A-78E show additional characteristics of certain FNI antibodies. FIG. 78A shows an alignment of FNI3, FNI9, FNI17, and FNI19 VH amino acid sequences with that of the unmutated common ancestor, “UCA”, wherein the rectangles indicate positively charged Lys13 and Lys19 residues in the UCA sequence and corresponding residues at the same position in FNI3, FNI9, FNI17, and FNI19. Overall surface charge maps generated using PyMOL are shown for FNI3 (FIG. 78B), FNI9 (FIG. 78C), FNI17 (FIG. 78D), and FNI19 (FIG. 78E) along with pK values and resolution (reported in A).

FIGS. 79A-79B show pK data for FNI17-LS, FNI19-LS, FNI17-v19-LS, and FNI19-v3-LS in tg32 mice. Mice were intravenously injected with 5 mg/kg antibody. The table in FIG. 79A shows inter-experiment values for half-life, area-under-the-curve (AUC), steady state clearance (CLss), and total volume analyzed (Volume) for FNI17-LS and FNI19-LS (Experiment 1 “PK1”), and FNI17-v19-LS and FNI19-v3-LS (Experiment 2 “PK2”). FIG. 79B shows average half-life (reported in days) plus standard error for FNI17-LS, FNI19-LS, FNI17-v19-LS, and FNI19-v3-LS.

FIG. 80 shows the design of an in vivo study to evaluate prophylactic activity of FNI17-v19-rIgG1-LS compared with oseltamivir (OSE) in BALB/c mice infected with IAVs or IBVs. Mice were pre-administered FNI17-v19-rIgG1-LS (9, 3, 0.9, or 0.3 MPK) 24 hours prior to infection at LD90 (90% lethal dose). OSE was orally administered daily at 10 mg/kg from 2 hours before infection to 3 or 4 days post-challenge. Mice were administered IAVs (H1N1 A/Puerto Rico/8/34 or H3N2 A/Hong Kong/8/68) or IBVs (B/Victoria/504/2000 (Yamagata) or B/Brisbane/60/2008 (Victoria)). A version of FNI17-v19 containing a Fc mutation that abrogates binding by FcγRs and complement (FNI17-v19-rIgG1-GRLR) was also tested in groups receiving IAV viruses (H1N1 A/Puerto Rico/8/34 or H3N2 A/Hong Kong/8/68). Lung plaque forming units (PFU) were evaluated in mice euthanized at 3 days post-infection.

FIGS. 81A-81D show lung viral titres in BALB/c mice euthanized at 3 days post-infection from the in vivo study described in FIG. 80. Lung viral titers following infection with H1N1 A/Puerto Rico/8/34 (FIG. 81A) or H3N2 A/Hong Kong/8/68 (FIG. 81B) and IBVs B/Victoria/504/2000 (Yamagata; FIG. 81C) or B/Brisbane/60/2008 (Victoria; FIG. 81D) are shown.

FIG. 82 shows the design of an in vivo study to evaluate prophylactic activity of FNI17-v19 in humanized FcγR mice infected with H1N1 A/Puerto Rico/8/34. Mice were pre-administered antibody 24 hours prior to infection at 5LD50 (five times 50% lethal dose).

FIGS. 83A-83C show measurements of body weight over fourteen days in humanized FcgR mice infected with H1N1 A/Puerto Rico/8/34 following pre-treatment with FNI17-v19. Antibody was administered at 0.9 mg/kg (FIG. 83A), 0.3 mg/kg (FIG. 83B), or 0.09 mg/kg (FIG. 83C), one day prior to infection with 5LD50 of A/Puerto Rico/8/34. Body weight of mice administered a vehicle control was also measured (left graph in each figure).

FIG. 84 shows the pre-infection concentration of human IgG in sera from humanized FcγR mice pre-treated with FNI17-v19 from the study described in FIG. 82. Sera was collected from mice 2 hours prior to infection with 5LD50 H1N1 A/Puerto Rico/8/34.

FIG. 85 shows binding energy between FNI antibodies FNI3, FNI9, FNI17, and FNI19 with highly conserved residues on NA that are involved with interacting with sialic acid.

FIG. 86 shows binding of FNI3, FNI9, FNI17, and FNI19 to NA expressed on mammalian cells infected with a H1N1 Swine Eurasian avian-like (EA) strain, A/Swine/Jiangsu/J004/2018, measured by flow cytometry. Mock antibody staining is shown as a negative control.

FIGS. 87A-87D show in vitro inhibition of sialidase activity (reported as IC50 in nM) by FNI17-v19 or OSE against group II H7N3 A/chicken/Jalisco/PAVX17170/2017 IAV (FIG. 87A), group II H5N6 A/chicken/Suzhou/j6/2019 IAV (FIG. 87B), group II H7N7 A/chicken/Netherlands/621572/03 IAV (FIG. 87C), and group I H5N8 A/chicken/Russia/3-29/2020 IAV (FIG. 87D) NAs.

FIG. 88 shows binding kinetics of FNI3, FNI9, and FNI17 to N9 NA, as measured by Bio-Layer Interferometry (BLI). KD was calculated from the ratio of kdis/kon, wherein kdis is dissociation calculated as (1/s) and kon is association calculated as (1/Ms).

FIG. 89 shows in vitro inhibition of sialidase activity (reported in ng/ml) by FNI3, FNI9, FNI17, FNI17-v19, FNI19, and FNI19-v3 against group II H7N9 A/Anhui/1/2013 IAV NA.

FIG. 90 shows antibody activation of FcγRIIIa (V158 allele) following incubation with group II H7N9 A/Anhui/1/2013 IAV. Activation was measured using an NFAT-mediated luciferase reporter in engineered Jurkat cells following incubation with Expi-CHO cells transiently transfected with plasmids encoding N9 from A/Anhui/1/2013 IAV. FNI3, FNI9, FNI17, and FNI19 were tested, along with a negative control antibody (FY1-GRLR).

FIGS. 91A-91B show prevalence of OSE-resistant mutations within the FNI NA binding site in group I H1N1 IAVs (FIG. 91A) and group II H3N2 IAVs (FIG. 91B) from 2007 to 2019.

FIGS. 92A-92B show in vitro neutralizing activity by FNI17, FNI19, and Oseltamivir (OSE) against group I H1N1 IAV strains (FIG. 92A) and group II H3N2 IAV strains (FIG. 92B) optionally, bearing one or more OSE-resistant mutations. FIG. 92A shows activity against A/Puerto Rico/8/34 (“PR8” in the figure) and A/California/07/2009 (“Cal/09” in the figure), as well as A/California/07/2009 engineered with reverse genetics to harbor OSE-resistant mutations H275Y, E119D, or both S247N and H275Y. FIG. 92B shows activity against A/Hong Kong/8/68 (“HK/68” in the figure) and A/Hong Kong/8/68 engineered with reverse genetics to harbor OSE-resistant mutations I222V or N294S.

FIG. 93 shows binding of FNI17-v19 to NAs from N1_Vic_2019, N2_HK_2019, B/Phuket/3073/2013 (Yamagata) (“B/Phuket_2013(Yam)” in the figure), B/Malaysia/2506/2004 (Victoria) (“B/Malaysia_2004(Vic)” in the figure), and B/Washington/02/2019 (Victoria) (“B/Wash_2019(Vic)” in the figure) as measured by flow cytometry and reported in mean fluorescence intensity (MFI). Cells were mock-stained as a negative control.

FIGS. 94A-94B show viral titer in lung homogenates from BALB/c mice treated with varying doses of FNI17 or OSE and infected with H1N1 A/Puerto Rico/8/34 (FIG. 94A) or H3N2 A/Hong Kong/8/68 (FIG. 94B). Lung tissue was collected at four (FIG. 94A) or three days (FIG. 94B) post-infection. Titer is reported as log 50% tissue culture infectious dose per gram tissue (Log TCID50/g) in FIG. 94A. Titer is reported as log plaque-forming units per gram tissue (Log pfu/g) in FIG. 94B. In FIGS. 94A and 94B, the left-to-right arrangement of dot plots in the graph corresponds to the top-to-bottom orientation in the figure key. For example, Vehicle is the left-most cluster of dots in the graph, and OSE is the right-most cluster of dots in the graph.

FIGS. 95A-95B show body weight loss from day 0 to 14 post-infection (reported as negative area-under-the-curve peak values) from area-under-the-curve analysis of body weight loss in BALB/c mice infected with H1N1 A/Puerto Rico/8/34 (FIG. 95A) or H3N2A/Hong Kong/8/68 (FIG. 95B) following treatment with FNI17 or OSE at the indicated dose. In FIGS. 95A and 95B, the left-to-right arrangement of dot plots and bars in the graph corresponds to the top-to-bottom orientation in the figure key. For example, Vehicle is the left-most cluster of dots (and accompanying bar) in the graph, and OSE is the right-most cluster of dots (and accompanying bar) in the graph.

FIGS. 96A-96B show negative area-under-the-curve peak values compared with IgG in serum from area-under-the-curve analysis of body weight loss in BALB/c mice infected with H1N1 A/Puerto Rico/8/34 (FIG. 96A) or H3N2 A/Hong Kong/8/68 (FIG. 96B) following treatment with FNI17 or OSE. IC50, IC70, and IC90 are reported in μg/ml.

FIGS. 97A-97B show oxygen saturation in the blood as measured by pulse oximetry for BALB/c mice infected with H1N1 A/Puerto Rico/8/34 (FIG. 97A) or H3N2A/Hong Kong/8/68 (FIG. 97B) following treatment with FNI17 or OSE (reported in peripheral capillary oxygen saturation (SpO₂)). In FIGS. 97A and 97B, the left-to-right arrangement of each group of five bars (and related dot plot clusters) in the graph corresponds to the top-to-bottom orientation in the figure key. For example, Vehicle is the left-most bar in each set of bars, and OSE is the right-most bar in each set of bars.

FIGS. 98A-98B show correlation between oxygen saturation (at Day 8 post-infection) and viral lung titer (at Day 4 post-infection), in BALB/c mice infected with H1N1 A/Puerto Rico/8/34 (FIG. 98A) or H3N2 A/Hong Kong/8/68 (FIG. 98B) following treatment with FNI17. Pearson coefficients were calculated to quantify correlation.

FIGS. 99A-99C show in vivo pharmacokinetics of FNI17-v19 and FNI19-v3 for three individual mice (“1501”-“1503”; “2501”-“2503”). Data for individual mice over a span of 1500 hours is shown for FNI17-v19 (FIG. 99A) and FNI19v3 (FIG. 99B) treatment groups, and combined in FIG. 99C over 64 days.

FIG. 100 summarizes in vivo pharmacokinetic properties of FNI17-v19 and FNI19-v3 as evaluated in mice. FM08_LS is shown as a comparator antibody.

FIGS. 101A-101B show lack of off-target binding by FNI17-v19 (FIG. 101A) and FNI19-v3 (FIG. 101B), as measured using an array of 6,000 human membrane proteins.

FIG. 102 shows lack of specific positive staining by FNI17-v19 and FNI19-v3 in human tissues as measured using non-Good Laboratory Practice Tissue Cross Reactivity Testing (Non-GLP-TCR). IgG was tested to assess background staining.

FIG. 103A-103C show antibody activation of FcγRIIa (H131 allele) by “GAALIE” Fc variant antibodies (comprising G236A/A330L/I332E mutations in the Fc). Activation was measured using an NFAT-mediated luciferase reporter in engineered Jurkat cells following incubation with Expi-CHO cells transiently transfected with plasmids encoding different IAV (H1N1 A/California/07/2009 in FIG. 103A; H3N2 A/Hong Kong/8/68 in FIG. 103B) and IBV (B/Malaysia/2506/2004 in FIG. 103C) NAs. FNI3, FNI9, FNI17, and FNI19 were tested, along with FNI3, FNI9, FNI17, and FNI19 antibodies bearing GAALIE mutations (suffix “-GAALIE” in the figure). A comparator antibody “FM08_LS” and a negative control antibody (FY1-GRLR) were also tested. FM08_LS and FY1-GRLR had the lowest measured values in FIGS. 103A-103C.

FIG. 104 shows in vitro inhibition of sialidase activity by FNI17-v19 of group I (H1N1) IAV, group II (H3N2) IAV, Victoria-lineage IBV, and Yamagata-lineage IBV NAs as measured by ViroSpot microneutralization assay.

FIG. 105 shows in vitro inhibition of sialidase activity by FNI17-v19 of group I (H1N1) IAV, group II (H3N2) IAV, Victoria-lineage IBV, and Yamagata-lineage IBV 15 NAs as measured by ViroSpot microneutralization assay. B/Brisbane/2008 is highlighted by a rectangle.

FIGS. 106A-106B shows viral titer in lung homogenates from BALB/c mice treated with varying doses of FNI17 or OSE and infected with H3N2 A/Hong Kong/8/68 (FIG. 106A) or H1N1 A/Puerto Rico/8/34 (FIG. 106B). Lung tissue was collected at three (FIG. 106A) or four days (FIG. 106B) post-infection. Titer is reported as log plaque-forming units per gram tissue (Log pfu/g) in FIG. 106A. Titer is reported as log 50% tissue culture infectious dose per gram tissue (Log TCID50/g) in FIG. 106B. Ovals highlight FNI17 dose (mg/kg) capable of producing same viral lung reduction as OSE. In FIGS. 106A and 106B, the left-to-right arrangement of dot plot clusters in the graph corresponds to the top-to-bottom orientation in the figure key. For example, Vehicle is the left-most cluster of dots in the graph, and OSE is the right-most cluster of dots in the graph.

FIG. 107 shows “% Protection” compared with IgG in serum in BALB/c mice infected with influenza and treated with FNI17 or OSE. IC50, IC70, and IC90 are reported in μg/ml.

FIG. 108 shows body weight loss from day 0 to 14 post-infection (reported as negative area-under-the-curve peak values) in mice infected with H1N1 A/Puerto Rico/8/3 following pre-treatment with FNI17 or FM08_LS. Body weight loss in mice pre-treated with a vehicle control was also measured. For the 1 mg/kg dose (left-most set of three bars), the left-to-right order of the bars corresponds to the top-to-bottom orientation in the figure key (i.e., Vehicle is the left-most bar in the 1 mg/kg quadrant; FM08_LS is right-most bar). At the other doses, the left bar represents FNI17 and the right bar represents FM08_LS.

FIG. 109 shows survival over thirteen days in BALB/c mice infected with H1N1 A/Puerto Rico/8/34 following treatment with FNI17 or FM08_LS. Survival in mice pre-treated with a vehicle control (shortest survival curve) was also measured.

FIG. 110 shows antibody titers of certain FNI3, FNI9, FNI17, or FNI19 mAbs, including gain/loss for variants as compared to wild-type.

FIG. 111 shows binding to group I IAV, group II IAV, and IBV NAs as measured by flow cytometry (reported as MFI) for FNI3 and 11 FNI3 variants (FNI3-v8 to FNI3-v18). MFI values for variants were normalized to MFI values for wild-type FNI3.

FIG. 112 shows binding to group I IAV, group II IAV, and IBV NAs as measured by flow cytometry (reported as MFI) for FNI9 and five FNI9 variants (FNI9-v5 to FNI9-v9). MFI values for variants were normalized to MFI values for wild-type FNI9.

FIG. 113 shows binding to group I IAV, group II IAV, and IBV NAs as measured by flow cytometry (reported as MFI) for FNI17 and 11 FNI17 variants (FNI17-v6 to FNI17-v16). MFI values for variants were normalized to MFI values for wild-type FNI17.

FIG. 114 shows binding to group I IAV, group II IAV, and IBV NAs as measured by flow cytometry (reported as MFI) for FNI19 and five FNI19 variants (FNI19-v1 to FNI19-v5). MFI values for variants were normalized to MFI values for wild-type FNI19.

FIGS. 115A-115D show binding kinetics of FNI3-LS, FNI9-LS, FNI17-LS, and FNI19-LS, along with FNI3-LS, FNI9-LS, FNI17-LS, and FNI19-LS antibodies bearing GAALIE mutations (suffix “-GAALIE” in the figure) to different FcγRs, as measured by Bio-Layer Interferometry (BLI). Arrows indicate curves for FNI17-LS and FNI17-LS-GAALIE. FIG. 115A shows binding to FcγRIIA(H), FIG. 115B shows binding to FcγRIIA(R), FIG. 115C shows binding to FcγRIIIA(F), and FIG. 115D shows binding to FcγRIIIA(V).

### DETAILED DESCRIPTION

Provided herein are antibodies and antigen-binding fragments that can bind to and potently neutralize infection by various influenza viruses, such as influenza A viruses (IAVs) and influenza B viruses (IBVs). Also provided are polynucleotides that encode the antibodies and antigen-binding fragments, vectors, host cells, and related compositions, as well as methods of using the antibodies, nucleic acids, vectors, host cells, and related compositions to treat (e.g., reduce, delay, eliminate, or prevent) an influenza virus infection in a subject and/or in the manufacture of a medicament for treating an influenza infection in a subject.

As taught in the present examples, a number of clonally related antibodies were identified that bind to a breadth of IAV and IBV NAs, and have neutralizing/inhibitory functions against IAV and IBV viruses. Sequence variants of the antibodies were generated and characterized. Certain disclosed embodiments relate to such antibodies, antigen-binding fragments of the same, and related compositions and uses.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA, also referred to as deoxyribonucleic acid), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine. These features are known in the art and are discussed in, for example, Zhang et al. Front. Immunol., DOI=10.3389/fimmu.2019.00594 (2019); Eyler et al. PNAS 116(46): 23068-23071; DOI: 10.1073/pnas.1821754116 (2019); Nance and Meier, ACS Cent. Sci. 2021, 7, 5, 748-756; doi.org/10.1021/acscentsci.1c00197 (2021), and van Hoecke and Roose, J. Translational Med 17:54 (2019); https://doi.org/10.1186/s12967-019-1804-8, which modified nucleosides and mRNA features are incorporated herein by reference. Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, antigen-binding fragment, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurs et al., Mol. Ther. 8:108, 2003: Mites et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Method Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of an influenza infection, a “host” refers to a cell or a subject infected with the influenza.

“Antigen” or “Ag”, as used herein, refers to an immunogenic molecule that provokes an immune response. This immune response may involve antibody production, activation of specific immunologically-competent cells, activation of complement, antibody dependent cytotoxicicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in an influenza NA antigen, such as present in a virion, or expressed or presented on the surface of a cell infected by the influenza.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, and Compositions

In one aspect, the present disclosure provides an isolated an antibody, or an antigen-binding fragment thereof, that is capable of binding to a neuraminidase (NA) from: (i) an influenza A virus (IAV), wherein the IAV comprises a Group 1 IAV, a Group 2 IAV, or both; and (ii) an influenza B virus (IBV).

In certain embodiments, an antibody or antigen-binding fragment of the present disclosure associates with or unites with a NA while not significantly associating or uniting with any other molecules or components in a sample.

In certain embodiments, an antibody or antigen-binding fragment of the present disclosure specifically binds to a IAV NA. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a K. of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, biolayer interferometry, and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain examples, binding can be determined by recombinantly expressing a influenza NA antigen in a host cell (e.g., by transfection) and immunostaining the (e.g., fixed, or fixed and permeabilized) host cell with antibody and analyzing binding by flow cytometery (e.g., using a ZE5 Cell Analyzer (BioRad®) and FlowJo software (TreeStar). In some embodiments, positive binding can be defined by differential staining by antibody of influenza NA-expressing cells versus control (e.g., mock) cells.

In some embodiments an antibody or antigen-binding fragment of the present disclosure binds to an influenza NA protein, as measured using biolayer interferometry, or by surface plasmon resonance.

Certain characteristics of presently disclosed antibodies or antigen-binding fragments may be described using IC50 or EC50 values. In certain embodiments, the IC50 is the concentration of a composition (e.g., antibody) that results in half-maximal inhibition of the indicated biological or biochemical function, activity, or response. In certain embodiments, the EC50 is the concentration of a composition that provides the half-maximal response in the assay. In some embodiments, e.g., for describing the ability of a presently disclosed antibody or antigen-binding fragment to neutralize infection by influenza, IC50 and EC50 are used interchangeably.

In certain embodiments, an antibody of the present disclosure is capable of neutralizing infection by influenza. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment can be capable of preventing and/or neutralizing an influenza infection in an in vitro model of infection and/or in an in vivo animal model of infection and/or in a human.

In certain embodiments, the antibody, or antigen-binding fragment thereof, is human, humanized, or chimeric.

In certain embodiments, (i) the Group 1 IAV NA comprises a N1, a N4, a N5, and/or a N8; and/or (ii) the Group 2 IAV NA comprises a N2, a N3, a N6, a N7, and/or a N9. In some embodiments; (i) the N1 is a N1 from any one or more of: A/California/07/2009, A/California/07/2009 I223R/H275Y, A/Swine/Jiangsu/J004/2018, A/Stockholm/18/2007, A/Brisbane/02/2018, A/Michigan/45/2015, A/Mississippi/3/2001, A/Netherlands/603/2009, A/Netherlands/602/2009, A/Vietnam/1203/2004, A/G4/SW/Shangdong/1207/2016, A/G4/SW/Henan/SN13/2018, and A/New Jersey/8/1976; (ii) the N4 is from A/mallard duck/Netherlands/30/2011; (iii) the N5 is from A/aquatic bird/Korea/CN5/2009; (iv) the N8 is from A/harbor seal/New Hampshire/179629/2011; (v) the N2 is a N2 from any one or more of: A/Washington/01/2007, A/HongKong/68, A/South Australia/34/2019, A/Switzerland/8060/2017, A/Singapore/INFIMIH-16-0019/2016, A/Switzerland/9715293/2013, A/Leningrad/134/17/57, A/Florida/4/2006, A/Netherlands/823/1992, A/Norway/466/2014, A/Switzerland/8060/2017, A/Texas/50/2012, A/Victoria/361/2011, A/HongKong/2671/2019, A/SW/Mexico/SG1444/2011, A/Tanzania/205/2010, A/Aichi/2/1968, A/Bilthoven/21793/1972, A/Netherlands/233/1982, A/Shanghai/11/1987, A/Nanchang/933/1995, A/Fukui/45/2004, and A/Brisbane/10/2007 (vi) the N3 is from A/Canada/rv504/2004; (v) the N6 is from A/swine/Ontario/01911/1/99; (vi) the N7 is 20 from A/Netherlands/078/03; and/or (vii) the N9 is a N9 from any one or more of: A/Anhui/2013 and A/Hong Kong/56/2015. In certain embodiments, the IBV NA is a NA from any one or more of: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008; B/Colorado/06/2017; B/Hubei-wujiang/158/2009; B/Massachusetts/02/2012; B/Netherlands/234/2011; B/Perth/211/2001; B/Phuket/3073/2013; B/Texas/06/2011 (Yamagata); B/Perth/211/2011; B/HongKong/05/1972; B/Harbin/7/1994 (Victoria); and B/Washington/02/2019 (Victoria).

In certain embodiments, the antibody or antigen-binding fragment is capable of binding to each of: (i) a Group 1 IAV NA; (ii) a Group 2 IAV NA; and (iii) a IBV NA with an EC₅₀ in a range of from about 0.1 μg/mL to about 50 μg/mL, or in a range of from about 0.1 μg/mL to about 2 μg/mL, or in a range of from 0.1 μg/mL to about 10 μg/mL, or in a range of from 2 μg/mL to about 10 μg/mL, or in a range of from about 0.4 μg/mL to about 50 μg/mL, or in a range of from about 0.4 μg/mL to about 2 μg/mL, or in a range of from 0.4 μg/mL to about 10 μg/mL, or in a range of from 2 μg/mL to about 10 μg/mL, or in a range of from 0.4 μg/mL to about 1 μg/mL, or 0.4 μg/mL or less.

In certain embodiments, the antibody or antigen-binding fragment is capable of binding to: (i) the Group 1 IAV NA with an EC₅₀ in a range of: from about 0.4 μg/mL to about 50 μg/mL, from about 0.4 μg/mL to about 10 μg/mL, from about 0.4 μg/mL to about 2 μg/mL, from about 2 μg/mL to about 50 μg/mL, from about 2 μg/mL to about 10 μg/mL, or from about 10 μg/mL to about 50 μg/mL; (ii) the Group 2 IAV NA with an EC₅₀ in a range from about 0.4 μg/mL to about 50 μg/mL, or from about 0.4 μg/mL to about 10 μg/mL, or from about 0.4 μg/mL to about 2 μg/mL, or from about 2 μg/mL to about 50 μg/mL, or from about 2 μg/mL to about 10 μg/mL, or from about 10 μg/mL to about 50 μg/mL; and/or (iii) the IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL. In further embodiments, the antibody or antigen-binding fragment is capable of binding to: (i) a N1 with an EC₅₀ of about 0.4 μg/mL, or in a range from about 0.4 μg/mL to about 50 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (ii) a N4 with an EC₅₀ of about 0.4 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (iii) a N5 with an EC₅₀ in a range of: from about 0.4 μg/mL to about 2 μg/mL; (iv) a N8 with an EC₅₀ of about 50 μg/mL; (v) a N2 with an EC₅₀ in a range of: from about 0.4 μg/mL to about 20 μg/mL, or from about 0.4 μg/mL to about 10 μg/mL, or from about 0.4 μg/mL to about 2 μg/mL, from about 1 μg/mL to about 10 μg/mL, or from about 1 μg/mL to about 20 μg/mL, or from about 1 μg/mL to about 5 μg/mL, or about 1, 2, 3, 4, 5, 6, 7, 8, 9, 10, 15, or 20 μg/mL; (vi) a N3 with an EC₅₀ of about 0.4 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (vii) a N6 with an EC₅₀ of about 0.4 μg/mL, or in a range of from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (viii) a N7 with an EC₅₀ in a range of: from about 2 μg/mL to about 50 μg/mL; (ix) a N9 with an EC₅₀ of about 0.4 μg/mL, or in a range of: from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; and/or (xi) a IBV NA with an EC₅₀ of about 0.4 μg/mL, or in a range of: from about 0.1 μg/mL to about 1.9 μg/mL, or from about 0.1 g/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL.

In certain embodiments, the antibody or antigen-binding fragment is capable of binding to: (i) one or more of: N1 A/California/07/2009, N1 A/California/07/2009 I223R/H275Y, N1 A/Swine/Jiangsu/J004/2008, N1 A/Stockholm/18/2007, N4 A/mallard duck/Netherlands/30/2011, N5 A/aquatic bird/Korea/CN5/2009, N2 A/Hong Kong/68, N2 A/Leningrad/134/17/57, N3 A/Canada/rv504/2004, N6 A/Swine/Ontario/01911/1/99, N9 A/Anhui/1/2013, B/Lee/10/1940 (Ancestral), B/Brisbane/60/2008 (Victoria), B/Malaysia/2506/2004 (Victoria), B/Malaysia/3120318925/2013 (Yamagata), B/Wisconsin/1/2010 (Yamagata), and B/Yamanashi/166/1998 (Yamagata), with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (ii) N5 A/aquatic bird/Korea/CN5/2009 with an EC₅₀ of about 2 μg/mL, or in a range from about 2 μg/mL to about 10 μg/mL; (iii) N8 A/harbor seal/New Hampshire/179629/2011 with an EC₅₀ of about 50 μg/mL; (iv) N2 A/Washington/01/2007 with an EC₅₀ in a range from about 2 μg/mL to about 10 μg/mL; (v) N7 A/Netherlands/078/03 with an EC₅₀ in a range from about 2 μg/mL to about 50 μg/mL; (vi) N2 A/South Australia/34/2019 with an EC₅₀ in a range from about 0.4 μg/mL to about 50 μg/mL; (vii) N2 A/Switzerland/8060/2017 with an EC₅₀ in a range from about 9.5 μg/mL to about 3.8 μg/mL; (viii) N2 A/Singapore/INFIMH-16-0019/2016 with an EC₅₀ in a range from about 18.4 μg/mL to about 2.2 μg/mL; (iv)

N2 A/Switzerland/9715293/2013 with an EC₅₀ in a range from about 1.6 μg/mL to about 1.2 μg/mL; and/or (v) N1 A/Swine/Jiangsu/J004/2018 with an EC₅₀ in a range from about 0.4 μg/mL to about 50 μg/m L, or about 0.4, about 2, about 10, or about 50 μg/mL.

In certain embodiments, wherein the NA is expressed on the surface of a host cell (e.g., a CHO cell) and binding to NA is according to flow cytometry.

In certain embodiments, the antibody or antigen-binding fragment is capable of binding to the NA with a KD of less than 1.0E-12 M, less than 1.0E-11 M, less than 1.0 E-11 M, or of 1.0E-12M or less, 1.0E-11M or less, or 1.0E-10 or less, or with a K_{D} between 1.0E-10 and 1.0E-13, or with a KD between 1.0E-11 and 1.0E-13, wherein, optionally, the binding is as assessed by biolayer interferometry (BLI).

In certain embodiments, the NA is a N1, a N2, and/or a N9.

In certain embodiments, the antibody or antigen-binding fragment is capable of binding to: (1) a NA epitope that comprises any one or more of the following amino acids (N1 NA numbering): R368, R293, E228, E344, S247, D198, D151, R118; and/or (2) a NA epitope that comprises any one or more of the following amino acids (N2 NA numbering): R371, R292, E227, E344, S247, D198, D151, R118. It will be understood that the antibodies and antigen-binding fragments may also bind to influenza neuraminidases which may not follow N1 or N2 amino acid numbering conventions; amino acids of these epitopes may correspond to herein-indicated N1 or N2 amino acid residues, such as by being the same amino acid residue at an equivalent (e.g., by alignment, 3-D structure, conservation, or combinations of these) but differently numbered, position in the NA. Accordingly, reference to N1 or N2 numbering will be understood as the amino acid corresponding to the enumerated amino acid.

An example showing N1 vs N2 position numbering (using H1N1_California.07.2009 and H3N2_NewYork.392.2004) is provided in Table 3.

In certain embodiments, the antibody or antigen-binding fragment is capable of binding to: (1) a NA epitope that comprises the amino acids R368, R293, E228, D151, and R118 (N1 NA numbering); and/or (2) a NA epitope that comprises the amino acids R371, R292, E227, D151, and R118 (N2 NA numbering).

In certain embodiments, the antibody or antigen-binding fragment is capable of binding to an epitope comprised in or comprising a NA active site (as described herein, the NA active site comprises functional amino acids that form the catalytic core and directly contact sialic acid, as well as structural amino acids that form the active site framework), wherein, optionally, the NA active site comprises the following amino acids (N2 numbering): R118, D151, R152, R224, E276, R292, R371, Y406, E119, R156, W178, S179, D/N198, I222, E227, H274, E277, D293, E425. In certain embodiments, R118, D151, R152, R224, E276, R292, R371, and Y406 form the catalytic core and directly contact sialic acid. In certain embodiments, E119, R156, W178, S179, D/N198, I222, E227, H274, E277, D293, and E425 form the active site framework.

In certain embodiments, the epitope comprises or further comprises any one or more of the following NA amino acids (N2 numbering): E344, E227, S247, and D198.

In certain embodiments, the antibody or antigen-binding fragment is capable of binding to a NA comprising a S245N amino acid mutation and/or a E221D amino acid mutation (N2 numbering).

In certain embodiments, the NA comprises an IBV NA. In certain embodiments, the antibody or antigen-binding fragment is capable of binding to an IBV NA epitope that comprises any one or more of the following amino acids (IBV numbering; e.g., as for FluB Victoria and FluB Yamagata): R116, D149, E226, R292, and R374. In some embodiments, the epitope comprises the amino acids R116, D149, E226, R292, and R374.

In certain embodiments, the antibody or antigen-binding fragment is capable of inhibiting a sialidase activity of (i) an IAV NA, wherein the IAV NA comprises a Group 1 IAV NA, a Group 2 IAV NA, or both, and/or of (ii) an IBV NA, in an in vitro model of infection, an in vivo animal model of infection, and/or in a human. In further embodiments: (i) the Group 1 IAV NA comprises a H1N1 and/or a H5N1; (ii) the Group 2 IAV NA comprises a H3N2 and/or a H7N9; and/or (iii) the IBV NA comprises one or more of: B/Lee/10/1940 (Ancestral); B/HongKong/05/1972; B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006(Yamagata); B/Jiangsu/10/2003 (Yamagata); B/Texas/06/2011 (Yamagata); B/Perth/211/2011; B/Harbin/7/1994 (Victoria); B/Colorado/06/2017 (Victoria); B/Washington/02/2019 (Victoria); B/Perth/211/2001 (Yamagata); B/Hubei-wujiagang/158/2009 (Yamagata); B/Wisconsin/01/2010 (Yamagata); B/Massachusetts/02/2012 (Yamagata); and B/Phuket/3073/2013 (Yamagata).

In certain embodiments, the antibody or antigen-binding fragment is capable of inhibiting a sialidase activity by: a Group 1 IAV NA; a Group 2 IAV NA; and/or a IBV NA, with an IC50 in a range of: from about 0.0008 μg/mL to about 4 μg/mL, from about 0.0008 μg/mL to about 3 μg/mL, from about 0.0008 μg/mL to about 2 μg/mL, from about 0.0008 μg/mL to about 1 μg/mL, from about 0.0008 μg/mL to about 0.9 μg/mL, from about 0.0008 μg/mL to about 0.8 μg/mL, from about 0.0008 μg/mL to about 0.7 μg/mL, from about 0.0008 μg/mL to about 0.6 μg/mL, from about 0.0008 μg/mL to about 0.5 μg/mL, from about 0.0008 μg/mL to about 0.4 μg/mL, from about 0.0008 μg/mL to about 0.3 μg/mL, from about 0.0008 μg/mL to about 0.2 μg/mL, from about 0.0008 μg/mL to about 0.1 μg/mL, from about 0.0008 μg/mL to about 0.09 μg/mL, from about 0.0008 μg/mL to about 0.08 μg/mL, from about 0.0008 μg/mL to about 0.07 μg/mL, from about 0.0008 μg/mL to about 0.06 μg/mL, about 0.0008 μg/mL to about 0.05 μg/mL, about 0.0008 μg/mL to about 0.04 μg/mL, about 0.0008 μg/mL to about 0.03 μg/mL, about 0.0008 μg/mL to about 0.02 μg/mL, about 0.0008 μg/mL to about 0.01 μg/mL, from 0.002 μg/mL to about 4 μg/mL, from about 0.001 μg/mL to 50 μg/mL, from about 0.1 μg/mL to about 30 μg/mL, from about 0.1 μg/mL to about 20 μg/mL, from about 0.1 μg/mL to about 10 μg/mL, from about 0.1 μg/mL to about 9 μg/mL, from about 0.1 μg/mL to about 8 μg/mL, from about 0.1 μg/mL to about 7 μg/mL, from about 0.1 μg/mL to about 6 μg/mL, from about 0.1 μg/mL to about 5 μg/mL, from about 0.1 μg/mL to about 4 μg/mL, from about 0.1 μg/mL to about 3 μg/mL, from about 0.1 μg/mL to about 2 μg/mL, from about 0.1 μg/mL to about 1 μg/mL, from about 0.1 μg/mL to about 0.9 μg/mL, from about 0.1 μg/mL to about 0.8 μg/mL, from about 0.1 μg/mL to about 0.7 μg/mL, from about 0.1 μg/mL to about 0.6 μg/mL, from about 0.1 μg/mL to about 0.5 μg/mL, from about 0.1 μg/mL to about 0.4 μg/mL, from about 0.1 μg/mL to about 0.3 μg/mL, from about 0.1 μg/mL to about 0.2 μg/mL, from about 0.8 μg/mL to about 30 μg/mL, from about 0.8 μg/mL to about 20 μg/mL, from about 0.8 μg/mL to about 10 μg/mL, from about 0.8 μg/mL to about 9 μg/mL, from about 0.8 μg/mL to about 8 μg/mL, from about 0.8 μg/mL to about 7 μg/mL, from about 0.8 μg/mL to about 6 μg/mL, from about 0.8 μg/mL to about 5 μg/mL, from about 0.8 μg/mL to about 4 μg/mL, from about 0.8 μg/mL to about 3 μg/mL, from about 0.8 μg/mL to about 2 μg/mL, of from about 0.8 μg/mL to about 1 μg/mL, or of about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1.0 μg/mL, about 1.5 μg/mL, about 2.0 μg/mL, about 2.5 μg/mL, about 3.0 μg/mL, about 3.5 μg/mL, about 4.0 μg/mL, about 4.5 μg/mL, about 5.0 μg/mL, about 5.5 μg/mL, about 6.0 μg/mL, about 6.5 μg/mL, about 7.0 μg/mL, about 7.5 μg/mL, about 8.0 μg/mL, about 8.5 μg/mL, about 9.0 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 25 μg/mL, and/or about 30 μg/mL. In further embodiments, the antibody or antigen-binding fragment is capable of inhibiting NA sialidase activity of one or more Group 1 and/or Group 2 IAV, and/or of one or more IBV, with an IC50 in a range of: from about 0.00001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.0001 μg/ml to about 0.001 μg/ml, or about 0.0001 μg/ml to about 0.0001 μg/ml, or about 0.0001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.001 μg/mi to about 25 μg/ml, or about 0.001 μg/ml to about 10 μg/ml, or about 0.001 μg/ml to about 1 μg/ml, or about 0.001 μg/ml to about 0.1 μg/ml, or about 0.001 μg/ml to about 0.01 μg/mi, or about 0.01 μg/ml to about 25 μg/ml, or about 0.01 μg/ml to about 10 μg/ml, or about 0.01 μg/ml to about 1 μg/ml, or about 0.01 μg/ml to about 0.1 μg/ml, or about 1 μg/ml to about 25 μg/ml, or about 1 μg/ml to about 10 μg/ml, or of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 μg/ml.

In certain embodiments, the antibody or antigen-binding fragment is capable of activating a human FcγRIIIa. In further embodiments, activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation (e.g., of 23 hours) of the antibody or antigen-binding fragment with a target cell (e.g., a A549 cell) infected with a IAV. In still further embodiments, activation is as determined following an incubation (optionally, for about 23 hours) of the antibody or antigen-binding fragment with the target cell infected with a H1N1 IAV, wherein, optionally, the H1N1 IAV is A/PR8/34, and/or wherein, optionally, the infection has a multiplicity of infection (MOI) of 6.

In certain embodiments, the antibody or antigen-binding fragment is capable of neutralizing infection by an IAV and/or an IBV. In certain embodiments, the IAV and/or the IBV is antiviral-resistant, wherein, optionally, the antiviral is oseltamivir.

In certain embodiments, the IAV comprises a N1 NA that comprises the amino acid mutation(s): H275Y; E1119D+H275Y; S247N+H275Y; I222V; and/or N294S wherein, optionally, the IAV comprises CA09 or A/Aichi. In certain embodiments, the IAV comprises a N2 NA that comprises the amino acid mutation(s) E119V, Q136K, and/or R292K.

In certain embodiments, the antibody or antigen-binding fragment is capable of treating and/or preventing (i) an IAV infection and/or (ii) an IBV infection in a subject.

In certain embodiments, the antibody or antigen-binding fragment is capable of treating and/or attenuating an infection by: (i) a H1N1 virus, wherein, optionally, the H1N1 virus comprises A/PR8/34; and/or (ii) a H3N2 virus, wherein, optionally, the H3N2 virus optionally comprises A/Hong Kong/68.

In certain embodiments, the antibody or antigen-binding fragment is capable of preventing weight loss in a subject infected by the IAV and/or IBV, optionally for (i) up to 15 days, or (ii) more than 15 days, following administration of an effective amount of the antibody or antigen-binding fragment.

In certain embodiments, the antibody or antigen-binding fragment is capable of preventing a loss in body weight of greater than 10% in a subject having an IAV infection and/or an IBV infection, as determined by reference to the subject's body weight just prior to the IAV and/or IBV infection.

In certain embodiments, the antibody or antigen-binding fragment is capable extending survival of a subject having an IAV infection and/or an IBV infection.

In certain embodiments, the antibody or antigen-binding fragment has an in vivo half-life in a mouse (e.g., a tg32 mouse): (i) in a range of: from about 10 days to about 14 days, about 10.2 days to about 13.8 days, about 10.5 days to about 13.5 days, about 11 days to about 13 days, about 11.5 days to about 12.5 days, between 10 days and 14 days, or between 10.5 days and 13.5 days, or between 11 days and 13 days, or of about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0 days; or (ii) in a range of: from about 12 days to about 16 days, about 12.5 days to 15.5 days, about 13 days to 15 days, about 13.5 days to about 14.5 days, or between 12 days and 16 days, or between 13 days and 15 days, or between 13.5 days and 14.5 days, or of about 12.0, 12.1, 12.2, 12.3, 12.4, 12.5, 12.6, 12.7, 12.8, 12.9, 13.0, 13.1, 13.2, 13.3, 13.4, 13.5, 1.36, 13.7, 13.8, 13.9, 14.0, 14.1, 14.2, 14.3, 14.4, 14.5, 14.6, 14.7, 14.8, 14.9, 15.0 15.1, 15.2, 15.3, 15.4, 15.5, 1.56, 15.7, 15.8, 15.9, or 16.0 days.

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region from an antibody light chain and an antibody heavy chain, respectively. In certain embodiments, a VL is a kappa (κ) class (also “VK” herein). In certain embodiments, a VL is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FRI-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs. In certain embodiments, one or more CDRs do not contact antigen and/or do not contribute energetically to antigen binding.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme, such as the Kabat, Chothia, EU, IMGT, Contact, North, Martin, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Pluckthun, J. Mol. Bio. 309:657-670 (2001); North et al. J Mol Biol. (2011) 406:228-56; doi:10.1016/j.jmb.2010.10.030; Abhinandan and Martin, Mol Immunol. (2008) 45:3832-9. 10.1016/j.molimm.2008.05.022). The antibody and CDR numbering systems of these references are incorporated herein by reference. Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. In certain embodiments, an antibody or antigen-binding fragment is provided that comprises CDRs of in a VH sequence according to any one of SEQ ID NOs.: 2, 14, 26, 171, 38, 50, 62, 74, 86, 183, 98, 110, 122, 134, 146, 158, 199, 203, 207, 216, and 228, and in a VL sequence according to any one of SEQ ID NOs.: 26, 36, 46, 56, 66, 76, 86, 96, 8, 20, 32, 44, 56, 68, 80, 92, 104, 116, 128, 140, 152, 174, 177, 180, 186, 189, 192, 164, 201, 205, 209, 217, and 230, in accordance with any known CDR numbering method, including the Kabat, Chothia, EU, IMGT, Martin (Enhanced Chothia), Contact, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software (www.chemcomp.com).

In certain embodiments, an antibody or an antigen-binding fragment of the present disclosure comprises a CDRH1, a CDRH2, a CDRH3, a CDRL1, a CDRL2, and a CDRL3, wherein each CDR is independently selected from a corresponding CDR of an NA-specific antibody as provided in Table 1 and/or Table 2. That is, all combinations of CDRs from NA-specific antibodies provided in Table 1 and/or Table 2 are contemplated.

In some embodiments, CDRs are in accordance with the IMGT numbering method.

In certain embodiments, the present disclosure provides an antibody, or antigen-binding fragment thereof, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein: (i) optionally, the CDRH1 comprises or consists of the amino acid sequence set forth in any one of SEQ ID NOs.: 3, 15, 27, 39, 51, 63, 75, 87, 99, 111, 123, 135, 147, 159, and 231, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in any one of SEQ ID NOs.: 4, 16, 28, 40, 52, 64, 76, 88, 100, 112, 124, 136, 148, 160, and 232, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in any one of SEQ ID NOs.: 5, 17, 29, 172, 41, 53, 65, 77, 89, 184, 101, 113, 125, 137, 149, 161, and 233, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in any one of SEQ ID NOs.: 9, 21, 33, 45, 57, 69, 81, 93, 105, 117, 129, 141, 153, 165, and 234, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in any one of SEQ ID NOs.: 10, 22, 34, 46, 58, 70, 82, 94, 106, 118, 130, 142, 154, 166, and 235, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in any one of SEQ ID NOs.: 11, 23, 35, 175, 178, 181, 47, 59, 71, 83, 95, 187, 193, 107, 119, 131, 143, 155, 190, 167, and 236, or a functional variant thereof comprising having one, two, or three amino acid substitutions, one or more of which substitutions is optionally a conservative substitution and/or is a substitution to a germline-encoded amino acid.

In further embodiments, CDRH1, CDRH2, CDRH3, CDRL1, CDRL2, and CDRL3 comprise or consist of the amino acid sequences set forth in SEQ ID NOs.: (i) 3-5 and 9-11, respectively; (ii) 15-17 and 21-23, respectively; (iii) 27-29 and 33-35, respectively; (iv) 27, 28, 172, and 33-35, respectively; (v) 27-29, 33, 34, and 175, respectively; (vi) 27-29, 33, 34, and 178, respectively; (vii) 27-29, 33, 34, and 181, respectively; (viii) 27, 28, 172, 33, 34, and 175, respectively; (ix) 27, 28, 172, 33, 34, and 178, respectively; (x) 27, 28, 172, 33, 34, and 181, respectively; (xi) 39-41 and 45-47, respectively; (xii) 51-53 and 57-59, respectively; (xiii) 63-65 and 69-71, respectively; (xiv) 75-77 and 81-83, respectively; (xv) 87-89 and 93-95, respectively; (xvi) 87, 88, 184 and 93-95, respectively; (xvii) 87-89, 93, 94, and 187, respectively; (xviii) 87-89, 93, 94, and 190, respectively; (xix) 87-89 93, 94, and 193, respectively; (xx) 87, 88, 184, 93, 94, and 187, respectively; (xxi) 87, 88, 184, 93, 94, and 190, respectively; (xxii) 87, 88, 184, 93, 94, and 193, respectively; (xxiii) 87-89, 141, 142, and 131, respectively; (xxiv) 99-101 and 105-107, respectively; (xxv) 111-113 and 117-119, respectively; (xxvi) 123-125 and 129-131, respectively; (xxvii) 135-137 and 141-143, respectively; (xxviii) 147-149 and 153-155, respectively; (xxix) 159-161 and 165-167, respectively; or (xxx) 231-233 and 234-236, respectively.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype, into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In any of the presently disclosed embodiments, an antibody or antigen-binding fragment of the present disclosure may comprise any one or more of CL, a CH1, a CH2, and a CH3. In certain embodiments, a CL comprises an amino acid sequence having 90%, 91%, 92%, 93%, 94%, 95%, 96%, 975, 98%, 99%, or 100% identity to the amino acid sequence of SEQ ID NO.:211. In certain embodiments, a CH1-CH2-CH3 comprises an amino acid sequence having 90%, 91%, 92%, 93%, 94%, 95%, 96%, 97%, 98%, 99%, or 100% identity to the amino acid sequence of SEQ ID NO.:210 or SEQ ID NO.:215. It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. In some embodiments, the scFv polypeptide comprises a polypeptide linker disposed between and linking the VH and VL domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (Bird et al., Science 242:423-426 (1988)) and the pentamer Gly-Gly-Gly-Gly-Ser when present in a single iteration or repeated 1 to 5 or more times, or more. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human. ScFvs can be constructed using any combination of the VH and VL sequences or any combination of the CDRH1, CDRH2, CDRH3, CDRL1, CDRL2, and CDRL3 sequences disclosed herein. In some embodiments, linker sequences are not required; for example, when the first and second polypeptides have non-essential N-terminal amino acid regions that can be used to separate the functional domains and prevent steric interference.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a influenza NA antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises at least one more germline-encoded amino acid in a variable region as compared to a parent antibody or antigen-binding fragment, provided that the parent antibody or antigen binding fragment comprises one or more somatic mutations. Variable region and CDR amino acid sequences of exemplary anti-NA antibodies of the present disclosure are provided in Table 1 herein.

In some embodiments, the VH is encoded by or derived from human IGHV1-69*01F or IGHV1-69D*OF, IGHJ4*02F, and IGHD1-26*01F, and/or the VL is encoded by or derived from human IGKV3D-15*01 F and Homsap IGK2*02 (F). Polynucleotide sequences and other information of these and related human IG alleles are available at, for example, IMGT.org (see e.g. www.imgt.org/IMGT_vquest/analysis).

In certain embodiments, an antibody or antigen-binding fragment comprises an amino acid modification (e.g., a substitution mutation) to remove an undesired risk of oxidation, deamidation, and/or isomerization.

Also provided herein are variant antibodies that comprise one or more amino acid alterations in a variable region (e.g., VH, VL, framework or CDR) as compared to a presently disclosed (“parent”) antibody, wherein the variant antibody is capable of binding to a NA antigen.

In certain embodiments, (i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 2, 14, 26, 171, 38, 50, 62, 74, 86, 183, 98, 110, 122, 134, 146, 158, 199, 203, 207, 216, and 228, wherein sequence variation is optionally limited to one or more framework regions and/or sequence variation comprises comprises one or more substitution to a germline-encoded amino acid, and/or (ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 8, 20, 32, 44, 56, 68, 80, 92, 104, 116, 128, 140, 152, 174, 177, 180, 186, 189, 192, 164, 201, 205, 209, 217, and 230, wherein sequence variation is optionally limited to one or more framework regions and/or sequence variation comprises one or more substitution to a germline-encoded amino acid.

In some embodiments, the VH and the VL comprise or consist of amino acid sequences having at least 80%, at least 85%, at least 90%, at least 91%, at least 92%, at least 93%, at least 94%, at least 95%, at least 96%, at least 97%, at least 98%, or at least 99%, to SEQ ID NOs.: (i) 2 and 8, respectively; (ii) 14 and 20, respectively; (iii) 26 and 32, respectively; (iv) 26 and 174, respectively; (v) 26 and 177, respectively; (vi) 26 and 180, respectively; (vii) 171 and 32, respectively; (viii) 171 and 174, respectively; (ix) 171 and 177, respectively; (x) 171 and 180, respectively; (xi) 38 and 44, respectively; (xii) 50 and 56, respectively; (xiii) 62 and 68, respectively; (xiv) 74 and 80, respectively; (xv) 86 and 92, respectively; (xvi) 86 and 186, respectively; (xvii) 86 and 189, respectively; (xviii) 86 and 192, respectively; (xix) 183 and 92, respectively; (xx) 183 and 186, respectively; (xxi) 183 and 189, respectively; (xxii) 183 and 192, respectively; (xxiii) 98 and 104, respectively; (xxiv) 110 and 116, respectively; (xxv) 122 and 128, respectively; (xxvi) 134 and 140, respectively; (xxvii) 146 and 152, respectively; (xxviii) 158 and 164, respectively; (xxix) 199 and 201, respectively; (xxx) 203 and 205, respectively; (xxxi) 207 and 209, respectively; (xxxii) 216 and 217, respectively; or (xxxiii) 228 and 230, respectively.

In certain embodiments, the VH comprises or consists of any VH amino acid sequence set forth in Table 1 and/or Table 2, and the VL comprises or consists of any VL amino acid sequence set forth in Table 1 and/or Table 2.

In some embodiments, the VH and the VL comprise or consist of the amino acid sequences according to SEQ ID NOs.: (i) 2 and 8, respectively; (ii) 14 and 20, respectively; (iii) 26 and 32, respectively; (iv) 26 and 174, respectively; (v) 26 and 177, respectively; (vi) 26 and 180, respectively; (vii) 171 and 32, respectively; (viii) 171 and 174, respectively; (ix) 171 and 177, respectively; (x) 171 and 180, respectively; (xi) 38 and 44, respectively; (xii) 50 and 56, respectively; (xiii) 62 and 68, respectively; (xiv) 74 and 80, respectively; (xv) 86 and 92, respectively; (xvi) 86 and 186, respectively; (xvii) 86 and 189, respectively; (xviii) 86 and 192, respectively; (xix) 183 and 92, respectively; (xx) 183 and 186, respectively; (xxi) 183 and 189, respectively; (xxii) 183 and 192, respectively; (xxiii) 98 and 104, respectively; (xxiv) 110 and 116, respectively; (xxv) 122 and 128, respectively; (xxvi) 134 and 140, respectively; (xxvii) 146 and 152, respectively; (xxviii) 158 and 164, respectively; (xxix) 199 and 201, respectively; (xxx) 203 and 205, respectively; (xxxi) 207 and 209, respectively; (xxxii) 216 and 217, respectively; or (xxxiii) 228 and 230, respectively.

Also provided herein is a polypeptide comprising an amino acid sequence sequence according to SEQ ID NO.:219, wherein the polypeptide is capable of binding to an influenza virus neuraminidase (NA). As demonstrated in the present Examples, a CDRH3 according to the exemplified clonally related antibodies binds in an active site cavity (i.e., enzymatic pocket) in NA.

In some embodiments, the polypeptide comprises an antibody heavy chain variable domain (VH), or a fragment thereof, and the amino acid sequence sequence according to SEQ ID NO.:219 is optionally comprised in the VH or fragment thereof. In further embodiments, the amino acid sequence according to SEQ ID NO.:219 comprises any one of SEQ ID NOs.: 149, 5, 17, 29, 172, 41, 53, 65, 77, 89, 184, 101, 113, 125, 137, and 161. In certain embodiments, the polypeptide or VH further comprises: (i) an amino acid sequence sequence according to SEQ ID NO.:220; and/or (ii) an amino acid sequence according to SEQ ID NO.:221.

In certain embodiments, the polypeptide further comprises an antibody light chain variable domain (VL), wherein, optionally, the VL comprises: (i) an amino acid sequence according to SEQ ID NO.:222; (ii) an amino acid sequence according to SEQ ID NO.:223; and/or (iii) an amino acid sequence according to SEQ ID NO.:224.

In certain embodiments, the VH comprises or consists of an amino acid sequence having at least 90%, at least 92%, at least 95%, at least 97%, or at least 99% identity to the amino acid sequence of of any one of SEQ ID NOs.: 199, 2, 14, 26, 171, 38, 50, 62, 74, 86, 183, 98, 110, 122, 134, 146, 158, 203, 207, 216, and 228.

In some embodiments, the VL comprises or consists of an amino acid sequence having at least 90%, at least 92%, at least 95%, at least 97%, or at least 99% identity to the amino acid sequence of any one of SEQ ID NOs.: 201, 8, 20, 32, 44, 56, 68, 80, 92, 104, 116, 128, 140, 152, 174, 177, 180, 186, 189, 192, 164, 205, 209, 217, and 230.

In certain embodiments, the VH comprises or consists of an amino acid sequence having at least 90%, at least 92%, at least 95%, at least 97%, or at least 99% identity to the amino acid sequence of SEQ ID NO.: 199, and the VL comprises or consists of an amino acid sequence having at least 90%, at least 92%, at least 95%, at least 97%, or at least 99% identity to the amino acid sequence of any one of SEQ ID NO.: 201.

In certain embodiments, the polypeptide comprises an antibody or an antigen-binding fragment thereof.

Also provided is an antibody or an antigen-binding fragment thereof, comprising a heavy chain variable domain (VH) amino acid sequence and a light chain variable domain (VL) amino acid sequence, wherein the VH comprises or consists of an amino acid sequence having at least 90%, at least 92%, at least 95%, at least 97%, or at least 99% identity to the amino acid sequence of any one of SEQ ID NOs.: 199, 2, 14, 26, 171, 38, 50, 62, 74, 86, 183, 98, 110, 122, 134, 146, 158, 203, 207, 216, and 228, and wherein the VL comprises or consists of an amino acid sequence having at least 90%, at least 92%, at least 95%, at least 97%, or at least 99% identity to the amino acid sequence of any one of SEQ ID NOs.: 201, 8, 20, 32, 44, 56, 68, 80, 92, 104, 116, 128, 140, 152, 174, 177, 180, 186, 189, 192, 164, 205, 209, 217, and 230, wherein the antibody or antigen-binding fragment thereof is capable of binding to a neuraminidase (NA) from: (i) an influenza A virus (IAV), wherein the IAV comprises a Group 1 IAV, a Group 2 IAV, or both; and/or (ii) an influenza B virus (IBV).

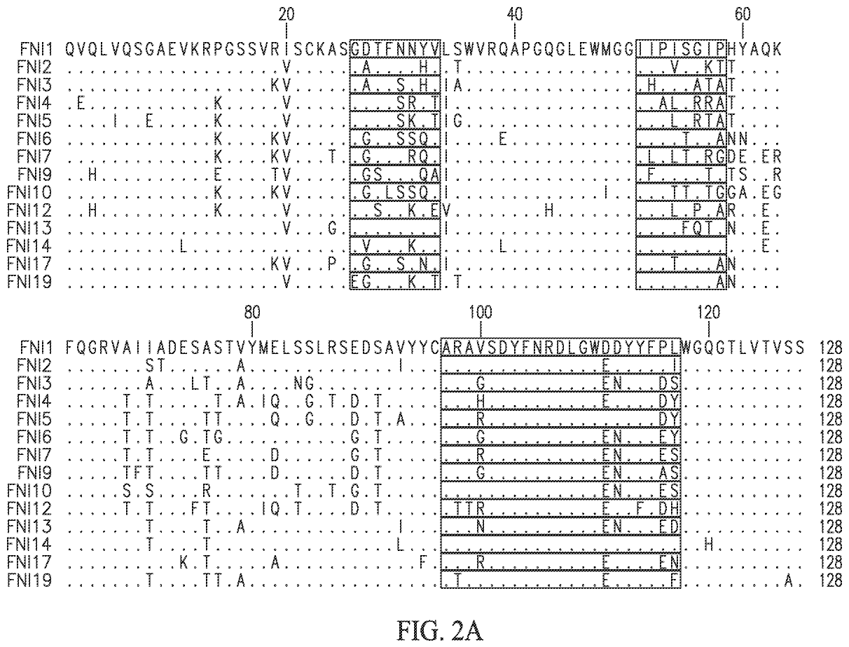

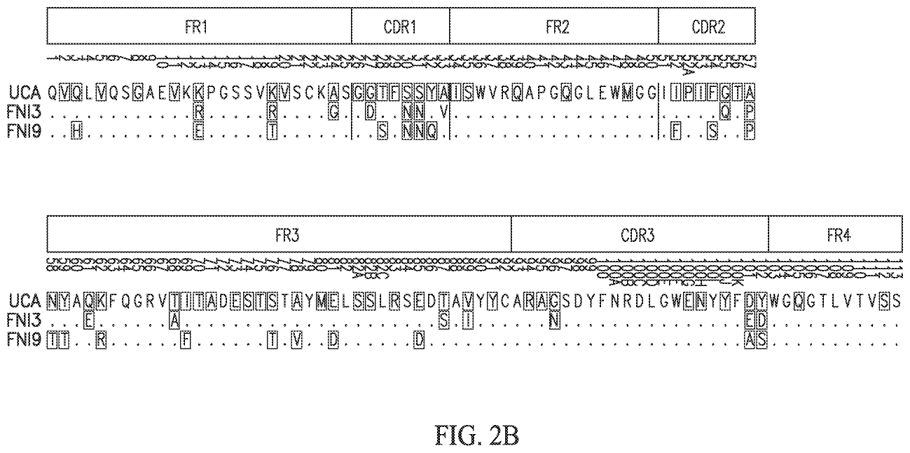

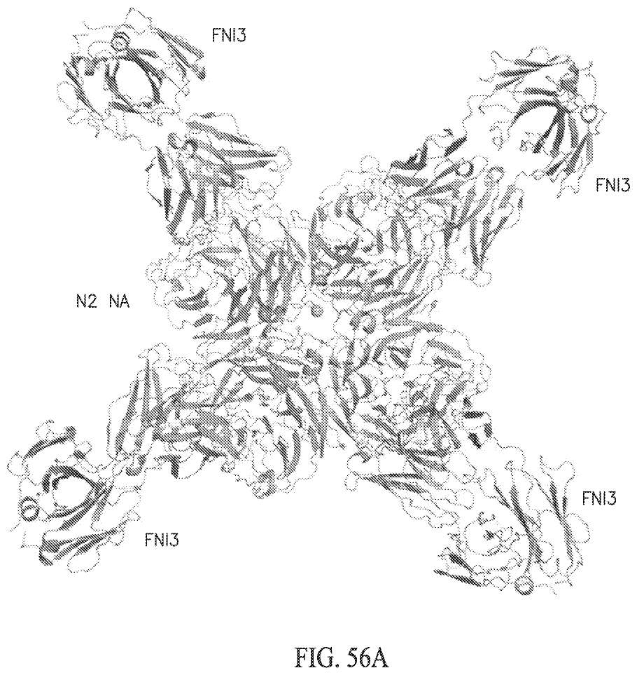

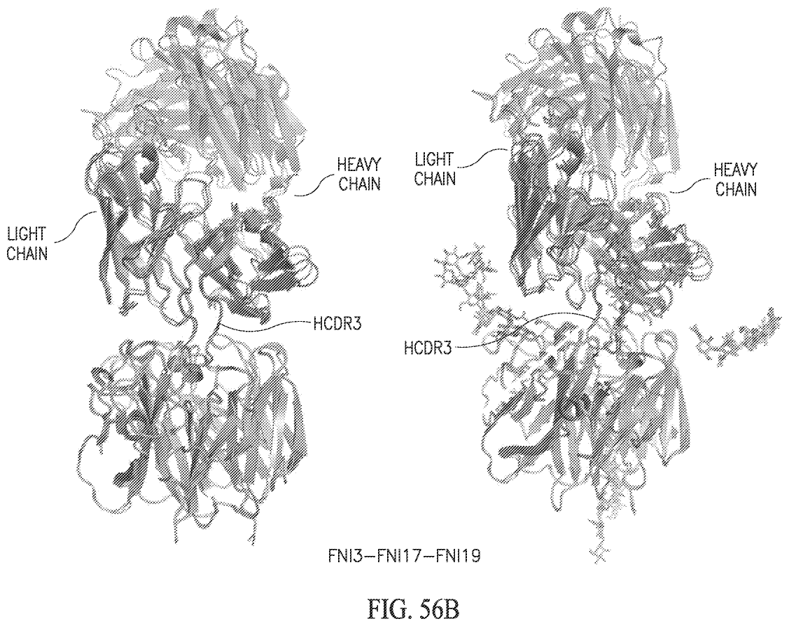

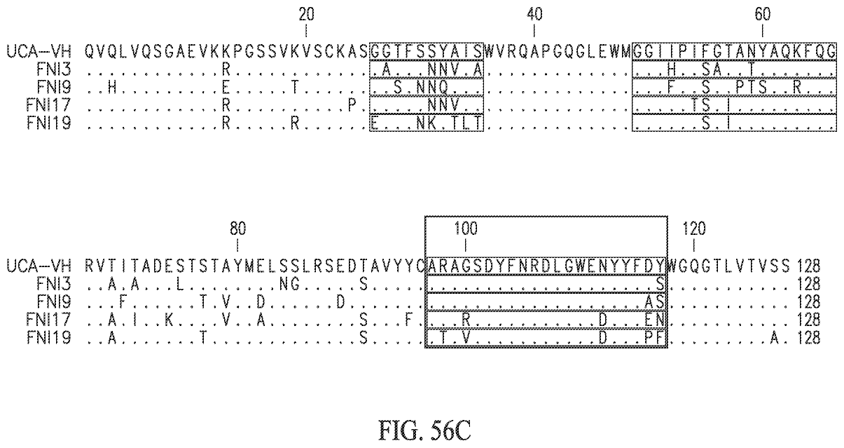

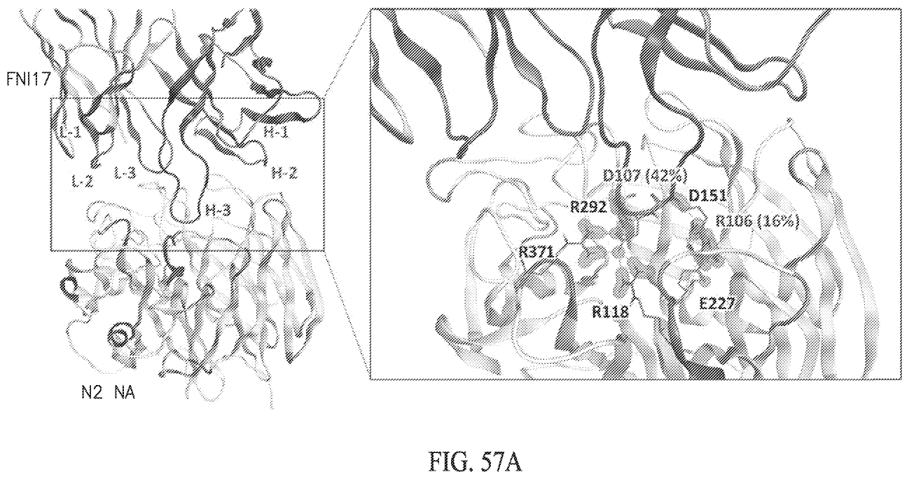

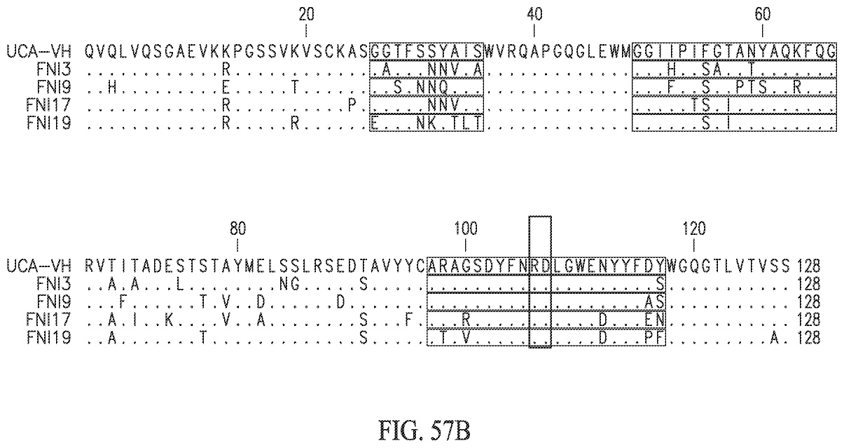

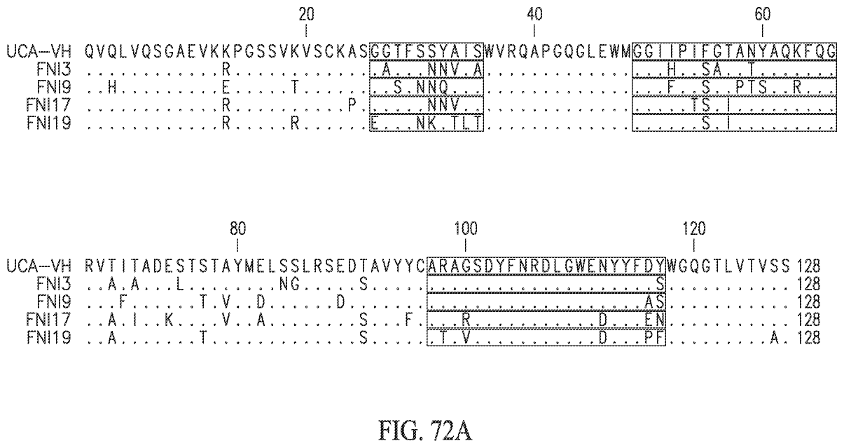

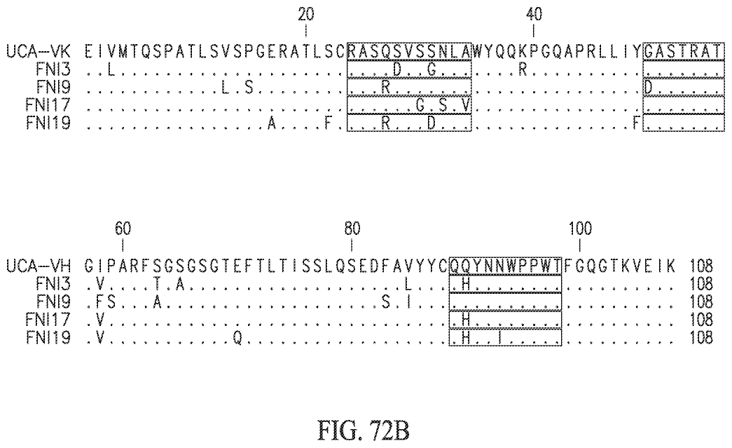

Also provided is an antibody, or an antigen-binding fragment thereof, that comprises a VH and a VL, wherein the VH comprises any combination of the VH amino acid residues shown in FIG. 2A, FIG. 56C, FIGS. 57B, and 72A, and the VL comprises any combination of the VL amino acid residues shown in FIG. 72B. Briefly, the clonally related FNI antibodies shown in these figures all recognize NA. Certain of the FNI antibodies comprise a different amino acid at a VH or a VL position compared to one or more other FNI antibodies. Accordingly, disclosed embodiments include those antibodies and antigen-binding fragments that include a consensus VH amino acid sequence that encompasses all variations and combinations of the VH amino acid residues shown in the foregoing figures, and a VL amino acid sequences that encompass all variations and combinations of the VL amino acid residues shown in the foregoing figures. Also provided is an antibody, or an antigen-binding fragment thereof, that is capable of binding to: (i) a NA epitope that comprises any one or more of the following amino acids (N1 NA numbering): R368, R293, E228, E344, S247, D198, D151, R118; and/or (ii) a NA epitope that comprises any one or more of the following amino acids (N2 NA numbering): R371, R292, E227, E344, S247, D198, D151, R118.

Also provided is an antibody, or an antigen-binding fragment thereof, that is capable of binding to: (i) a NA epitope that comprises the amino acids R368, R293, E228, D151, and R118 (N1 NA numbering); and/or (ii) a NA epitope that comprises the amino acids R371, R292, E227, D151, and R118 (N2 NA numbering).

Also provided is an antibody, or an antigen-binding fragment thereof, that is capable of binding to an epitope comprised in or comprising a NA active site, wherein, optionally, the NA active site comprises the following amino acids (N2 numbering): R118, D151, R152, R224, E276, R292, R371, Y406, E119, R156, W178, S179, D/N198, I222, E227, H274, E277, D293, E425. In some embodiments, the epitope further comprises any one or more of the following NA amino acids (N2 numbering): E344, E227, S247, and D198. In some embodiments, the antibody or antigen-binding fragment is capable of binding to a NA comprising a S245N amino acid mutation and/or a E22ID amino acid mutation.

Also provided is an antibody, or an antigen-binding fragment thereof, that is capable of binding to an IBV NA epitope that comprises any one or more of the following amino acids: R116, D149, E226, R292, and R374.

Also provided is an antibody, or an antigen-binding fragment thereof, that is capable of binding to an IBV NA epitope that comprises the amino acids R116, D149, E226, R292, and R374.

In some embodiments, the influenza comprises an influenza A virus, an influenza B virus, or both.

In certain embodiments, an antibody or antigen-binding fragment of the present disclosure is monospecific (e.g., binds to a single epitope) or is multispecific (e.g., binds to multiple epitopes and/or target molecules). Antibodies and antigen binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different eptiopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein. A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different NA epitope, and a bispecific or multispecific antibody or antigen-binding fragment as provided herein can, in some embodiments, comprise a further NA-specific binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In certain embodiments, the antibody or antigen-binding fragment comprises a Fc polypeptide, or a fragment thereof. The “Fc” fragment or Fc polypeptide comprises the carboxy-terminal portions (i.e., the CH2 and CH3 domains of IgG) of both antibody H chains held together by disulfides. An Fc may comprise a dimer comprised of two Fc polypeptides (i.e., two CH2-CH3 polypeptides). Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/I332E, P257I/Q311, K326W/E333S, S239D/I332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc Regions”, published by InvivoGen (2011) and available online at invivogen.com/PDF/review/review-Engineered-Fc-Regions-invivogen.pdf9utm_source=review&utm_medium=pdf&utm_campaign=review&utm_content=Engineered-Fc-Regions, and are incorporated herein by reference.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with higher affinity, and the “R131” variant, which binds to IgG1 Fc with low affinityer. See, e.g., Bruhns et al., Blood 113:3716-3725 (2009).

Regarding FcγRII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with lower affinity, and the “V158” variant, which binds to IgG1 Fc with higher affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D. In some embodiments, the Fc polypeptide or fragment thereof comprises S at position 239 (EU numbering).

In certain embodiments, the Fc polypeptide or fragment thereof may comprise or consist of at least a portion of an Fc polypeptide or fragment thereof that is involved in FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”, “LS”, “_LS”, and “-LS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody or antigen-binding fragment includes a Fc moiety that comprises the substitution mtuations M428I/N434S. In some embodiments, an antibody or antigen-binding fragment includes a Fc polypeptide or fragment thereof that comprises the substitution mtuations G236A/A330L/I332E. In certain embodiments, an antibody or antigen-binding fragment includes a (e.g., IgG) Fc moiety that comprises a G236A mutation, an A330L mutation, and a I332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D (e.g., comprises S at 239). In certain embodiments, an antibody or antigen-binding fragment includes a Fc polypeptide or fragment thereof that comprises the substitution mutations: M428L/N434S and G236A/S239D/A330L/I332E.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. MAbs 6:1-12 (2018)).

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and I332E, that are capable of activating dendritic cells that may induce, e.g., T cell immunity to the antigen.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc of the present disclosure comprises two CH2-CH3 polypeptides that associate to form a dimer.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., He domains.

A “human antibody” is an antibody containing only sequences that are present in an antibody that is produced by a human (i.e., sequences that are encoded by human antibody-encoding genes). However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

In some embodiments, various pharmacokinetic (“PK”) parameters are used to describe or characterize the antibodies or antigen-binding fragments provided herein. Details regarding collection of antibody serum concentrations for purpose of evaluating PK parameters are described in association with the Examples herein. The term “ti” or “half-life” refers to the elimination half-life of the antibody or antigen-binding fragment included in the pharmaceutical composition administered to a subject. The term “Ca” generally refers to the last measurable plasma concentration (i.e., subsequent thereto, the substance is not present at a measurable concentration in plasma).

In any of the presently disclosed embodiments, an antibody or antigen-binding fragment can comprise the CH1-CH3 amino acid sequence set forth in SEQ ID NO.:210 and/or the CH1-CH3 amino acid sequence set forth in SEQ ID NO.:215.

In any of the presently disclosed embodiments, an antibody or antigen-binding fragment can comprise the CL amino acid sequence set forth in SEQ ID NO.:211.

In some embodiments, an antibody is provided that comprises the heavy chain amino acid sequence set forth in SEQ ID NO.:212:

(SEQ ID NO.: 212)
QVQLVQSGAEVKEPGSSVTVSCKASGGTFSNNVISWVRQAPGQGLEW
MGGIIPTSGIANYAQKFQGRVAIIADKSTSTVYMALSSLRSEDSAVYFC
ARARSDYFNRDLGWEDYYFENWGQGTLVTVSSASTKGPSVFPLAPSSKS
TSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVL
HEALHSHYTQKSLSLSPGK

In certain embodiments, the antibody further comprises the light chain amino acid sequence set forth in SEQ ID NO.:214:

(SEQ ID NO.: 214)
EIVMTQSPATLSVSPGERATLSCRASQSVGSSLAWYQQKPGQAPRLLIY
GASTRATGVPARFSGSGSGTEFTLTISSLQSEDFAVYYCQHYNNWPPWT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

In some embodiments, an antibody is provided that comprises the heavy chain amino acid sequence set forth in SEQ ID NO.:213.

(SEQ ID NO.: 213)
QVQLVQSGAEVKEPGSSVTVSCKASGGTFSNNVISWVRQAPGQGLEW
MGGIIPTSGIANYAQKFQGRVAIIADKSTSTVYMALSSLRSEDSAVYFC
ARARSDYFNRDLGWEDYYFENWGQGTLVTVSSASTKGPSVFPLAPSSKS
TSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAP
ELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVL
HEALHSHYTQKSLSLSPG

In certain embodiments, the antibody further comprises the light chain amino acid sequence set forth in SEQ ID NO.:214.

(SEQ ID NO .: 214)
EIVMTQSPATLSVSPGERATLSCRASQSVGSSLAWYQQKPGQAPRLLIYG
ASTRATGVPARFSGSGSGTEFTLTISSLQSEDFAVYYCQHYNNWPPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

In some embodiments, an antibody is provided that comprises (1) two heavy chains, each comprising the amino acid sequence set forth in SEQ ID NO.:212, and (2) two light chains, each comprising the amino acid sequence set forth in SEQ ID NO.:214.

In some embodiments, an antibody is provided that comprises (1) two heavy chains, each comprising the amino acid sequence set forth in SEQ ID NO.:213, and (2) 5 two light chains, each comprising the amino acid sequence set forth in SEQ ID NO.:214.

Polynucleotides, Vectors, and Host Cells

In another aspect, the present disclosure provides isolated polynucleotides that encode any of the presently disclosed antibodies or an antigen-binding fragment thereof, or a portion thereof (e.g., a CDR, a VH, a VL, a heavy chain, or a light chain, or a heavy chain and a light chain), or that encode a presently disclosed polypeptide.

In certain embodiments, the polynucleotide comprises deoxyribonucleic acid (DNA) or ribonucleic acid (RNA), wherein the RNA optionally comprises messenger RNA (mRNA).

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine.

In certain embodiments, a polynucleotide comprises a polynucleotide having at least 50% (e.g., 50%, 55%, 60%, 65%, 70%, 75%, 80%, 85%, 90%, 91%, 92%, 94%, 94%, 95%, 96%, 97%, 98%, 99%, or more) identity to the polynucleotide sequence set forth in any one or more of SEQ ID NOs.: 198, 200, 1, 13, 25, 170, 37, 49, 61, 73, 85, 182, 97, 109, 121, 133, 145, 157, 6, 18, 30, 42, 54, 66, 78, 90, 102, 114, 126, 138, 150, 162, 7, 19, 31, 173, 176, 179, 43, 55, 67, 79, 91, 185, 188, 191, 103, 115, 127, 139, 151, 163, 12, 24, 36, 48, 60, 72, 84, 96, 108, 120, 132, 144, 156, 168, 202, 206, 204, 208, 227, and 229.

In certain embodiments, the polynucleotide is codon-optimized for expression in a host cell (e.g., a human cell, or a CHO cell). Once a coding sequence is known or identified, codon optimization can be performed using known techniques and tools, e.g., using the GenScript® OptimiumGene™ tool, or the like). Codon-optimized sequences include sequences that are partially codon-optimized (i.e., one or more codon is optimized for expression in the host cell) and those that are fully codon-optimized.

In particular embodiments, a polynucleotide comprises the polynucleotide sequence of SEQ ID NO.:198 and the polynucleotide sequence of SEQ ID NO.:200.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In any of the presently disclosed embodiments, the polynucleotide can comprise deoxyribonucleic acid (DNA) or ribonucleic acid (RNA). In some embodiments, the RNA comprises messenger RNA (mRNA).

Vectors are also provided, wherein the vectors comprise or contain a polynucleotide as disclosed herein (e.g., a polynucleotide that encodes an antibody or antigen-binding fragment or polypeptide that binds to IAV NA). A vector can comprise any one or more of the vectors disclosed herein. In particular embodiments, a vector is provided that comprises a DNA plasmid construct encoding the antibody or antigen-binding fragment, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH1, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

In some embodiments, a method is provided that comprises administering to a subject a first polynucleotide (e.g., mRNA) encoding an antibody heavy chain, a VH, or a Fd (VH+CH1), and administering to the subject a second polynucleotide (e.g., mRNA) encoding the cognate antibody light chain, VL, or VL+CL.

In some embodiments, a polynucleotide (e.g., mRNA) is provided that encodes a heavy chain and a light chain of an antibody or antigen-binding fragment thereof. In some embodiments, a polynucleotide (e.g., mRNA) is provided that encodes two heavy chains and two light chains of an antibody or antigen-binding fragment thereof. See, e.g. Li, J Q., Zhang, Z R., Zhang, H Q. et al. Intranasal delivery of replicating mRNA encoding neutralizing antibody against SARS-CoV-2 infection in mice. Sig Transduct Target Ther 6, 369 (2021). https://doi.org/10.1038/s41392-021-00783-1, the antibody-encoding mRNA constructs, vectors, and related techniques of which are incorporated herein by reference. In some embodiments, a polynucleotide is delivered to a subject via an alphavius replicon particle (VRP) delivery system. In some embodiments, a replicon comprises a modified VEEV replicon comprising two subgenomic promoters. In some embodiments, a polynucleotide or replicon can translate simultaneously the heavy chain (or VH, or VH+1) and the light chain (or VL, or VL+CL) of an antibody or antigen-binding fragment thereof. In some embodiments, a method is provided that comprises delivering to a subject such a polynucleotide or replicon.

In a further aspect, the present disclosure also provides a host cell expressing an antibody or antigen-binding fragment according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells, such as human B cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biolog, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody, or an antigen-binding fragment thereof, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glysocylation or fucosylation), or a lack thereof, on the antibody or antigen-binding fragment that is not present in a native state of the antibody or antigen-binding fragment (or in a native state of a parent antibody from which the antibody or antigen binding fragment was engineered or derived). Such a PTM, or a lack thereof, may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a host cell can comprise one or more post-translational modification, or can include fewer post-translational modification(s), such that it is distinct from the antibody when isolated from the human and/or produced by the native human B cell or plasma cell).

Insect cells useful expressing a binding protein of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody or antigen-binding fragment of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

In a related aspect, the present disclosure provides methods for producing an antibody, or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Compositions

Also provided herein are compositions that comprise a presently disclosed antibody, antigen-binding fragment, polypeptide, polynucleotide, vector, or host cell, singly or in any combination, and can further comprise a pharmaceutically acceptable carrier, excipient, or diluent. Such compositions, as well as carriers, excipients, and diluents, are discussed in further detail herein.

In certain embodiments, a composition comprises a first vector comprising a first plasmid, and a second vector comprising a second plasmid, wherein the first plasmid comprises a polynucleotide encoding a heavy chain, VH, or VH+CH1, and a second plasmid comprises a polynucleotide encoding the cognate light chain, VL, or VL+CL of the antibody or antigen-binding fragment thereof. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Iterdiscip Rev. Nanomed Nanobiotechnol. 11(2):e1530 (2019)). Principles, reagents, and techniques for designing appropriate mRNA and and formulating mRNA-LNP and delivering the same are described in, for example, Pardi el al. (J Control Re/ease 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019): and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

In certain embodiments, a composition comprises a first antibody or antigen-binding fragment of the present disclosure and a second antibody or antigen-binding fragment of the present disclosure, wherein of the first antibody or antigen-binding fragment and the second antibody or antigen-binding fragment are different.

### Methods and Uses

Also provided herein are methods for use of an antibody or antigen-binding fragment, nucleic acid, vector, cell, or composition of the present disclosure in the diagnosis of an influenza infection (e.g., in a human subject, or in a sample obtained from a human subject).

Methods of diagnosis (e.g., in vitro, ex vivo) may include contacting an antibody, antibody fragment (e.g., antigen binding fragment) with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e. without any contact to the human or animal body. Examples of detection methods are well-known to the person skilled in the art and include, e.g., ELISA (enzyme-linked immunosorbent assay), including direct, indirect, and sandwich ELISA.

Also provided herein are methods of treating a subject using an antibody or antigen-binding fragment of the present disclosure, or a composition comprising the same, wherein the subject has, is believed to have, or is at risk for having an infection by influenza. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of an influenza infection (i.e., in a statistically significant manner). In certain embodiments, therapeutic or prophylactic/preventive benefit includes a reduced duration of hospitalization for treatment of an influenza infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, antigen-binding fragment, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

Accordingly, in certain embodiments, methods are provided for treating an influenza infection in a subject, wherein the methods comprise administering to the subject an effective amount of an antibody, antigen-binding fragment, polynucleotide, vector, host cell, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with an influenza infection. These include, but are not limited to, age, occupation, general health, pre-existing health conditions, locale, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are can be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a subject treated according to the present disclosure has received a vaccine for influenza and the vaccine is determined to be ineffective, e.g., by post-vaccine infection or symptoms in the subject, by clinical diagnosis or scientific or regulatory consensus.

Prophylaxis of infection with influenza virus refers in particular to prophylactic settings, wherein the subject was not diagnosed with infection with influenza virus (either no diagnosis was performed or diagnosis results were negative) and/or the subject does not show or experience symptoms of infection with influenza virus. Prophylaxis of infection with influenza virus is particularly useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza virus is also particularly useful in subjects at greater risk acquiring influenza virus infection, e.g., due to increased exposure, for example subjects working or staying in public areas, in particular health care workers.

In certain embodiments, treatment is administered as peri-exposure or pre-exposure prophylaxis. In certain embodiments, treatment is administered as pos-exposure prophylaxis.

In therapeutic settings, in contrast, the subject is typically infected with influenza virus, diagnosed with influenza virus infection, and/or showing symptoms of influenza virus infection. Of note, the terms “treatment” and “therapy”/“therapeutic” of influenza virus infection can refer to (complete) cure as well as attenuation/reduction of influenza virus infection and/or related symptoms (e.g., attenuation/reduction of severity of infection and/or symptoms, number of symptoms, duration of infection and/or symptoms, or any combination thereof).

It will be understood that reference herein to a reduced number and/or severity of symptoms, which reduction results from administration of a presently disclosed pharmaceutical composition, describes a comparison with a reference subject who did not receive a disclosed pharmaceutical composition. A reference subject can be, for example, (i) the same subject during an earlier period of time (e.g., a prior influenza A virus season), (ii) a subject of a same or a similar: age or age group; gender; pregnancy status; chronic medical condition (such as chronic cardiac, pulmonary, renal, metabolic, neurodevelopmental, liver or hematologic diseases) or lack thereof; and/or immunosuppressive condition or lack thereof; or (iii) a typical subject within a population (e.g., local, regional, or national, including of a same or similar age or age range and/or general state of health) during an influenza virus season. Prophylaxis can be determined by, for example, the failure to develop a diagnosed influenza infection and/or the lack of symptoms associated with influenza infection during a part of a full influenza season, or over a full influenza season.

In certain embodiments, the methods provided herein include administering a therapeutically effective amount of a composition according to the present disclosure to a subject at immediate risk of influenza infection. An immediate risk of influenza infection typically occurs during an influenza epidemic. Influenza viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza epidemic is high during November, December, January, February and March, while in the southern hemisphere the risk of an influenza epidemic is high during May, June, July, August and September.

In some embodiments, treatment and/or prevention comprises post-exposure prophylaxis.

In some embodiments, the subject has received, is receiving, or will receive an antiviral agent. In some embodiments, the antiviral agent comprises a neuraminidase inhibitor, an influenza polymerase inhibitor, or both. In certain embodiments, the antiviral agent comprises oseltamivir, lanamivir, peramivir, zanamivir, baloxavir, or any combination thereof.

Typical routes of administering the presently disclosed compositions include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat an influenza virus infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, tollowing administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In certain embodiments, a method comprises administering the antibody, antigen-binding fragment, polynucleotide, vector, host cell, or composition to the subject at 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the subject being infected by influenza.

Compositions comprising an antibody, antigen-binding fragment, polynucleotide, vector, host cell, or composition of the present disclosure may also be administered simultaneously with, prior to, or after administration of one or more other therapeutic agents, such as, for example, a neuraminidase inhibitor, e.g., oseltamivir, zanamivir, peramivir, or laninamivir. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In some embodiments, an antibody (or one or more nucleic acid, host cell, vector, or composition) is administered to a subject who has previously received one or more anti-inflammatory agent and/or one or more antiviral agent. In some embodiments, the antiviral is a neuramidase inhibitor (NAI), such as, for example, oseltamivir, zanamivir, peramivir, or laninamivir. In some embodiments, one or more anti-inflammatory agent and/or one or more antiviral agent is administered to a subject who has previously received an antibody (or one or more nucleic acid, host cell, vector, or composition). In some embodiments, the antiviral is a neuramidase inhibitor (NAI), such as, for example, oseltamivir, zanamivir, peramivir, or laninamivir.

In a related aspect, uses of the presently disclosed antibodies, antigen-binding fragments, vectors, host cells, and compositions (e.g., in the diagnosis, prophylaxis, and/or treatment of an influenza infection, in the manufacture of a medicament for preventing or treating an influenza infection) are provided.

In certain embodiments, an antibody, antigen-binding fragment, polynucleotide, vector, host cell, or composition is provided for use in a method of treating or preventing an influenza infection in a subject.

In certain embodiments, an antibody, antigen-binding fragment, or composition is provided for use in a method of manufacturing or preparing a medicament for treating or preventing a influenza infection in a subject.

The present disclosure also provides the following non-limiting embodiments.

Embodiment 1. An antibody, or an antigen-binding fragment thereof, that is capable of binding to a neuraminidase (NA) from: (i) an influenza A virus (IAV), wherein the IAV comprises a Group 1 IAV, a Group 2 IAV, or both; and (ii) an influenza B virus (IBV).

Embodiment 2. The antibody or antigen-binding fragment of Embodiment 1, which is human, humanized, or chimeric.

Embodiment 3. The antibody or antigen-binding fragment of Embodiment 1 or 2, wherein: (i) the Group 1 IAV NA comprises a N1, a N4, a N5, and/or a N8; and/or (ii) the Group 2 IAV NA comprises a N2, a N3, a N6, a N7, and/or a N9.

Embodiment 4. The antibody or antigen-binding fragment of Embodiment 3, wherein: (i) the N1 is a N1 from any one or more of: A/California/07/2009, A/California/07/2009 I223R/H275Y, A/Swine/Jiangsu/J004/2018, A/Stockholm/18/2007, A/Brisbane/02/2018, A/Michigan/45/2015, A/Mississippi/3/2001, A/Netherlands/603/2009, A/Netherlands/602/2009, A/Vietnam/1203/2004, A/G4/SW/Shangdong/1207/2016, A/G4/SW/Henan/SN13/2018, A/G4/SW/Jiangsu/J004/2018, and A/New Jersey/8/1976; (ii) the N4 is from A/mallard duck/Netherlands/30/2011; (iii) the N5 is from A/aquatic bird/Korea/CN5/2009; (iv) the N8 is from A/harbor seal/New Hampshire/179629/2011; (v) the N2 is a N2 from any one or more of: A/Washington/01/2007, A/HongKong/68, A/South Australia/34/2019, A/Switzerland/8060/2017, A/Singapore/INFIMH-16-0019/2016, A/Switzerland/9715293/2013, A/Leningrad/134/17/57, A/Florida/4/2006, A/Netherlands/823/1992, A/Norway/466/2014, A/Switzerland/8060/2017, A/Texas/50/2012, A/Victoria/361/2011, A/HongKong/2671/2019, A/SW/Mexico/SG1444/2011, A/Tanzania/205/2010, A/Aichi/2/1968, A/Bilthoven/21793/1972, A/Netherlands/233/1982, A/Shanghai/11/1987, A/Nanchang/933/1995, A/Fukui/45/2004, and A/Brisbane/10/2007; (vi) the N3 is from A/Canada/rv504/2004; (v) the N6 is from A/swine/Ontario/01911/1/99; (vi) the N7 is from A/Netherlands/078/03; and/or (vii) the N9 is a N9 from any one or more of: A/Anhui/2013 and A/Hong Kong/56/2015.

Embodiment 5. The antibody or antigen-binding fragment of any one of Embodiments 1-4, wherein the IBV NA is a NA from any one or more of: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008; B/Colorado/06/2017; B/Hubei-wujiang/158/2009; B/Massachusetts/02/2012; B/Netherlands/234/2011; B/Perth/211/2001; B/Texas/06/2011 (Yamagata); B/Perth/211/2011; B/HongKong/05/1972; B/Phuket/3073/2013, B/Harbin/7/1994 (Victoria), and B/Washington/02/2019 (Victoria).

Embodiment 6. The antibody or antigen-binding fragment of any one of Embodiments 1-5, wherein the antibody or antigen-binding fragment is capable of binding to each of: (i) a Group 1 IAV NA; (ii) a Group 2 IAV NA; and (iii) a IBV NA
with an EC₅₀ in a range from about 0.1 μg/mL to about 50 μg/mL, or in a range from about 0.1 μg/mL to about 2 μg/mL, or in a range from 0.1 μg/mL to about 10 μg/mL, or in a range from 2 μg/mL to about 10 μg/mL, or in a range from about 0.4 μg/mL to about 50 μg/mL, or in a range from about 0.4 μg/mL to about 2 μg/mL, or in a range from 0.4 μg/mL to about 10 μg/m L, or in a range from 2 μg/mL to about 10 μg/mL, or in a range from 0.4 μg/mL to about 1 μg/mL, or 0.4 μg/mL or less.

Embodiment 7. The antibody or antigen-binding fragment of Embodiment 6, wherein the antibody or antigen-binding fragment is capable of binding to: (i) the Group 1 IAV NA with an EC₅₀ in a range from about 0.4 μg/mL to about 50 μg/mL, from about 0.4 μg/mL to about 10 μg/m L, from about 0.4 μg/mL to about 2 μg/mL, from about 2 μg/mL to about 50 μg/mL, from about 2 μg/mL to about 10 μg/mL, or from about 10 μg/mL to about 50 μg/mL; (ii) the Group 2 IAV NA with an EC₅₀ in a range from about 0.4 μg/mL to about 50 μg/mL, or from about 0.4 μg/mL to about 10 μg/mL, or from about 0.4 μg/mL to about 2 μg/mL, or from about 2 μg/mL to about 50 μg/mL, or from about 2 μg/mL to about 10 μg/mL, or from about 10 μg/mL to about 50 μg/mL; and/or (iii) the IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL.

Embodiment 8. The antibody or antigen-binding fragment of Embodiment 7, wherein the antibody or antigen-binding fragment is capable of binding to: (i) a N1 with an EC₅₀ of about 0.4 μg/mL, or in a range from about 0.4 μg/mL to about 50 μg/mL, or in a range from about 0.1 μg/m L to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (ii) a N4 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (iii) a N5 with an EC₅₀ in a range from about 0.4 μg/mL to about 2 μg/mL; (iv) a N8 with an EC₅₀ of about 50 μg/mL; (v) a N2 with an EC₅₀ in a range from about 0.4 μg/mL to about 20 μg/mL, or from about 0.4 μg/mL to about 10 μg/mL, or from about 0.4 μg/mL to about 2 μg/mL, from about 1 μg/mL to about 10 μg/mL, or from about 1 μg/mL to about 20 μg/mL, or from about 1 μg/mL to about 5 μg/mL, or about 1, 2, 3, 4, 5, 6, 7, 8, 9, 10, 15, or 20 μg/mL; (vi) a N3 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (vii) a N6 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; (viii) a N7 with an EC₅₀ in a range from about 2 μg/mL to about 50 μg/mL; (ix) a N9 with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL; and/or (xi) a IBV NA with an EC₅₀ of about 0.4 μg/mL, or in a range from about 0.1 μg/mL to about 1.9 μg/mL, or from about 0.1 μg/mL to about 1.5 μg/mL, or from about 0.1 μg/mL to about 1.0 μg/mL, or about 0.1, 0.2, 0.3, 0.4, 0.5, 0.6, 0.7, 0.8, 0.9, or 1.0 μg/mL.

Embodiment 9. The antibody or antigen-binding fragment of Embodiment 7 or 8, wherein the antibody or antigen-binding fragment is capable of binding to: (i) one or more of: N1 A/California/07/2009, N1 A/California/07/2009 I223R/H275Y, N1 A/Stockholm/18/2007, N1 A/Swine/Jiangsu/J004/2008, N4 A/mallard duck/Netherlands/30/2011, N5 A/aquatic bird/Korea/CN5/2009, N2 A/Hong Kong/68, N2 A/Leningrad/134/17/57, N3 A/Canada/rv504/2004, N6 A/Swine/Ontario/01911/1/99, N9 A/Anhui/1/2013, B/Lee/10/1940 (Ancestral), B/Brisbane/60/2008 (Victoria), B/Malaysia/2506/2004 (Victoria), B/Malaysia/3120318925/2013 (Yamagata), B/Wisconsin/1/2010 (Yamagata), and B/Yamanashi/166/1998 (Yamagata), with an EC₅₀ of about 0.4 μg/mL, or in a range of from about 0.1 μg/mL to about 1.9 μg/mL, or of from about 0.1 μg/mL to about 1.5 μg/mL, or of from about 0.1 μg/mL to about 1.0 μg/mL, or about 0.1, 0.2, 0.3, 0.4, 0.5, 0.6, 0.7, 0.8, 0.9, or 1.0 μg/mL; (ii) N5 A/aquatic bird/Korea/CN5/2009 with an EC₅₀ of about 2 μg/mL, or in a range of from about 2 μg/mL to about 10 μg/mL; (iii) N8 A/harbor seal/New Hampshire/179629/2011 with an EC₅₀ of about 50 μg/mL; (iv) N2 A/Washington/01/2007 with an EC₅₀ in a range from about 2 μg/mL to about 10 μg/mL; (v) N7 A/Netherlands/078/03 with an EC₅₀ in a range from about 2 μg/mL to about 50 μg/mL; (vi) N2 A/South Australia/34/2019 with an EC₅₀ in a range of from about 0.4 μg/mL to about 50 μg/mL; (vii) N2 A/Switzerland/8060/2017 with an EC₅₀ in a range of from about 9.5 μg/mL to about 3.8 μg/mL; (viii) N2 A/Singapore/INFIMH-16-0019/2016 with an EC₅₀ in a range of from about 18.4 μg/mL to about 2.2 μg/mL; (iv) N2 A/Switzerland/9715293/2013 with an EC₅₀ in a range of from about 1.6 μg/mL to about 1.2 μg/mL; and/or (v) N1 A/Swine/Jiangsu/J004/2018 with an EC₅₀ in a range of from about 0.4 μg/mL to about 50 μg/mL, or about 0.4, about 2, about 10, or about 50 μg/mL.

Embodiment 10. The antibody or antigen-binding fragment of any one of Embodiments 1-9, wherein the NA is expressed on the surface of a host cell (e.g., a CHO cell) and binding to NA is according to flow cytometry.

Embodiment 11. The antibody or antigen-binding fragment of any one of Embodiments 1-10, which is capable of binding to a NA with a KD of less than 1.0E-12 M, less than 1.0E-11 M, less than 1.0 E-11 M, or of 1.0E-12M or less, 1.0E-11M or less, or 1.0E-10 or less, or with a KD between 1.0E-10 and 1.0E-13, or with a KD between 1.0E-11 and 1.0E-13, wherein, optionally, the binding is as assessed by biolayer interferometry (BLI).

Embodiment 12. The antibody or antigen-binding fragment of Embodiment 11, wherein the NA is a N1, a N2, and/or a N9.

Embodiment 13. The antibody or antigen-binding fragment of any one of Embodiments 1-12, which is capable of binding to: (1) (i) a NA epitope that comprises any one or more of the following amino acids (N1 NA numbering): R368, R293, E228, E344, S247, D198, D151, R118; and/or (ii) a NA epitope that comprises any one or more of the following amino acids (N2 NA numbering): R371, R292, E227, E344, S247, D198, D151, R118; and/or (2)(i) a NA epitope that comprises the amino acids R368, R293, E228, D151, and R118 (N1 NA numbering); and/or (ii) a NA epitope that comprises the amino acids R371, R292, E227, D151, and R118 (N2 NA numbering); and/or (3) an epitope comprised in or comprising a NA active site, wherein, optionally, the NA active site comprises the following amino acids (N2 numbering): R118, D151, R152, R224, E276, R292, R371, Y406, E119, R156, W178, S179, D/N198, I222, E227, H274, E277, D293, E425; and/or (4) an IBV NA epitope that comprises: (i) any one or more of the following amino acids: R116, D149, E226, R292, and R374; or (ii) the amino acids R116, D149, E226, R292, and R374.

Embodiment 14. The antibody or antigen-binding fragment of Embodiment 13, wherein: (1) the epitope further comprises any one or more of the following NA amino acids (N2 numbering): E344, E227, S247, and D198; and/or (2) the antibody or antigen-binding fragment is capable of binding to a NA comprising a S245N amino acid mutation and/or a E221D amino acid mutation.

Embodiment 15. The antibody or antigen-binding fragment of any one of Embodiments 1-14, which is capable of binding to a NA comprising a S245N amino acid mutation and/or a E221D amino acid mutation.

Embodiment 16. The antibody or antigen-binding fragment of any one of Embodiments 1-15, wherein the antibody or antigen-binding fragment is capable of inhibiting a sialidase activity of (i) an IAV NA, wherein the IAV NA comprises a Group 1 IAV NA, a Group 2 IAV NA, or both, and/or of (ii) an IBV NA in an in vitro model of infection, an in vivo animal model of infection, and/or in a human.

Embodiment 17. The antibody or antigen-binding fragment of Embodiment 16, wherein: (i) the Group 1 IAV NA comprises a H1N1 and/or a H5N1; (ii) the Group 2 IAV NA comprises a H3N2 and/or a H7N9; and/or (iii) the IBV NA comprises one or more of: B/Lee/10/1940 (Ancestral); B/HongKong/05/1972; B/Taiwan/2/1962 (Ancestral); B/Brisbane/33/2008 (Victoria); B/Brisbane/60/2008 (Victoria); B/Malaysia/2506/2004 (Victoria); B/New York/1056/2003 (Victoria); B/Florida/4/2006(Yamagata); B/Jiangsu/10/2003 (Yamagata); B/Texas/06/2011 (Yamagata); B/Perth/211/2011; B/Harbin/7/1994 (Victoria); B/Colorado/06/2017 (Victoria); B/Washington/02/2019 (Victoria); B/Perth/211/2001 (Yamagata); B/Hubei-wujiagang/158/2009 (Yamagata); B/Wisconsin/01/2010 (Yamagata); B/Massachusetts/02/2012 (Yamagata); and B/Phuket/3073/2013 (Yamagata).

Embodiment 18. The antibody or antigen-binding fragment of any one of Embodiments 1-17, wherein the antibody or antigen-binding fragment is capable of inhibiting a sialidase activity by: a Group 1 IAV NA; a Group 2 IAV NA; and/or a IBV NA, with an IC50 in a range of from about 0.0008 μg/mL to about 4 μg/mL, from about 0.0008 μg/mL to about 3 μg/mL, from about 0.0008 μg/mL to about 2 μg/mL, from about 0.0008 μg/m L to about 1 μg/mL, from about 0.0008 g/m L to about 0.9 μg/mL, from about 0.0008 μg/mL to about 0.8 μg/mL, from about 0.0008 μg/mL to about 0.7 μg/mL, from about 0.0008 μg/mL to about 0.6 μg/mL, from about 0.0008 μg/mL to about 0.5 μg/mL, from about 0.0008 μg/mL to about 0.4 μg/mL, from about 0.0008 μg/mL to about 0.3 μg/mL, from about 0.0008 μg/mL to about 0.2 μg/mL, from about 0.0008 μg/mL to about 0.1 μg/mL, from about 0.0008 μg/mL to about 0.09 μg/mL, from about 0.0008 μg/mL to about 0.08 μg/mL, from about 0.0008 μg/mL to about 0.07 μg/mL, from about 0.0008 μg/mL to about 0.06 μg/mL, about 0.0008 μg/mL to about 0.05 μg/mL, about 0.0008 μg/mL to about 0.04 μg/m L, about 0.0008 μg/mL to about 0.03 μg/mL, about 0.0008 μg/mL to about 0.02 μg/mL, about 0.0008 μg/mL to about 0.01 μg/mL, from 0.002 μg/mL to about 4 μg/mL, from about 0.001 μg/mL to 50 μg/mL, from about 0.1 μg/mL to about 30 μg/mL, from about 0.1 μg/mL to about 20 μg/mL, from about 0.1 μg/mL to about 10 μg/mL, from about 0.1 μg/mL to about 9 μg/mL, from about 0.1 μg/mL to about 8 μg/mL, from about 0.1 μg/mL to about 7 μg/mL, from about 0.1 μg/mL to about 6 μg/mL, from about 0.1 μg/mL to about 5 μg/mL, from about 0.1 μg/mL to about 4 μg/mL, from about 0.1 μg/mL to about 3 μg/mL, from about 0.1 μg/mL to about 2 μg/mL, from about 0.1 μg/mL to about 1 μg/mL, from about 0.1 μg/mL to about 0.9 μg/mL, from about 0.1 μg/mL to about 0.8 μg/mL, from about 0.1 μg/mL to about 0.7 μg/mL, from about 0.1 μg/mL to about 0.6 μg/mL, from about 0.1 μg/mL to about 0.5 μg/mL, from about 0.1 μg/mL to about 0.4 μg/mL, from about 0.1 gtg/mL to about 0.3 μg/mL, from about 0.1 μg/mL to about 0.2 μg/mL, from about 0.8 μg/mL to about 30 μg/mL, from about 0.8 μg/mL to about 20 μg/mL, from about 0.8 μg/mL to about 10 μg/mL, from about 0.8 μg/mL to about 9 μg/mL, from about 0.8 μg/mL to about 8 μg/mL, from about 0.8 μg/mL to about 7 μg/mL, from about 0.8 μg/mL to about 6 μg/mL, from about 0.8 μg/mL to about 5 μg/mL, from about 0.8 μg/mL to about 4 μg/mL, from about 0.8 μg/mL to about 3 μg/mL, from about 0.8 μg/mL to about 2 μg/mL, from about 0.8 μg/mL to about 1 μg/mL, or of about 0.1 μg/mL, about 0.2 μg/mL, about 0.3 μg/mL, about 0.4 μg/mL, about 0.5 μg/mL, about 0.6 μg/mL, about 0.7 μg/mL, about 0.8 μg/mL, about 0.9 μg/mL, about 1.0 μg/mL, about 1.5 μg/mL, about 2.0 μg/mL, about 2.5 μg/mL, about 3.0 μg/mL, about 3.5 μg/mL, about 4.0 μg/mL, about 4.5 μg/mL, about 5.0 μg/mL, about 5.5 μg/mL, about 6.0 μg/mL, about 6.5 μg/mL, about 7.0 μg/mL, about 7.5 μg/mL, about 8.0 μg/mL, about 8.5 μg/mL, about 9.0 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 25 μg/mL, and/or about 30 μg/mL.

Embodiment 19. The antibody or antigen-binding fragment of Embodiment 18, which is capable of inhibiting NA sialidase activity of one or more Group 1 and/or Group 2 IAV, and/or of one or more IBV, with an IC50 in a range of from: about 0.00001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.0001 μg/ml to about 0.001 μg/ml, or about 0.0001 μg/ml to about 0.0001 μg/ml, or about 0.0001 μg/ml to about 25 μg/ml, or about 0.0001 μg/ml to about 10 μg/ml, or about 0.0001 μg/ml to about 1 μg/ml, or about 0.0001 μg/ml to about 0.1 μg/ml, or about 0.0001 μg/ml to about 0.01 μg/ml, or about 0.001 μg/ml to about 25 μg/ml, or about 0.001 μg/ml to about 10 μg/ml, or about 0.001 μg/ml to about 1 μg/ml, or about 0.001 μg/ml to about 0.1 μg/ml, or about 0.001 μg/ml to about 0.01 μg/ml, or about 0.01 μg/ml to about 25 μg/ml, or about 0.01 μg/ml to about 10 μg/ml, or about 0.01 μg/ml to about 1 μg/ml, or about 0.01 μg/ml to about 0.1 μg/ml, or about 1 μg/ml to about 25 μg/ml, or about 1 μg/ml to about 10 μg/ml,
or of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 μg/ml.

Embodiment 20. The antibody or antigen-binding fragment of any one of Embodiments 1-19, which is capable of activating a human FcγRIIIa.

Embodiment 21. The antibody or antigen-binding fragment of Embodiment 20, wherein activation is as determined using a host cell (optionally, a Jurkat cell) comprising: (i) the human FcγRIIIa (optionally, a F158 allele); and (ii) a NFAT expression control sequence operably linked to a sequence encoding a reporter, such as a luciferase reporter, following incubation (e.g., of 23 hours) of the antibody or antigen-binding fragment with a target cell (e.g., a A549 cell) infected with a IAV.

Embodiment 22. The antibody or antigen-binding fragment of Embodiment 21, wherein activation is as determined following an incubation (optionally, for about 23 hours) of the antibody or antigen-binding fragment with the target cell infected with a H1N1 IAV, wherein, optionally, the H1N1 IAV is A/PR8/34, and/or wherein, optionally, the infection has a multiplicity of infection (MOI) of 6.

Embodiment 23. The antibody or antigen-binding fragment of any one of Embodiments 1-22, which is capable of neutralizing infection by an IAV and/or an IBV.

Embodiment 24. The antibody or antigen-binding fragment of Embodiment 23, wherein the IAV and/or the IBV is antiviral-resistant, wherein, optionally, the antiviral is oseltamivir.

Embodiment 25. The antibody or antigen-binding fragment of any one of Embodiments 1-24, wherein the IAV comprises a N1 NA that comprises the amino acid mutation(s). H275Y; E119D+H275Y; S247N+H275Y; I222V; and/or N294S, wherein, optionally, the IAV comprises CA09 or A/Aichi.

Embodiment 26. The antibody or antigen-binding fragment of any one of Embodiments 1-25, wherein the IAV comprises a N2 NA that comprises the amino acid mutation(s) E119V, Q136K, and/or R292K.

Embodiment 27. The antibody or antigen-binding fragment of any one of Embodiments 1-26, wherein the antibody or antigen-binding fragment is capable of treating and/or preventing (i) an IAV infection and/or (ii) an IBV infection, in a subject.

Embodiment 28. The antibody or antigen-binding fragment of any one of Embodiments 1-27, wherein the antibody or antigen-binding fragment is capable of treating and/or attenuating an infection by: (i) a H1N1 virus, wherein, optionally, the H1N1 virus comprises A/PR8/34; and/or (ii) a H3N2 virus, wherein, optionally, the H3N2 virus optionally comprises A/Hong Kong/68.

Embodiment 29. The antibody or antigen-binding fragment of any one of Embodiments 1-28, wherein the antibody or antigen-binding fragment is capable of preventing weight loss in a subject infected by the IAV and/or IBV, optionally for (i) up to 15 days, or (ii) more than 15 days, following administration of an effective amount of the antibody or antigen-binding fragment.

Embodiment 30. The antibody or antigen-binding fragment of any one of Embodiments 1-29, wherein the antibody or antigen-binding fragment is capable of preventing a loss in body weight of greater than 10% in a subject having an IAV infection and/or an IBV infection, as determined by reference to the subject's body weight just prior to the IAV and/or IBV infection.

Embodiment 31. The antibody or antigen-binding fragment of any one of Embodiments 1-30, wherein the antibody or antigen-binding fragment is capable extending survival of a subject having an IAV infection and/or an IBV infection.

Embodiment 32. The antibody or antigen-binding fragment of any one of Embodiments 1-31, wherein the antibody or antigen-binding fragment has an in vivo half-life in a mouse (e.g., a tg32 mouse). (i) in a range of from: about 10 days to about 14 days, about 10.2 days to about 13.8 days, about 10.5 days to about 13.5 days, about 11 days to about 13 days, about 11.5 days to about 12.5 days, between 10 days and 14 days, or between 10.5 days and 13.5 days, or between 11 days and 13 days, or of about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, or 14.0 days; or (ii) in a range of from about 12 days to about 16 days, about 12.5 days to 15.5 days, about 13 days to 15 days, about 13.5 days to about 14.5 days, or between 12 days and 16 days, or between 13 days and 15 days, or between 13.5 days and 14.5 days, or of about 12.0, 12.1, 12.2, 12.3, 12.4, 12.5, 12.6, 12.7, 12.8, 12.9, 13.0, 13.1, 13.2, 13.3, 13.4, 13.5, 1.36, 13.7, 13.8, 13.9, 14.0, 14.1, 14.2, 14.3, 14.4, 14.5, 14.6, 14.7, 14.8, 14.9, 15.0 15.1, 15.2, 15.3, 15.4, 15.5, 1.56, 15.7, 15.8, 15.9, or 16.0 days.

Embodiment 33. The antibody or antigen-binding fragment of any one of Embodiments 1-32, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein: (i) optionally, the CDRH1 comprises or consists of the amino acid sequence set forth in any one of SEQ ID NOs.: 147, 3, 15, 27, 39, 51, 63, 75, 87, 99, 111, 123, 135, 159, and 231, or a functional variant thereof comprising one, two, or three acid substitutions, one or more of which substitutions is optionally a conservative substitution and/or is a substitution to a germline-encoded amino acid; (ii) optionally, the CDRH2 comprises or consists of the amino acid sequence set forth in any one of SEQ ID NOs.: 148, 4, 16, 28, 40, 52, 64, 76, 88, 100, 112, 124, 136, 160, and 232, or a functional variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence set forth in any one of SEQ ID NOs.: 149, 5, 17, 29, 172, 41, 53, 65, 77, 89, 184, 101, 113, 125, 137, 161, and 233, or a functional variant thereof comprising one, two, or three amino acid substitutions, one or more of which substitutions is optionally a conservative substitution and/or is a substitution to a germline-encoded amino acid; (iv) optionally, the CDRL1 comprises or consists of the amino acid sequence set forth in any one of SEQ ID NOs.: 153, 9, 21, 33, 45, 57, 69, 81, 93, 105, 117, 129, 141, 165, and 234, or a functional variant thereof comprising one, two, or three amino acid substitutions, one or more of which substitutions is optionally a conservative substitution and/or is a substitution to a germline-encoded amino acid; (v) optionally, the CDRL2 comprises or consists of the amino acid sequence set forth in any one of SEQ ID NOs.: 154, 10, 22, 34, 46, 58, 70, 82, 94, 106, 118, 130, 142, 166, and 235, or a functional variant thereof comprising one, two, or three amino acid substitutions, one or more of which substitutions is optionally a conservative substitution and/or is a substitution to a germline-encoded amino acid; and/or (vi) optionally, the CDRL3 comprises or consists of the amino acid sequence set forth in any one of SEQ ID NOs.: 155, 11, 23, 35, 175, 178, 181, 47, 59, 71, 83, 95, 187, 193, 107, 119, 131, 143, 190, 167, and 236, or a functional variant thereof comprising having one, two, or three amino acid substitutions, one or more of which substitutions is optionally a conservative substitution and/or is a substitution to a germline-encoded amino acid.

Embodiment 34. The antibody or antigen-binding fragment of Embodiment 33, comprising CDRH1, CDRH2, CDRH3, CDRL1, CDRL2, and CDRL3 amino acid sequences set forth in SEQ ID NOs.: (i) 147-149 and 153-155, respectively; (ii) 15-17 and 21-23, respectively; (iii) 27-29 and 33-35, respectively; (iv) 27, 28, 172, and 33-35, respectively; (v) 27-29, 33, 34, and 175, respectively; (vi) 27-29, 33, 34, and 178, respectively; (vii) 27-29, 33, 34, and 181, respectively; (viii) 27, 28, 172, 33, 34, and 175, respectively; (ix) 27, 28, 172, 33, 34, and 178, respectively; (x) 27, 28, 172, 33, 34, and 181, respectively; (xi) 39-41 and 45-47, respectively; (xii) 51-53 and 57-59, respectively; (xiii) 63-65 and 69-71, respectively; (xiv) 75-77 and 81-83, respectively; (xv) 87-89 and 93-95, respectively; (xvi) 87, 88, 184 and 93-95, respectively; (xvii) 87-89, 93, 94, and 187, respectively; (xviii) 87-89, 93, 94, and 190, respectively; (xix) 87-89, 93, 94, and 193, respectively; (xx) 87, 88, 184, 93, 94, and 187, respectively; (xxi) 87, 88, 184, 93, 94, and 190, respectively; (xxii) 87, 88, 184, 93, 94, and 193, respectively; (xxiii) 87-89, 141, 142, and 131, respectively; (xxiv) 99-101 and 105-107, respectively; (xxv) 111-113 and 117-119, respectively; (xxvi) 123-125 and 129-131, respectively; (xxvii) 135-137 and 141-143, respectively; (xxviii) 3-5 and 9-11, respectively; (xxix) 159-161 and 165-167, respectively; or (xxx) 231-233 and 234-236, respectively.

Embodiment 35. The antibody or antigen-binding fragment of any one of Embodiments 1-34, wherein: (i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 199, 2, 14, 26, 171, 38, 50, 62, 74, 86, 183, 98, 110, 122, 134, 146, 158, 203, 207, 216, and 228, wherein sequence variation is optionally limited to one or more framework regions and/or sequence variation comprises comprises one or more substitution to a germline-encoded amino acid; and/or (ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 201, 8, 20, 32, 44, 56, 68, 80, 92, 104, 116, 128, 140, 152, 174, 177, 180, 186, 189, 192, 164, 205, 209, 217, and 230, wherein sequence variation is optionally limited to one or more framework regions and/or sequence variation comprises one or more substitution to a germline-encoded amino acid.

Embodiment 36. The antibody or antigen-binding fragment of Embodiments 1-35, wherein the VH and the VL comprise or consist of the amino acid sequences set forth in SEQ ID NOs.: (i) 199 and 201, respectively; (ii) 14 and 20, respectively; (iii) 26 and 32, respectively; (iv) 26 and 174, respectively; (v) 26 and 177, respectively; (vi) 26 and 180, respectively; (vii) 171 and 32, respectively; (viii) 171 and 174, respectively; (ix) 171 and 177, respectively; (x) 171 and 180, respectively; (xi) 38 and 44, respectively; (xii) 50 and 56, respectively; (xiii) 62 and 68, respectively; (xiv) 74 and 80, respectively; (xv) 86 and 92, respectively; (xvi) 86 and 186, respectively; (xvii) 86 and 189, respectively; (xviii) 86 and 192, respectively; (xix) 183 and 92, respectively; (xx) 183 and 186, respectively; (xxi) 183 and 189, respectively; (xxii) 183 and 192, respectively; (xxiii) 98 and 104, respectively; (xxiv) 110 and 116, respectively; (xxv) 122 and 128, respectively; (xxvi) 134 and 140, respectively; (xxvii) 146 and 152, respectively; (xxviii) 158 and 164, respectively; (xxix) 2 and 8, respectively; (xxx) 203 and 205, respectively; (xxxi) 207 and 209, respectively; (xxxii) 216 and 217, respectively; or (xxxiii) 228 and 230, respectively.

Embodiment 37. The antibody or antigen-binding fragment of any one of Embodiments 1-36, comprising: (1) a CH1-CH3 comprising or consisting of the amino acid sequence set forth in SEQ ID NO.:210 or SEQ ID NO.:215; and/or (2) a CL comprising or consisting of the amino acid sequence set forth in SEQ ID NO.:211.

Embodiment 38. The antibody or antigen-binding fragment of any one of Embodiments 1-37, comprising: (1) a heavy chain comprising or consisting of the amino acid sequence set forth in SEQ ID NO.:212 or 213; and (2) a light chain comprising or consisting of the amino acid sequence set forth in SEQ ID NO.:214.

Embodiment 39. The antibody or antigen-binding fragment of any one of Embodiments 1-38, comprising: (1) a heavy chain comprising or consisting of the amino acid sequence set forth in SEQ ID NO.:212; and (2) a light chain comprising or consisting of the amino acid sequence set forth in SEQ ID NO.:214.

Embodiment 40. The antibody or antigen-binding fragment of any one of Embodiments 1-39, comprising: (1) a heavy chain comprising or consisting of the amino acid sequence set forth in SEQ ID NO.: 213; and (2) a light chain comprising or consisting of the amino acid sequence set forth in SEQ ID NO.:214.

Embodiment 41. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1, CDRH2, and CDRH3 comprise or consist of the amino acid sequences set forth in SEQ ID NOs: 147-149, respectively, and the CDRL1, CDRL2, and CDRL3 comprise or consist of the amino acid sequences set forth in SEQ ID NOs: 153-155, respectively; (ii) the CDRH1, CDRH2, and CDRH3 comprise or consist of the amino acid sequences set forth in SEQ ID NOs: 15-17, respectively, and the CDRL1, CDRL2, and CDRL3 comprise or consist of the amino acid sequences set forth in SEQ ID NOs: 21-23, respectively; (iii) the CDRH1, CDRH2, and CDRH3 comprise or consist of the amino acid sequences set forth in SEQ ID NOs: 27, 28, and 29 or 172, respectively, and the CDRL1, CDRL2, and CDRL3 comprise or consist of the amino acid sequences set forth in SEQ ID NOs: 33, 34, and 35 or 175 or 178 or 181, respectively; (iv) the CDRH1, CDRH2, and CDRH3 comprise or consist of the amino acid sequences set forth in SEQ ID NOs: 39-41, respectively, and the CDRL1, CDRL2, and CDRL3 comprise or consist of the amino acid sequences set forth in SEQ ID NOs: 45-47, respectively; (v) the CDRH1, CDRH2, and CDRH3 comprise or consist of the amino acid sequences set forth in SEQ ID NOs: 51-53, respectively, and the CDRL1, CDRL2, and CDRL3 comprise or consist of the amino acid sequences set forth in SEQ ID NOs: 57-59, respectively; (vi) the CDRH1, CDRH2, and CDRH3 comprise or consist of the amino acid sequences set forth in SEQ ID NOs: 63-65, respectively, and the CDRL1, CDRL2, and CDRL3 comprise or consist of the amino acid sequences set forth in SEQ ID NOs: 69-71, respectively; (vii) the CDRH1, CDRH2, and CDRH3 comprise or consist of the amino acid sequences set forth in SEQ ID NOs: 75-77, respectively, and the CDRL1, CDRL2, and CDRL3 comprise or consist of the amino acid sequences set forth in SEQ ID NOs: 81-83, respectively; (viii) the CDRH1, CDRH2, and CDRH3 comprise or consist of the amino acid sequences set forth in SEQ ID NOs: 87, 88, and 89 or 184, respectively, and the CDRL1, CDRL2, and CDRL3 comprise or consist of the amino acid sequences set forth in SEQ ID NOs: 93, 94, and 95 or 187 or 190 or 193, respectively; (ix) the CDRH1, CDRH2, and CDRH3 comprise or consist of the amino acid sequences set forth in SEQ ID NOs: 87, 88, 184, respectively, and the CDRL1, CDRL2, and CDRL3 comprise or consist of the amino acid sequences set forth in SEQ ID NOs: 93-95, respectively; (x) wherein the CDRH1, CDRH2, and CDRH3 comprise or consist of the amino acid sequences set forth in SEQ ID NOs: 99-101, respectively, and the CDRL1, CDRL2, and CDRL3 comprise or consist of the amino acid sequences set forth in SEQ ID NOs: 105-107, respectively; (xi) the CDRH1, CDRH2, and CDRH3 comprise or consist of the amino acid sequences set forth in SEQ ID NOs: 111-113, respectively, and the CDRL1, CDRL2, and CDRL3 comprise or consist of the amino acid sequences set forth in SEQ ID NOs: 117-119, respectively; (xii) the CDRH1, CDRH2, and CDRH3 comprise or consist of the amino acid sequences set forth in SEQ ID NOs: 123-125, respectively, and the CDRL1, CDRL2, and CDRL3 comprise or consist of the amino acid sequences set forth in SEQ ID NOs: 129-131, respectively; (xiii) the CDRH1, CDRH2, and CDRH3 comprise or consist of the amino acid sequences set forth in SEQ ID NOs: 135-137, respectively, and the CDRL1, CDRL2, and CDRL3 comprise or consist of the amino acid sequences set forth in SEQ ID NOs: 141-143, respectively; (xiv) the CDRH1, CDRH2, and CDRH3 comprise or consist of the amino acid sequences set forth in SEQ ID NOs: 3-5, respectively, and the CDRL1, CDRL2, and CDRL3 comprise or consist of the amino acid sequences set forth in SEQ ID NOs: 9-11, respectively; (xv) the CDRH1, CDRH2, and CDRH3 comprise or consist of the amino acid sequences set forth in SEQ ID NOs: 159-161, respectively, and the CDRL1, CDRL2, and CDRL3 comprise or consist of the amino acid sequences set forth in SEQ ID NOs: 165-167, respectively; (xvi) the CDRH1, CDRH2, and CDRH3 comprise or consist of the amino acid sequences set forth in SEQ ID NOs: 87-89, respectively, and the CDRL1, CDRL2, and CDRL3 comprise or consist of the amino acid sequences set forth in SEQ ID NOs: 141, 142, and 131, respectively; or (xvii) the CDRH1, CDRH2, and CDRH3 comprise or consist of the amino acid sequences set forth in SEQ ID NOs: 231-233, respectively, and the CDRL1, CDRL2, and CDRL3 comprise or consist of the amino acid sequences set forth in SEQ ID NOs: 234-236, respectively, and wherein the antibody or antigen-binding fragment is capable of binding to a neuraminidase (NA) from: (i) an influenza A virus (IAV), wherein the IAV comprises a Group 1 IAV, a Group 2 IAV, or both; and/or (ii) an influenza B virus (IBV).

Embodiment 42. An antibody, or antigen-binding fragment thereof, comprising a heavy chain variable domain (VH) and a light chain variable domain (VL), wherein: (i) the VH comprises or consists of the amino acid sequence set forth in SEQ ID NO: 199 and the VL comprises or consists of the amino acid sequence set forth in SEQ ID NO: 201; (ii) the VH comprises or consists of the amino acid sequence set forth in SEQ ID NO: 14 and the VL comprises or consists of the amino acid sequence set forth in SEQ ID NO: 20; (iii) the VH comprises or consists of the amino acid sequence set forth in SEQ ID NO: 26 or 171 and the VL comprises or consists of the amino acid sequence set forth in SEQ ID NO: 32, 174, 177, or 180; (iv) the VH comprises or consists of the amino acid sequence set forth in SEQ ID NO: 38 and the VL comprises or consists of the amino acid sequence set forth in SEQ ID NO: 44; (v) the VH comprises or consists of the amino acid sequence set forth in SEQ ID NO: 50 and the VL comprises or consists of the amino acid sequence set forth in SEQ ID NO: 56; (vi) the VH comprises or consists of the amino acid sequence set forth in SEQ ID NO: 62 and the VL comprises or consists of the amino acid sequence set forth in SEQ ID NO: 68; (vii) the VH comprises or consists of the amino acid sequence set forth in SEQ ID NO: 74 and the VL comprises or consists of the amino acid sequence set forth in SEQ ID NO: 80; (viii) the VH comprises or consists of the amino acid sequence set forth in SEQ ID NO: 86 or 183 and the VL comprises or consists of the amino acid sequence set forth in SEQ ID NO: 92, 186, 189, or 192; (ix) the VH comprises or consists of the amino acid sequence set forth in SEQ ID NO: 98 and the VL comprises or consists of the amino acid sequence set forth in SEQ ID NO: 104; (x) the VH comprises or consists of the amino acid sequence set forth in SEQ ID NO: 110 and the VL comprises or consists of the amino acid sequence set forth in SEQ ID NO: 116; (xi) the VH comprises or consists of the amino acid sequence set forth in SEQ ID NO: 122 and the VL comprises or consists of the amino acid sequence set forth in SEQ ID NO: 128; (xii) the VH comprises or consists of the amino acid sequence set forth in SEQ ID NO: 134 and the VL comprises or consists of the amino acid sequence set forth in SEQ ID NO: 140; (xiii) the VH comprises or consists of the amino acid sequence set forth in SEQ ID NO: 146 and the VL comprises or consists of the amino acid sequence set forth in SEQ ID NO: 152; (xiv) the VH comprises or consists of the amino acid sequence set forth in SEQ ID NO: 158 and the VL comprises or consists of the amino acid sequence set forth in SEQ ID NO: 164; (xv) the VH comprises or consists of the amino acid sequence set forth in SEQ ID NO: 2 and the VL comprises or consists of the amino acid sequence set forth in SEQ ID NO: 8; (xvi) the VH comprises or consists of the amino acid sequence set forth in SEQ ID NO: 203 and the VL comprises or consists of the amino acid sequence set forth in SEQ ID NO: 205; (xvii) the VH comprises or consists of the amino acid sequence set forth in SEQ ID NO: 207 and the VL comprises or consists of the amino acid sequence set forth in SEQ ID NO: 209; or (xviii) the VH comprises or consists of the amino acid sequence set forth in SEQ ID NO: 228 and the VL comprises or consists of the amino acid sequence set forth in SEQ ID NO: 230.

Embodiment 43. An antibody, or antigen-binding fragment thereof, comprising a heavy chain variable domain (VH) and a light chain variable domain (VL), wherein the VH comprises or consists of the amino acid sequence set forth in SEQ ID NO.:216 and the VL comprises comprises or consists of the amino acid sequence set forth in SEQ ID NO.:217.

Embodiment 44. The antibody or antigen-binding fragment of Embodiment 42 or 43, wherein the antibody or antigen-binding fragment is capable of binding to a neuraminidase (NA) from: (i) an influenza A virus (IAV), wherein the IAV comprises a Group 1 IAV, a Group 2 IAV, or both; and/or (ii) an influenza B virus (IBV), and wherein, optionally, the antibody or antigen-binding fragment is capable of (1) inhibiting NA sialidase activity and/or (2) neutralizing infection by the IAV and/or IBV.

Embodiment 45. A polypeptide comprising an amino acid sequence sequence according to SEQ ID NO.:219, wherein the polypeptide is capable of binding to an influenza virus neuraminidase (NA).

Embodiment 46. The polypeptide of Embodiment 45, wherein the polypeptide comprises an antibody heavy chain variable domain (VH), or a fragment thereof, and the amino acid sequence sequence according to SEQ ID NO.:219 is optionally comprised in the VH or fragment thereof.

Embodiment 47. The polypeptide of Embodiment 45 or 46, wherein the amino acid sequence according to SEQ ID NO.:219 comprises any one of SEQ ID NOs.: 149, 5, 17, 29, 172, 41, 53, 65, 77, 89, 184, 101, 113, 125, 137, and 161.

Embodiment 48. The polypeptide of any one of Embodiments 45-47, wherein the polypeptide or VH further comprises: an amino acid sequence sequence according to SEQ ID NO.:220; and/or an amino acid sequence according to SEQ ID NO.:221.

Embodiment 49. The polypeptide of any one of Embodiments 45-48, further comprising an antibody light chain variable domain (VL), wherein, optionally, the VL comprises: (i) an amino acid sequence according to SEQ ID NO.:222; (ii) an amino acid sequence according to SEQ ID NO.:223; and/or (iii) an amino acid sequence according to SEQ ID NO.:224.

Embodiment 50. The polypeptide of any one of Embodiments 46-49, wherein the VH comprises or consists of an amino acid sequence having at least 90%, at least 92%, at least 95%, at least 97%, or at least 99% identity to the amino acid sequence of any one of SEQ ID NOs.: 199, 2, 14, 26, 171, 38, 50, 62, 74, 86, 183, 98, 110, 122, 134, 146, 158, 203, 207, 216, and 228.

Embodiment 51. The polypeptide of Embodiment 49 or 50, wherein the VL comprises or consists of an amino acid sequence having at least 90/6, at least 92%, at least 95%, at least 97%, or at least 99% identity to the amino acid sequence of any one of SEQ ID NOs.: 201, 8, 20, 32, 44, 56, 68, 80, 92, 104, 116, 128, 140, 152, 174, 177, 180, 186, 189, 192, 164, 205, 209, 217, and 230.

Embodiment 52. The polypeptide of any one of Embodiments 45-51, wherein the polypeptide comprises an antibody or an antigen-binding fragment thereof.

Embodiment 53. An antibody or an antigen-binding fragment thereof, comprising a heavy chain variable domain (VH) amino acid sequence and a light chain variable domain (VL) amino acid sequence, wherein the VH comprises or consists of an amino acid sequence having at least 90%, at least 92%, at least 95%, at least 97%, or at least 99% identity to the amino acid sequence of any one of SEQ ID NOs.: 199, 2, 14, 26, 171, 38, 50, 62, 74, 86, 183, 98, 110, 122, 134, 146, 158, 203, 207, 216, and 228, and wherein the VL comprises or consists of an amino acid sequence having at least 90%, at least 92%, at least 95%, at least 97%, or at least 99% identity to the amino acid sequence of any one of SEQ ID NOs.: 201, 8, 20, 32, 44, 56, 68, 80, 92, 104, 116, 128, 140, 152, 174, 177, 180, 186, 189, 192, 164, 205, 209, 217, and 230, wherein the antibody or antigen-binding fragment thereof is capable of binding to a neuraminidase (NA) from: (i) an influenza A virus (IAV), wherein the IAV comprises a Group 1 IAV, a Group 2 IAV, or both; and/or (ii) an influenza B virus (IBV).

Embodiment 54. The polypeptide of any one of Embodiments 45-52 or the antibody or antigen-binding fragment of Embodiment 53, which is capable of binding to a neuraminidase (NA) from: (i) an influenza A virus (IAV), wherein the IAV comprises a Group 1 IAV, a Group 2 IAV, or both; and/or (ii) an influenza B virus (IBV), and wherein, optionally, the antibody or antigen-binding fragment is capable of (1) inhibiting NA sialidase activity and/or (2) neutralizing infection by the IAV and/or IBV.

Embodiment 55. An antibody, or an antigen-binding fragment thereof, that is capable of binding to: (i) a NA epitope that comprises any one or more of the following amino acids (N1 NA numbering): R368, R293, E228, E344, S247, D198, D151, R118; and/or (ii) a NA epitope that comprises any one or more of the following amino acids (N2 NA numbering): R371, R292, E227, E344, S247, D198, D151, R118.

Embodiment 56. An antibody, or an antigen-binding fragment thereof, that is capable of binding to: (i) a NA epitope that comprises the amino acids R368, R293, E228, D151, and R118 (N1 NA numbering); and/or (ii) a NA epitope that comprises the amino acids R371, R292, E227, D151, and R118 (N2 NA numbering).

Embodiment 57. An antibody, or an antigen-binding fragment thereof, that is capable of binding to an epitope comprised in or comprising a NA active site, wherein, optionally, the NA active site comprises the following amino acids (N2 numbering): R118, D151, R152, R224, E276, R292, R371, Y406, E119, R156, W178, S179, D/N198, I222, E227, H274, E277, D293, E425.

Embodiment 58. The antibody or antigen-binding fragment of any one of Embodiments 83-85 wherein the epitope further comprises any one or more of the following NA amino acids (N2 numbering): E344, E227, S247, and D198.

Embodiment 59. The antibody or antigen-binding fragment of any one of Embodiments 55-58, which is capable of binding to a NA comprising a S245N amino acid mutation and/or a E221D amino acid mutation.

Embodiment 60. An antibody, or an antigen-binding fragment thereof, that is capable of binding to an IBV NA epitope that comprises any one or more of the following amino acids: R116, D149, E226, R292, and R374.

Embodiment 61. An antibody, or an antigen-binding fragment thereof, that is capable of binding to an IBV NA epitope that comprises the amino acids R116, D149, E226, R292, and R374.

Embodiment 62. The antibody or antigen-binding fragment of any one of Embodiments 55-61, wherein the influenza comprises an influenza A virus, an influenza B virus, or both.

Embodiment 63. The antibody or antigen-binding fragment of any one of Embodiments 1-44 and 53-62, or the polypeptide of Embodiment 52, which is a IgG, IgA, IgM, IgE, or IgD isotype.

Embodiment 64. The antibody or antigen-binding fragment of any one of Embodiments 1-44 and 53-63, or the polypeptide of Embodiment 52, which is an IgG isotype selected from IgG1, IgG2, IgG3, and IgG4.

Embodiment 65. The antibody or antigen-binding fragment of any one of Embodiments 1-44 and 53-64, or the polypeptide of Embodiment 52, wherein the antibody, or the antigen-binding fragment, comprises a human antibody, a monoclonal antibody, a purified antibody, a single chain antibody, a Fab, a Fab′, a F(ab′)2, or Fv.

Embodiment 66. The antibody or antigen-binding fragment of any one of Embodiments 1-44 and 53-65, or the polypeptide of Embodiment 52, wherein the antibody or antigen-binding fragment is a multi-specific antibody or antigen-binding fragment.

Embodiment 67. The antibody or antigen-binding fragment of Embodiment 66, or the polypeptide of Embodiment 66, wherein the antibody or antigen-binding fragment is a bispecific antibody or antigen-binding fragment.

Embodiment 68. The antibody or antigen-binding fragment of Embodiment 66 or 67, comprising: (i) a first VH and a first VT; and (ii) a second VH and a second VL, wherein the first VH and the second VH are different and each independently comprise an amino acid sequence having at least 85% identity to the amino acid sequence set forth in any one of SEQ ID NOs.: 199, 2, 14, 26, 171 38, 50, 62, 74, 86, 183, 98, 110, 122, 134, 146, 158, 203, 207, 216, and 228 and wherein the first VL and the second VL are different and each independently comprise an amino acid sequence having at least 85% identity to the amino acid sequence set forth in any one of SEQ ID NOs.: 201, 8, 20, 32, 174, 177, 180, 44, 56, 68, 80, 92, 186, 189, 192, 104, 116, 128, 140, 152, 164, 205, 209, 217, and 230, and wherein the first VH and the first VL together form a first antigen-binding site, and wherein the second VH and the second VL together form a second antigen-binding site.

Embodiment 69. The antibody or antigen-binding fragment of any one of Embodiments 1-44 and 53-68, or the polypeptide of Embodiment 52, wherein the 5 antibody or antigen-binding fragment comprises an (e.g., IgG1) Fc polypeptide or a fragment thereof.

Embodiment 70. The antibody or antigen-binding fragment of Embodiment 69, or the polypeptide of Embodiment 69, wherein the Fc polypeptide or fragment thereof comprises: (i) a mutation that increases binding affinity to a human FcRn (e.g., as measured using surface plasmon resonance (SPR) (e.g., Biacore, e.g., T200 instrument, using manufacturer's protocols)), as compared to a reference Fc polypeptide that does not comprise the mutation; and/or (ii) a mutation that increases binding affinity to a human Fc-7R (e.g., as measured using surface plasmon resonance (SPR) (e.g., Biacore, e.g., T200 instrument, using manufacturer's protocols)) as compared to a reference Fc polypeptide that does not comprise the mutation.

Embodiment 71. The antibody or antigen-binding fragment of Embodiment 70, or the polypeptide of Embodiment 70, wherein the mutation that increases binding affinity to a human FcRn comprises: M428L; N434S; N434H; N434A; N434S; M252Y; S254T; T256E; T250Q; P257I; Q311I; D376V; T307A; E380A; or any combination thereof.

Embodiment 72. The antibody or antigen-binding fragment of Embodiment 70 or 71, or the polypeptide of Embodiment 70 or 71, wherein the mutation that increases binding affinity to a human FcRn comprises: (i) M428L/N434S; (ii) M252Y/S254T/T256E; (iii) T250Q/M428L; (iv) P257I/Q311I; (v) P257I/N434H; (vi) D376V/N434H; (vii) T307A/E380A/N434A; or (viii) any combination of (i)-(vii).

Embodiment 73. The antibody or antigen-binding fragment of any one of Embodiments 70-72, or the polypeptide of any one of Embodiments 70-72, wherein the mutation that increases binding affinity to a human FcRn comprises M428L/N434S.

Embodiment 74. The antibody or antigen-binding fragment of any one of Embodiments 70-73, or the polypeptide of any one of Embodiments 70-73, wherein the mutation that enhances binding to a FcγR comprises S239D; I332E; A330L; G236A; or any combination thereof.

Embodiment 75. The antibody or antigen-binding fragment of any one of Embodiments 70-74, or the polypeptide of any one of Embodiments 70-74, wherein the mutation that enhances binding to a FcγR comprises: (i) S239D/I332E; (ii) S239D/A330L/I332E; (iii) G236A/S239D/I332E; or (iv) G236A/A330L/I332E, wherein the Fc polypeptide or fragment thereof optionally comprises Ser at position 239.

Embodiment 76. The antibody or antigen-binding fragment of any one of Embodiments 1-44 and 53-75, or the polypeptide of any one of Embodiments 45-52 and 63-75, which comprises a mutation that alters glycosylation, wherein the mutation that alters glycosylation comprises N297A, N297Q, or N297G, and/or which is aglycosylated and/or afucosylated.

Embodiment 77. An antibody comprising: (1) a heavy chain comprising or consisting of the amino acid sequence set forth in SEQ ID NO.:212; and (2) a light chain comprising or consisting of the amino acid sequence set forth in SEQ ID NO.:214.

Embodiment 78. An antibody comprising: (1) a heavy chain comprising or consisting of the amino acid sequence set forth in SEQ ID NO.: 213; and (2) a light chain comprising or consisting of the amino acid sequence set forth in SEQ ID NO.:214.

Embodiment 79. An antibody comprising: (1) two heavy chains, each comprising or consisting of the amino acid sequence set forth in SEQ ID NO.:212; and (2) two light chains, each comprising or consisting of the amino acid sequence set forth in SEQ ID NO.:214.

Embodiment 80. An antibody comprising: (1) two heavy chains, each comprising or consisting of the amino acid sequence set forth in SEQ ID NO.: 213; and (2) two light chains, each comprising or consisting of the amino acid sequence set forth in SEQ ID NO.:214.

Embodiment 81. An isolated polynucleotide encoding the antibody or antigen-binding fragment of any one of Embodiments 1-44 and 53-80, or encoding a VH, a heavy chain, a VL, and/or a light chain of the antibody or the antigen-binding fragment.

Embodiment 82. An isolated polynucleotide encoding the polypeptide of any one of Embodiments 45-52 and 63-76.

Embodiment 83. The polynucleotide of Embodiment 81 or 82, wherein the polynucleotide comprises deoxyribonucleic acid (DNA) or ribonucleic acid (RNA), wherein the RNA optionally comprises messenger RNA (mRNA).

Embodiment 84. The polynucleotide of any one of Embodiments 81-83, comprising a modified nucleoside, a cap-1 structure, a cap-2 structure, or any combination thereof.

Embodiment 85. The polynucleotide of Embodiment 84, wherein the polynucleotide comprises a pseudouridine, a N6-methyladenonsine, a 5-methylcytidine, a 2-thiouridine, or any combination thereof.

Embodiment 86. The polynucleotide of Embodiment 84, wherein the pseudouridine comprises N1-methylpseudouridine.

Embodiment 87. The polynucleotide of any one of Embodiments 81-86, which is codon-optimized for expression in a host cell.

Embodiment 88. The polynucleotide of Embodiment 87, wherein the host cell comprises a human cell.

Embodiment 89. The polynucleotide of any one of Embodiments 81-88, comprising a polynucleotide having at least 50% (e.g., 50%, 55%, 60%, 65%, 70/6, 75%, 80%, 85%, 90%, 91%, 92%, 94%, 94%, 95%, 96%, 97%, 98%, 99/6, or more) 25 identity to the polynucleotide sequence set forth in any one or more of SEQ ID NOs.: 198, 200, 1, 13, 25, 170, 37, 49, 61, 73, 85, 182, 97, 109, 121, 133, 145, 157, 6, 18, 30, 42, 54, 66, 78, 90, 102, 114, 126, 138, 150, 162, 7, 19, 31, 173, 176, 179, 43, 55, 67, 79, 91, 185, 188, 191, 103, 115, 127, 139, 151, 163, 12, 24, 36, 48, 60, 72, 84, 96, 108, 120, 132, 144, 156, 168, 202, 206, 204, 208, 227, and 229.

Embodiment 90. The polynucleotide of Embodiment 89, comprising the polynucleotide sequence of SEQ ID NO.:198 and/or the polynucleotide sequence of SEQ ID NO.:200.

Embodiment 91. A recombinant vector comprising the polynucleotide of 5 any one of Embodiments 81-90.

Embodiment 92. A host cell comprising the polynucleotide of any one of Embodiments 81-90 and/or the vector of Embodiment 91, wherein the polynucleotide is optionally heterologous to the host cell and/or wherein the host cell is capable of expressing the encoded antibody or antigen-binding fragment or polypeptide.

Embodiment 93. An isolated human B cell comprising the polynucleotide of any one of Embodiments 81-90 and/or the vector of Embodiment 91, wherein polynucleotide is optionally heterologous to the human B cell and/or wherein the human B cell is immortalized.

Embodiment 94. A composition comprising: (i) the antibody or antigen-binding fragment of any one of Embodiments 1-44 and 53-80; (ii) the polypeptide of any one of Embodiments 45-52 and 63-76; (iii) the polynucleotide of any one of Embodiments 81-90; (iv) the recombinant vector of Embodiment 91; (v) the host cell of Embodiment 92; and/or (vi) the human B cell of Embodiment 93, and a pharmaceutically acceptable excipient, carrier, or diluent.

Embodiment 95. The composition of Embodiment 94, comprising a first antibody or antigen-binding fragment and a second antibody or antigen-binding fragment, wherein each of the first antibody or antigen-binding fragment and the second antibody or antigen-binding fragment are different and are each according any one of Embodiments 1-44 and 53-80.

Embodiment 96. A composition comprising the polynucleotide of any one of Embodiments 81-90 or the vector of Embodiment 91 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

Embodiment 97. A method of making an antibody or antigen-binding fragment of any one of Embodiments 1-44 and 53-80, comprising culturing the host cell of Embodiment 92 or the human B cell of Embodiment 93 for a time and under conditions sufficient for the host cell or human B cell, respectively, to express the antibody or antigen-binding fragment.

Embodiment 98. The method of Embodiment 97, further comprising isolating the antibody or antigen-binding fragment.

Embodiment 99. A method of treating or preventing an IAV infection and/or an IBV infection in a subject, the method comprising administering to the subject an effective amount of: (i) the antibody or antigen-binding fragment of any one of Embodiments 1-44 and 53-80; (ii) the polypeptide of any one of Embodiments 45-52 and 63-76; (iii) the polynucleotide of any one of Embodiments 81-90; (iv) the recombinant vector of Embodiment 91; (v) the host cell of Embodiment 92; (vi) the human B cell of Embodiment 93; and/or (vii) the composition of any one of Embodiments 94-96.

Embodiment 100. A method of treating or preventing an influenza infection in a human subject, the method comprising administering to the subject the 20 polynucleotide of any one of Embodiments 81-90, the recombinant vector of Embodiment 91, or the composition of Embodiment 96, wherein the polynucleotide comprises mRNA.

Embodiment 101. The method of Embodiment 100, wherein the influenza infection comprises an IAV infection and/or an IBV infection.

Embodiment 102. The method of any one of Embodiments 99-101, comprising administering a single dose of the antibody or antigen-binding fragment, polypeptide, polynucleotide, recombinant vector, host cell, or composition to the subject.

Embodiment 103. The method of any one of Embodiments 99-101, comprising administering two or more doses of the antibody or antigen-binding fragment, polypeptide, polynucleotide, recombinant vector, host cell, or composition to the subject.

Embodiment 104. The method of any one of Embodiments 99-103, comprising administering a dose of the antibody or antigen-binding fragment, polypeptide, polynucleotide, recombinant vector, host cell, or composition to the subject once yearly, optionally in advance of or during an influenza season.

Embodiment 105. The method of any one of Embodiments 99-103, comprising administering a dose of the antibody or antigen-binding fragment, polypeptide, polynucleotide, recombinant vector, host cell, or composition to the subject two or more times per year; e.g. about once every 6 months.

Embodiment 106. The method of any one of Embodiments 99-105, comprising administering the antibody or antigen-binding fragment, polypeptide, polynucleotide, recombinant vector, host cell, or composition intramuscularly, subcutaneously, or intravenously.

Embodiment 107. The method of any one of Embodiments 99-106, wherein the treatment and/or prevention comprises post-exposure prophylaxis.

Embodiment 108. The method of any one of Embodiments 99-107, wherein the subject has received, is receiving, or will receive an antiviral.

Embodiment 109. The method of Embodiment 108, wherein the antiviral comprises a neuraminidase inhibitor, an influenza polymerase inhibitor, or both.

Embodiment 110. The method of Embodiment 108 or 109, wherein the antiviral comprises oseltamivir, zanamivir, baloxavir, peramivir, laninamivir, or any combination thereof.

Embodiment 111. The antibody or antigen-binding fragment of any one of Embodiments 1-44 and 53-80, the polypeptide of any one of Embodiments 45-52 and 63-76, the polynucleotide of any one of Embodiments 81-90, the recombinant vector of Embodiment 91, the host cell of Embodiment 92, the human B cell of Embodiment 93, and/or the composition of any one of Embodiments 94-96, for use in a method of treating or preventing an IAV infection and/or an IBV infection in a subject.

Embodiment 112. The antibody or antigen-binding fragment of any one of Embodiments 1-44 and 53-80, the polypeptide of any one of Embodiments 45-52 and 63-76, the polynucleotide of any one of Embodiments 81-90, the recombinant vector of Embodiment 91, the host cell of Embodiment 92, the human B cell of Embodiment 93, and/or the composition of any one of Embodiments 94-96, for use in the preparation of a medicament for the treatment or prevention of an IAV infection and/or an IBV infection in a subject.

Embodiment 113. A method for in vitro diagnosis of an IAV infection and/or an IBV infection, the method comprising: (i) contacting a sample from a subject with an antibody or antigen-binding fragment of any one of Embodiments 1-44 and 53-80; and (ii) detecting a complex comprising an antigen and the antibody, or comprising an antigen and the antigen-binding fragment.

Embodiment 114. The method of any one of Embodiments 99-110 and 113 or the antibody or antigen-binding fragment, the polypeptide, the polynucleotide, the recombinant vector, the host cell, the human B cell, and/or the composition for use of any one of Embodiments 111 and 112, wherein. (i) the IAV comprises a Group 1 IAV, a Group 2 IAV, or both, wherein, optionally, the Group 1 IAV NA comprises a N1, a N4, a N5, and/or a N8; and/or the Group 2 IAV NA comprises a N2, a N3, a N6, a N7, and/or a N9, wherein, further optionally, the N1 is from A/California/07/2009, is from A/California/07/2009 I223R/H275Y, is from A/Swine/Jiangsu/J004/2018, is from A/Stockholm/18/2007, is from A/Brisbane/02/2018, is from A/Michigan/45/2015, is from A/Mississippi/3/2001, is from A/Netherlands/603/2009, is from A/Netherlands/602/2009, is from A/Vietnam/1203/2004, is from A/G4/SW/Shangdong/1207/2016, is from A/G4/SW/Henan/SN13/2018, is from A/G4/SW/Jiangsu/J004/2018, and/or is from A/New Jersey/8/1976; the N4 is from A/mallard duck/Netherlands/30/2011; the N5 is from A/aquatic bird/Korea/CN5/2009; the N8 is from A/harbor seal/New Hampshire/179629/2011; the N2 is from A/Washington/01/2007, is from A/HongKong/68, is from A/HongKong/2671/2019, is from A/South Australia/34/2019, is from A/Switzerland/8060/2017, is from A/Singapore/INFIMH-16-0019/2016, is from A/Switzerland/9715293/2013, is from A/Leningrad/134/17/57, is from A/Florida/4/2006, is from A/Netherlands/823/1992, is from A/Norway/466/2014, is from is from A/Texas/50/2012, is from A/Victoria/361/2011, is from A/SW/Mexico/SG1444/2011, is from A/Aichi/2/1968, is from A/Bilthoven/21793/1972, is from A/Netherlands/233/1982, is from A/Shanghai/l 1/1987, is from A/Nanchang/933/1995, is from A/Fukui/45/2004, A/Brisbane/10/2007, is from A/Tanzania/205/2010; the N3 is from A/Canada/rv504/2004; the N6 is from A/swine/Ontario/01911/1/99; the N7 is from A/Netherlands/078/03; and/or the N9 is from A/Anhui/2013, is from A/Hong Kong/56/2015; and/or (ii) the IBV NA is from: B/Lee/10/1940 (Ancestral); B/Brisbane/60/2008 (Victoria); B/Malaysia/2506/2004 (Victoria); B/Malaysia/3120318925/2013 (Yamagata); B/Wisconsin/1/2010 (Yamagata); B/Yamanashi/166/1998 (Yamagata); B/Brisbane/33/2008 (Victoria); B/Colorado/06/2017 (Victoria); B/Hubei-wujiang/158/2009 (Yamagata); B/Massachusetts/02/2012 (Yamagata); B/Netherlands/234/2011; B/Perth/211/2001(Yamagata); B/Phuket/3073/2013 (Yamagata); B/Texas/06/2011 (Yamagata); B/HongKong/05/1972; B/Harbin/7/1994 (Victoria); B/Washington/02/2019 (Victoria); B/Perth/211/2011, or any combination thereof.

TABLE 1
TABLE OF CERTAIN SEQUENCES AND SEQ ID NUMBERS

| SEQ | | |
|---|---|---|
| NO | Sequence | Identifier |
| 1 | CAAGTTCAGCTGGTGCAGTCTGGGGCTGAGGTGA | FNI1 VH (wt-nt) |
| | AGAGGCCTGGGTCCTCGGTGAGGATCTCCTGCAA | |
| | GGCCTCTGGTGACACCTTCAACAACTATGTTCTC | |
| | AGCTGGGTGCGACAGGCCCCTGGACAAGGGCTTG | |
| | AGTGGATGGGGGGAATCATCCCTATCTCTGGTA | |
| | TCCCACATTACGCACAGAAGTTCCAGGGCAGAGT | |
| | CGCAATTATCGCGGACGAATCCGCGAGCACAGTC | |
| | TACATGGAGTTGAGCAGCCTACGATCTGAGGACTC | |
| | GGCCGTATATTACTGTGCGAGAGCGGTTTCCGAT | |
| | TATTTTAATCGAGACCTCGGCTGGGATGATTAC | |
| | TACTTTCCTTTGTGGGGCCAGGGCACCCTGGTCA | |
| | CCGTCTCCTCAG | |
| 2 | QVQLVQSGAEVKRPGSSVRISCKASGDTFNNYVLS | FNI1 VH (aa) |
| | WVRQAPGQGLEWMGGIIPISGIPHYAQKFQGRVAII | |
| | ADESASTVYMELSSLRSEDSAVYYCARAVSDYFNR | |
| | DLGWDDYYFPLWGQGTLVTVSS | |
| 3 | GDTFNNYV | FNI1 CDRH1 (aa) |
| 4 | IPISGIP | FNI1 CDRH2 (aa) |
| 5 | ARAVSDYFNRDLGWDDYYFPL | FNI1 CDRH3 (aa) |
| 6 | CAGGTGCAGCTGGTGCAGTCTGGAGCTGAGGTGAAGA | FNI1 VH (co-nt) |
| | GGCCAGGATCCAGCGTGCGGATCAGCTGCAAGGCTTC | |
| | TGGCGACACCTTCAACAATTACGTGCTGTCCTGGGTGA | |
| | GGCAGGCTCCAGGACAGGGACTGGAGTGGATGGGCGG | |
| | CATCATCCCCATCAGCGGCATCCCTCACTACGCCCAGA | |
| | AGTTTCAGGGCAGGGTGGCCATCATCGCTGACGAGTC | |
| | CGCTAGCACAGTGTATATGGAGCTGTCTTCCCTGAGAT | |
| | CTGAGGATTCCGCCGTGTACTATTGTGCCAGAGCCGTG | |
| | TCCGACTATTTCAACCGCGATCTGGGCTGGGACGATTA | |
| | CTATTTTCCACTGTGGGGACAGGGCACCCTGGTGACAG | |
| | TGAGCTCT | |
| 7 | GAAATAGTGATGACGCAGTCTCCAGCCACCCTGT | FNI1 Vk (wt-nt) |
| | CTGTGTCTCCAGGGGAAAGAGCCACCCTCTTCTGC | |
| | AGGGCCAGTCGGAGTGTTAGTGACAACTTAGCC | |
| | TGGTACCAGCAGAAACCTGGCCAGGCTCCCAGGC | |
| | TCCTCATCTTTGGTGCCTCCACCAGGGCCACTGG | |
| | TGTCCCAGCCAGGTTCGGTGGCAGTGGGTCTGGG | |
| | ACACAGTTCACTCTCACCATCAGCAGCCTGCAGTC | |
| | TGAAGATTTTGCAGTTTATTACTGTCAGCATTATA | |
| | ATACCTGGCCTCCGTGGACCTTCGGCCAAGGGA | |
| | CCAAGGTGGAAATCAAAC | |
| 8 | EIVMTQSPATLSVSPGERATLFCRASRSVSDNLAWY | FNI1 VK (aa) |
| | QQKPGQAPRLLIFGASTRATGVPARFGGSGSGTQFT | |
| | LTISSLQSEDFAVYYCQHYNTWPPWTFGQGTKVEI | |
| | K | |
| 9 | RSVSDN | FNI1 CDRL1 (aa) |
| 10 | GAS | FNI1 CDRL2 (aa) |
| 11 | QHYNTWPPWT | FNI1 CDRL3 (aa) |
| 12 | GAGATCGTGATGACCCAGTCTCCTGCCACACTGTC | FNI1 Vk (co-nt) |
| | CGTGTCCCCAGGCGAGAGGGCCACACTGTTCTGC | |
| | AGGGCTAGCAGGTCCGTGTCCGACAACCTGGCCT | |
| | GGTACCAGCAGAAGCCAGGCCAGGCTCCCAGACT | |
| | GCTGATCTTTGGAGCTTCCACCAGAGCTACAGGC | |
| | GTGCCAGCTAGGTTCGGAGGAAGCGGATCTGGCA | |
| | CCCAGTTTACCCTGACAATCTCCAGCCTGCAGAGC | |
| | GAGGATTTCGCCGTGTACTATTGTCAGCACTATAA | |
| | TACCTGGCCCCCTTGGACATTTGGCCAGGGCACC | |
| | AAGGTGGAGATCAAG | |
| 13 | CAGGTTCAGCTGGTGCAGTCTGGGGCTGAGGTGAAGA | FNI2 VH (wt-nt) |
| | GGCCTGGGTCCTCGGTGAGGGTCTCCTGCAAGGCTTCT | |
| | GGAGCCACCTTCAATAACCATGTTCTCACCTGGGTG | |
| | CGACAGGCCCCTGGACAAGGGCTTGAGTGGATGGGAG | |
| | GGATCATCCCTGTCTCTGGAAAAACAACCTACGCAC | |
| | AGAAGTTCCAGGGCAGAGTCGCGATAAGCACGGACGA | |
| | ATCCGCGAGCACAGCCTATATGGAGTTGAGCAGCCTG | |
| | AGATCTGAGGACTCGGCCATATATTACTGTGCGAGAG | |
| | CGGTTTCCGATTACTTTAATCGAGACCTCGGCTGG | |
| | GAAGATTATTACTTTCCGATCTGGGGCCAGGGCACC | |
| | CTGGTCACCGTCTCTTCAG | |
| 14 | QVQLVQSGAEVKRPGSSVRVSCKASGATfNNHVLT | FNI2 VH (aa) |
| | WVRQAPGQGLEWMGGIIPVSGKTTYAQKFQGRVAI | |
| | STDESASTAYMELSSLRSEDSAIYYCARAVSDYfNR | |
| | DLGWEDYYFPIWGQGTLVTVSS | |
| 15 | GATfNNHV | FNI2 CDRH1 (aa) |
| 16 | IIPVSGKT | FNI2 CDRH2 (aa) |
| 17 | ARAVSDYFNRDLGWEDYYFPI | FNI2 CDRH3 (aa) |
| 18 | CAGGTGCAGCTGGTGCAGTCTGGAGCTGAGGTGA | FNI2 VH (co-nt) |
| | AGAGGCCAGGATCCAGCGTGCGGGTGAGCTGCAA | |
| | GGCTTCTGGAGCTACCTTCAACAATCACGTGCTGA | |
| | CATGGGTGAGGCAGGCTCCAGGACAGGGACTGGA | |
| | GTGGATGGGCGGCATCATCCCCGTGTCCGGCAAG | |
| | ACCACATACGCCCAGAAGTTTCAGGGCAGGGTGG | |
| | CTATCAGCACCGATGAGTCCGCCAGCACAGCTTA | |
| | TATGGAGCTGTCTTCCCTGAGATCTGAGGACTCCG | |
| | CCATCTACTATTGTGCCAGAGCCGTGTCCGACTAC | |
| | TTCAACCGCGATCTGGGCTGGGAGGACTACTATTT | |
| | TCCCATCTGGGGCCAGGGCACCCTGGTGACAGTG | |
| | AGCTCT | |
| 19 | GACGTAGTGATGACGCAGTCTCCAGCCACCCTGT | FNI2 Vk (wt-nt) |
| | CTGTGTCTCCAGGGGAAAGAGCCACCCTCTCCTG | |
| | CAGGGCCAGTCAGAGTGTTAGTAGCAACTTGGC | |
| | CTGGTACCAGCAGAAACCTGGCCAGGCTCCCAGG | |
| | CTCCTCATCTATGGTGCATCCACCAGGGCCACTG | |
| | GTGTCCCAGCCAGGTTCAGTGGCAGTGGGTCTGG | |
| | GACACAGTTCACTCTCACCATCAGCAGCCTGCAG | |
| | TCTGAAGATTTTGCAGTTTATTACTGTCAGCACTA | |
| | TAATAACTGGCCTCCGTGGACGTTCGGCCAAGG | |
| | GACCAAGTTGGAAATCAAAC | |
| 20 | DVVMTQSPATLSVSPGERATLSCRASQSVSSNLAW | FNI2 VK (aa) |
| | YQQKPGQAPRLLIYGASTRATGVPARFSGSGSGTQF | |
| | TLTISSLQSEDFAVYYCQHYNNWPPWTFGQGTKLE | |
| | IK | |
| 21 | QSVSSN | FNI2 CDRL1 (aa) |
| 22 | GAS | FNI2 CDRL2 (aa) |
| 23 | QHYNNWPPWT | FNI2 CDRL3 (aa) |
| 24 | GACGTGGTCATGACCCAGTCTCCTGCCACACTGA | FNI2 Vk (co-nt) |
| | GCGTGTCTCCAGGAGAGAGGGCCACCCTGTCCTG | |
| | CAGGGCTTCCCAGAGCGTGTCCAGCAACCTGGCC | |
| | TGGTACCAGCAGAAGCCAGGCCAGGCTCCCAGGC | |
| | TGCTGATCTATGGAGCTAGCACCAGAGCTACAGG | |
| | CGTGCCAGCTCGCTTCTCTGGATCCGGAAGCGGC | |
| | ACACAGTTTACCCTGACAATCTCTTCCCTGCAGTC | |
| | TGAGGATTTCGCCGTGTACTATTGTCAGCACTACA | |
| | ACAATTGGCCCCCTTGGACCTTTGGCCAGGGCAC | |
| | AAAGCTGGAGATCAAG | |
| 25 | CAGGTTCAGCTGGTGCAGTCGGGGGCTGAGGTGA | FNI3 VH (wt-nt) |
| | AGAGGCCTGGGTCCTCGGTGAAGGTCTCCTGCAA | |
| | GGCTTCTGGAGCCACCTTCAGCAACAATGTTAT | |
| | AGCCTGGGTGCGACAGGCCCCTGGACAAGGGCTT | |
| | GAGTGGATGGGGGGGATCCACCCTATCTCTGCT | |
| | ACAGCAACCTACGCACAGAAGTTCCAGGGCAGAG | |
| | TCGCGATTGCCGCGGACGAATTAACGAGCACAGC | |
| | CTACATGGAGTTGAATGGCCTGAGATCTGAGGACT | |
| | CGGCCGTGTATTACTGTGCGAGAGCGGGGTCCG | |
| | ATTACTTTAATAGAGACCTCGGCTGGGAAAATT | |
| | ACTACTTTGACTCCTGGGGCCAGGGAACCCTGGT | |
| | CACCGTCTCGTCAG | |
| 26 | QVQLVQSGAEVKRPGSSVKVSCKASGATFSNNVIA | FNI3 VH (aa) |
| | WVRQAPGQGLEWMGGIHPISATATYAQKFQGRVA | |
| | IAADELTSTAYMELNGLRSEDSAVYYCARAGSDYF | |
| | NRDLGWENYYFDSWGQGTLVTVSS | |
| 27 | GATFSNNV | FNI3 CDRH1 (aa) |
| 28 | IHPISATA | FNI3 CDRH2 (aa) |
| 29 | ARAGSDYFNRDLGWENYYFDS | FNI3 CDRH3 (aa) |
| 30 | CAGGTGCAGCTGGTGCAGTCCGGAGCTGAGGTGA | FNI3 VH (co-nt) |
| | AGAGGCCAGGATCCAGCGTGAAGGTGTCCTGCAA | |
| | GGCCAGCGGCGCTACCTTCAGCAACAATGTGATC | |
| | GCTTGGGTGAGACAGGCTCCAGGACAGGGACTGG | |
| | AGTGGATGGGAGGAATCCACCCTATCAGCGCCAC | |
| | CGCTACATACGCCCAGAAGTTTCAGGGCAGAGTG | |
| | GCTATCGCCGCTGACGAGCTGACCTCTACAGCCT | |
| | ATATGGAGCTGAACGGCCTGCGCAGCGAGGATTC | |
| | CGCCGTGTACTATTGTGCCAGGGCTGGCTCTGACT | |
| | ACTTCAACCGGGATCTGGGCTGGGAGAATTACTA | |
| | TTTTGACTCCTGGGGCCAGGGCACCCTGGTGACA | |
| | GTGTCTTCC | |
| 31 | GAAATATTGATGACGCAGTCTCCAGCCACCCTGT | FNI3 Vk (wt-nt) |
| | CTGTGTCTCCAGGGGAAAGAGCCACCCTCTCCTG | |
| | CAGGGCCAGTCAGGATGTTAGCGGCAACTTAGC | |
| | CTGGTACCAGCAGAGACCTGGCCAGGCTCCCAGG | |
| | CTCCTTATCTATGGTGCATCCACGAGGGCCACTG | |
| | GTGTCCCAGCCAGGTTCACTGGCGCTGGGTCTGG | |
| | GACAGAGTTCACTCTCACCATCAGCAGCCTGCAG | |
| | TCTGAGGATTTTGCACTTTATTACTGTCAGCACTA | |
| | TAATAACTGGCCTCCGTGGACCTTCGGCCAAGG | |
| | GACCAAGGTGGAAATCAAAC | |
| 32 | EILMTQSPATLSVSPGERATLSCRASQDVSGNLAWY | FNI3 Vk (aa) |
| | QQRPGQAPRLLIYGASTRATGVPARFTGAGSGTEFT | |
| | LTISSLQSEDFALYYCQHYNNWPPWTFGQGTKVEI | |
| | K | |
| 33 | QDVSGN | FNI3 CDRL1 (aa) |
| 34 | GAS | FNI3 CDRL2 (aa) |
| 35 | QHYNNWPPWT | FNI3 CDRL3 (aa) |
| 36 | GAGATCCTGATGACCCAGTCCCCTGCCACACTGTC | FNI3 Vk (co-nt) |
| | CGTGTCCCCAGGAGAGAGGGCCACCCTGAGCTGC | |
| | AGGGCTTCTCAGGACGTGTCCGGCAACCTGGCCTG | |
| | GTACCAGCAGAGACCAGGACAGGCTCCAAGGCTG | |
| | CTGATCTATGGAGCTTCCACCAGGGCTACAGGCGT | |
| | GCCAGCTAGATTCACCGGCGCTGGAAGCGGCACA | |
| | GAGTTTACCCTGACAATCTCCAGCCTGCAGTCTGA | |
| | GGATTTCGCTCTGTACTATTGTCAGCACTACAACA | |
| | ATTGGCCCCCTTGGACCTTTGGCCAGGGCACAAAG | |
| | GTGGAGATCAAG | |
| 37 | CAGGAGCAGCTGGTACAGTCTGGGGCTGAGGTGA | FNI4 VH (wt-nt) |
| | AGAAGCCGGGGTCCTCGGTGAGGGTCTCCTGCAA | |
| | GGCCTCTGGAGACACCTTCAGCAGATATACTAT | |
| | CAGCTGGGTTCGACAGGCCCCCGGACAAGGACTT | |
| | GAGTGGATGGGAGGGATCATCGCTCTCTCTCGA | |
| | AGAGCGACATACGCACAGAAGTTCCAGGGCAGA | |
| | GTTACCATTACCGCGGACGAATCCGCGACCACAG | |
| | CCTACATACAACTGAGCGGCCTGACATCTGACGAC | |
| | ACGGCCGTATATTACTGTGCGAGAGCACACTCCG | |
| | ATTACTTTAATAGAGACCTCGGCTGGGAAGATT | |
| | ACTACTTTGACTACTGGGGCCAGGGAACCCTGGT | |
| | CACCGTCTCCTCAG | |
| 38 | QEQLVQSGAEVKKPGSSVRVSCKASGDTFSRYTIS | FNI4 VH (aa) |
| | WVRQAPGQGLEWMGGIIALSRRATYAQKFQGRVTI | |
| | TADESATTAYIQLSGLTSDDTAVYYCARAHSDYFN | |
| | RDLGWEDYYFDYWGQGTLVTVSS | |
| 39 | GDTFSRYT | FNI4 CDRH1 (aa) |
| 40 | IIALSRRA | FNI4 CDRH2 (aa) |
| 41 | ARAHSDYFNRDLGWEDYYFDY | FNI4 CDRH3 (aa) |
| 42 | CAGGAGCAGCTGGTGCAGTCCGGAGCTGAGGTGA | FNI4 VH (co-nt) |
| | AGAAGCCAGGATCCAGCGTGAGAGTGAGCTGCAA | |
| | GGCTTCTGGCGACACCTTCTCTAGATACACAATCT | |
| | CCTGGGTGCGCCAGGCTCCTGGACAGGGACTGGA | |
| | GTGGATGGGAGGAATCATCGCTCTGAGCAGGCGG | |
| | GCCACCTACGCTCAGAAGTTTCAGGGCCGCGTGA | |
| | CCATCACAGCCGATGAGTCTGCCACCACAGCTTA | |
| | TATCCAGCTGTCCGGCCTGACCAGCGACGATACA | |
| | GCCGTGTACTATTGTGCCAGGGCTCACAGCGACT | |
| | ACTTCAACCGGGATCTGGGCTGGGAGGACTACTA | |
| | TTTTGATTATTGGGGCCAGGGCACCCTGGTGACA | |
| | GTGTCTTCC | |
| 43 | GAAGTAGTGCTGACGCAGTCTCCAGCCACCCTGT | FNI4 Vk (wt-nt) |
| | CTGTGTCTCTAGGGGAAAGAGCCATCCTCTCCTGC | |
| | AGGGCCAGTCAGAGTGTTAGCACCAACTTAGCC | |
| | TGGTACCAGCAGAGACCTGGCCAGGCTCCCAGGC | |
| | TCCTCATCTCTGGTGCATCCACCAGGGCCACGGG | |
| | TATCCCAGCCAGGTTCAGTGGCAGTGGGTCTGGG | |
| | ACAGAGTTCACGCTCACCATCAGCAGCCTGCAGT | |
| | CTGAAGATTTTGCAGTTTATTACTGTCAGCAGTA | |
| | TAATAACTGGCCTCCGTGGACGTTCGGCCAAGG | |
| | GACCAAGGTGGAAATCAGAC | |
| 44 | EVVLTQSPATLSVSLGERAILSCRASQSVSTNLAWY | FNI4 VK (aa) |
| | QQRPGQAPRLLISGASTRATGIPARFSGSGSGTEFTL | |
| | TISSLQSEDFAVYYCQQYNNWPPWTFGQGTKVEIR | |
| 45 | QSVSTN | FNI4 CDRL1 (aa) |
| 46 | GAS | FNI4 CDRL2 (aa) |
| 47 | QQYNNWPPWT | FNI4 CDRL3 (aa) |
| 48 | GAGGTGGTGCTGACCCAGTCCCCTGCCACACTGT | IFNI4 Vk (co-nt) |
| | CCGTGTCCCTGGGAGAGAGGGCTATCCTGAGCTG | |
| | CAGGGCTAGCCAGTCCGTGTCCACCAACCTGGCC | |
| | TGGTACCAGCAGAGACCAGGACAGGCTCCAAGGC | |
| | TGCTGATCAGCGGAGCTTCTACCAGGGCTACAGG | |
| | CATCCCAGCCAGATTCAGCGGCTCTGGCTCCGGC | |
| | ACAGAGTTTACCCTGACAATCTCCAGCCTGCAGTC | |
| | TGAGGACTTCGCCGTGTACTATTGTCAGCAGTATA | |
| | ACAATTGGCCCCCTTGGACCTTTGGCCAGGGCAC | |
| | AAAGGTGGAGATCAGG | |
| 49 | CAGGTGCAGCTGATACAATCTGAGGCTGAGGTGAAGA | FNI5 VH (wt-nt) |
| | AGCCTGGGTCCTCGGTGAGGGTCTCCTGCAAGGCTTCT | |
| | GGAGACACCTTCAGCAAATATACTATCGGCTGGGTG | |
| | CGACAGGCCCCCGGACAAGGGCTTGAGTGGATGGGAG | |
| | GGATCATCCCTCTCTCTCGAACAGCGACCTACGCAC | |
| | AGAAGTTCCAGGGCAGAGTCACGATTACCGCGGACGA | |
| | ATCCACGACCACAGTTTACATGCAACTGAGCGGCCTG | |
| | AGATCTGACGACACGGCCGCATATTACTGTGCGAGAG | |
| | CACGCTCGGATTACTTTAATAGAGACCTCGGCTGG | |
| | GACGATTACTACTTTGATTACTGGGGCCAGGGAACC | |
| | CTGGTCACCGTCTCCTCAG | |
| 50 | QVQLIQSEAEVKKPGSSVRVSCKASGDTFSKYTIGW | FNI5 VH (aa) |
| | VRQAPGQGLEWMGGIIPLSRTATYAQKFQGRVTIT | |
| | ADESTTTVYMQLSGLRSDDTAAYYCARARSDYFN | |
| | RDLGWDDYYFDYWGQGTLVTVSS | |
| 51 | GDTFSKYT | FNI5 CDRH1 (aa) |
| 52 | IIPLSRTA | FNI5 CDRH2 (aa) |
| 53 | ARARSDYFNRDLGWDDYYFDY | FNI5 CDRH3 (aa) |
| 54 | CAGGTGCAGCTGATCCAGAGCGAGGCCGAGGTGA | FNI5 VH (co-nt) |
| | AGAAGCCAGGCTCCAGCGTGAGGGTGAGCTGCAA | |
| | GGCTTCTGGCGACACATTCTCTAAGTACACCATCG | |
| | GATGGGTGCGGCAGGCTCCAGGACAGGGCCTGGA | |
| | GTGGATGGGCGGCATCATCCCTCTGTCTAGAACA | |
| | GCCACCTACGCTCAGAAGTTTCAGGGCCGCGTGA | |
| | CAATCACCGCTGACGAGTCCACCACAACCGTGTA | |
| | TATGCAGCTGTCCGGCCTGAGAAGCGACGATACA | |
| | GCCGCTTACTATTGTGCCAGGGCTCGGTCCGACTA | |
| | CTTCAACCGCGATCTGGGCTGGGACGATTACTATT | |
| | TTGATTATTGGGGCCAGGGCACACTGGTGACCGT | |
| | GTCTTCC | |
| 55 | GAAATAGTGATGACGCAGTCTCCAGCCAACCTGT | FNI5 Vk (wt-nt) |
| | CTGTGTCTCCAGGGGAAAGAGCCACCCTCTCCTG | |
| | CAGGGCCAGTCAGACTGTTAGCACCAACTTAGC | |
| | CTGGTACCAGCAGAAGCCTGGCCAGGCTCCCAGG | |
| | CTCCTCATCTCTGGTGCATCCACCAGGGCCACTG | |
| | GTATCCCAGCCAGGTTCAGTGGCAGTGGGTCTGG | |
| | GACAGAGTTCACGCTCACCATCAGCAGCCTGCAG | |
| | TCTGAAGATTTTGCAGTTTATTACTGTCAGCAGTA | |
| | TAATAATTGGCCTCCGTGGACGTTCGGCCAAGG | |
| | GACCAAGGTGGAAATCAGAC | |
| 56 | EIVMTQSPANLSVSPGERATLSCRASQTVSTNLAWY | FNI5 VK (aa) |
| | QQKPGQAPRLLISGASTRATGIPARFSGSGSGTEFTL | |
| | TISSLQSEDFAVYYCQQYNNWPPWTFGQGTKVEIR | |
| 57 | QTVSTN | FNI5 CDRL1 (aa) |
| 58 | GAS | FNI5 CDRL2 (aa) |
| 59 | QQYNNWPPWT | FNI5 CDRL3 (aa) |
| 60 | GAGATCGTGATGACCCAGTCCCCTGCTAACCTGTC | FNI5 Vk (co-nt) |
| | CGTGTCCCCAGGAGAGAGGGCCACACTGTCCTGC | |
| | CGGGCTAGCCAGACCGTGTCTACAAATCTGGCCT | |
| | GGTACCAGCAGAAGCCAGGACAGGCTCCAAGGCT | |
| | GCTGATCAGCGGAGCTTCTACCAGAGCTACAGGC | |
| | ATCCCAGCTCGCTTCAGCGGATCTGGATCCGGCA | |
| | CCGAGTTTACCCTGACAATCTCCAGCCTGCAGAG | |
| | CGAGGACTTCGCCGTGTACTATTGTCAGCAGTATA | |
| | ACAATTGGCCCCCTTGGACCTTTGGCCAGGGCAC | |
| | AAAGGTGGAGATCAGA | |
| 61 | CAGGTGCAGCTGGTGCAGTCTGGGGCTGAGGTGA | FNI6 VH (wt-nt) |
| | AGAAGCCTGGGTCCTCGGTGAAGGTCTCCTGCAA | |
| | GGCCTCTGGAGGCACCTTCAGTAGTCAAGTTAT | |
| | CAGCTGGGTGCGAGAGGCCCCAGGACAAGGGCTT | |
| | GAGTGGATGGGAGGGATCATTCCTATCACTGGA | |
| | ATAGCGAACAACGCACAGAAGTTCCAGGGCAGA | |
| | GTCACGATTACCGCGGACGGATCCACGGGCACAG | |
| | TCTACATGGAGTTGAGCAGCCTGAGATCTGGGGA | |
| | CACGGCCGTCTATTACTGTGCGAGAGCGGGTTC | |
| | GGATTATTTTAATAGAGACCTCGGCTGGGAAA | |
| | ATTACTACTTTGAATATTGGGGCCAGGGAACCCT | |
| | GGTCACCGTCTCCTCAG | |
| 62 | QVQLVQSGAEVKKPGSSVKVSCKASGGTFSSQVIS | FN16 VH (aa) |
| | WVREAPGQGLEWMGGIIPITGIANNAQKFQGRVTI | |
| | TADGSTGTVYMELSSLRSGDTAVYYCARAGSDYFN | |
| | RDLGWENYYFEYWGQGTLVTVSS | |
| 63 | GGTFSSQV | FNI6 CDRH1 (aa) |
| 64 | IIPITGIA | FNI6 CDRH2 (aa) |
| 65 | ARAGSDYFNRDLGWENYYFEY | FNI6 CDRH3 (aa) |
| 66 | CAGGTGCAGCTGGTGCAGAGCGGAGCTGAGGTGA | FNI6 VH (co-nt) |
| | AGAAGCCAGGCTCCAGCGTGAAGGTGTCTTGCAA | |
| | GGCTTCCGGCGGCACCTTCTCTTCCCAGGTCATCT | |
| | CTTGGGTGAGGGAGGCTCCAGGACAGGGACTGGA | |
| | GTGGATGGGCGGCATCATCCCTATCACAGGCATCG | |
| | CCAACAATGCTCAGAAGTTTCAGGGCAGAGTGAC | |
| | CATCACAGCCGACGGCAGCACCGGCACAGTGTAC | |
| | ATGGAGCTGAGCTCTCTGCGCTCTGGCGATACCGC | |
| | CGTGTACTATTGTGCCAGGGCTGGCTCCGACTACT | |
| | TCAACCGGGATCTGGGCTGGGAGAATTACTATTTT | |
| | GAGTATTGGGGCCAGGGCACCCTGGTGACAGTGT | |
| | CCAGC | |
| 67 | GAAATCGTGATGACACAGTCTCCAGCCACCCTGT | FNI6 Vk (wt-nt) |
| | CTGTATCTCCAGGGGAAAGAGCCATCCTCTCCTGC | |
| | AGGGCCAGTCAGAGTGTTAGCACCCACTTAGCC | |
| | TGGTACCAGCAGAAACCTGGCCAGGCTCCCAGAC | |
| | TCCTCGTTTTTGATGCATCCACCAGGGCCACTGG | |
| | TGTCCCAGCCAGATTCGGTGGCAGTGGGTCTGGG | |
| | ACAGAGTTCACTCTCACCATCAGCAGCCTGCAGT | |
| | CTGAAGATTCTGCTGTTTATTACTGTCAACACTAT | |
| | AATAACTGGCCTCCGTGGACGTTCGGCCAAGGG | |
| | ACCAACGTGGAAATCAGAC | |
| 68 | EIVMTQSPATLSVSPGERAILSCRASQSVSTHLAWY | FNI6 VK (aa) |
| | QQKPGQAPRLLVFDASTRATGVPARFGGSGSGTEFT | |
| | LTISSLQSEDSAVYYCQHYNNWPPWTFGQGTNVEI | |
| | R | |
| 69 | QSVSTH | FNI6 CDRL1 (aa) |
| 70 | DAS | FNI6 CDRL2 (aa) |
| 71 | QHYNNWPPWT | FNI6 CDRL3 (aa) |
| 72 | GAGATCGTGATGACCCAGTCTCCTGCCACACTGTCCGT | FNI6 Vk (co-nt) |
| | GTCCCCAGGAGAGAGGGCTATCCTGTCCTGCAGGGCT | |
| | AGCCAGTCCGTGTCCACCCACCTGGCCTGGTACCAGCA | |
| | GAAGCCAGGCCAGGCTCCCAGGCTGCTGGTGTTCGAC | |
| | GCTAGCACCAGAGCTACAGGCGTGCCAGCTAGGTTCG | |
| | GAGGAAGCGGATCTGGCACAGAGTTTACCCTGACAAT | |
| | CTCCAGCCTGCAGTCCGAGGATTCCGCCGTGTACTATT | |
| | GTCAGCATTATAACAATTGGCCCCCTTGGACCTTTGGC | |
| | CAGGGCACAAACGTGGAGATCAGA | |
| 73 | CAAGTGCAGCTGGTGCAGTCTGGGGCTGAGGTGA | FNI7 VH (wt-nt) |
| | AGAAGCCTGGGTCCTCGGTGAAAGTCTCCTGTAA | |
| | GACTTCTGGAGGCACCTTCAATAGGCAAGTTAT | |
| | CAGCTGGGTGCGACAGGCCCCAGGACAAGGACTT | |
| | GAGTGGATGGGAGGGATCCTCCCTCTTACTGGT | |
| | AGAGGGGACGAGGCAGAGAGGTTTCAGGGCAGA | |
| | GTCACCATTACCGCGGACGAATCTGAGAGTACAG | |
| | TCTACATGGACTTGAGCAGCCTGAGATCTGGGGA | |
| | CACGGCCGTCTATTACTGTGCGAGAGCGCGTTC | |
| | GGATTACTTTAATAGAGACCTCGGCTGGGAAA | |
| | ATTACTACTTTGAATCTTGGGGCCAGGGAACCCT | |
| | GGTCACCGTCTCCTCAG | |
| 74 | QVQLVQSGAEVKKPGSSVKVSCKTSGGTFNRQVIS | FNI7 VH (aa) |
| | WVRQAPGQGLEWMGGILPLTGRGDEAERFQGRVT | |
| | ITADESESTVYMDLSSLRSGDTAVYYCARARSDYF | |
| | NRDLGWENYYFESWGQGTLVTVSS | |
| 75 | GGTFNRQV | FNI7 CDRH1 (aa) |
| 76 | ILPLTGRG | FNI7 CDRH2 (aa) |
| 77 | ARARSDYFNRDLGWENYYFES | FNI7 CDRH3 (aa) |
| 78 | CAGGTGCAGCTGGTGCAGTCCGGAGCTGAGGTGA | FNI7 VH (co-nt) |
| | AGAAGCCAGGCTCCAGCGTGAAGGTGTCTTGCAA | |
| | GACCTCCGGCGGCACATTCAACAGGCAGGTCATC | |
| | AGCTGGGTGCGGCAGGCTCCAGGACAGGGACTGG | |
| | AGTGGATGGGAGGAATCCTGCCTCTGACCGGCAG | |
| | GGGCGACGAGGCCGAGAGATTTCAGGGCCGCGTG | |
| | ACCATCACAGCTGATGAGTCCGAGAGCACCGTGT | |
| | ACATGGACCTGTCTTCCCTGAGAAGCGGCGATAC | |
| | AGCCGTGTACTATTGTGCCAGGGCTCGGTCTGACT | |
| | ATTTCAACCGCGATCTGGGCTGGGAGAATTACTA | |
| | TTTTGAGTCTTGGGGCCAGGGCACCCTGGTGACA | |
| | GTGAGCTCT | |
| 79 | GAAATCGTGATGACGCAGTCTCCAGCCACCCTGT | FNI7 Vk (wt-nt) |
| | CTGTATCTCCAGGGGAAAGAGCCACCCTCTCCTG | |
| | CAGGGCCAGTCAGAGTGTTAGTACCGACTTAGT | |
| | CTGGTACCAGCAGAAACCTGGCCAGGCTCCCCGG | |
| | CTCCTCATTTATGATGCATCCACTAGGGCCACTG | |
| | GTATCCCAGCCAGGTTCGGTGGCAGGGGGTCTGG | |
| | GACAGAGTTCACTCTCACCATCAGCAGCCTGCAG | |
| | TCTGAAGATTCTGCTGTTTATTACTGTCAGCACTA | |
| | TTCTTACTGGCCTCCGTGGACATTCGGCCAAGG | |
| | GACCAAAGTGGAAATCAATC | |
| 80 | EIVMTQSPATLSVSPGERATLSCRASQSVSTDLVWY | FNI7 VK (aa) |
| | QQKPGQAPRLLIYDASTRATGIPARFGGRGSGTEFTL | |
| | TISSLQSEDSAVYYCQHYSYWPPWTFGQGTKVEIN | |
| 81 | QSVSTD | FNI7 CDRL1 (aa) |
| 82 | DAS | FNI7 CDRL2 (aa) |
| 83 | QHYSYWPPWT | FNI7 CDRL3 (aa) |
| 84 | GAGATCGTGATGACCCAGTCCCCTGCCACACTGT | FNI7 Vk (co-nt) |
| | CCGTGTCCCCAGGAGAGAGAGCCACCCTGAGCTG | |
| | CAGGGCTAGCCAGTCCGTGTCCACAGACCTGGTG | |
| | TGGTACCAGCAGAAGCCAGGACAGGCTCCAAGGC | |
| | TGCTGATCTATGATGCCTCTACCAGAGCTACAGGC | |
| | ATCCCAGCTAGGTTCGGAGGAAGGGGATCCGGCA | |
| | CCGAGTTTACCCTGACAATCTCCAGCCTGCAGAG | |
| | CGAGGACTCCGCCGTGTACTATTGTCAGCACTAC | |
| | AGCTATTGGCCCCCTTGGACCTTCGGCCAGGGCA | |
| | CAAAGGTGGAGATCAAC | |
| 85 | CAGGTCCACCTGGTGCAGTCTGGGGCTGAGGTGAAGG | FN19 VH (wt-nt) |
| | AGCCTGGGTCCTCGGTGACGGTCTCCTGCAAGGCATCT | |
| | GGAGGCAGCTTCAACAACCAGGCTATTAGCTGGGTG | |
| | CGACAGGCCCCAGGACAAGGCCTTGAGTGGATGGGAG | |
| | GGATCTTCCCTATCTCTGGCACACCGACCAGCGCAC | |
| | AGAGGTTCCAGGGCAGAGTCACATTTACCGCGGACGA | |
| | GTCCACGACCACAGTCTACATGGATCTGAGCAGCCTG | |
| | AGATCTGACGACACGGCCGTCTACTACTGTGCGAGAG | |
| | CGGGTTCGGATTACTTTAATAGAGACCTCGGCTGG | |
| | GAAAACTACTACTTTGCGTCCTGGGGCCAGGGAACC | |
| | CTGGTCACCGTCTCCTCAG | |
| 86 | QVHLVQSGAEVKEPGSSVTVSCKASGGSFNNQAIS | FNI9 VH (aa) |
| | WVRQAPGQGLEWMGGIFPISGTPTSAQRFQGRVTF | |
| | TADESTTTVYMDLSSLRSDDTAVYYCARAGSDYFN | |
| | RDLGWENYYFASWGQGTLVTVSS | |
| 87 | GGSFNNQA | FNI9 CDRH1 (aa) |
| 88 | IFPISGTP | FNI9 CDRH2 (aa) |
| 89 | ARAGSDYFNRDLGWENYYFAS | FNI9 CDRH3 (aa) |
| 90 | CAGGTGCACCTGGTGCAGAGCGGAGCTGAGGTGA | FNI9 VH (co-nt) |
| | AGGAGCCAGGATCCAGCGTGACAGTGTCTTGCAA | |
| | GGCTTCCGGCGGCAGCTTCAACAATCAGGCTATC | |
| | TCCTGGGTGAGGCAGGCTCCAGGACAGGGACTGG | |
| | AGTGGATGGGCGGCATCTTTCCCATCTCTGGCACA | |
| | CCTACCTCCGCCCAGAGGTTCCAGGGAAGGGTGA | |
| | CCTTCACCGCTGACGAGAGCACCACAACCGTGTA | |
| | CATGGATCTGTCTTCCCTGAGATCTGACGATACCG | |
| | CCGTGTACTATTGTGCCAGAGCTGGCTCCGACTAT | |
| | TTCAACCGCGATCTGGGCTGGGAGAATTACTATTT | |
| | TGCTTCCTGGGGCCAGGGCACACTGGTGACCGTG | |
| | AGCTCT | |
| 91 | GAAATCGTGATGACGCAGTCTCCAGCCACCCTGT | FN19 Vk (wt-nt) |
| | CTCTATCTTCAGGGGAAAGAGCCACCCTCTCCTGC | |
| | AGGGCCAGTCGGAGTGTTAGTAGCAACTTAGCC | |
| | TGGTACCAGCAGAAACCTGGCCAGGCTCCCAGGC | |
| | TCCTCATTTATGATGCATCCACCAGGGCCACTGG | |
| | TTTTTCAGCCAGGTTCGCTGGCAGTGGGTCTGGGA | |
| | CAGAGTTCACTCTCACCATCAGCAGCCTGCAGTCT | |
| | GAAGATTCTGCTATTTATTACTGTCAGCAGTATA | |
| | ATAACTGGCCTCCGTGGACGTTCGGCCAAGGGA | |
| | CCAAGGTGGAAATCAAAC | |
| 92 | EIVMTQSPATLSLSSGERATLSCRASRSVSSNLAWY | FN19 VK (aa) |
| | QQKPGQAPRLLIYDASTRATGFSARFAGSGSGTEFT | |
| | LTISSLQSEDSAIYYCQQYNNWPPWTFGQGTKVEIK | |
| 93 | RSVSSN | FNI9 CDRL1 (aa) |
| 94 | DAS | FNI9 CDRL2 (aa) |
| 95 | QQYNNWPPWT | FNI9 CDRL3 (aa) |
| 96 | GAGATCGTGATGACCCAGTCCCCAGCCACACTGA | FN19 Vk (co-nt) |
| | GCCTGTCCAGCGGAGAGAGGGCCACCCTGTCCTG | |
| | CAGGGCTTCCCGGAGCGTGTCTTCCAACCTGGCCT | |
| | GGTACCAGCAGAAGCCAGGCCAGGCTCCCAGACT | |
| | GCTGATCTATGACGCCTCTACCAGAGCTACAGGC | |
| | TTCTCCGCCAGGTTTGCTGGATCTGGATCCGGCAC | |
| | AGAGTTCACCCTGACAATCAGCTCTCTGCAGAGC | |
| | GAGGATTCTGCTATCTACTATTGTCAGCAGTACAA | |
| | CAATTGGCCCCCTTGGACCTTTGGCCAGGGCACA | |
| | AAGGTGGAGATCAAG | |
| 97 | CAGGTGCAGCTGGTGCAGTCTGGGGCTGAGGTGA | FNI10 VH (wt-nt) |
| | AGAAGCCTGGGTCCTCGGTGAAAGTCTCCTGCAA | |
| | GGCTTCTGGAGGCACCTTGAGTAGTCAAGTTAT | |
| | TAGCTGGGTGCGACAGGCCCCAGGACAAGGACTG | |
| | GAGTGGATCGGAGGGATCATCCCCACCACTGGT | |
| | ACAGGGGGCGCGGCAGAGGGGTTCCAGGGCAGA | |
| | GTCTCCATTTCCGCGGACGAATCCAGGAGCACAG | |
| | TCTACATGGAACTGACCAGCCTGACTTCTGGGGA | |
| | CACGGCCGTCTATTATTGTGCGAGAGCGGTTTCG | |
| | GATTACTTTAATAGAGACCTCGGCTGGGAAAA | |
| | TTACTACTTTGAATCTTGGGGCCAGGGAACCCTG | |
| | GTCACCGTCTCCTCAG | |
| 98 | QVQLVQSGAEVKKPGSSVKVSCKASGGTLSSQVIS | FNI10 VH (aa) |
| | WVRQAPGQGLEWIGGIIPTTGTGGAAEGFQGRVSIS | |
| | ADESRSTVYMELTSLTSGDTAVYYCARAVSDYFNR | |
| | DLGWENYYFESWGQGTLVTVSS | |
| 99 | GGTLSSQV | FNI10 CDRH1 (aa) |
| 100 | IIPTTGTG | FNI10 CDRH2 (aa) |
| 101 | ARAVSDYFNRDLGWENYYFES | FNI10 CDRH3 (aa) |
| 102 | CAGGTGCAGCTGGTGCAGAGCGGAGCTGAGGTGA | FNI10 VH (co-nt) |
| | AGAAGCCAGGCTCCAGCGTGAAGGTGTCCTGCAA | |
| | GGCTAGCGGCGGCACCCTGTCTTCCCAGGTCATCT | |
| | CTTGGGTGAGGCAGGCTCCAGGACAGGGACTGGA | |
| | GTGGATCGGCGGCATCATCCCTACCACAGGCACA | |
| | GGCGGAGCTGCTGAGGGATTCCAGGGCAGAGTGT | |
| | CCATCAGCGCCGACGAGTCTCGCTCCACCGTGTAC | |
| | ATGGAGCTGACCAGCCTGACATCTGGCGATACAG | |
| | CCGTGTACTATTGTGCCAGGGCCGTGTCCGACTAT | |
| | TTCAACCGGGATCTGGGCTGGGAGAATTACTATTT | |
| | TGAGTCCTGGGGCCAGGGCACCCTGGTGACAGTG | |
| | AGCTCT | |
| 103 | GAAATCGTGATGACGCAGTCTCCAGCCACCCTGT | FNI10 Vk (wt-nt) |
| | CTGTGTCTCCAGGGGAAAGAGCCACCCTCTCTTGC | |
| | AGGGCCAGTCGGAGTGTTAGTATCAACTTAGCC | |
| | TGGTACCAACAGAAACCTGGCCAGGCTCCCCGGC | |
| | TCCTCATTTATGATGCATCTACGAGGGCCACTGG | |
| | CATCCCAGCCAGGTTCGGTGGCAGGGGGTCTGGA | |
| | ACAGAGTTCACTCTCACCATCAGCAGCCTGCAGT | |
| | CTGAAGATTCTGCTGTTTATTACTGTCAGCACTAT | |
| | AATAACTGGCCTCCGTGGACATTCGGCCAAGGG | |
| | ACCAGAGTGGAAATCAAAC | |
| 104 | EIVMTQSPATLSVSPGERATLSCRASRSVSINLAWYQ | FNI10 VK (aa) |
| | QKPGQAPRLLIYDASTRATGIPARFGGRGSGTEFTLTI | |
| | SSLQSEDSAVYYCQHYNNWPPWTFGQGTRVEIK | |
| 105 | RSVSIN | FNI10 CDRL1 (aa) |
| 106 | DAS | FNI10 CDRL2 (aa) |
| 107 | QHYNNWPPWT | FNI10 CDRL3 (aa) |
| 108 | GAGATCGTGATGACCCAGTCCCCTGCCACACTGTCCGT | FNI10 Vk (co-nt) |
| | GTCCCCAGGAGAGAGAGCCACCCTGAGCTGCAGGGCT | |
| | AGCAGGTCCGTGTCCATCAACCTGGCCTGGTACCAGCA | |
| | GAAGCCAGGCCAGGCTCCCAGGCTGCTGATCTATGACG | |
| | CTTCTACCAGGGCTACAGGCATCCCAGCTAGATTCGGA | |
| | GGAAGGGGATCCGGAACAGAGTTTACCCTGACAATCT | |
| | CCAGCCTGCAGAGCGAGGATTCCGCCGTGTACTATTGT | |
| | CAGCACTACAACAATTGGCCACCTTGGACCTTCGGCCA | |
| | GGGAACACGCGTGGAGATCAAG | |
| 109 | CAGGTGCACCTGGTACAGTCTGGGGCTGAGGTGA | FNI12 VH (wt-nt) |
| | AGAAGCCTGGGTCCTCGGTGAGGGTCTCCTGCAA | |
| | GGCTTCTGGAGACTCCTTCAACAAATATGAAGTC | |
| | AGCTGGGTGCGACAGGCCCCCGGACATGGACTTG | |
| | AGTGGATGGGAGGGATCATCCCTCTCTCTCCTAT | |
| | AGCGAGGTACGCAGAGAAATTTCAGGGCAGAGTC | |
| | ACGATTACCGCGGACGAATTCACGAGCACGGTCT | |
| | ATATACAACTGACCAGCCTGAGATCTGACGACAC | |
| | GGCCGTATACTACTGTGCGACAACACGTTCGGAT | |
| | TACTTTAATAGAGACCTCGGCTGGGAAGATTAC | |
| | TTCTTTGACCACTGGGGCCAGGGAACCCTGGTCA | |
| | CCGTCTCCTCAG | |
| 110 | QVHLVQSGAEVKKPGSSVRVSCKASGDSFNKYEVS | FNI12 VH (aa) |
| | WVRQAPGHGLEWMGGIIPLSPIARYAEKFQGRVTIT | |
| | ADEFTSTVYIQLTSLRSDDTAVYYCATTRSDYFNRD | |
| | LGWEDYFFDHWGQGTLVTVSS | |
| 111 | GDSFNKYE | FNI12 CDRH1 (aa) |
| 112 | IIPLSPIA | FNI12 CDRH2 (aa) |
| 113 | ATTRSDYFNRDLGWEDYFFDH | FNI12 CDRH3 (aa) |
| 114 | CAGGTGCACCTGGTGCAGTCTGGCGCCGAGGTGAAGA | FNI12 VH (co-nt) |
| | AGCCAGGCTCCAGCGTGAGGGTGTCCTGCAAGGCTAG | |
| | CGGCGACTCTTTCAACAAGTACGAGGTGAGCTGGGTGA | |
| | GACAGGCTCCAGGACATGGACTGGAGTGGATGGGCGG | |
| | CATCATCCCCCTGTCTCCTATCGCCAGATACGCTGAGA | |
| | AGTTCCAGGGCCGCGTGACCATCACAGCTGATGAGTTT | |
| | ACCTCCACAGTGTATATCCAGCTGACCTCCCTGAGGAG | |
| | CGACGATACAGCCGTGTACTATTGTGCTACCACAAGGA | |
| | GCGACTACTTTAATCGGGATCTGGGCTGGGAGGACTAT | |
| | TTCTTTGATCACTGGGGCCAGGGCACCCTGGTGACAGT | |
| | GTCTTCC | |
| 115 | GAAATAGTGATGACGCAGTCTCCAGCCACCCTGTC | FNI12 Vk (wt-nt) |
| | TGTGTCTCCAGGGGAAAGAGCCACCCTCTCCTGCA | |
| | GGGCCAGTCAGAGTATTAGCACCAACTTAGCCTG | |
| | GTACCAGCAGAAACCTGGCCAGGCTCCCAGGCTC | |
| | CTCATCTCTGGTGCATCCACCAGGGCCACTGGTA | |
| | TCCCAGCCAGGTTCAGTGGCAGTGGGTCTGGGAC | |
| | AGAGTTCACTCTCACCATCAGCAGCCTGCAGTCTG | |
| | AAGATTTTGGAGTTTATTACTGTCAGCACTATAAT | |
| | AACTGGCCTCCGTGGACGTTCGGCCAAGGGACC | |
| | AAGGTGGAAATCAAAC | |
| 116 | EIVMTQSPATLSVSPGERATLSCRASQSISTNLAWYQ | FNI12 VK (aa) |
| | QKPGQAPRLLISGASTRATGIPARFSGSGSGTEFTLTI | |
| | SSLQSEDFGVYYCQHYNNWPPWTFGQGTKVEIK | |
| 117 | QSISTN | FNI12 CDRL1 (aa) |
| 118 | GAS | FNI12 CDRL2 (aa) |
| 119 | QHYNNWPPWT | FNI12 CDRL3 (aa) |
| 120 | GAGATCGTGATGACCCAGTCCCCTGCCACACTGTC | FNI12 Vk (co-nt) |
| | CGTGTCCCCAGGAGAGAGGGCCACCCTGAGCTGC | |
| | CGGGCTAGCCAGTCTATCTCCACAAACCTGGCCTG | |
| | GTACCAGCAGAAGCCAGGACAGGCTCCAAGGCTG | |
| | CTGATCAGCGGAGCTTCTACCAGAGCTACAGGCAT | |
| | CCCAGCTCGCTTCAGCGGATCTGGATCCGGAACCG | |
| | AGTTTACCCTGACAATCTCCAGCCTGCAGTCTGAG | |
| | GACTTCGGCGTGTACTATTGTCAGCACTATAACAA | |
| | TTGGCCCCCTTGGACCTTTGGCCAGGGCACAAAGG | |
| | TGGAGATCAAG | |
| 121 | CAGGTTCAGCTGGTGCAATCTGGGGCTGAGGTGA | FNI13 VH (wt-nt) |
| | AGAGGCCTGGGTCCTCGGTGAGGGTCTCCTGCAA | |
| | GGGTTCTGGAGACACCTTCAACAACTATGTTATC | |
| | AGTTGGGTGCGACAGGCCCCTGGCCAAGGGCTTG | |
| | AGTGGATGGGGGGGATCATCCCTATCTTTCAAAC | |
| | ACCAAACTACGCAGAGAAGTTCCAGGGCAGAGTC | |
| | GCGATTACCGCGGACGAATCCACGAGCACGGCCT | |
| | ACATGGAGTTGAGCAGCCTGAGATCTGAGGACTC | |
| | GGCCATTTATTACTGTGCGAGAGCGAATTCCGAT | |
| | TACTTTAATAGAGACCTCGGCTGGGAAAATTAC | |
| | TACTTTGAAGACTGGGGCCAGGGAACCCTGGTCA | |
| | CCGTCTCCTCAG | |
| 122 | QVQLVQSGAEVKRPGSSVRVSCKGSGDTFNNYVIS | FNI13 VH (aa) |
| | WVRQAPGQGLEWMGGIIPIFQTPNYAEKFQGRVAI | |
| | TADESTSTAYMELSSLRSEDSAIYYCARANSDYFNR | |
| | DLGWENYYFEDWGQGTLVTVSS | |
| 123 | GDTFNNYV | FNI13 CDRH1 (aa) |
| 124 | IIPIFQTP | FNI13 CDRH2 (aa) |
| 125 | ARANSDYFNRDLGWENYYFED | FNI13 CDRH3 (aa) |
| 126 | CAGGTGCAGCTGGTGCAGTCCGGAGCTGAGGTGAAGA | FNI13 VH (co-nt) |
| | GGCCAGGATCCAGCGTGCGGGTGAGCTGCAAGGGATC | |
| | TGGCGACACCTTCAACAATTACGTGATCAGCTGGGTGA | |
| | GGCAGGCTCCAGGACAGGGACTGGAGTGGATGGGCGG | |
| | CATCATCCCCATCTTCCAGACCCCTAACTACGCTGAGA | |
| | AGTTTCAGGGCAGGGTGGCCATCACAGCTGACGAGTCC | |
| | ACCAGCACAGCCTATATGGAGCTGTCTTCCCTGAGATC | |
| | TGAGGATTCCGCTATCTACTATTGTGCCAGAGCTAACT | |
| | CTGACTATTTCAATCGCGATCTGGGCTGGGAGAATTAC | |
| | TATTTTGAGGATTGGGGCCAGGGCACCCTGGTGACAGT | |
| | GAGCTCT | |
| 127 | GAAAGAGTGATGACGCAGTCTCCAGCCACCCTTTC | FNI13 Vk (wt-nt) |
| | TGTGTCTCCAGGGGGAAGAGCCACCCTCTCCTGCA | |
| | GGGCCAGTCAGAGTGTTGGTAGCAACTTAGCCT | |
| | GGTACCAGCAGAAACCTGGCCAGGCTCCCAGGCT | |
| | CCTCATCTATGATGCTTCTGCCAGGGCCACTGGT | |
| | GTCCCAGCCAGGTTCAGTGGCAGTGGGTCTGGGA | |
| | CAGAGTTCTCTCTCTCCATCAACAGCCTGCAGTCT | |
| | GAAGATTCTGCAGTTTATTACTGTCAGCACTATAA | |
| | TATCTGGCCGCCGTGGACGTTCGGCCAAGGGAC | |
| | CAAGGTGGAAATCAAAC | |
| 128 | ERVMTQSPATLSVSPGGRATLSCRASQSVGSNLAW | FNI13 VK (aa) |
| | YQQKPGQAPRLLIYDASARATGVPARFSGSGSGTEFS | |
| | LSINSLQSEDSAVYYCQHYNIWPPWTFGQGTKVEIK | |
| 129 | QSVGSN | FNI13 CDRL1 (aa) |
| 130 | DAS | FNI13 CDRL2 (aa) |
| 131 | QHYNIWPPWT | FNI13 CDRL3 (aa) |
| 132 | GAGAGAGTGATGACCCAGTCTCCTGCTACACTGTC | FNI13 Vk (co-nt) |
| | CGTGAGCCCAGGAGGAAGGGCTACCCTGTCCTGC | |
| | AGGGCTTCTCAGTCCGTGGGAAGCAACCTGGCTTG | |
| | GTACCAGCAGAAGCCAGGCCAGGCCCCCAGACTG | |
| | CTGATCTATGACGCTTCCGCTAGAGCTACCGGCGT | |
| | GCCAGCTCGCTTCAGCGGATCTGGCTCCGGCACAG | |
| | AGTTTAGCCTGTCTATCAACTCCCTGCAGAGCGAG | |
| | GATTCTGCCGTGTACTATTGTCAGCACTACAATAT | |
| | CTGGCCACCTTGGACCTTCGGCCAGGGAACAAAG | |
| | GTGGAGATCAAG | |
| 133 | CAAGTTCAGTTGGTGCAGTCTGGGGCTGAGCTGAA | FNI14 VH (wt-nt) |
| | GCGGCCTGGGTCCTCGGTGAGGATCTCCTGCAAGG | |
| | CCTCTGGTGTCACCTTCAACAAGTATGTTCTCAG | |
| | CTGGGTGCGACTGGCCCCTGGACAAGGGCTTGAG | |
| | TGGATGGGAGGAATCATCCCTATTTCTGGTATAC | |
| | CACATTACGCAGAGAAGTTCCAGGGCAGAGTCGC | |
| | GATTACCGCGGACGAATCCACGAGCACAGTCTAC | |
| | ATGGAGTTGAGCAGCCTACGATCTGAGGACTCGG | |
| | CCCTATATTACTGTGCGAGAGCGGTCTCCGATTA | |
| | TTTTAATCGGGACCTCGGCTGGGATGATTACTA | |
| | CTTTCCTTTGTGGGGCCACGGCACCCTGGTCACC | |
| | GTCTCCTCAG | |
| 134 | QVQLVQSGAELKRPGSSVRISCKASGVTFNKYVLS | FNI14 VH (aa) |
| | WVRLAPGQGLEWMGGIIPISGIPHYAEKFQGRVAIT | |
| | ADESTSTVYMELSSLRSEDSALYYCARAVSDYFNRD | |
| | LGWDDYYFPLWGHGTLVTVSS | |
| 135 | GVTFNKYV | FNI14 CDRH1 (aa) |
| 136 | IIPISGIP | FNI14 CDRH2 (aa) |
| 137 | ARAVSDYFNRDLGWDDYYFPL | FNI14 CDRH3 (aa) |
| 138 | CAGGTGCAGCTGGTGCAGTCTGGAGCTGAGCTGAAGA | FNI14 VH (co-nt) |
| | GGCCAGGATCCAGCGTGCGGATCAGCTGCAAGGCTTCT | |
| | GGCGTGACCTTCAACAAGTACGTGCTGTCCTGGGTGAG | |
| | GCTGGCTCCAGGACAGGGACTGGAGTGGATGGGCGGC | |
| | ATCATCCCCATCAGCGGCATCCCTCACTACGCTGAGAA | |
| | GTTTCAGGGCAGGGTGGCCATCACAGCTGACGAGTCCA | |
| | CCAGCACAGTGTATATGGAGCTGTCTTCCCTGAGATCT | |
| | GAGGATTCCGCCCTGTACTATTGTGCCAGAGCCGTGTC | |
| | CGACTATTTCAATCGCGATCTGGGCTGGGACGATTACT | |
| | ATTTTCCCCTGTGGGGCCATGGCACCCTGGTGACAGTG | |
| | AGCTCT | |
| 139 | GAAATAGTGATGACGCAGTCTCCAGCCACCCTGTC | FNI14 Vk (wt-nt) |
| | TGTGTCTCCAGGGGAAAGCGCCACCCTCTTCTGCA | |
| | GGGCCAGTCGGAGTGTTAGTGACAACTTAGCCT | |
| | GGTACCAGCAGAAACCTGGCCAGGCTCCCAGGCT | |
| | CCTCATCTTTGGTGCTTCCACCAGGGCCACTGGT | |
| | GTCCCAGCCAGGTTCGGTGGCAGTGGGTCTGGGA | |
| | CACAGTTCACTCTCACCATCAGCAGCCTGCAGTCT | |
| | GAAGATTTTGCAGTTTATTACTGTCAGCATTATAA | |
| | TAACTGGCCTCCGTGGACGTTCGGCCAAGGGAC | |
| | CAAGGTGGAGATCAAAC | |
| 140 | EIVMTQSPATLSVSPGESATLFCRASRSVSDNLAWY | FNI14 VK (aa) |
| | QQKPGQAPRLLIFGASTRATGVPARFGGSGSGTQFTL | |
| | TISSLQSEDFAVYYCQHYNNWPPWTFGQGTKVEIK | |
| 141 | RSVSDN | FNI14 CDRL1 (aa) |
| 142 | GAS | FNI14 CDRL2 (aa) |
| 143 | QHYNNWPPWT | FNI14 CDRL3 (aa) |
| 144 | GAGATCGTGATGACCCAGTCCCCTGCCACACTGTC | FNI14 Vk (co-nt) |
| | CGTGTCCCCAGGAGAGAGCGCCACCCTGTTCTGCA | |
| | GGGCTAGCAGGTCCGTGTCCGACAACCTGGCCTG | |
| | GTACCAGCAGAAGCCAGGCCAGGCTCCCAGGCTG | |
| | CTGATCTTTGGCGCCTCTACCAGAGCTACAGGCGT | |
| | GCCAGCTAGGTTCGGAGGAAGCGGATCTGGCACA | |
| | CAGTTTACCCTGACAATCTCCAGCCTGCAGTCCGA | |
| | GGATTTCGCCGTGTACTATTGTCAGCACTATAACA | |
| | ATTGGCCCCCTTGGACCTTTGGCCAGGGCACAAAG | |
| | GTGGAGATCAAG | |
| 145 | CAGGTTCAACTGGTGCAGTCTGGGGCTGAGGTGAAGA | FNI17 VH (wt-nt) |
| | GGCCTGGGTCCTCGGTGAAGGTCTCCTGCAAGCCTTCC | |
| | GGAGGCACCTTCAGCAACAATGTTATCAGCTGGGTG | |
| | CGACAGGCCCCTGGACAAGGGCTTGAGTGGATGGGAG | |
| | GGATCATCCCCACCTCTGGTATAGCAAACTACGCGC | |
| | AGAAGTTCCAGGGCAGAGTCGCGATTATTGCGGACAA | |
| | ATCTACGAGCACAGTCTACATGGCGTTGAGCAGCCTGA | |
| | GATCTGAGGACTCGGCCGTGTATTTCTGTGCCAGAGC | |
| | GCGGTCCGACTACTTCAATAGAGACCTCGGCTGGG | |
| | AAGATTACTACTTTGAGAACTGGGGCCAGGGAACCCT | |
| | GGTCACCGTCTCCTCAG | |
| 146 | QVQLVQSGAEVKRPGSSVKVSCKPSGGTFSNNVISWI | FNI17 VH (aa) |
| | VRQAPGQGLEWMGGIIPTSGIANYAQKFQGRVAIIA | |
| | DKSTSTVYMALSSLRSEDSAVYFCARARSDYENRD | |
| | LGWEDYYFENWGQGTLVTVSS | |
| 147 | GGTFSNNV | FNI17 CDRH1 (aa) |
| 148 | IIPPTSGIA | FNI17 CDRH2 (aa) |
| 149 | ARARSDYFNRDLGWEDYYFEN | FNI17 CDRH3 (aa) |
| 150 | CAGGTGCAGCTGGTGCAGTCCGGAGCTGAGGTGA | FNI17 VH (co-nt) |
| | AGAGGCCAGGCTCCAGCGTGAAGGTGAGCTGCAA | |
| | GCCTTCTGGCGGCACCTTCTCCAACAATGTGATCA | |
| | GCTGGGTGAGACAGGCTCCAGGACAGGGACTGGA | |
| | GTGGATGGGAGGAATCATCCCCACATCTGGCATC | |
| | GCCAACTACGCTCAGAAGTTTCAGGGCAGGGTGG | |
| | CCATCATCGCTGATAAGTCCACCAGCACAGTGTAT | |
| | ATGGCCCTGTCTTCCCTGAGATCTGAGGACTCCGC | |
| | CGTGTACTTCTGTGCCAGGGCTCGGTCCGACTACT | |
| | TCAACCGCGATCTGGGCTGGGAGGACTACTATTTC | |
| | GAGAATTGGGGCCAGGGCACCCTGGTGACAGTGA | |
| | GCTCT | |
| 151 | GAAATAGTGATGACGCAGTCTCCAGCCACCCTGTC | FNI17 Vk (wt-nt) |
| | TGTGTCTCCAGGGGAAAGAGCCACCCTCTCCTGCA | |
| | GGGCCAGTCAGAGTGTTGGCAGCAGCTTAGTCT | |
| | GGTACCAGCAGAAACCTGGCCAGGCTCCCAGGCT | |
| | CCTCATCTATGGTGCATCCACCAGGGCCACTGGT | |
| | GTCCCAGCCAGGTTCAGTGGCAGTGGGTCTGGGA | |
| | CAGAGTTCACTCTCACCATCAGCAGCCTGCAGTCT | |
| | GAAGATTTTGCAGTTTATTACTGTCAGCACTATAA | |
| | TAACTGGCCTCCGTGGACGTTCGGCCAAGGGAC | |
| | CAAGGTGGAAATCAAAC | |
| 152 | EIVMTQSPATLSVSPGERATLSCRASQSVGSSLVWY | FNI17 VK (aa) |
| | QQKPGQAPRLLIYGASTRATGVPARFSGSGSGTEFTL | |
| | TISSLQSEDFAVYYCQHYNNWPPWTFGQGTKVEIK | |
| 153 | QSVGSS | FNI17 CDRL1 (aa) |
| 154 | GAS | FNI17 CDRL2 (aa) |
| 155 | QHYNNWPPWT | FNI17 CDRL3 (aa) |
| 156 | GAGATCGTGATGACCCAGTCTCCTGCCACACTGAG | FNI17 Vk (co-nt) |
| | CGTGTCTCCAGGAGAGAGGGCCACCCTGTCCTGCA | |
| | GGGCTTCCCAGAGCGTGGGATCCAGCCTGGTGTG | |
| | GTACCAGCAGAAGCCAGGACAGGCTCCAAGGCTG | |
| | CTGATCTATGGAGCTAGCACCAGAGCTACAGGCG | |
| | TGCCAGCTCGCTTCTCTGGATCCGGAAGCGGCACA | |
| | GAGTTTACCCTGACAATCTCTTCCCTGCAGTCTGA | |
| | GGACTTCGCCGTGTACTATTGTCAGCACTACAACA | |
| | ATTGGCCCCCTTGGACCTTTGGCCAGGGCACAAAG | |
| | GTGGAGATCAAG | |
| 157 | CAAGTTCAGCTGGTGCAGTCTGGGGCTGAGGTGA | FNI19 VH (wt-nt) |
| | AGAGGCCTGGGTCCTCGGTGAGGGTCTCCTGCAA | |
| | GGCTTCTGAAGGCACCTTCAACAAGTATACTCTC | |
| | ACCTGGGTGCGACAGGCCCCTGGACAGGGACTTG | |
| | AGTGGATGGGAGGAATCATCCCTATCTCCGGTA | |
| | TAGCAAACTACGCACAGAAGTTCCAGGGCAGAGT | |
| | CGCGATTACCGCGGACGAATCCACGACCACAGCC | |
| | TACATGGAATTGAGCAGCCTAAGATCTGAAGACT | |
| | CGGCCGTATATTACTGTGCGACAGCGGTCTCCGA | |
| | TTATTTTAATCGAGACCTCGGCTGGGAAGATTA | |
| | CTACTTTCCGTTCTGGGGCCAGGGCACCCTGGTC | |
| | ACCGTCGCCTCAG | |
| 158 | QVQLVQSGAEVKRPGSSVRVSCKASEGTFNKYTLT | FNI19 VH (aa) |
| | WVRQAPGQGLEWMGGIIPISGIANYAQKFQGRVAIT | |
| | ADESTTTAYMELSSLRSEDSAVYYCATAVSDYFNR | |
| | DLGWEDYYFPFWGQGTLVTVAS | |
| 159 | EGTFNKYT | FNI19 CDRH1 (aa) |
| 160 | IIPISGIA | FNI19 CDRH2 (aa) |
| 161 | ATAVSDYFNRDLGWEDYYFPF | FNI19 CDRH3 (aa) |
| 162 | CAGGTGCAGCTGGTGCAGTCCGGAGCTGAGGTGA | FNI19 VH (co-nt) |
| | AGAGGCCAGGATCCAGCGTGCGGGTGTCCTGCAA | |
| | GGCTAGCGAGGGCACATTCAACAAGTACACACTG | |
| | ACCTGGGTGAGGCAGGCTCCAGGACAGGGACTGG | |
| | AGTGGATGGGCGGCATCATCCCTATCTCTGGCATC | |
| | GCCAATTACGCTCAGAAGTTTCAGGGCAGAGTGG | |
| | CCATCACAGCTGATGAGTCCACCACAACCGCCTAT | |
| | ATGGAGCTGTCTTCCCTGAGAAGCGAGGACTCCGC | |
| | CGTGTACTATTGTGCCACCGCTGTGAGCGACTATT | |
| | TCAACCGCGATCTGGGCTGGGAGGACTACTATTTC | |
| | CCCTTTTGGGGCCAGGGCACACTGGTGACCGTGGC | |
| | TTCT | |
| 163 | GAAATAGTGATGACGCAGTCTCCAGCCACCCTGTC | FNI19 Vk (wt-nt) |
| | TGTGTCTCCGGGGGCCAGAGCCACCCTCTTCTGCA | |
| | GGGCCAGTCGGAGTGTTAGTGACAACTTAGCCT | |
| | GGTACCAGCAGAAACCTGGCCAGGCTCCCAGGCT | |
| | CCTCATCTTTGGTGCATCCACCAGGGCCACTGGT | |
| | GTCCCAGCCAGGTTCAGTGGAAGTGGGTCTGGGA | |
| | CACAGTTCACTCTCACCATCAGCAGCCTGCAGTCC | |
| | GAAGATTTTGCAGTTTATTACTGTCAGCATTATAA | |
| | TATTTGGCCTCCGTGGACGTTCGGCCAAGGGAC | |
| | CAAGGTGGAGATCAAAC | |
| 164 | EIVMTQSPATLSVSPGARATLFCRASRSVSDNLAWY | FNI19 VK (aa) |
| | QQKPGQAPRLLIFGASTRATGVPARFSGSGSGTQFTL | |
| | TISSLQSEDFAVYYCQHYNIWPPWTFGQGTKVEIK | |
| 165 | RSVSDN | FNI19 CDRL1 (aa) |
| 166 | GAS | FNI19 CDRL2 (aa) |
| 167 | QHYNIWPPWT | FNI19 CDRL3 (aa) |
| 168 | GAGATCGTGATGACCCAGTCCCCTGCTACACTGTC | FNI19 Vk (co-nt) |
| | CGTGTCCCCAGGAGCTAGGGCTACCCTGTTCTGCA | |
| | GGGCTAGCAGGTCCGTGTCCGACAACCTGGCTTGG | |
| | TACCAGCAGAAGCCAGGCCAGGCCCCCAGACTGC | |
| | TGATCTTTGGAGCTAGCACCAGAGCTACAGGCGTG | |
| | CCAGCTCGCTTCAGCGGATCTGGATCCGGCACACA | |
| | GTTTACCCTGACAATCTCCAGCCTGCAGTCTGAGG | |
| | ATTTCGCCGTGTACTATTGTCAGCACTATAATATC | |
| | TGGCCCCCTTGGACCTTTGGCCAGGGCACAAAGGT | |
| | GGAGATCAAG | |
| 169 | [Reserved] | |
| 170 | CAGGTGCAGCTGGTGCAGTCCGGAGCTGAGGTGA | FNI3-VH-W110F |
| | AGAGGCCAGGATCCAGCGTGAAGGTGTCCTGCAA | (nt) |
| | GGCCAGCGGCGCTACCTTCAGCAACAATGTGAT | |
| | CGCTTGGGTGAGACAGGCTCCAGGACAGGGACTG | |
| | GAGTGGATGGGAGGAATCCACCCTATCAGCGCC | |
| | ACCGCTACATACGCCCAGAAGTTTCAGGGCAGAG | |
| | TGGCTATCGCCGCTGACGAGCTGACCTCTACAGCC | |
| | TATATGGAGCTGAACGGCCTGCGCAGCGAGGATT | |
| | CCGCCGTGTACTATTGTGCCAGGGCTGGCTCTGA | |
| | CTACTTCAACCGGGATCTGGGCTTCGAGAATTA | |
| | CTATTTTGACTCCTGGGGCCAGGGCACCCTGGTG | |
| | ACAGTGTCTTCC | |
| 171 | QVQLVQSGAEVKRPGSSVKVSCKASGATFSNNVIA | FNI3-VH-W110F |
| | WVRQAPGQGLEWMGGIHPISATATYAQKFQGRVAI | (aa) |
| | AADELTSTAYMELNGLRSEDSAVYYCARAGSDYEN | |
| | RDLGFENYYFDSWGQGTLVTVSS | |
| 172 | ARAGSDYFNRDLGFENYYFDS | FNI3-VH-W110F |
| | | CDRH3 (aa) |
| 173 | GAGATCCTGATGACCCAGTCCCCTGCCACACTGTC | FNI3-VK-W94F (nt) |
| | CGTGTCCCCAGGAGAGAGGGCCACCCTGAGCTGC | |
| | AGGGCTTCTCAGGACGTGTCCGGCAACCTGGCCT | |
| | GGTACCAGCAGAGACCAGGACAGGCTCCAAGGCT | |
| | GCTGATCTATGGAGCTTCCACCAGGGCTACAGGC | |
| | GTGCCAGCTAGATTCACCGGCGCTGGAAGCGGCA | |
| | CAGAGTTTACCCTGACAATCTCCAGCCTGCAGTCT | |
| | GAGGATTTCGCTCTGTACTATTGTCAGCACTACAA | |
| | CAATTTTCCCCCTTGGACCTTTGGCCAGGGCACA | |
| | AAGGTGGAGATCAAG | |
| 174 | EILMTQSPATLSVSPGERATLSCRASQDVSGNLAWY | FNI3-VK-W94F (aa) |
| | QQRPGQAPRLLIYGASTRATGVPARFTGAGSGTEFT | |
| | LTISSLQSEDFALYYCQHYNNFPPWTFGQGTKVEIK | |
| 175 | QHYNNFPPWT | FNI3-VK-W94F |
| | | CDRL3 (aa) |
| 176 | GAGATCCTGATGACCCAGTCCCCTGCCACACTGTC | FNI3-VK-W97F (nt) |
| | CGTGTCCCCAGGAGAGAGGGCCACCCTGAGCTGC | |
| | AGGGCTTCTCAGGACGTGTCCGGCAACCTGGCCT | |
| | GGTACCAGCAGAGACCAGGACAGGCTCCAAGGCT | |
| | GCTGATCTATGGAGCTTCCACCAGGGCTACAGGC | |
| | GTGCCAGCTAGATTCACCGGCGCTGGAAGCGGCA | |
| | CAGAGTTTACCCTGACAATCTCCAGCCTGCAGTCT | |
| | GAGGATTTCGCTCTGTACTATTGTCAGCACTACAA | |
| | CAATTGGCCCCCTTTCACCTTTGGCCAGGGCACA | |
| | AAGGTGGAGATCAAG | |
| 177 | EILMTQSPATLSVSPGERATLSCRASQDVSGNLAWY | FNI3-VK-W97F (aa) |
| | QQRPGQAPRLLIYGASTRATGVPARFTGAGSGTEFT | |
| | LTISSLQSEDFALYYCQHYNNWPPFTFGQGTKVEIK | |
| 178 | QHYNNWPPFT | FNI3-VK-W97F |
| | | CDRL3 (aa) |
| 179 | GAGATCCTGATGACCCAGTCCCCTGCCACACTGTC | FNI3-VK-W94F- |
| | CGTGTCCCCAGGAGAGAGGGCCACCCTGAGCTGC | W97F (nt) |
| | AGGGCTTCTCAGGACGTGTCCGGCAACCTGGCCT | |
| | GGTACCAGCAGAGACCAGGACAGGCTCCAAGGCT | |
| | GCTGATCTATGGAGCTTCCACCAGGGCTACAGGC | |
| | GTGCCAGCTAGATTCACCGGCGCTGGAAGCGGCA | |
| | CAGAGTTTACCCTGACAATCTCCAGCCTGCAGTCT | |
| | GAGGATTTCGCTCTGTACTATTGTCAGCACTACAA | |
| | CAATTTTCCCCCTTTCACCTTTGGCCAGGGCACA | |
| | AAGGTGGAGATCAAG | |
| 180 | EILMTQSPATLSVSPGERATLSCRASQDVSGNLAWY | FNI3-VK-W94F- |
| | QQRPGQAPRLLIYGASTRATGVPARFTGAGSGTEFT | W97F (aa) |
| | LTISSLQSEDFALYYCQHYNNFPPFTFGQGTKVEIK | |
| 181 | QHYNNFPPFT | FNI3-VK-W94F- |
| | | W97F CDRL3 (aa) |
| 182 | CAGGTGCACCTGGTGCAGAGCGGAGCTGAGGTGA | FNI9-VH-W110F |
| | AGGAGCCAGGATCCAGCGTGACAGTGTCTTGCAA | (nt) |
| | GGCTTCCGGCGGCAGCTTCAACAATCAGGCTAT | |
| | CTCCTGGGTGAGGCAGGCTCCAGGACAGGGACTG | |
| | GAGTGGATGGGCGGCATCTTTCCCATCTCTGGCA | |
| | CACCTACCTCCGCCCAGAGGTTCCAGGGAAGGGT | |
| | GACCTTCACCGCTGACGAGAGCACCACAACCGTG | |
| | TACATGGATCTGTCTTCCCTGAGATCTGACGATAC | |
| | CGCCGTGTACTATTGTGCCAGAGCTGGCTCCGAC | |
| | TATTTCAACCGCGATCTGGGCTTCGAGAATTAC | |
| | TATTTTGCTTCCTGGGGCCAGGGCACACTGGTGA | |
| | CCGTGAGCTCT | |
| 183 | QVHLVQSGAEVKEPGSSVTVSCKASGGSFNNQAIS | FNI9-VH-W110F |
| | WVRQAPGQGLEWMGGIFPISGTPTSAQRFQGRVTF | (aa) |
| | TADESTTTVYMDLSSLRSDDTAVYYCARAGSDYFN | |
| | RDLGFENYYFASWGQGTLVTVSS | |
| 184 | ARAGSDYFNRDLGFENYYFAS | FNI9-VH-W110F |
| | | CDRH3 (aa) |
| 185 | GAGATCGTGATGACCCAGTCCCCAGCCACACTGA | FNI9-VK-W94F (nt) |
| | GCCTGTCCAGCGGAGAGAGGGCCACCCTGTCCTG | |
| | CAGGGCTTCCCGGAGCGTGTCTTCCAACCTGGCC | |
| | TGGTACCAGCAGAAGCCAGGCCAGGCTCCCAGAC | |
| | TGCTGATCTATGACGCCTCTACCAGAGCTACAGG | |
| | CTTCTCCGCCAGGTTTGCTGGATCTGGATCCGGCA | |
| | CAGAGTTCACCCTGACAATCAGCTCTCTGCAGAGC | |
| | GAGGATTCTGCTATCTACTATTGTCAGCAGTACA | |
| | ACAATTTCCCCCCTTGGACCTTTGGCCAGGGCAC | |
| | AAAGGTGGAGATCAAG | |
| 186 | EIVMTQSPATLSLSSGERATLSCRASRSVSSNLAWYQ | FNI9-VK-W94F( aa) |
| | QKPGQAPRLLIYDASTRATGFSARFAGSGSGTEFTLT | |
| | ISSLQSEDSAIYYCQQYNNFPPWTFGQGTKVEIK | |
| 187 | QQYNNFPPWT | FNI9-VK-W94F |
| | | CDRL3 (aa) |
| 188 | GAGATCGTGATGACCCAGTCCCCAGCCACACTGA | FNI9-VK-W97F (nt) |
| | GCCTGTCCAGCGGAGAGAGGGCCACCCTGTCCTG | |
| | CAGGGCTTCCCGGAGCGTGTCTTCCAACCTGGCC | |
| | TGGTACCAGCAGAAGCCAGGCCAGGCTCCCAGAC | |
| | TGCTGATCTATGACGCCTCTACCAGAGCTACAGG | |
| | CTTCTCCGCCAGGTTTGCTGGATCTGGATCCGGCA | |
| | CAGAGTTCACCCTGACAATCAGCTCTCTGCAGAGC | |
| | GAGGATTCTGCTATCTACTATTGTCAGCAGTACA | |
| | ACAATTGGCCCCCTTTCACCTTTGGCCAGGGCAC | |
| | AAAGGTGGAGATCAAG | |
| 189 | EIVMTQSPATLSLSSGERATLSCRASRSVSSNLAWYQ | FNI9-VK-W97F (aa) |
| | QKPGQAPRLLIYDASTRATGFSARFAGSGSGTEFTLT | |
| | ISSLQSEDSAIYYCQQYNNWPPFTFGQGTKVEIK | |
| 190 | QQYNNWPPFT | FNI9-VK-W97F |
| | | CDRL3 (aa) |
| 191 | GAGATCGTGATGACCCAGTCCCCAGCCACACTGA | FNI9-VK-W94F- |
| | GCCTGTCCAGCGGAGAGAGGGCCACCCTGTCCTG | W97F (nt) |
| | CAGGGCTTCCCGGAGCGTGTCTTCCAACCTGGCC | |
| | TGGTACCAGCAGAAGCCAGGCCAGGCTCCCAGAC | |
| | TGCTGATCTATGACGCCTCTACCAGAGCTACAGG | |
| | CTTCTCCGCCAGGTTTGCTGGATCTGGATCCGGCA | |
| | CAGAGTTCACCCTGACAATCAGCTCTCTGCAGAGC | |
| | GAGGATTCTGCTATCTACTATTGTCAGCAGTACA | |
| | ACAATTTTCCCCCTTTCACCTTTGGCCAGGGCAC | |
| | AAAGGTGGAGATCAAG | |
| 192 | EIVMTQSPATLSLSSGERATLSCRASRSVSSNLAWYQ | FNI9-VK-W94F-W97F |
| | QKPGQAPRLLIYDASTRATGFSARFAGSGSGTEFTLT | (aa) |
| | ISSLQSEDSAIYYCQQYNNFPPFTFGQGTKVEIK | |
| 193 | QQYNNFPPFT | FNI9-VK-W94F- |
| | | W97F CDRL3 (aa) |
| 194 | QVQLQQSGPGLVKPSQTLSLTCAISGDSVSSYNAVW | FM08 VH |
| | NWIRQSPSRGLEWLGRTYYRSGWYNDYAESVKSRIT | |
| | INPDTSKNQFSLQLNSVTPEDTAVYYCARSGHITVFG | |
| | VNVDAFDMWGQGTMVTVSS | |
| 195 | DIQMTQSPSSLSASVGDRVTITCRTSQSLSSYTHWYQ | FM08 VL |
| | QKPGKAPKLLIYAASSRGSGVPSRFSGSGSGTDFTLTI | |
| | SSLQPEDFATYYCQQSRTFGQGTKVEIK | |
| 196 | APELLGGPSVFLFPPKPKDTLMISRTPEVTCVVVDVS | WT hIgG1 Fc |
| | HEDPEVKFNWYVDGVEVHNAKTKPREEQYNSTYRV | |
| | VSVLTVLHQDWLNGKEYKCKVSNKALPAPIEKTISK | |
| | AKGQPREPQVYTLPPSRDELTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPG | |
| | K | |
| 197 | ESKYGPPCPPCPAPPVAGP | Chimeric hinge |
| | | sequence |
| 198 | CAGGTCCAGCTGGTCCAGAGTGGGGCAGAGGTCA | FNI17-v19-VH (co- |
| | AAGAGCCAGGGTCTTCAGTCACAGTCTCATGCAA | nt) |
| | AGCAAGCGGAGGAACATTTTCCAACAATGTGATC | |
| | AGCTGGGTGAGGCAGGCTCCAGGACAGGGACTGG | |
| | AGTGGATGGGCGGCATCATCCCTACCTCTGGCATC | |
| | GCCAACTACGCTCAGAAGTTCCAGGGCAGAGTGG | |
| | CCATCATCGCTGACAAGTCTACCTCCACAGTGTAT | |
| | ATGGCCCTGTCCAGCCTGAGAAGCGAGGATTCCG | |
| | CCGTGTACTTCTGCGCCAGGGCTCGGTCCGACTAC | |
| | TTCAACCGCGATCTGGGTTGGGAGGACTATTACTT | |
| | TGAAAACTGGGGGCAGGGCACACTGGTCACTGTC | |
| | TCATCAGC | |
| 199 | QVQLVQSGAEVKEPGSSVTVSCKASGGTFSNNVISW | FNI17-v19-VH (aa) |
| | VRQAPGQGLEWMGGIIPTSGIANYAQKFQGRVAIIA | |
| | DKSTSTVYMALSSLRSEDSAVYFCARARSDYFNRD | |
| | LGWEDYYFENWGQGTLVTVSS | |
| 200 | GAAATTGTGATGACCCAGTCTCCAGCCACTCTGTC | FNI17-v19-VK (co- |
| | AGTCTCTCCAGGGGAACGAGCCACTCTGTCATGTC | ht) |
| | GGGCCTCTCAGTCCGTCGGCTCCAGCCTGGCTTGG | |
| | TACCAGCAGAAGCCAGGACAGGCTCCTAGGCTGC | |
| | TGATCTATGGAGCTAGCACCAGGGCTACAGGCGT | |
| | GCCAGCTCGGTTCAGCGGATCTGGATCCGGCACCG | |
| | AGTTTACCCTGACAATCTCTTCCCTGCAGTCTGAG | |
| | GACTTCGCCGTGTACTATTGCCAGCACTACAATAA | |
| | CTGGCCTCCTTGGACATTCGGGCAGGGGACAAAA | |
| | GTCGAGATTAAG | |
| 201 | EIVMTQSPATLSVSPGERATLSCRASQSVGSSLAWY | FNI17-v19-VK (aa) |
| | QQKPGQAPRLLIYGASTRATGVPARFSGSGSGTEFTL | |
| | TISSLQSEDFAVYYCQHYNNWPPWTFGQGTKVEIK | |
| 202 | CAGGTCCAGCTGGTGCAGAGTGGTGCCGAGGTCA | FNI19-v3-VH (co- |
| | AAAAGCCAGGGTCAAGTGTCAAAGTCAGTTGTAA | nt) |
| | AGCATCAGAGGGAACATTCAACAAGTACACAATC | |
| | AGCTGGGTGAGACAGGCTCCAGGACAGGGACTGG | |
| | AGTGGATGGGCGGCATCATCCCTATCTCTGGCATC | |
| | GCCAATTACGCTCAGAAGTTCCAGGGCCGCGTGG | |
| | CCATCACAGCTGACGAGTCCACCACAACCGCCTAT | |
| | ATGGAGCTGTCCAGCCTGAGGTCTGAGGATTCCGC | |
| | CGTGTACTATTGCGCCACCGCTGTGAGCGACTACT | |
| | TCAACCGGGATCTGGGCTGGGAGGACTATTATTTT | |
| | CCATTCTGGGGTCAGGGGACACTGGTCACCGTCTC | |
| | TTCC | |
| 203 | QVQLVQSGAEVKKPGSSVKVSCKASEGTFNKYTIS | FNI19-v3-VH (aa) |
| | WVRQAPGQGLEWMGGIIPISGIANYAQKFQGRVAIT | |
| | ADESTTTAYMELSSLRSEDSAVYYCATAVSDYFNR | |
| | DLGWEDYYFPFWGQGTLVTVSS | |
| 204 | GAGATCGTGATGACCCAGTCCCCTGCTACACTGTC | FNI19-v3-VK (co- |
| | CGTGTCCCCAGGAGCTAGGGCTACCCTGTTCTGCA | (nt) |
| | GGGCTAGCAGGTCCGTGTCCGACAACCTGGCTTGG | |
| | TACCAGCAGAAGCCAGGCCAGGCCCCCAGACTGC | |
| | TGATCTTTGGAGCTAGCACCAGAGCTACAGGCGTG | |
| | CCAGCTCGCTTCAGCGGATCTGGATCCGGCACACA | |
| | GTTTACCCTGACAATCTCCAGCCTGCAGTCTGAGG | |
| | ATTTCGCCGTGTACTATTGTCAGCACTATAATATC | |
| | TGGCCCCCTTGGACCTTTGGCCAGGGCACAAAGGT | |
| | GGAGATCAAG | |
| 205 | EIVMTQSPATLSVSPGARATLFCRASRSVSDNLAWY | FNI19-v3-VK (aa) |
| | QQKPGQAPRLLIFGASTRATGVPARFSGSGSGTQFTL | |
| | TISSLQSEDFAVYYCQHYNIWPPWTFGQGTKVEIK | |
| 206 | CAGGTGCACCTGGTGCAGAGCGGAGCTGAGGTGA | FN19-v5-VH (co-nt) |
| | AGGAGCCAGGATCCAGCGTGACAGTGTCTTGCAA | |
| | GGCTTCCGGCGGCAGCTTCAACAATCAGGCTATCT | |
| | CCTGGGTGAGGCAGGCTCCAGGACAGGGACTGGA | |
| | GTGGATGGGCGGCATCTTTCCCATCTCTGGCACAC | |
| | CTACCTCCGCCCAGAGGITCCAGGGAAGGGTGAC | |
| | CTTCACCGCTGACGAGAGCACCACAACCGTGTAC | |
| | ATGGATCTGTCTTCCCTGAGATCTGACGATACCGC | |
| | CGTGTACTATTGTGCCAGAGCTGGCTCCGACTATT | |
| | TCAACCGCGATCTGGGCTGGGAGAATTACTATTTT | |
| | GCTTCCTGGGGCCAGGGCACACTGGTGACCGTGA | |
| | GCTCT | |
| 207 | QVHLVQSGAEVKEPGSSVTVSCKASGGSFNNQAIS | FNI9-v5-VH (aa) |
| | WVRQAPGQGLEWMGGIFPISGTPTSAQRFQGRVTF | |
| | TADESTTTVYMDLSSLRSDDTAVYYCARAGSDYFN | |
| | RDLGWENYYFASWGQGTLVTVSS | |
| 208 | GAGATTGTGATGACCCAGTCCCCTGCTACCCTGAG | FNI9-v5-VK (co-nt) |
| | CGTGTCCCCCGGAGAGAGAGCTACCCTGAGTTGCC | |
| | GCGCCAGCCGCAGTGTCTCTGACAACCTGGCTTGG | |
| | TACCAGCAGAAGCCAGGACAGGCTCCTAGGCTGC | |
| | TGATCTATGGCGCCTCCACCAGGGCTACAGGCATC | |
| | CCAGCTCGGTTCTCTGGATCCGGAAGCGGCACCGA | |
| | GTTTACCCTGACAATCTCCAGCCTGCAGAGCGAGG | |
| | ATTTCGCCGTGTACTATTGCCAGCATTACAACATC | |
| | TGGCCTCCTTGGACATTCGGTCAGGGAACTAAAGT | |
| | GGAAATTAAG | |
| 209 | EIVMTQSPATLSVSPGERATLSCRASRSVSDNLAWY | FNI9-v5-VK (aa) |
| | QQKPGQAPRLLIYGASTRATGIPARFSGSGSGTEFTL | |
| | TISSLQSEDFAVYYCQHYNIWPPWTFGQGTKVEIK | |
| 210 | ASTKGPSVFPLAPSSKSTSGGTAALGCLVKDYFPEPV | IgHG1*01, G1m3 |
| | TVSWNSGALTSGVHTFPAVLQSSGLYSLSSVVTVPSS | CH1-CH3 with |
| | SLGTQTYICNVNHKPSNTKVDKRVEPKSCDKTHTCP | M428L and N434S |
| | PCPAPELLGGPSVFLFPPKPKDTLMISRTPEVTCVVV | mutations and C- |
| | DVSHEDPEVKFNWYVDGVEVHNAKTKPREEQYNST | terminal lysine |
| | YRVVSVLTVLHQDWLNGKEYKCKVSNKALPAPIEK | |
| | TISKAKGQPREPQVYTLPPSREEMTKNQVSLTCLVK | |
| | GFYPSDIAVEWESNGQPENNYKTTPPVLDSDGSFFLY | |
| | SKLTVDKSRWQQGNVFSCSVLHEALHSHYTQKSLSL | |
| | SPGK | |
| 211 | RTVAAPSVFIFPPSDEQLKSGTASVVCLLNNFYPREA | Kappa light chain CL |
| | KVQWKVDNALQSGNSQESVTEQDSKDSTYSLSSTLT | |
| | LSKADYEKHKVYACEVTHQGLSSPVTKSFNRGEC | |
| 212 | QVQLVQSGAEVKEPGSSVTVSCKASGGTFSNNVISW | FNI17-v19 heavy |
| | VRQAPGQGLEWMGGIIPTSGIANYAQKFQGRVAIIA | chain with M428L |
| | DKSTSTVYMALSSLRSEDSAVYFCARARSDYFNRD | and N434S mutations |
| | LGWEDYYFENWGQGTLVTVSSASTKGPSVFPLAPS | in CH3 and a C- |
| | SKSTSGGTAALGCLVKDYFPEPVTVSWNSGALTSGV | terminal lysine |
| | HTFPAVLQSSGLYSLSSVVTVPSSSLGTQTYICNVNH | |
| | KPSNTKVDKRVEPKSCDKTHTCPPCPAPELLGGPSVF | |
| | LFPPKPKDTLMISRTPEVTCVVVDVSHEDPEVKFNW | |
| | YVDGVEVHNAKTKPREEQYNSTYRVVSVLTVLHQD | |
| | WLNGKEYKCKVSNKALPAPIEKTISKAKGQPREPQV | |
| | YTLPPSREEMTKNQVSLTCLVKGFYPSDIAVEWESN | |
| | GQPENNYKTTPPVLDSDGSFFLYSKLTVDKSRWQQG | |
| | NVFSCSVLHEALHSHYTQKSLSLSPGK | |
| 213 | QVQLVQSGAEVKEPGSSVTVSCKASGGTFSNNVISW | FNI17-v19 heavy |
| | VRQAPGQGLEWMGGIIPTSGIANYAQKFQGRVAIIA | chain with M428L |
| | DKSTSTVYMALSSLRSEDSAVYFCARARSDYFNRD | and N434S mutations |
| | LGWEDYYFENWGQGTLVTVSSASTKGPSVFPLAPS | In CH3, without C- |
| | SKSTSGGTAALGCLVKDYFPEPVTVSWNSGALTSGV | terminal lysine |
| | HTFPAVLQSSGLYSLSSVVTVPSSSLGTQTYICNVNH | |
| | KPSNTKVDKRVEPKSCDKTHTCPPCPAPELLGGPSVF | |
| | LFPPKPKDTLMISRTPEVTCVVVDVSHEDPEVKFNW | |
| | YVDGVEVHNAKTKPREEQYNSTYRVVSVLTVLHQD | |
| | WLNGKEYKCKVSNKALPAPIEKTISKAKGQPREPQV | |
| | YTLPPSREEMTKNQVSLTCLVKGFYPSDIAVEWESN | |
| | GQPENNYKTTPPVLDSDGSFFLYSKLTVDKSRWQQG | |
| | NVFSCSVLHEALHSHYTQKSLSLSPG | |
| 214 | EIVMTQSPATLSVSPGERATLSCRASQSVGSSLAWY | FNI17-v19 light |
| | QQKPGQAPRLLIYGASTRATGVPARFSGSGSGTEFTL | chain |
| | TISSLQSEDFAVYYCQHYNNWPPWTFGQGTKVEIK | |
| | RTVAAPSVFIFPPSDEQLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |
| 215 | ASTKGPSVFPLAPSSKSTSGGTAALGCLVKDYFPEPV | IgHG1*01, G1m3 |
| | TVSWNSGALTSGVHTFPAVLQSSGLYSLSSVVTVPSS | CH1-CH3 with |
| | SLGTQTYICNVNHKPSNTKVDKRVEPKSCDKTHTCP | M428L and N434S |
| | PCPAPELLGGPSVFLFPPKPKDTLMISRTPEVTCVVV | mutations, without |
| | DVSHEDPEVKFNWYVDGVEVHNAKTKPREEQYNST | C-terminal lysine |
| | YRVVSVLTVLHQDWLNGKEYKCKVSNKALPAPIEK | |
| | TISKAKGQPREPQVYTLPPSREEMTKNQVSLTCLVK | |
| | GFYPSDIAVEWESNGQPENNYKTTPPVLDSDGSFFLY | |
| | SKLTVDKSRWQQGNVFSCSVLHEALHSHYTQKSLSL | |
| | SPG | |
| 216 | QVQLVQSGARVKEPGSSVKVSCKASGGTFSNNVIS | FNI17-v13 VH (aa) |
| | WVRQAPGQGLEWMGGIIPTSGIANYAQKFQGRVAII | |
| | ADKSTSTVYMALSSLRSEDSAVYFCARARSDYFNR | |
| | DLGWEDYYFENWGQGTLVTVSS | |
| 217 | EIVMTQSPATLSVSPGERATLSCRASQSVGSSLAWY | FNI17-v13 VK (aa) |
| | QQKPGQAPRLLIYGASTRATGVPARFSGSGSGTEFTL | |
| | TISSLQSEDFAVYYCQHYNNWPPWTFGQGTKVEIK | |
| 218 | ACCGGTGTACATTCTCAGGTCCAGCTGGTCCAGAG | Codon-optimized |
| | TGGGGCAGAGGTCAAAgAGCCAGGGTCTTCAGTC | nucleotide sequence |
| | AcAGTCTCATGCAAAGCAAGCGGAGGAACATTTTC | encoding FNI17- |
| | CAACAATGTGATCAGCTGGGTGAGGCAGGCTCCA | v19-VH with N- |
| | GGACAGGGACTGGAGTGGATGGGCGGCATCATCC | terminal amino acids |
| | CTACCTCTGGCATCGCCAACTACGCTCAGAAGTTC | T-G-V-H-S and C- |
| | CAGGGCAGAGTGGCCATCATCGCTGACAAGTCTA | terminal amino acids |
| | CCTCCACAGTGTATATGGCCCTGTCCAGCCTGAGA | I-S |
| | AGCGAGGATTCCGCCGTGTACTTCTGCGCCAGGGC | |
| | TCGGTCCGACTACTTCAACCGCGATCTGGGTTGGG | |
| | AGGACTATTACTTTGAAAACTGGGGGCAGGGCAC | |
| | ACTGGTCACTGTCTCATCAGCGTCGAC | |
| 219 | Ax₁x₂x₃SDYFNRDLGx₄x₅x₆Yx₇Fx₈x₉ | FNI antibody |
| | wherein | consensus CDRH3 |
| | x₁ = R or T; x₂ = A or T; x₃ = V, G, H, R, or N; | amino acid sequence |
| | x₄ = W or F; x₅ = D or E; x₆ = D or N; x₇ = Y or F; | |
| | x₈ = P, D, E, or A; and x₉ = L, I, S, Y, H, D, N, or F | |
| 220 | x₁x₂x₃x₄x₅x₆x₇x₈ | FNI antibody |
| | wherein | consensus CDRH1 |
| | x₁ = G or E; x₂ = D, A, G, or V; x₃ = T or S; x₄ = F or | amino acid sequence |
| | L; x₅ = N or S; x₆ = N, R, K, or S; x₇ = Y, H, Q, or N; | |
| | and x₈ = V, T, A, or E | |
| 221 | Ix₁x₂x₃x₄x₅x₆x₇ | FNI antibody |
| | wherein | consensus CDRH2 |
| | x₁ = I, H, L, or F; x₂ = P or A; x₃ = I, V, L, or T; | amino acid sequence |
| | x₄ = S, T, or F; x₅ = G, A, R, P, or Q; x₆ = I, K, R, | |
| | or T; and x₇ = P, T, A, or G | |
| 222 | x₁x₂x₃x₄x₅x₆ | FNI antibody |
| | wherein | consensus CDRLI |
| | α₁ = Q or R; α₂ = T, S or D; α₃ = V or I; α₄ = S or | amino acid sequence |
| | G; α₅ = S, G, I, T, or D; and α₆ = N, D, H, or S | |
| 223 | x₁AS | FNI antibody |
| | wherein | consensus CDRL2 |
| | x₁ = G or D | amino acid sequence |
| 224 | Qx₁YNx₂x₃PPx₄T | FNI antibody |
| | wherein | consensus CDRL3 |
| | x₁ = Q or H; x₂ = N, T, or I; x₃ = W or F; and x₄ = W | amino acid sequence |
| | or F; | |
| 225 | | [Reserved] |
| 226 | | [Reserved] |
| 227 | CAGGTGCAGCTGGTGCAGTCTGGCGCCGAGGTGAAGA | FNI-UCA-IGH |
| | AGCCAGGCTCCAGCGTGAAGGTGAGCTGCAAGGCTTCT | (wt-nt) |
| | GGCGGCACCTTCTCTTCCTACGCTATCTCCTGGGTGAG | |
| | GCAGGCTCCAGGACAGGGACTGGAGTGGATGGGCGGC | |
| | ATCATCCCTATCTTCGGCACAGCCAACTACGCTCAGAA | |
| | GTTTCAGGGCAGAGTGACCATCACAGCCGACGAGTCTA | |
| | CCTCCACAGCTTATATGGAGCTGAGCTCTCTGCGCTCC | |
| | GAGGATACCGCCGTGTACTATTGTGCCAGGGCTGGCAG | |
| | CGACTACTTCAACCGGGATCTGGGCTGGGAGAATTACT | |
| | ATTTTGACTATTGGGGCCAGGGCACCCTGGTGACAGTG | |
| | TCCAGC | |
| 228 | QVQLVQSGAEVKKPGSSVKVSCKASGGTFSSYAIS | FNI-UCA VH (aa) |
| | WVRQAPGQGLEWMGGIIPIFGTANYAQKFQGRVTI | |
| | TADESTSTAYMELSSLRSEDTAVYYCARAGSDYFN | |
| | RDLGWENYYFDYWGQGTLVTVSS | |
| 229 | GAGATCGTGATGACCCAGTCTCCTGCCACACTGAG | FNI-UCA-IGK |
| | CGTGTCTCCAGGAGAGAGGGCCACCCTGTCCTGCA | (wt-nt) |
| | GGGCTTCCCAGAGCGTGTCCAGCAACCTGGCCTGG | |
| | TACCAGCAGAAGCCAGGCCAGGCTCCCAGGCTGC | |
| | TGATCTATGGCGCCAGCACCAGAGCTACAGGCAT | |
| | CCCAGCTCGCTTCTCTGGATCCGGAAGCGGCACAG | |
| | AGTTTACCCTGACAATCTCTTCCCTGCAGTCTGAG | |
| | GACTTCGCCGTGTACTATTGTCAGCAGTACAACAA | |
| | TTGGCCCCCTTGGACCTTTGGCCAGGGCACAAAGG | |
| | TGGAGATCAAG | |
| 230 | EIVMTQSPATLSVSPGERATLSCRASQSVSSNLAWY | FNI-UCA VK (aa) |
| | QQKPGQAPRLLIYGASTRATGIPARFSGSGSGTEFTL | |
| | TISSLQSEDFAVYYCQQYNNWPPWTFGQGTKVEIK | |
| 231 | GGTFSSYA | FNI-UCA CDRH1 |
| | | (aa) |
| 232 | IPIFGTA | FNI-UCA CDRH2 |
| | | (aa) |
| 233 | ARAGSDYFNRDLGWENYYFDY | FNI-UCA CDRH3 |
| | | (aa) |
| 234 | QSVSSN | FNI-UCA CDRL1 |
| | | (aa) |
| 235 | GAS | FNI-UCA CDRL2 |
| | | (aa) |
| 236 | QQYNNWPPWT | FNI-UCA CDRL3 |
| | | (aa) |

TABLE 2
Sequence Key - SEQ ID NOs. of certain antibodies

| | SEQ ID NO.: | | | | | | | | | | | |
|---|---|---|---|---|---|---|---|---|---|---|---|---|
| | VH | VH | CDRH1 | CDRH2 | CDRH3 | VH | VL (Vk) | VL (Vk) | CDRL1 | CDRL2 | CDRL3 | VL (Vk) |
| Ab | (nt) | (aa) | (aa) | (aa) | (aa) | (co-nt) | (nt) | (aa) | (aa) | (aa) | (aa) | (co-nt) |
| FNI1 | 1 | 2 | 3 | 4 | 5 | 6 | 7 | 8 | 9 | 10 | 11 | 12 |
| FNI2 | 13 | 14 | 15 | 16 | 17 | 18 | 19 | 20 | 21 | 22 | 23 | 24 |
| FNI3 | 25 | 26 | 27 | 28 | 29 | 30 | 31 | 32 | 33 | 34 | 35 | 36 |
| FNI3-VH- | | 171 | 27 | 28 | 172 | 170 | | 32 | 33 | 34 | 35 | 36 |
| W110F | | | | | | | | | | | | |
| FNI3-VK- | 25 | 26 | 27 | 28 | 29 | 30 | | 174 | 33 | 34 | 175 | 173 |
| W94F | | | | | | | | | | | | |
| FNI3-VK- | 25 | 26 | 27 | 28 | 29 | 30 | | 177 | 33 | 34 | 178 | 176 |
| W97F | | | | | | | | | | | | |
| FNI3-VK- | 25 | 26 | 27 | 28 | 29 | 30 | | 180 | 33 | 34 | 181 | 179 |
| W94F-W97F | | | | | | | | | | | | |
| FNI4 | 37 | 38 | 39 | 40 | 41 | 42 | 43 | 44 | 45 | 46 | 47 | 48 |
| FNI5 | 49 | 50 | 51 | 52 | 53 | 54 | 55 | 56 | 57 | 58 | 59 | 60 |
| FNI6 | 61 | 62 | 63 | 64 | 65 | 66 | 67 | 68 | 69 | 70 | 71 | 72 |
| FNI7 | 73 | 74 | 75 | 76 | 77 | 78 | 79 | 80 | 81 | 82 | 83 | 84 |
| FNI9 | 85 | 86 | 87 | 88 | 89 | 90 | 91 | 92 | 93 | 94 | 95 | 96 |
| FNI9-VH- | | 183 | 87 | 88 | 184 | 182 | 91 | 92 | 93 | 94 | 95 | 96 |
| W110F | | | | | | | | | | | | |
| FNI9-VK- | 85 | 86 | 87 | 88 | 89 | 90 | | 186 | 93 | 94 | 187 | 185 |
| W94F | | | | | | | | | | | | |
| FNI9-VK- | 85 | 86 | 87 | 88 | 89 | 90 | | 189 | 93 | 94 | 190 | 188 |
| W97F | | | | | | | | | | | | |
| FNI9-VK- | 85 | 86 | 87 | 88 | 89 | 90 | | 192 | 93 | 94 | 193 | 191 |
| W94F-W97F | | | | | | | | | | | | |
| FNI10 | 97 | 98 | 99 | 100 | 101 | 102 | 103 | 104 | 105 | 106 | 107 | 108 |
| FNI12 | 109 | 110 | 111 | 112 | 113 | 114 | 115 | 116 | 117 | 118 | 119 | 120 |
| FNI13 | 121 | 122 | 123 | 124 | 125 | 126 | 127 | 128 | 129 | 130 | 131 | 132 |
| FNI14 | 133 | 134 | 135 | 136 | 137 | 138 | 139 | 140 | 141 | 142 | 143 | 144 |
| FNI17 | 145 | 146 | 147 | 148 | 149 | 150 | 151 | 152 | 153 | 154 | 155 | 156 |
| FNI19 | 157 | 158 | 159 | 160 | 161 | 162 | 163 | 164 | 165 | 166 | 167 | 168 |
| FNI17-v19 | | 199 | 147 | 148 | 149 | 198 | | 201 | 153 | 154 | 155 | 200 |
| FNI19-v3 | | 203 | 159 | 160 | 161 | 202 | | 205 | 165 | 166 | 167 | 204 |
| FNI9-v5 | | 207 | 87 | 88 | 89 | 206 | | 209 | 141 | 142 | 131 | 208 |
| FNI17-v13 | | 216 | 147 | 148 | 149 | | | 217 | 153 | 154 | 155 | |
| FNI-UCA | 227 | 228 | 231 | 232 | 233 | | 229 | 230 | 234 | 235 | 236 | |

TABLE 3
Neuraminidase Amino Acid Position Comparison (H1N1
California.07.2009 to H3N2 New York.392.2004)

| residue_N1 | position_N1 | residue_N2 | position_N2 |
|---|---|---|---|
| M | 1 | M | 1 |
| N | 2 | N | 2 |
| P | 3 | P | 3 |
| N | 4 | N | 4 |
| Q | 5 | Q | 5 |
| K | 6 | K | 6 |
| I | 7 | I | 7 |
| I | 8 | I | 8 |
| T | 9 | T | 9 |
| I | 10 | I | 10 |
| G | 11 | G | 11 |
| S | 12 | S | 12 |
| V | 13 | V | 13 |
| C | 14 | S | 14 |
| M | 15 | L | 15 |
| T | 16 | T | 16 |
| I | 17 | I | 17 |
| G | 18 | S | 18 |
| M | 19 | T | 19 |
| A | 20 | I | 20 |
| N | 21 | C | 21 |
| L | 22 | F | 22 |
| I | 23 | F | 23 |
| L | 24 | M | 24 |
| Q | 25 | Q | 25 |
| I | 26 | I | 26 |
| G | 27 | A | 27 |
| N | 28 | I | 28 |
| I | 29 | L | 29 |
| I | 30 | I | 30 |
| S | 31 | T | 31 |
| I | 32 | T | 32 |
| W | 33 | V | 33 |
| I | 34 | T | 34 |
| S | 35 | L | 35 |
| H | 36 | H | 36 |
| S | 37 | F | 37 |
| I | 38 | K | 38 |
| Q | 39 | Q | 39 |
| L | 40 | Y | 40 |
| G | 41 | E | 41 |
| N | 42 | F | 42 |
| Q | 43 | N | 43 |
| N | 44 | S | 44 |
| Q | 45 | P | 45 |
| I | 46 | P | 46 |
| E | 47 | N | 47 |
| T | 48 | — | NA |
| C | 49 | — | NA |
| N | 50 | N | 48 |
| Q | 51 | Q | 49 |
| S | 52 | V | 50 |
| V | 53 | M | 51 |
| I | 54 | L | 52 |
| T | 55 | C | 53 |
| Y | 56 | E | 54 |
| E | 57 | P | 55 |
| N | 58 | T | 56 |
| N | 59 | I | 57 |
| T | 60 | I | 58 |
| W | 61 | E | 59 |
| V | 62 | R | 60 |
| N | 63 | N | 61 |
| Q | 64 | I | 62 |
| T | 65 | T | 63 |
| — | NA | E | 64 |
| Y | 66 | I | 65 |
| V | 67 | V | 66 |
| N | 68 | Y | 67 |
| I | 69 | L | 68 |
| S | 70 | T | 69 |
| N | 71 | N | 70 |
| T | 72 | T | 71 |
| N | 73 | T | 72 |
| F | 74 | I | 73 |
| A | 75 | E | 74 |
| A | 76 | K | 75 |
| G | 77 | E | 76 |
| Q | 78 | M | 77 |
| S | 79 | C | 78 |
| V | 80 | P | 79 |
| V | 81 | K | 80 |
| S | 82 | L | 81 |
| V | 83 | A | 82 |
| K | 84 | E | 83 |
| L | 85 | Y | 84 |
| A | 86 | R | 85 |
| G | 87 | N | 86 |
| N | 88 | W | 87 |
| S | 89 | S | 88 |
| S | 90 | K | 89 |
| — | NA | P | 90 |
| L | 91 | Q | 91 |
| C | 92 | C | 92 |
| P | 93 | D | 93 |
| V | 94 | I | 94 |
| S | 95 | T | 95 |
| G | 96 | G | 96 |
| W | 97 | F | 97 |
| A | 98 | A | 98 |
| I | 99 | P | 99 |
| Y | 100 | F | 100 |
| S | 101 | S | 101 |
| K | 102 | K | 102 |
| D | 103 | D | 103 |
| N | 104 | N | 104 |
| S | 105 | S | 105 |
| V | 106 | I | 106 |
| R | 107 | R | 107 |
| I | 108 | L | 108 |
| G | 109 | S | 109 |
| S | 110 | A | 110 |
| K | 111 | G | 111 |
| G | 112 | G | 112 |
| D | 113 | D | 113 |
| V | 114 | I | 114 |
| F | 115 | W | 115 |
| V | 116 | V | 116 |
| I | 117 | T | 117 |
| R | 118 | R | 118 |
| E | 119 | E | 119 |
| P | 120 | P | 120 |
| F | 121 | Y | 121 |
| I | 122 | V | 122 |
| S | 123 | S | 123 |
| C | 124 | C | 124 |
| S | 125 | D | 125 |
| P | 126 | P | 126 |
| L | 127 | D | 127 |
| E | 128 | K | 128 |
| C | 129 | C | 129 |
| R | 130 | Y | 130 |
| T | 131 | Q | 131 |
| F | 132 | F | 132 |
| F | 133 | A | 133 |
| L | 134 | L | 134 |
| T | 135 | G | 135 |
| Q | 136 | Q | 136 |
| G | 137 | G | 137 |
| A | 138 | T | 138 |
| L | 139 | T | 139 |
| L | 140 | L | 140 |
| N | 141 | N | 141 |
| D | 142 | N | 142 |
| K | 143 | V | 143 |
| H | 144 | H | 144 |
| S | 145 | S | 145 |
| N | 146 | N | 146 |
| G | 147 | D | 147 |
| T | 148 | T | 148 |
| I | 149 | V | 149 |
| K | 150 | H | 150 |
| D | 151 | D | 151 |
| R | 152 | R | 152 |
| S | 153 | T | 153 |
| P | 154 | P | 154 |
| Y | 155 | Y | 155 |
| R | 156 | R | 156 |
| T | 157 | T | 157 |
| L | 158 | L | 158 |
| M | 159 | L | 159 |
| S | 160 | M | 160 |
| C | 161 | N | 161 |
| P | 162 | E | 162 |
| I | 163 | L | 163 |
| G | 164 | G | 164 |
| E | 165 | — | NA |
| V | 166 | V | 165 |
| P | 167 | P | 166 |
| S | 168 | F | 167 |
| P | 169 | H | 168 |
| Y | 170 | L | 169 |
| N | 171 | G | 170 |
| S | 172 | T | 171 |
| R | 173 | K | 172 |
| F | 174 | Q | 173 |
| E | 175 | V | 174 |
| S | 176 | C | 175 |
| V | 177 | I | 176 |
| A | 178 | A | 177 |
| W | 179 | W | 178 |
| S | 180 | S | 179 |
| A | 181 | S | 180 |
| S | 182 | S | 181 |
| A | 183 | S | 182 |
| C | 184 | C | 183 |
| H | 185 | H | 184 |
| D | 186 | D | 185 |
| G | 187 | G | 186 |
| I | 188 | K | 187 |
| N | 189 | A | 188 |
| W | 190 | W | 189 |
| L | 191 | L | 190 |
| T | 192 | H | 191 |
| I | 193 | V | 192 |
| G | 194 | C | 193 |
| I | 195 | V | 194 |
| S | 196 | T | 195 |
| G | 197 | G | 196 |
| P | 198 | D | 197 |
| D | 199 | D | 198 |
| N | 200 | K | 199 |
| G | 201 | N | 200 |
| A | 202 | A | 201 |
| V | 203 | T | 202 |
| A | 204 | A | 203 |
| V | 205 | S | 204 |
| L | 206 | F | 205 |
| K | 207 | I | 206 |
| Y | 208 | Y | 207 |
| N | 209 | N | 208 |
| G | 210 | G | 209 |
| I | 211 | R | 210 |
| I | 212 | L | 211 |
| T | 213 | V | 212 |
| D | 214 | D | 213 |
| T | 215 | S | 214 |
| I | 216 | I | 215 |
| K | 217 | V | 216 |
| S | 218 | S | 217 |
| W | 219 | W | 218 |
| R | 220 | S | 219 |
| N | 221 | K | 220 |
| N | 222 | K | 221 |
| I | 223 | I | 222 |
| L | 224 | L | 223 |
| R | 225 | R | 224 |
| T | 226 | T | 225 |
| Q | 227 | Q | 226 |
| E | 228 | E | 227 |
| S | 229 | S | 228 |
| E | 230 | E | 229 |
| C | 231 | C | 230 |
| A | 232 | V | 231 |
| C | 233 | C | 232 |
| V | 234 | I | 233 |
| N | 235 | N | 234 |
| G | 236 | G | 235 |
| S | 237 | T | 236 |
| C | 238 | C | 237 |
| F | 239 | T | 238 |
| T | 240 | V | 239 |
| V | 241 | V | 240 |
| M | 242 | M | 241 |
| T | 243 | T | 242 |
| D | 244 | D | 243 |
| G | 245 | G | 244 |
| P | 246 | S | 245 |
| S | 247 | A | 246 |
| N | 248 | S | 247 |
| G | 249 | G | 248 |
| Q | 250 | K | 249 |
| A | 251 | A | 250 |
| S | 252 | D | 251 |
| Y | 253 | T | 252 |
| K | 254 | K | 253 |
| I | 255 | I | 254 |
| F | 256 | L | 255 |
| R | 257 | F | 256 |
| I | 258 | I | 257 |
| E | 259 | E | 258 |
| K | 260 | E | 259 |
| G | 261 | G | 260 |
| K | 262 | K | 261 |
| I | 263 | I | 262 |
| V | 264 | I | 263 |
| K | 265 | H | 264 |
| S | 266 | T | 265 |
| V | 267 | S | 266 |
| E | 268 | T | 267 |
| M | 269 | L | 268 |
| N | 270 | S | 269 |
| A | 271 | G | 270 |
| P | 272 | S | 271 |
| N | 273 | A | 272 |
| Y | 274 | Q | 273 |
| H | 275 | H | 274 |
| Y | 276 | V | 275 |
| E | 277 | E | 276 |
| E | 278 | E | 277 |
| C | 279 | C | 278 |
| S | 280 | S | 279 |
| C | 281 | C | 280 |
| Y | 282 | Y | 281 |
| P | 283 | P | 282 |
| D | 284 | R | 283 |
| S | 285 | Y | 284 |
| S | 286 | P | 285 |
| E | 287 | G | 286 |
| I | 288 | V | 287 |
| T | 289 | R | 288 |
| C | 290 | C | 289 |
| V | 291 | V | 290 |
| C | 292 | C | 291 |
| R | 293 | R | 292 |
| D | 294 | D | 293 |
| N | 295 | N | 294 |
| W | 296 | W | 295 |
| H | 297 | K | 296 |
| G | 298 | G | 297 |
| S | 299 | S | 298 |
| N | 300 | N | 299 |
| R | 301 | R | 300 |
| P | 302 | P | 301 |
| W | 303 | I | 302 |
| V | 304 | V | 303 |
| S | 305 | D | 304 |
| F | 306 | I | 305 |
| N | 307 | N | 306 |
| — | NA | I | 307 |
| Q | 308 | K | 308 |
| N | 309 | D | 309 |
| L | 310 | Y | 310 |
| E | 311 | S | 311 |
| Y | 312 | I | 312 |
| Q | 313 | V | 313 |
| I | 314 | S | 314 |
| G | 315 | S | 315 |
| Y | 316 | Y | 316 |
| I | 317 | V | 317 |
| C | 318 | C | 318 |
| S | 319 | S | 319 |
| G | 320 | G | 320 |
| I | 321 | L | 321 |
| F | 322 | V | 322 |
| G | 323 | G | 323 |
| D | 324 | D | 324 |
| N | 325 | T | 325 |
| P | 326 | P | 326 |
| R | 327 | R | 327 |
| P | 328 | K | 328 |
| N | 329 | N | 329 |
| D | 330 | D | 330 |
| K | 331 | S | 331 |
| T | 332 | S | 332 |
| G | 333 | S | 333 |
| S | 334 | S | 334 |
| C | 335 | S | 335 |
| — | NA | H | 336 |
| — | NA | C | 337 |
| — | NA | L | 338 |
| G | 336 | D | 339 |
| P | 337 | P | 340 |
| V | 338 | N | 341 |
| S | 339 | N | 342 |
| S | 340 | E | 343 |
| N | 341 | E | 344 |
| G | 342 | G | 345 |
| A | 343 | G | 346 |
| N | 344 | H | 347 |
| G | 345 | G | 348 |
| V | 346 | V | 349 |
| K | 347 | K | 350 |
| G | 348 | G | 351 |
| F | 349 | W | 352 |
| S | 350 | A | 353 |
| F | 351 | F | 354 |
| K | 352 | D | 355 |
| Y | 353 | D | 356 |
| G | 354 | G | 357 |
| N | 355 | N | 358 |
| G | 356 | D | 359 |
| V | 357 | V | 360 |
| W | 358 | W | 361 |
| I | 359 | M | 362 |
| G | 360 | G | 363 |
| R | 361 | R | 364 |
| T | 362 | T | 365 |
| K | 363 | I | 366 |
| S | 364 | S | 367 |
| I | 365 | E | 368 |
| S | 366 | K | 369 |
| S | 367 | L | 370 |
| R | 368 | R | 371 |
| N | 369 | S | 372 |
| G | 370 | G | 373 |
| F | 371 | Y | 374 |
| E | 372 | E | 375 |
| M | 373 | T | 376 |
| I | 374 | F | 377 |
| W | 375 | K | 378 |
| D | 376 | V | 379 |
| P | 377 | I | 380 |
| N | 378 | E | 381 |
| G | 379 | G | 382 |
| W | 380 | W | 383 |
| T | 381 | S | 384 |
| G | 382 | K | 385 |
| T | 383 | P | 386 |
| D | 384 | N | 387 |
| N | 385 | S | 388 |
| N | 386 | K | 389 |
| F | 387 | L | 390 |
| S | 388 | Q | 391 |
| I | 389 | I | 392 |
| — | NA | N | 393 |
| K | 390 | R | 394 |
| Q | 391 | Q | 395 |
| D | 392 | V | 396 |
| I | 393 | I | 397 |
| V | 394 | V | 398 |
| G | 395 | D | 399 |
| I | 396 | R | 400 |
| N | 397 | G | 401 |
| E | 398 | N | 402 |
| W | 399 | R | 403 |
| S | 400 | S | 404 |
| G | 401 | G | 405 |
| Y | 402 | Y | 406 |
| S | 403 | S | 407 |
| G | 404 | G | 408 |
| S | 405 | I | 409 |
| F | 406 | F | 410 |
| V | 407 | — | NA |
| Q | 408 | — | NA |
| H | 409 | — | NA |
| P | 410 | — | NA |
| E | 411 | S | 411 |
| L | 412 | V | 412 |
| T | 413 | E | 413 |
| G | 414 | G | 414 |
| L | 415 | K | 415 |
| D | 416 | S | 416 |
| C | 417 | C | 417 |
| I | 418 | I | 418 |
| R | 419 | N | 419 |
| P | 420 | R | 420 |
| C | 421 | C | 421 |
| F | 422 | F | 422 |
| W | 423 | Y | 423 |
| V | 424 | V | 424 |
| E | 425 | E | 425 |
| L | 426 | L | 426 |
| I | 427 | I | 427 |
| R | 428 | R | 428 |
| G | 429 | G | 429 |
| R | 430 | R | 430 |
| P | 431 | K | 431 |
| K | 432 | E | 432 |
| E | 433 | E | 433 |
| N | 434 | T | 434 |
| T | 435 | E | 435 |
| I | 436 | V | 436 |
| — | NA | L | 437 |
| W | 437 | W | 438 |
| T | 438 | T | 439 |
| S | 439 | S | 440 |
| G | 440 | N | 441 |
| S | 441 | S | 442 |
| S | 442 | I | 443 |
| I | 443 | V | 444 |
| S | 444 | V | 445 |
| F | 445 | F | 446 |
| C | 446 | C | 447 |
| G | 447 | G | 448 |
| V | 448 | T | 449 |
| N | 449 | S | 450 |
| S | 450 | G | 451 |
| D | 451 | T | 452 |
| T | 452 | Y | 453 |
| V | 453 | G | 454 |
| G | 454 | T | 455 |
| W | 455 | G | 456 |
| S | 456 | S | 457 |
| W | 457 | W | 458 |
| P | 458 | P | 459 |
| D | 459 | D | 460 |
| G | 460 | G | 461 |
| A | 461 | A | 462 |
| E | 462 | D | 463 |
| L | 463 | I | 464 |
| P | 464 | N | 465 |
| F | 465 | L | 466 |
| T | 466 | — | NA |
| I | 467 | M | 467 |
| D | 468 | P | 468 |
| K | 469 | I | 469 |

### EXAMPLES

### Example 1

### Identification and Testing of Anti-NA Monoclonal Antibodies

Peripheral blood mononuclear cells (PBMCs) from anonymous donors were selected based on binding of the corresponding serum against N1 and N4 (G1); and N2, N3 and N9 (G2) influenza pseudoviruses. Donors were selected by screening serum from tonsillar donor samples (n=50) for reactivity against neuraminidase subtype N1 and N2 antigens, and serum from PBMC donor samples (n=124) for reactivity against neuraminidase subtype N4, N3, and N9. Neuraminidase antigens for screening were expressed in mammalian cells and binding was evaluated by flow cytometry.

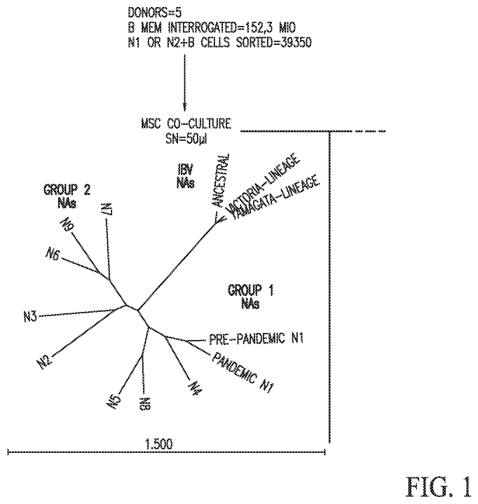

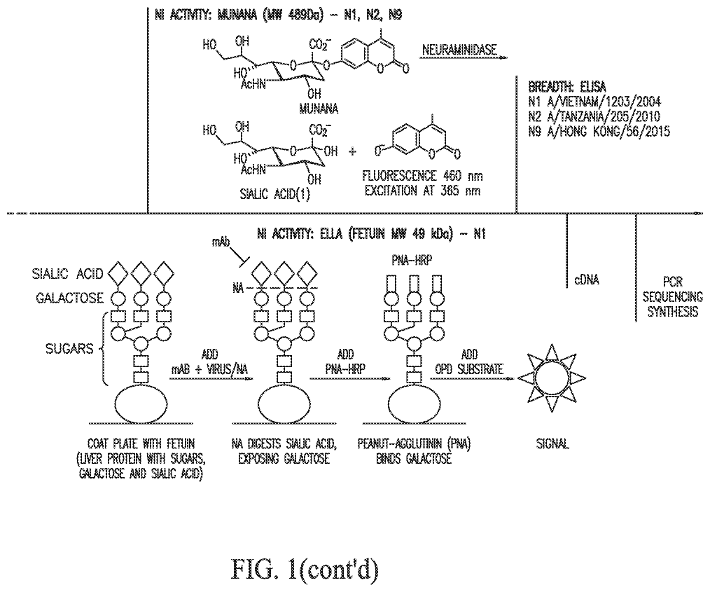

B memory cells from five donors were sorted by flow cytometry for input into the discovery workflow (FIG. 1). Single sorted B cells (n=39,350) were co-cultured with mesenchymal stromal cells (MSC) in 50 μl cultures to stimulate antibody secretion. Secreted antibodies were evaluated by binding and NA inhibition assays. Inhibition of N1 sialidase activity was evaluated using ELLA (enzyme-linked lectin assay), an absorbance-based assay that utilizes a large glycoprotein substrate, fetuin, as a substrate for sialic acid cleavage by NA (Lambre et al. J Immunol Methods. 1990). Inhibition of N1, N2, and N9 sialidase activity was measured using a fluorescence-based assay that measures cleavage of the 2′-(4-Methylumbelliferyl)-α-D-N-acetylneuraminic acid (MUNANA) by the NA enzyme (Potier et al. Anal. Biochem. 1979.).

Binding to NAs from group 1 IAV N1 A/Vietnam/1203/2004, and group 2 IAVs N2 A/Tanzania/205/2010 and N9 A/Hong Kong/56/2015 was evaluated by ELISA to determine breadth. Antibody sequences from selected B cells were cloned as cDNAs and sequenced.

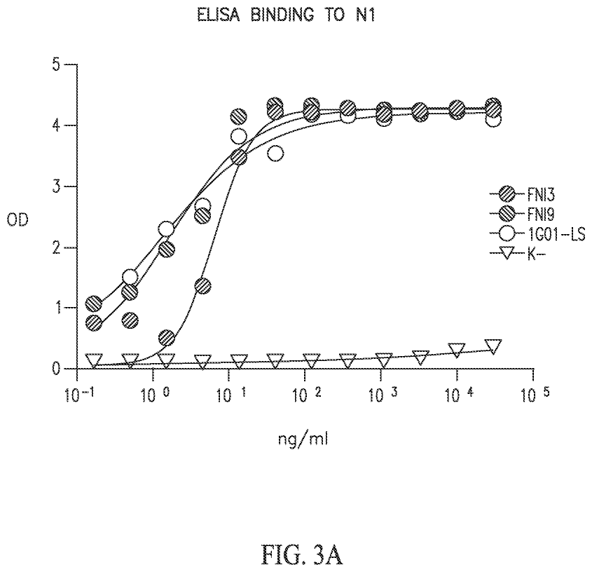

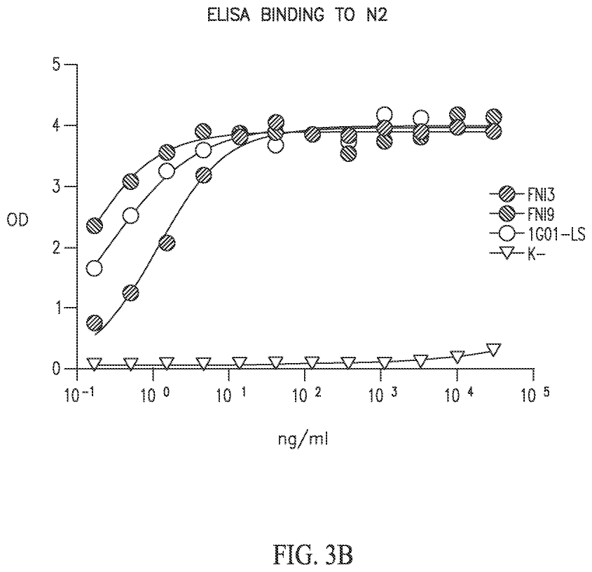

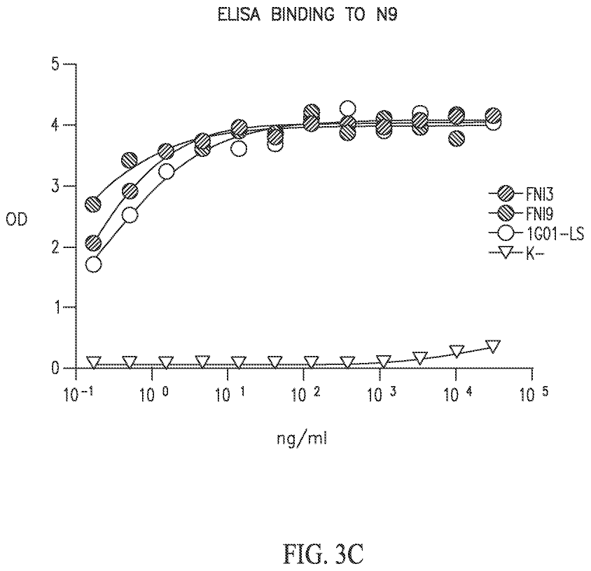

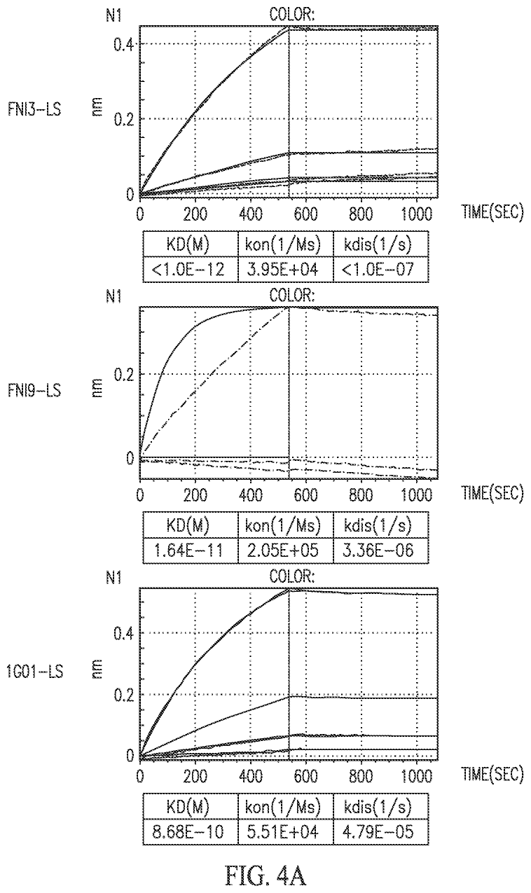

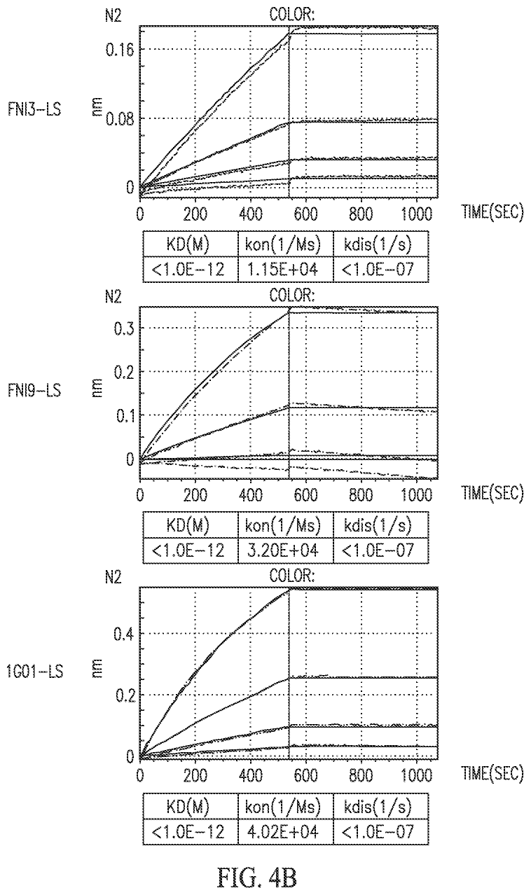

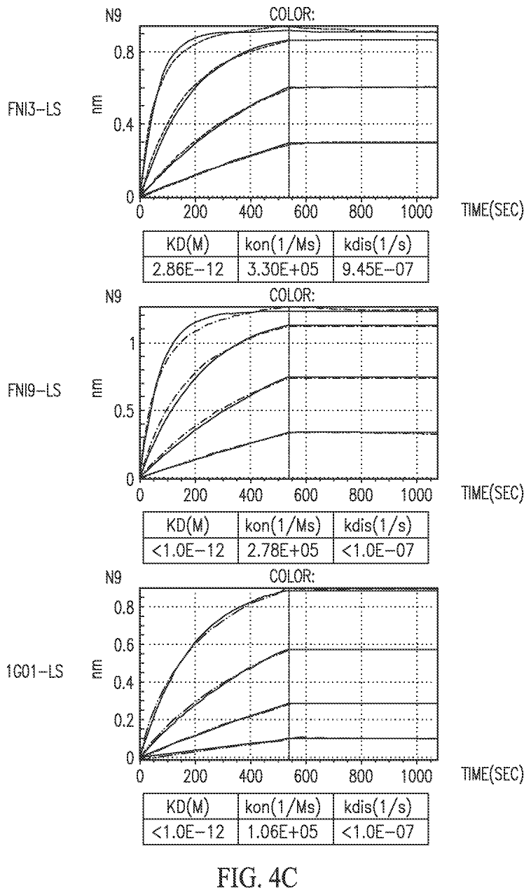

Fourteen clonally related monoclonal antibodies resulted from the discovery workflow (FIG. 2A). FNI3 (VH: SEQ ID NO.:26, VL: SEQ ID NO.: 32) and FNI9 (VH: SEQ ID NO.:86; VL: SEQ ID NO.: 92) were selected for further evaluation and testing. Alignment of FNI3 and FNI9 VH with that of the unmutated common ancestor, “UCA”, is shown in FIG. 2B. The UCA binds to a breadth of IAV and IBV NAs (data not shown). Binding of FNI3 and FNI9 to NA subtypes was evaluated. ELISA (enzyme-linked immunosorbent assay) was used to measure binding of FNI3 and FNI9 to N1 (FIG. 3A), N2 (FIG. 3B), and N9 (FIG. 3C) and reported as optical density (OD) versus concentration in ng/ml. Bio-Layer Interferometry (BLI) was used to measure KD, association (kon), and dissociation (kdis) of FNI3 and FNI9 for N1 binding (FIG. 4A), N2 (FIG. 4B), and N9 (FIG. 4C). Binding by a comparator antibody, 1G01-LS (1G01 is described by Stadlbauer et al. (Science 366(6464):499-504 (2019); see FIG. 1B; the VH and VL amino acid sequences of antibody 1G01, as well as 1E01, and 1G04, are incorporated herein by reference), and in these experiments bore M428L and N434S Fc mutations), was also measured by ELISA and BLI assays. A negative control antibody, K−, tested in the ELISA assays.

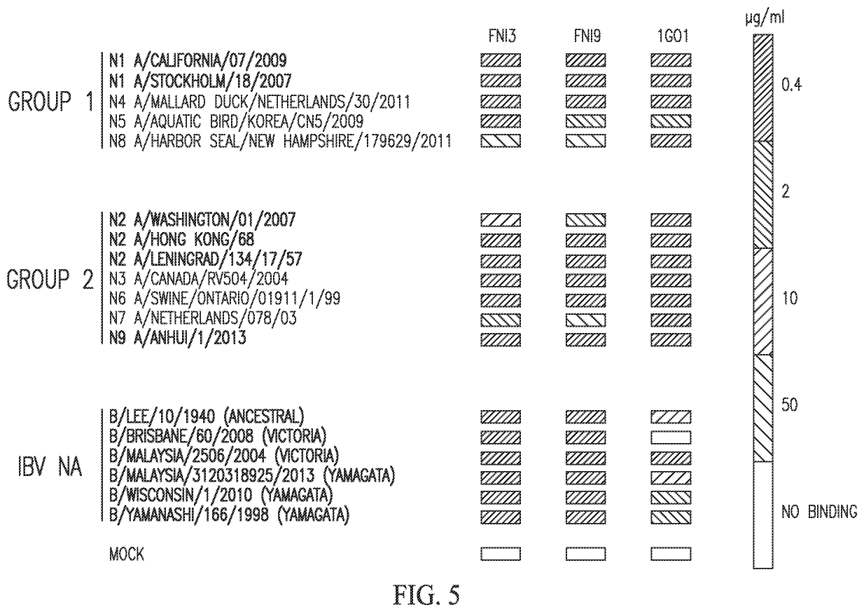

Binding of FNI3 and FNI9 to NAs from group I IAVs, group II IAVs, and IBVs is summarized in FIG. 5 (with comparator 1G01). Binding was quantified using a FACS-based assay in which NAs were expressed on the surface of mammalian cells. Briefly, Expi-CHO cells were transiently transfected with plasmids encoding different IAV and IBV NAs. At 48 hours post-transfection cells were incubated with the serial dilutions of the different mAbs. After 60 minutes incubation, the cells were washed and then incubated with an anti-Human IgG-AF647 secondary antibody. Cells were then washed twice and antibody binding was evaluated at the FACS. 1G01 was used as a comparator.

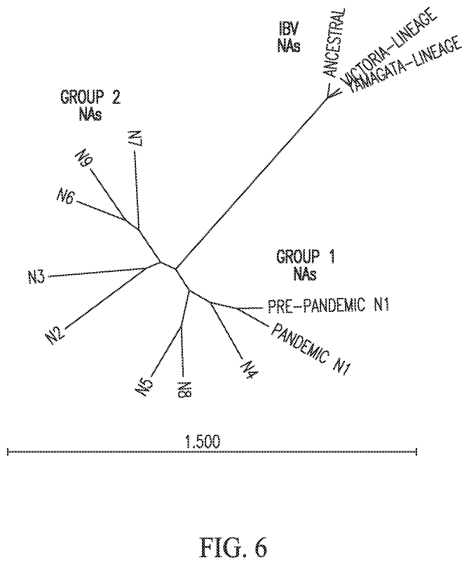

Phylogenetic relatedness of NAs from group 1 IAVs, group 2 IAVs, and Influenza B Viruses is shown in FIG. 6.

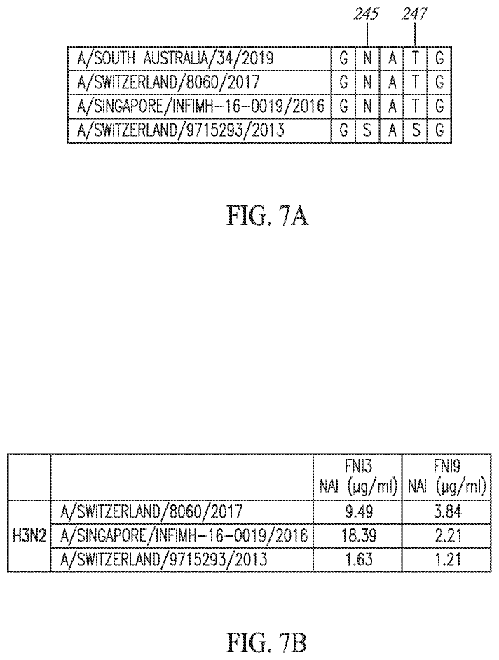

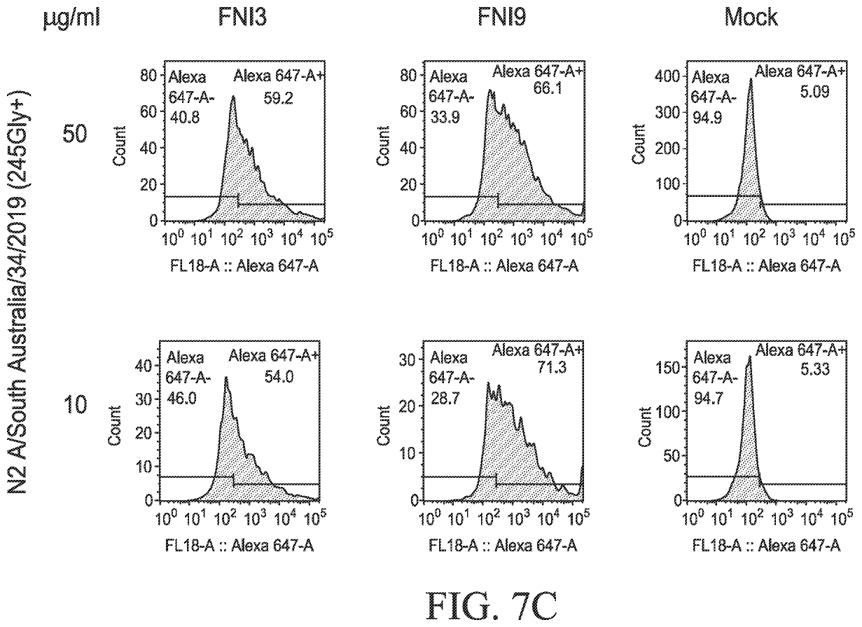

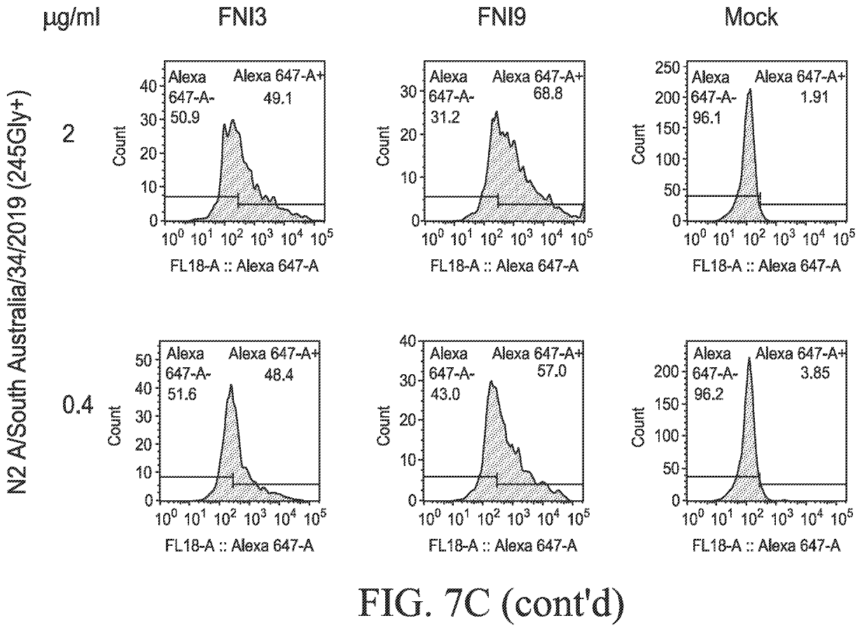

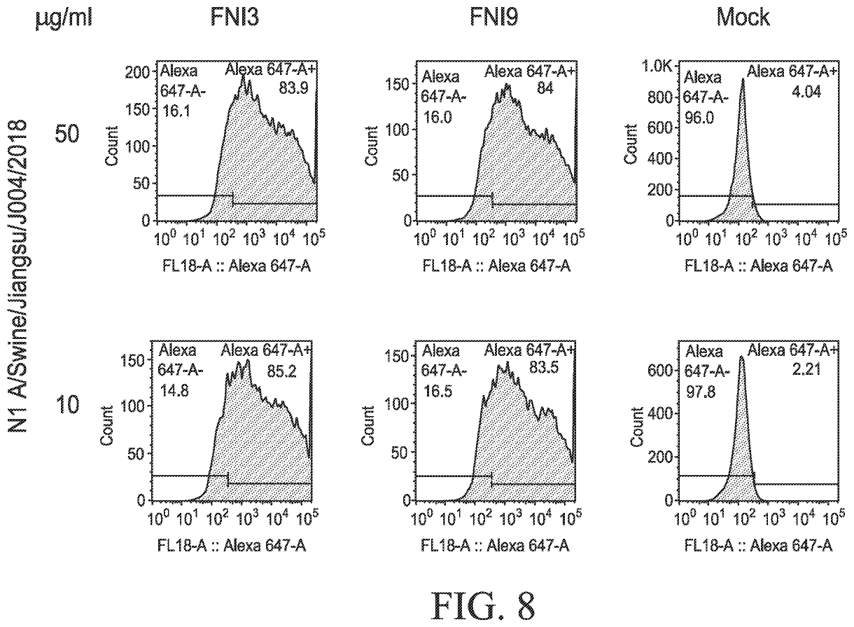

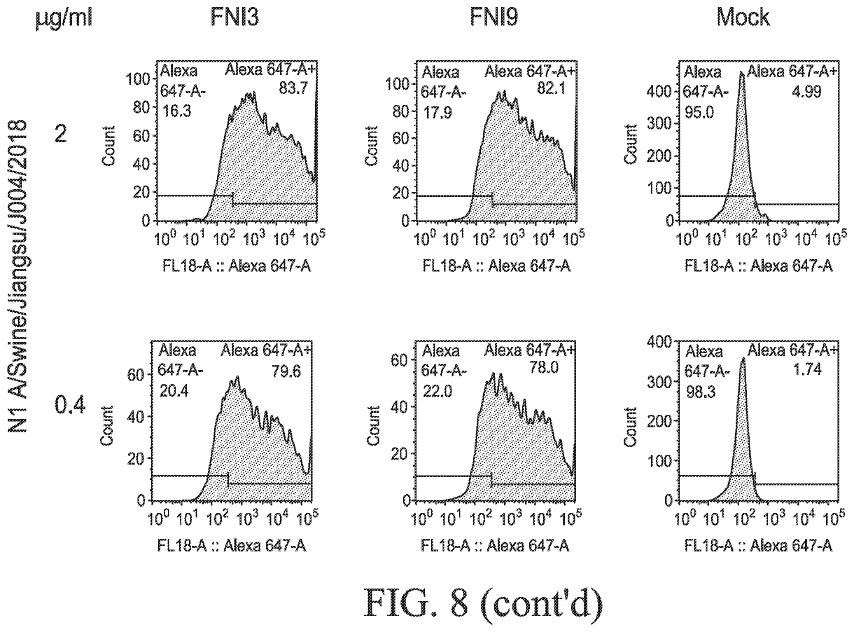

Glycosylation of influenza neuraminidase has implications for immune evasion and viral fitness in a host population. Glycosylation sites can occur at positions 245 (245Gly+) and 247 (247Gly+) (Wan et al. Nat Microbiology. 2019). Exemplary 245Gly+ and 247+Gly modification sites in A/South Australia/34/2019, A/Switzerland/8060/2017, A/Singapore/INFIMH-16-0019/2016, and A/Switzerland/9715293/2013 are shown in FIG. 7A. FIG. 7B shows inhibition of sialidase activity (NAI) activity against A/Switzerland/8060/2017, A/Singapore/INFIMH-16-0019/2016, and A/Switzerland/9715293/2013 live virus stocks, reported as EC50 in μg/ml. Binding of FNI3 and FNI9 to N2 in mammalian cells infected with A/South Australia/34/2019 (245Gly+) was measured by flow cytometry (FIG. 7C). Eurasian avian-like influenza virus strains isolated from swine are genetically diverse (Sun et al. Proc Natl Acad Sci USA. 2020). Binding of FNI3 and FNI9 to NA in mammalian cells infected with a H1N1 Swine Eurasian avian-like (EA) strain, A/Swine/Jiangsu/J004/2018 was measured by flow cytometry, as shown in FIG. 8.

The potential for polyreactivity of FNI3 and FNI9 was evaluated in human epithelial type 2 (HEP-2) cells (FIG. 9). A comparator anti-HA antibody, FI6v3, was used as a positive control, and anti-paramyxovirus antibody “MPE8” (Corti et al. Nature 501(7467):439-43 (2013)) was included as a negative control.

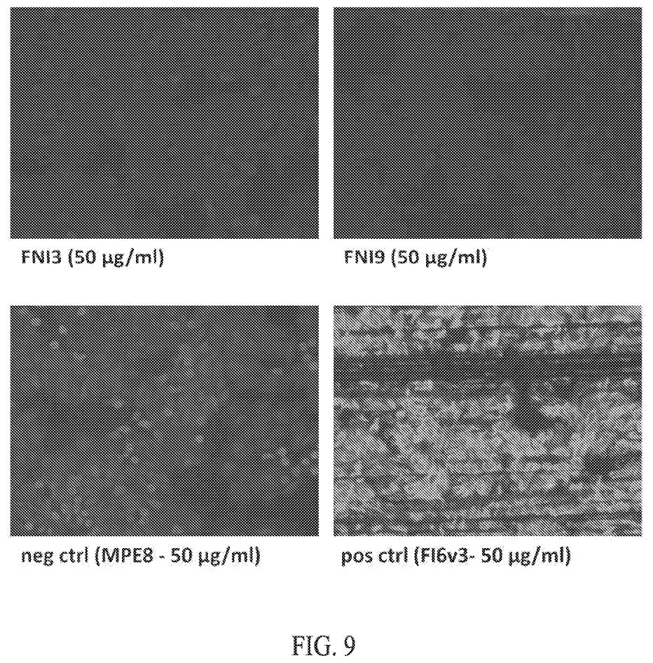

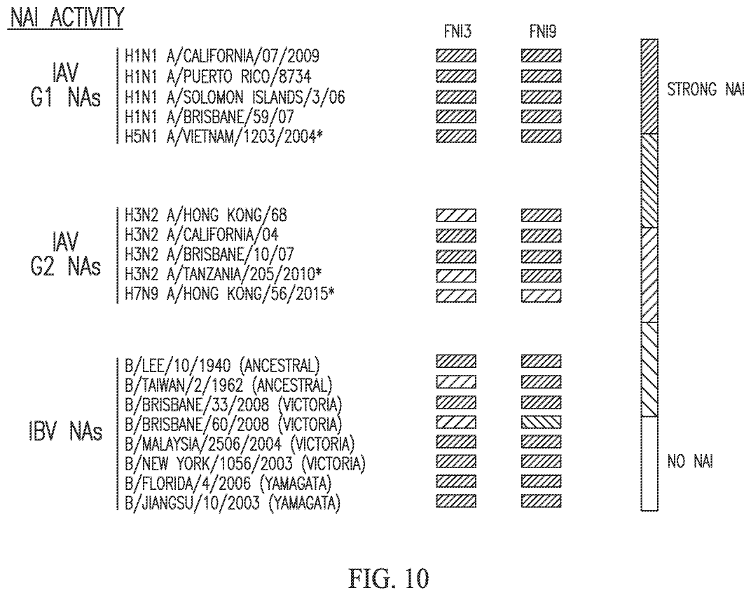

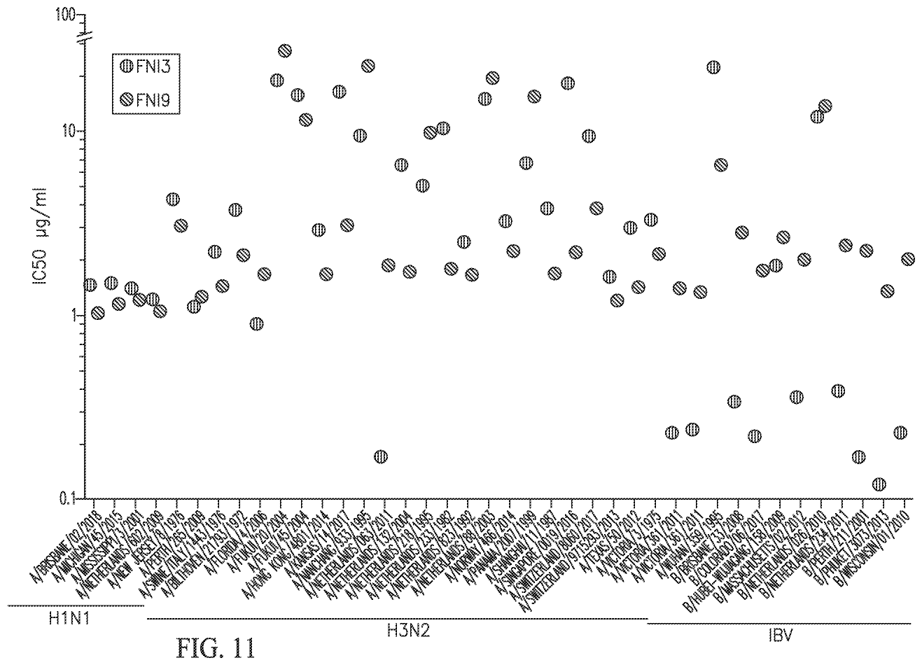

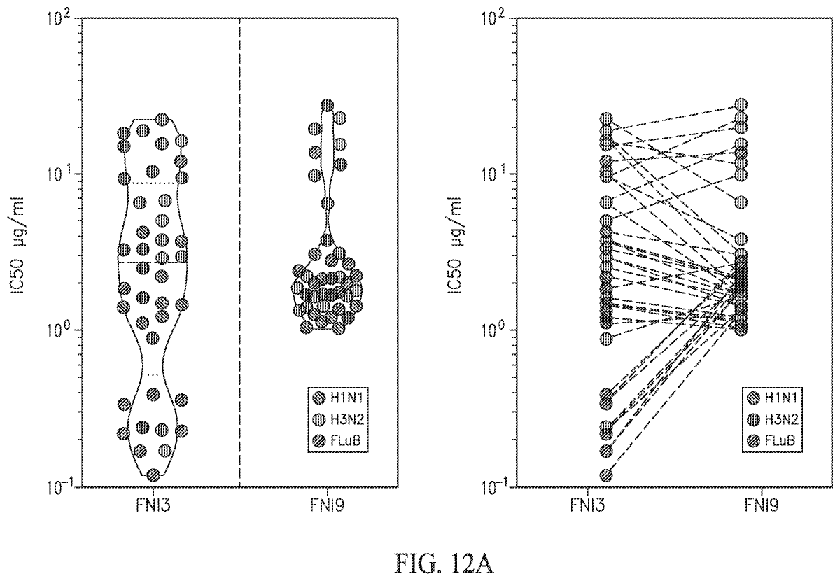

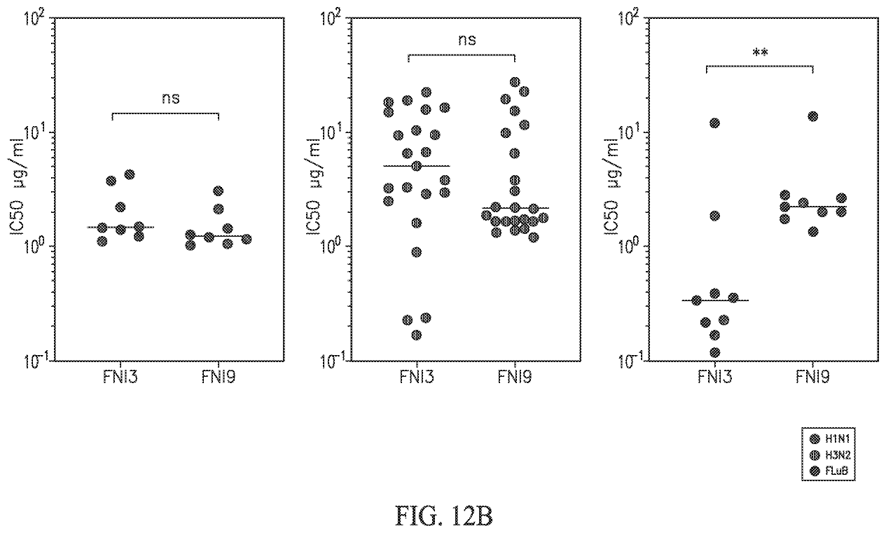

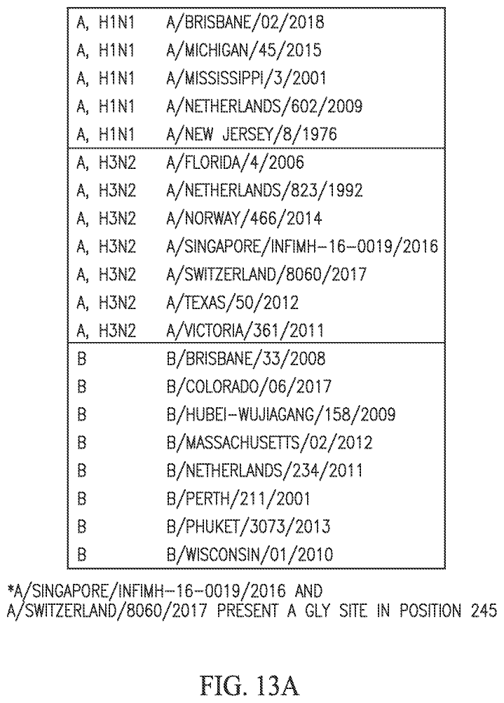

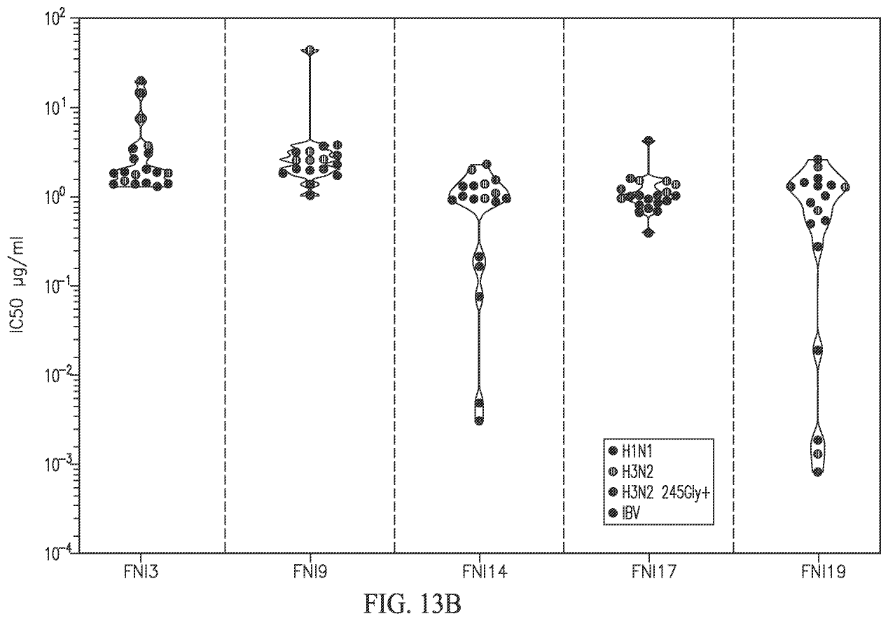

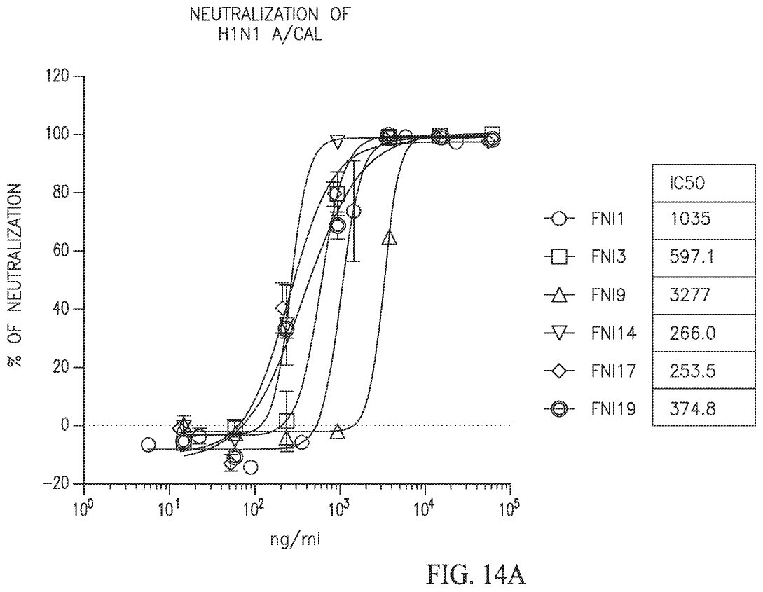

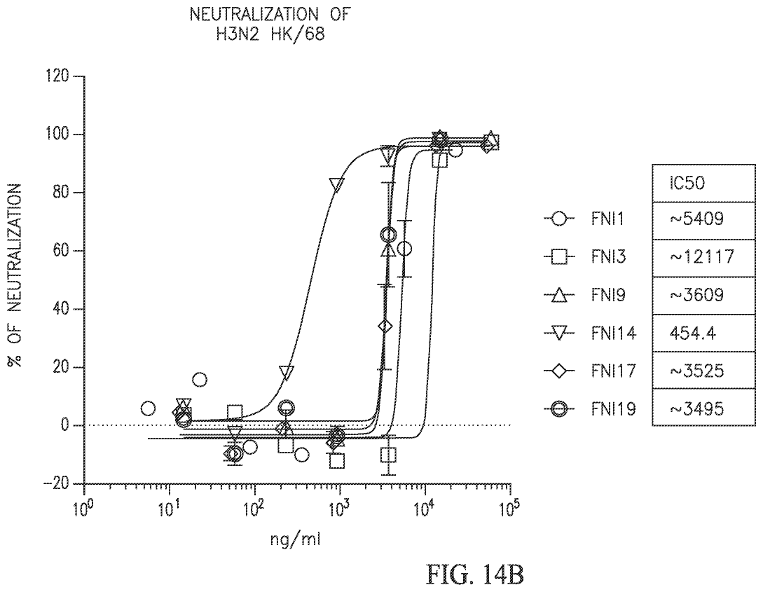

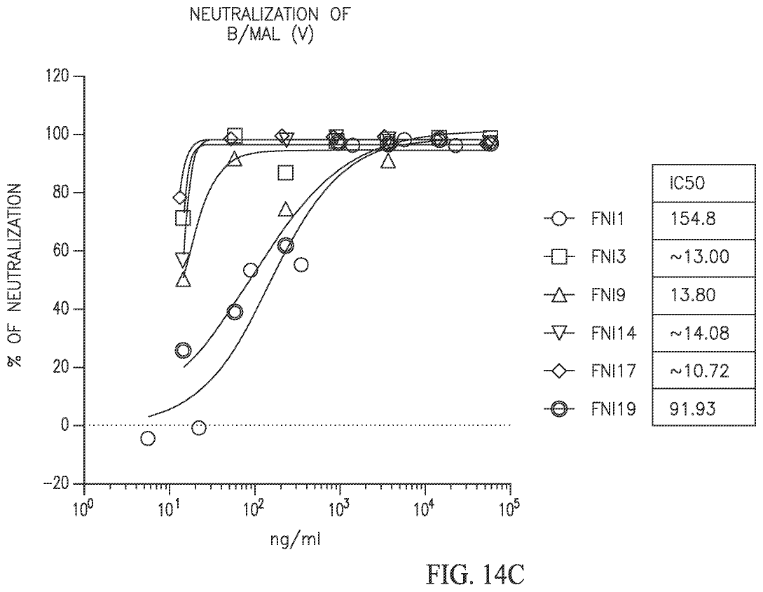

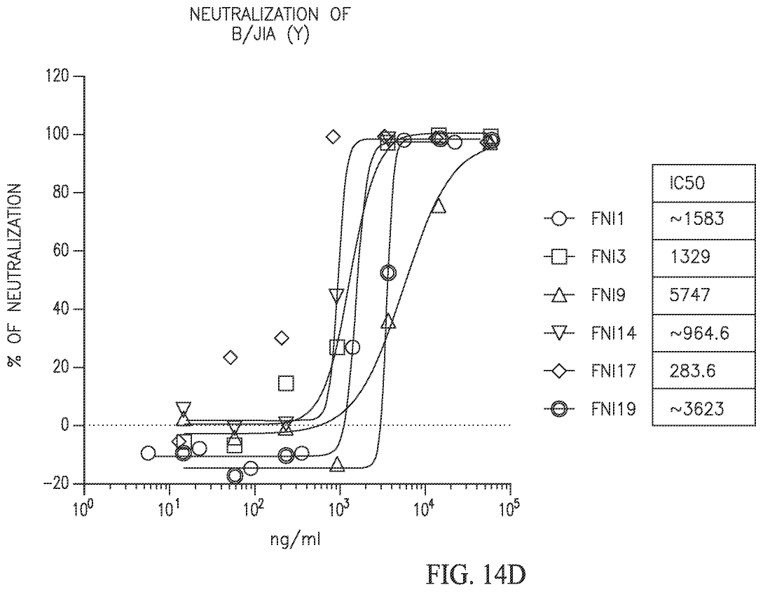

Inhibition of sialidase activity in NAs was measured using a MUNANA assay against group I IAVs, group II IAVs, and IBVs, with results summarized in FIG. 10. Sialidase inhibition of antibody (reported as IC50 in μg/ml) against multiple group I IAVs, group II IAVs, and IBVs strains is summarized in FIG. 11. FIGS. 12A and 12B show in vitro inhibition of sialidase activity (reported as IC50 in μg/ml) by FNI3 or FNI9 against group I (H1N1) IAV, group II (H3N2) IAV, and IBV NAs. FIG. 12A depicts group I IAVs, group II IAVs, and IBVs within the same plot, and FIG. 12B depicts the groups in separate plots. FNI3, FNI9, FNI14 (VH: SEQ ID NO.:134; VL: SEQ ID NO.: 140), FNI17 (VH: SEQ ID NO.:146; VL: SEQ ID NO.: 152), and FNI19 (VH: SEQ ID NO.:158; VL: SEQ ID NO.: 164) were also evaluated for their ability to inhibit sialidase activity (FIG. 13B) of NAs from a panel IAV and IBV strains (FIG. 13A), some of which harbor a glycosylation site at position 245, as indicated by an asterisk. FIGS. 14A-14D show neutralization curves for FNI1 (VH: SEQ ID NO.:2; VL: SEQ ID NO.: 8), FNI3, FNI9, FNI14, FNI17, and FNI19 against H1N1 A/California/07/2009 (FIG. 14A), H3N2 A/Hong Kong/8/68 (FIG. 14B), B/Malaysia/2506/2004 (FIG. 14C), and B/Jiangsu/10/2003 (FIG. 14D) NAs (reported as IC50 (μg/ml).

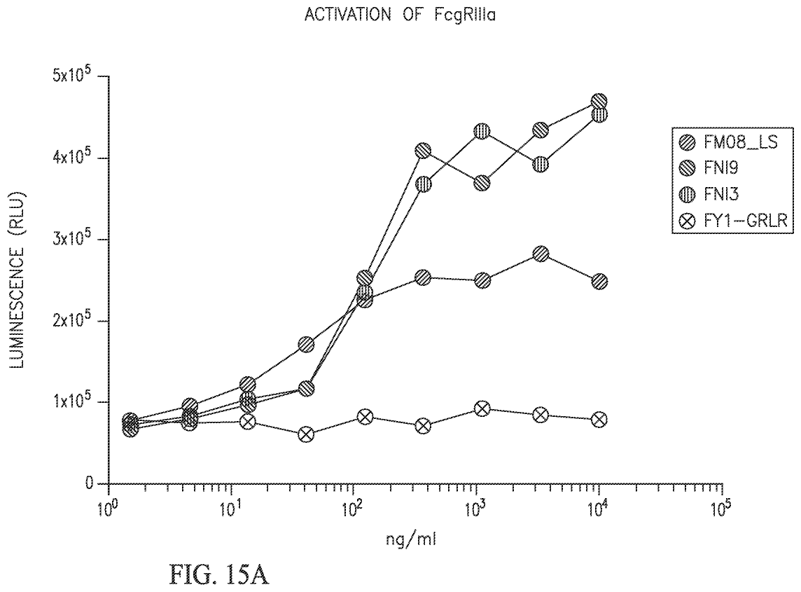

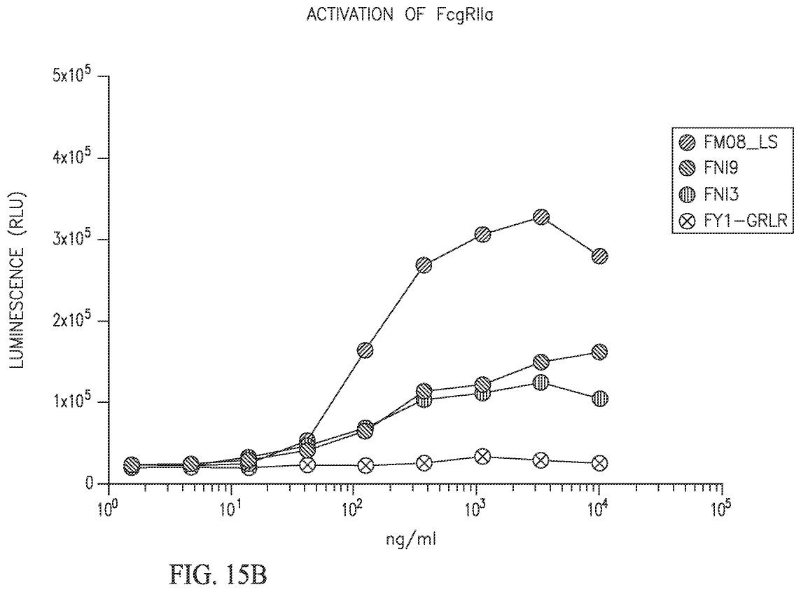

FNI3 and FNI9 were evaluated for activation of FcγRIIIa (FIG. 15A) and FcγRIIa (FIG. 15B) using a NFAT-driven luciferase reporter assay. Activation of Jurkat-FcγRIIIa (F158 allele) and Jurkat-FcγRIIa (H131 allele) cell lines was assessed following a 23 hour incubation with A549 cells infected with H1N1 influenza strain A/Puerto Rico/8/1934 at a multiplicity of infection (MOI) of 6. Comparator antibodies FY1-GRLR and IgG1 antibody FM08_LS, the latter having a VH of SEQ ID NO.:194 and a VL of SEQ ID NO.:195, and comprising M428L and N434S (EU numbering) Fc mutations, were also tested.

### Example 2

### Structural and Functional Studies of Anti-NA Antibodies

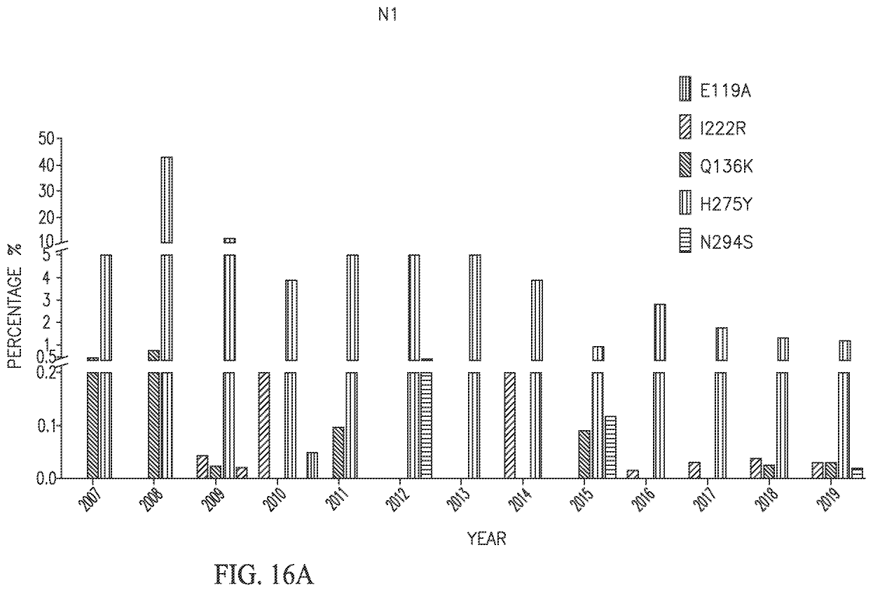

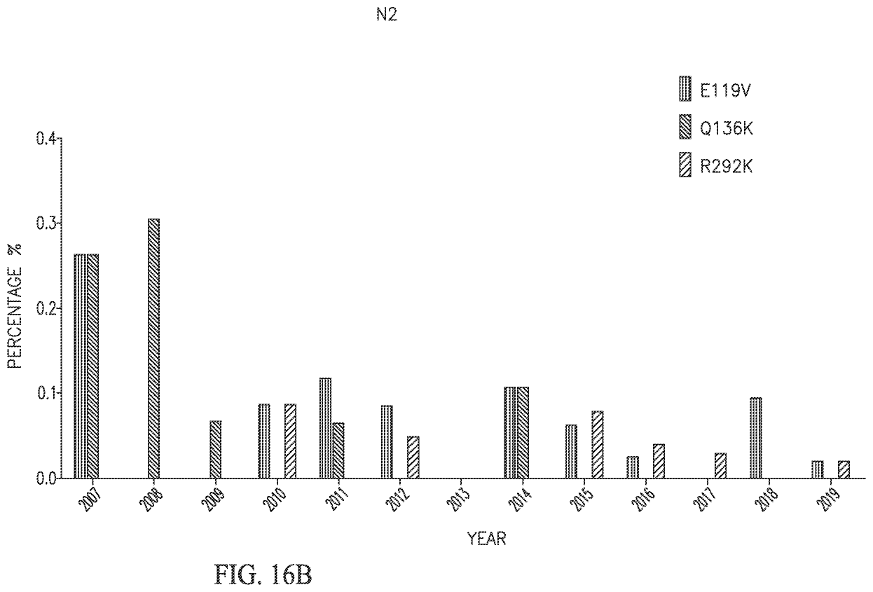

Neuraminidase (NA) mutations responsible for influenza resistance to oseltamivir can vary according to the NA subtype (see, e.g., Hussain et al., Infection and Drug Resistance 10:121-134 (2017)). FIGS. 16A and 16B show frequency by year of NA antiviral-resistant mutations in (FIG. 16A) N1 (H1N1, swine H1N1, and avian H5N1) and (FIG. 16B) N2 (H3N2, H2N2).

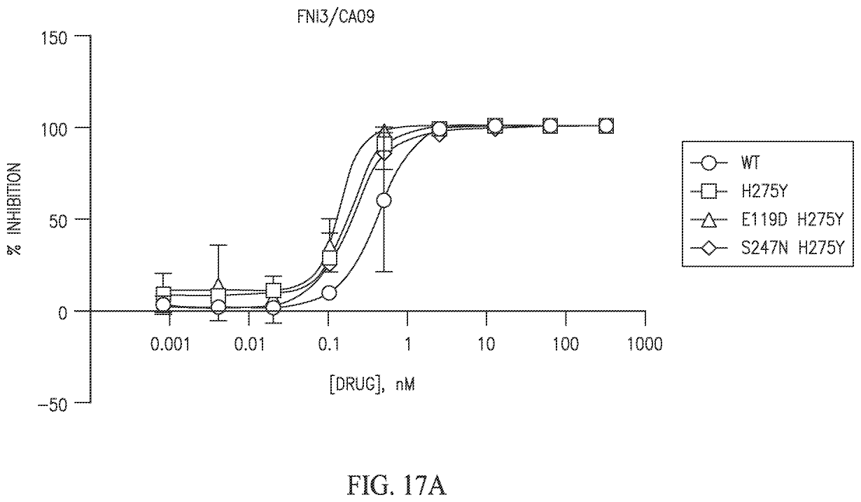

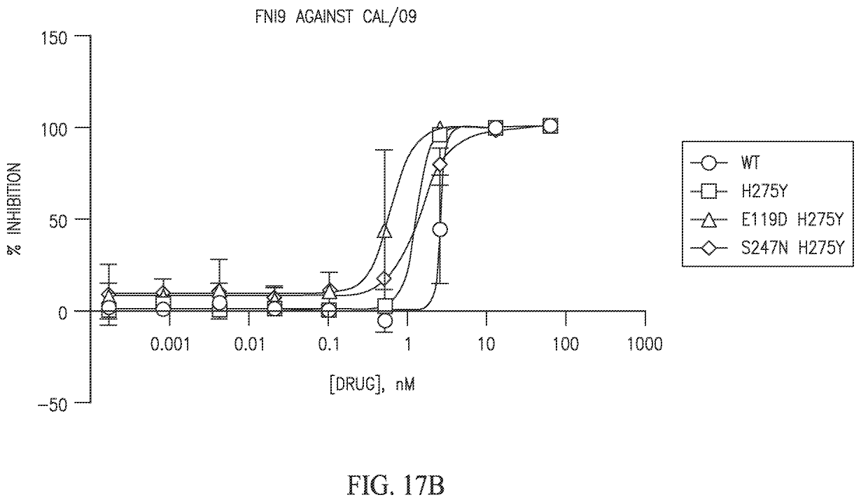

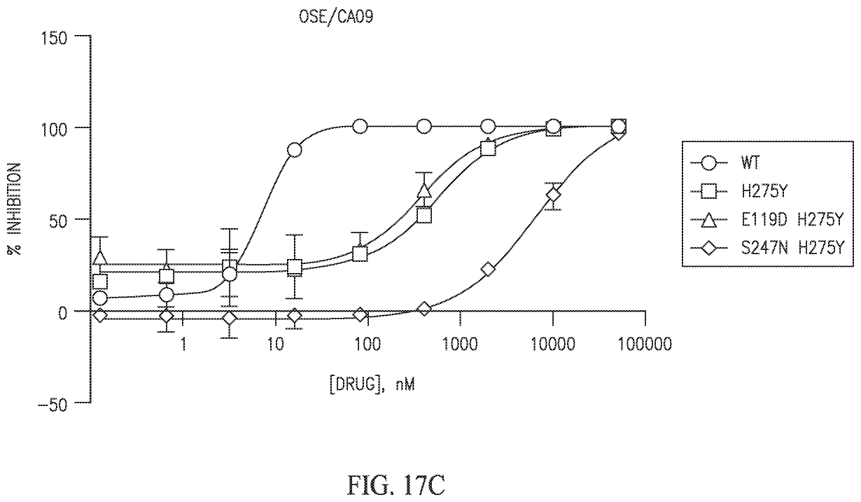

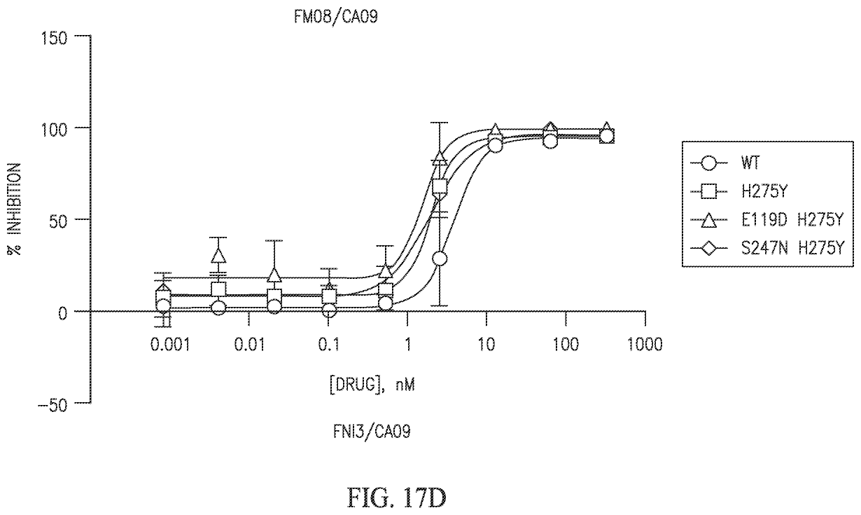

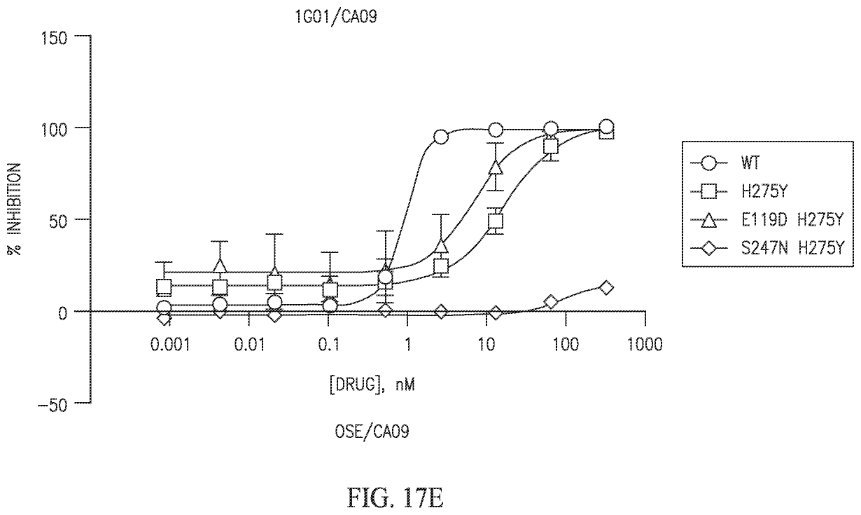

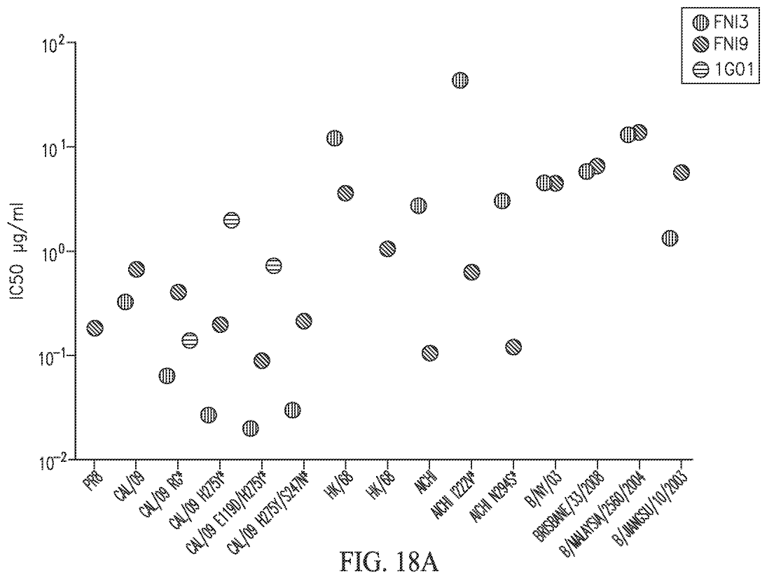

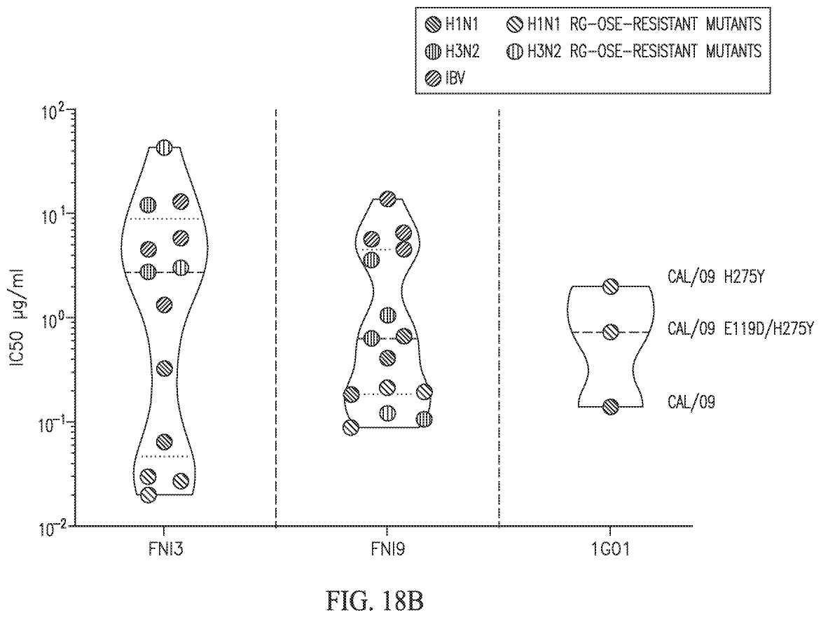

A reverse genetics approach was used to engineer H1N1 A/California/07/2009 to harbor oseltamivir (OSE)-resistant mutations (H275Y, E119D and H275Y, S247N and H275Y). Neutralization of reverse-engineered H1N1 A/California/07/2009 virus by FNI3 (FIG. 17A), FNI9 (FIG. 17B), and oseltamivir (FIG. 17C) was measured, along with neutralization by comparator antibodies FM08 (FIG. 17D) and 1G01 (FIG. 17E) antibodies and reported as % inhibition in nM. These data suggest a structural basis for the lack of susceptibility of FNI3 and FNI9 to OSE-resistant NA mutations. Next, additional viruses, including Group I (H1N1) IAV, group II (H3N2) IAV, and IBV viruses were engineered with reverse genetics to bear OSE-resistant mutations (H275Y, E119D/H275Y, H275Y/S247N, I222V, and N294S). Neutralization activity of FNI3, FNI9, and comparator antibody 1G01 was measured and reported as IC50 in μg/ml. FIG. 18A depicts neutralization of individual viral strains and FIG. 18B depicts neutralization of viral strains grouped by neutralizing anti-NA antibody.

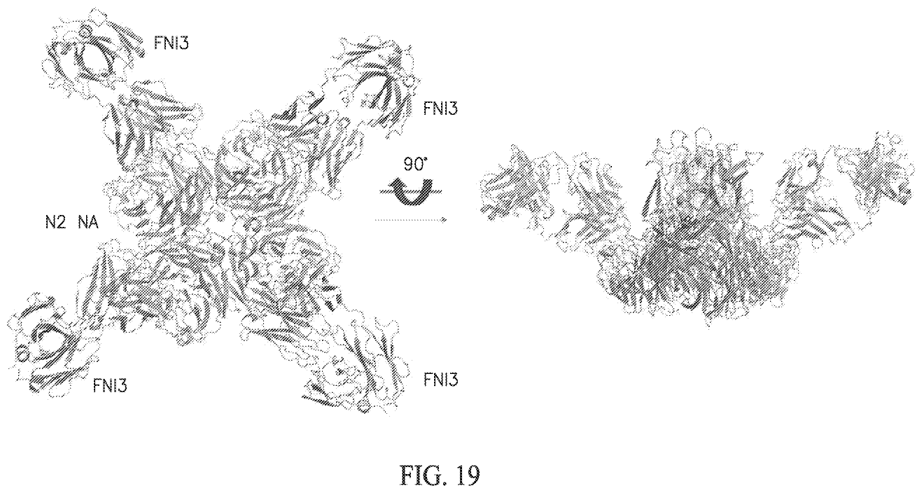

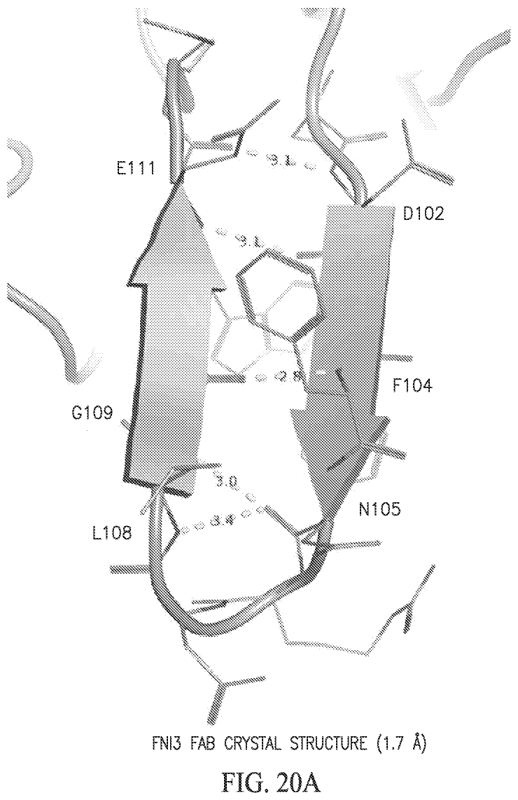

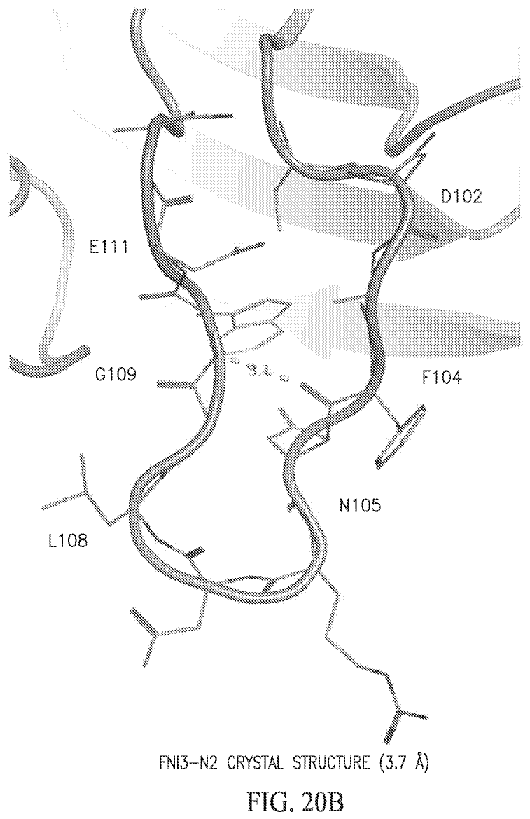

The crystal structure of FNI3 (alone or in complex with NA) was determined to investigate binding function. A relatively flat docking angle of the FNI3 antigen-binding fragment (Fab) domain in complex with NA is shown in FIG. 19. Crystal structure analysis of the complementarity-determining region 3 (CDR3) of the FNI3 heavy chain was performed for unbound (FIG. 20A) or N2 NA-bound states (FIG. 20B). From these studies, unbound FNI3 crystal structure (FIG. 20A) shows a beta sheet conformation and intact main chain hydrogen bonds between carboxylic acid groups (CO) and amino groups (NH) of residues E111 (CO)-D102 (NH), E111 (NH)-D102 (CO), G109 (CO)-F104 (NH), G109 (NH)-N105 (CO), and L108 (NH)-N105 (CO); bound FNI3-N2 crystal structure (FIG. 20B) shows disruption of the beta sheet conformation and one intact main chain hydrogen bond between G109 (CO)-F104 (NH). Without being bound by theory, absence of beta sheet structure in the FNI3-N2 crystal structure might be explained by two potential scenarios: (1) disruption of beta sheet may occur due to induced fit by binding to N2 NA; (2) beta sheet formation may occur due to induced fit by crystal contacts for the Fab domain alone.

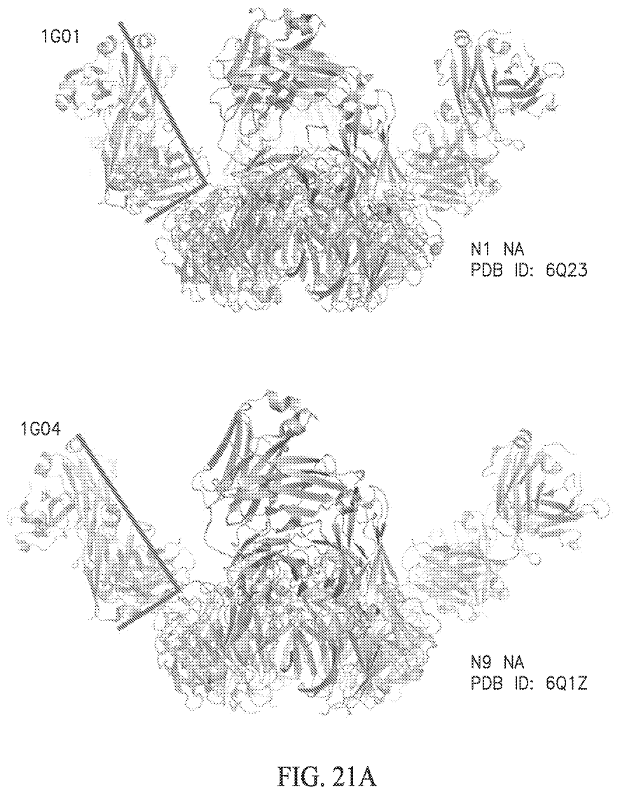

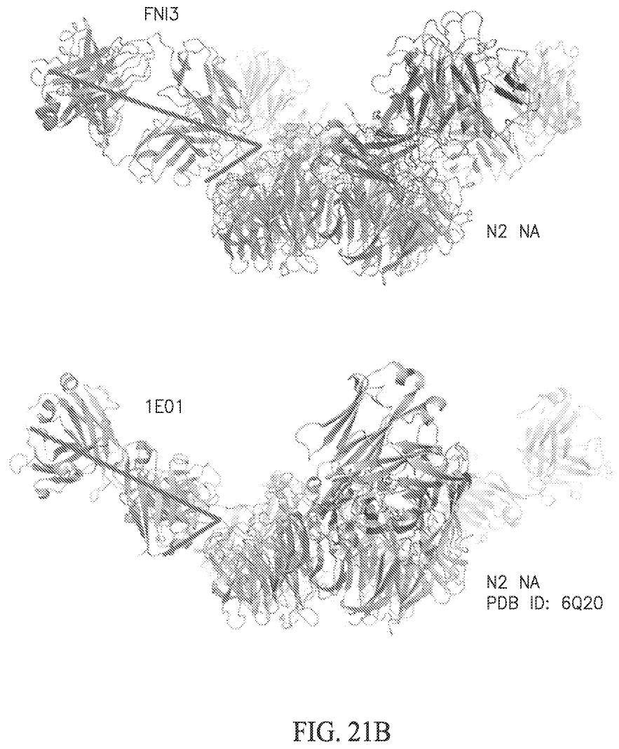

Crystal structure and angle of docking of the Fab domain of the FNI3 antibody in complex with NA subtypes was compared to analogous properties of other anti-NA antibodies to further characterize docking properties of FNI3. FIG. 21A shows comparator antibodies: 1G01 in complex with N1 NA (upper panel); and 1G04 (Stadlbauer et al., supra) in complex with N9 NA (lower panel). FIG. 21B shows FNI3 in complex with N2 NA (upper panel) wherein the docking angle is the same as shown in FIG. 19, but the Fab domain is in a different orientation. FIG. 21B also shows a comparator antibody, 1E01 (Stadlbauer et al., supra), in complex with N2 NA (lower panel). Lines indicate angle of docking and Protein Data Bank (PDB) identification codes are shown for comparator antibodies. From these studies, FNI3 has a similar docking angle to 1E01, but a different Fab orientation.

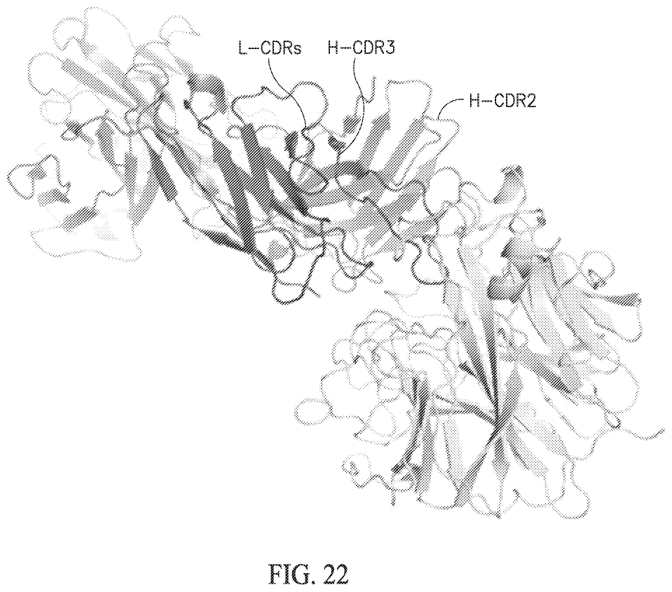

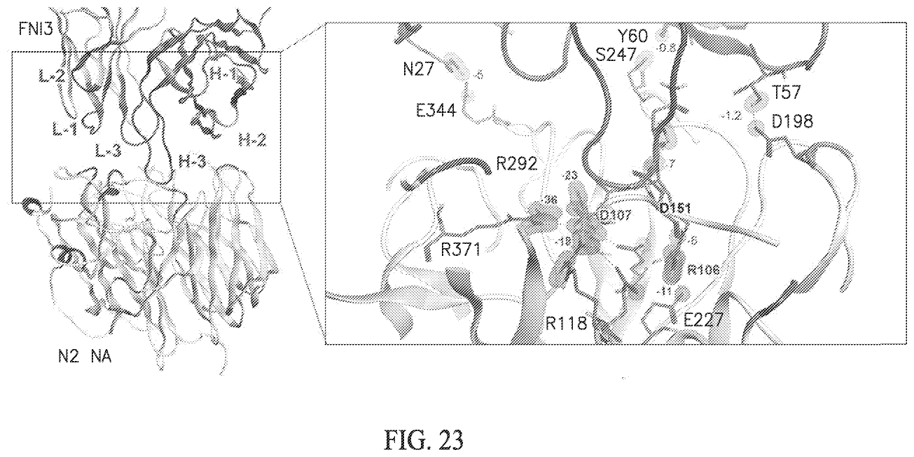

FNI3 complementarity-determining region (CDR) interactions are shown schematically in FIG. 22, from “quick prepped” protein using MOE (Molecular Operating Environment by the Chemical Computing Group; www.chemcomp.com). From this analysis, CDRH3 bends at an almost 90° angle to occupy the NA binding pocket and CDRH2 lays flat on the NA surface. From this analysis, CDRH2 does not appear to energetically contribute to binding and CDRLs do not appear to contribute to the binding interaction. From this study, within the CDRH3, D107 and R106 appear to contribute to N2 NA binding (FIG. 23). Negative numbers are interaction energy in kcal/mol.

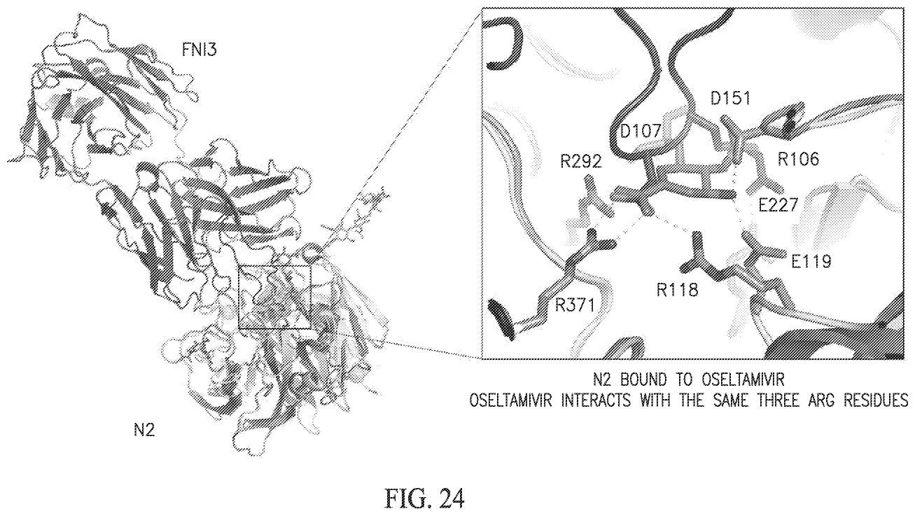

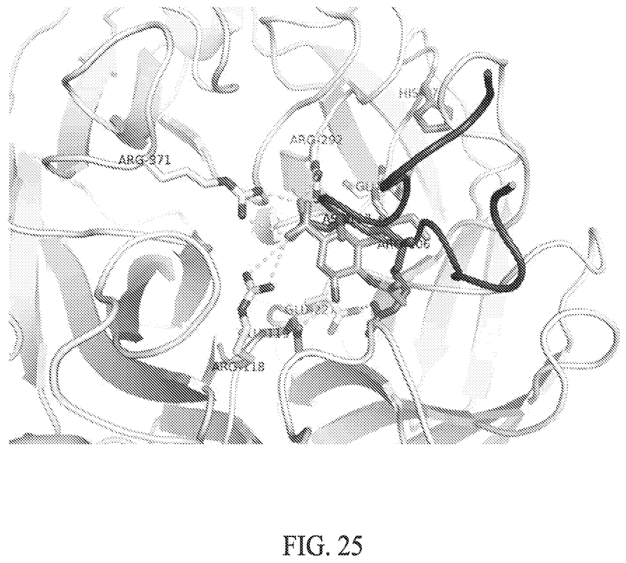

The crystal structure of FNI3 was overlaid on the structure of oseltamivir-bound N2 NA (FIG. 24, FIG. 25), showing that oseltamivir interacts with R118, R292, and R371.

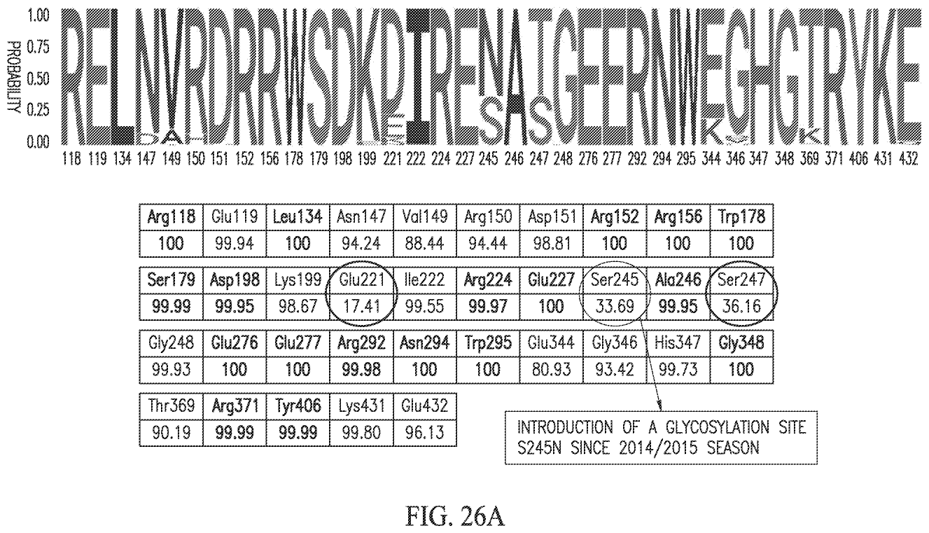

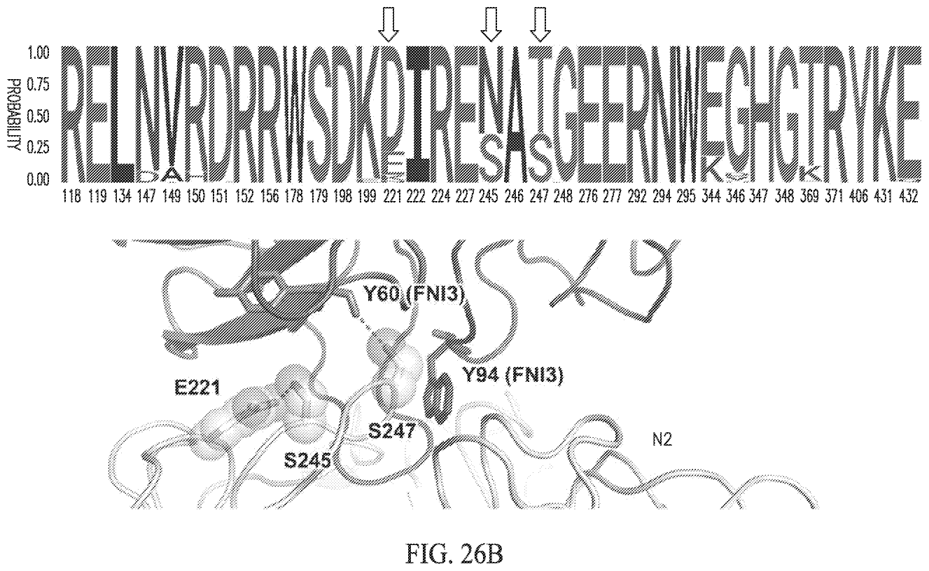

Conservation of the FNI3 epitope was investigated using N2 NA sequences from H3N2 viruses (n=60,597) isolated between the years 2000 and 2020. The epitope region consensus amino acid sequence is shown in FIG. 26A, with a table showing the frequency of an amino acid at a particular position in the group of analyzed N2 NA sequences. Circled values indicate amino acids appearing at the lowest three frequencies, Glu221 (E221, 17.41%), Ser245 (S245, 33.69%), and Ser247 (S247, 36.16%). FIG. 26B (lower portion) shows interaction of Y60 and Y94 from FNI3 with residues E221, S245, and S247 of N2 NA. Using simple modeling, a S245N mutation increased binding, a S247T mutation decreased binding, and a E221D mutation was neutral in effect (data not shown).

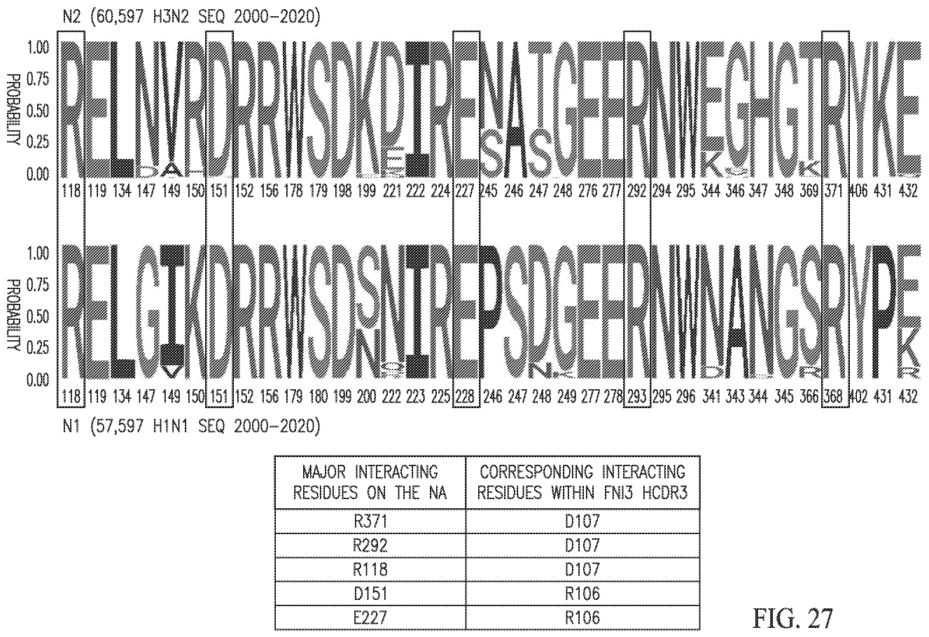

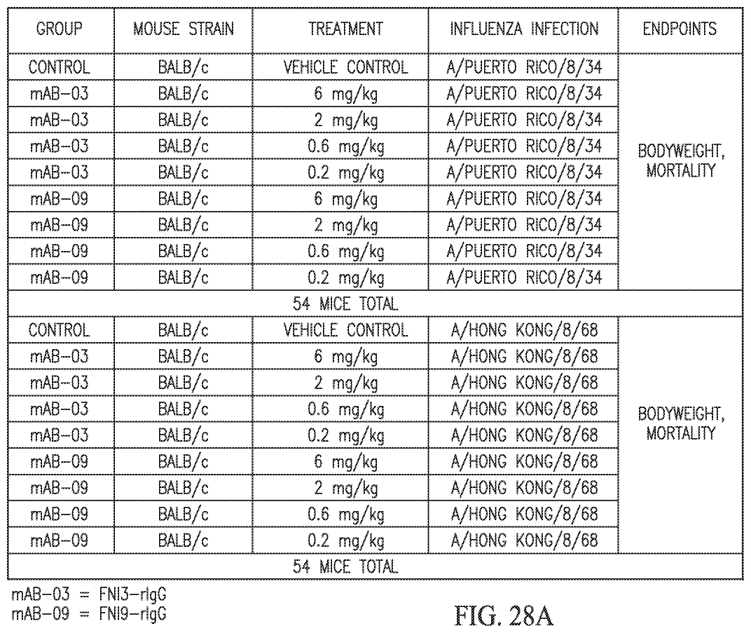

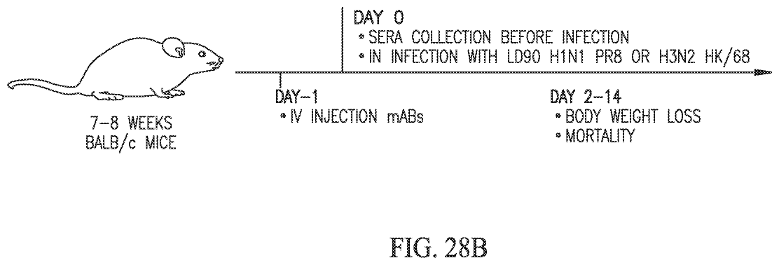

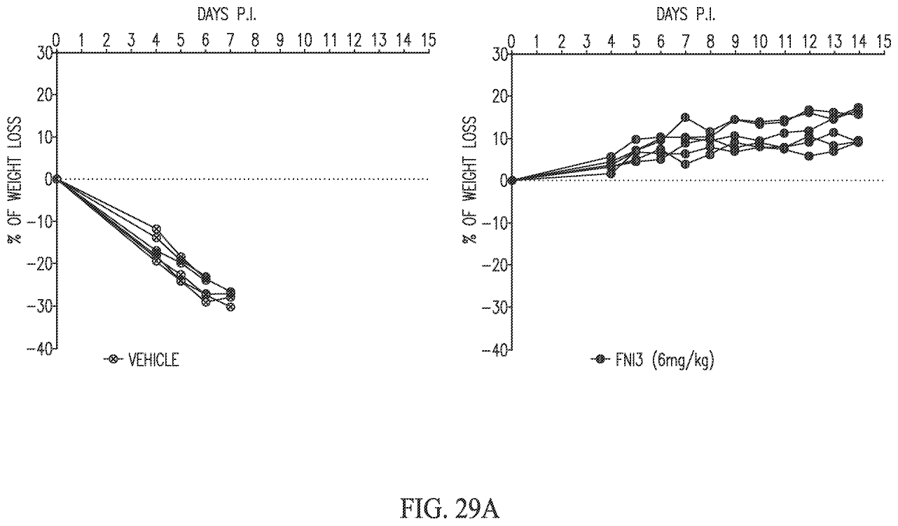

Conservation of the FNI3 epitope was investigated using N1 NA sequences from H1N1 viruses (n=57,597) isolated between the years 2000 and 2020. FIG. 27 shows a comparison of N2 NA FNI3 epitope conservation analysis (shown in FIGS. 26A and 26B) with analysis of FNI3 epitope conservation in N1 NA sequences from H1N1. Pairs of consensus residues were identified, R118 (N2) and R118 (N1), D151 (N2) and D151 (N1), E227 (N2) and E228 (N1), R292 (N2) and R293 (N1), and R371 (N2) and R368 (N1). Important FNI3-interacting residues within N2 NA and counterpart FNI3 CDRH3 residues are shown in the table in the lower panel. Residues R371, R292, and R118 interact with D107 of FNI3 CDRH3 and residues D151 and E227 interact with R106 of FNI3 CDRH3.

### Example 3

### Prophylactic Activity of Anti-NA Monoclonal Antibodies

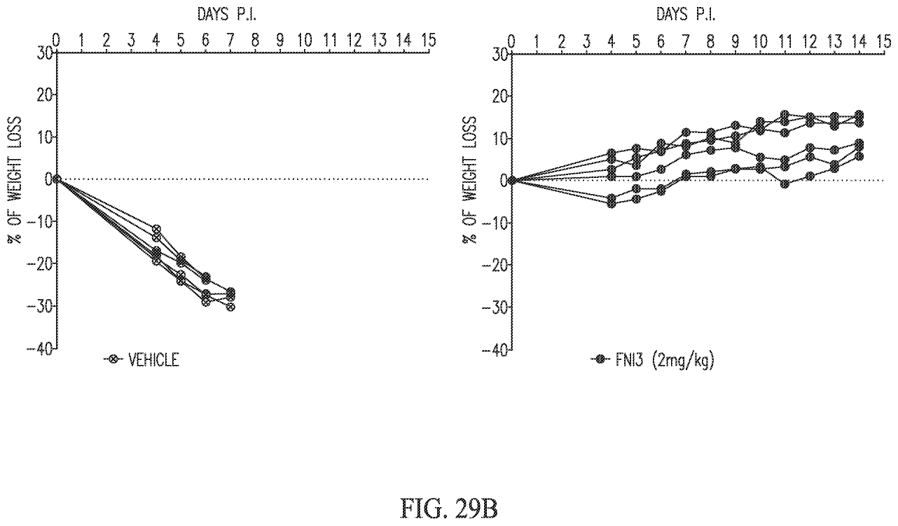

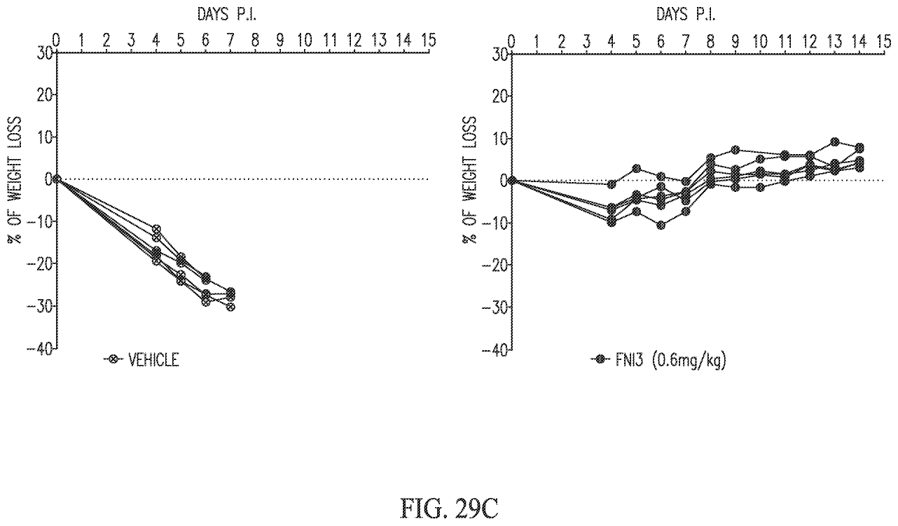

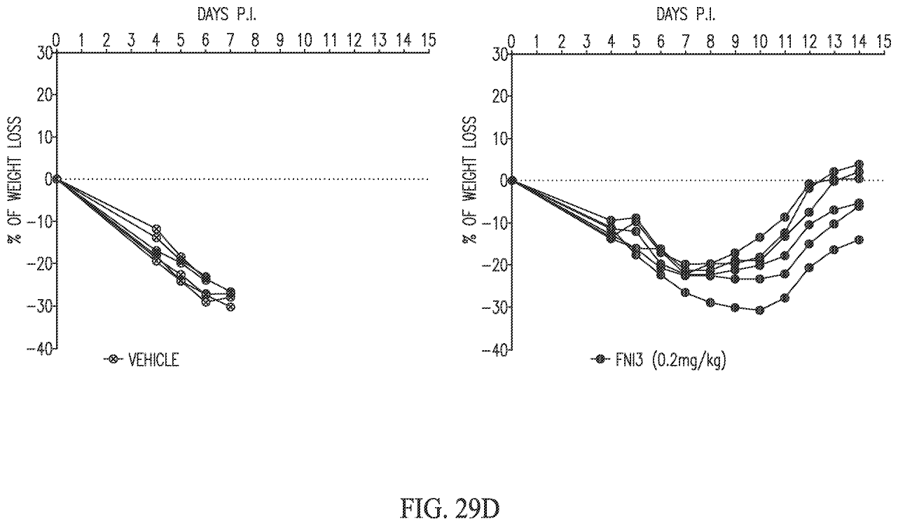

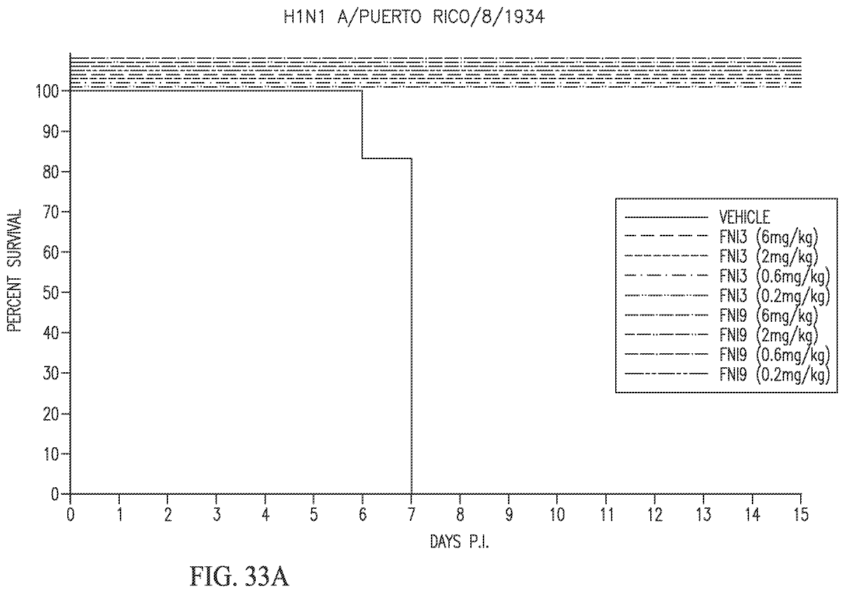

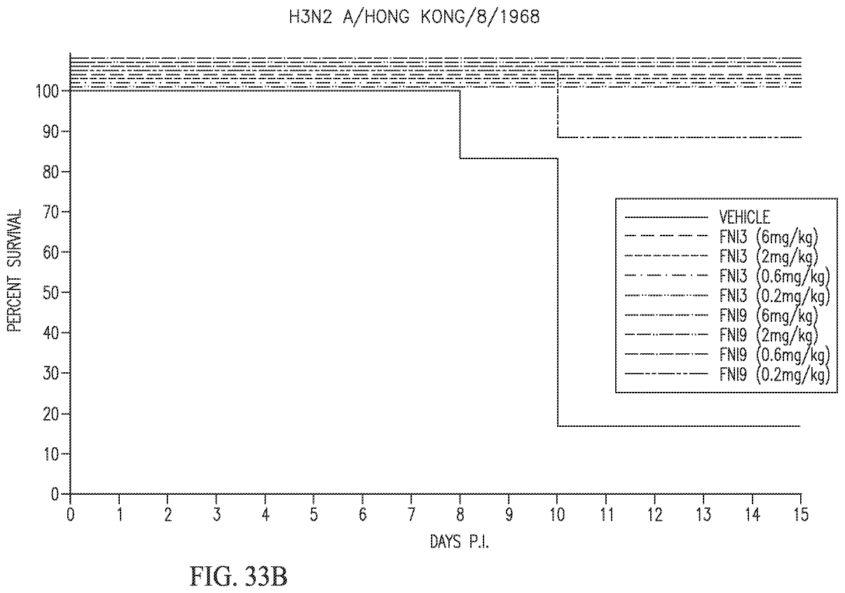

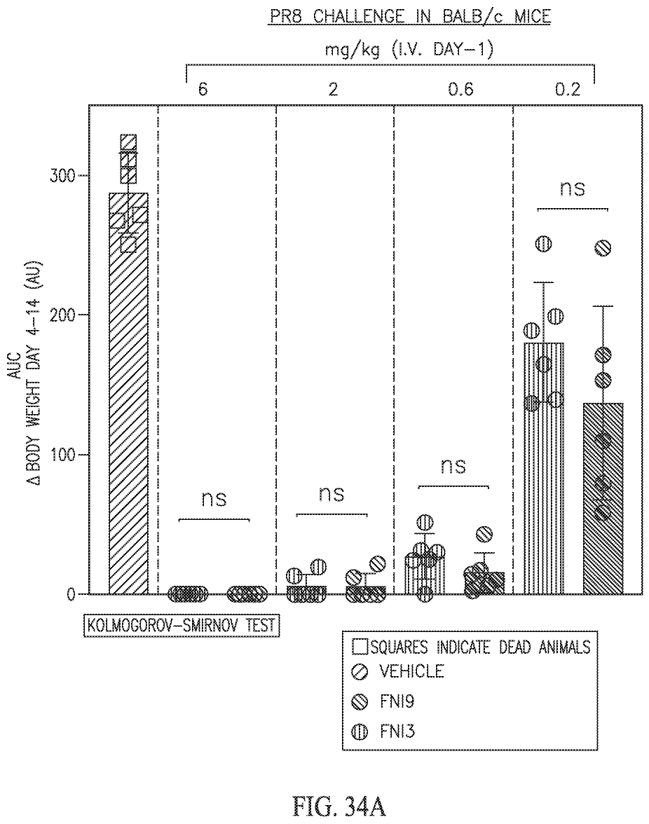

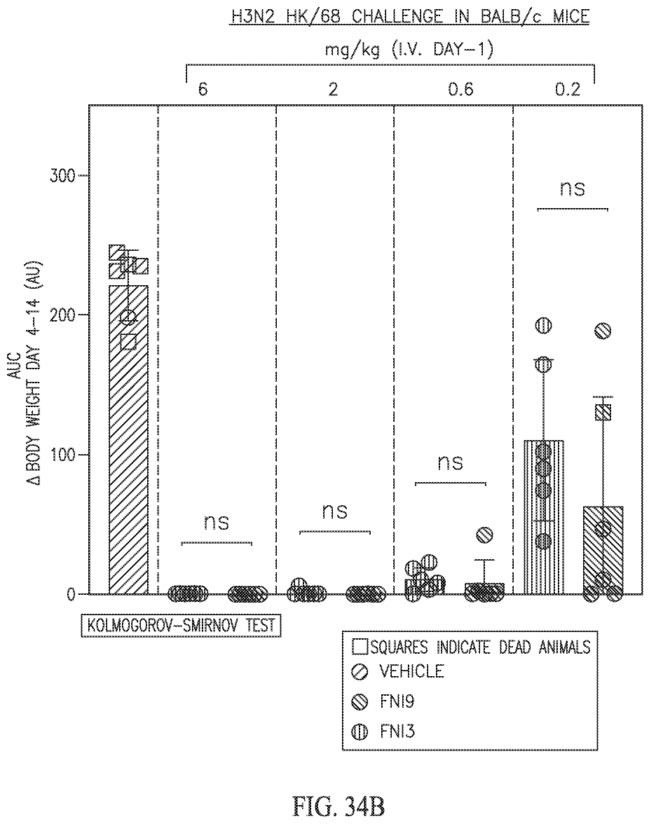

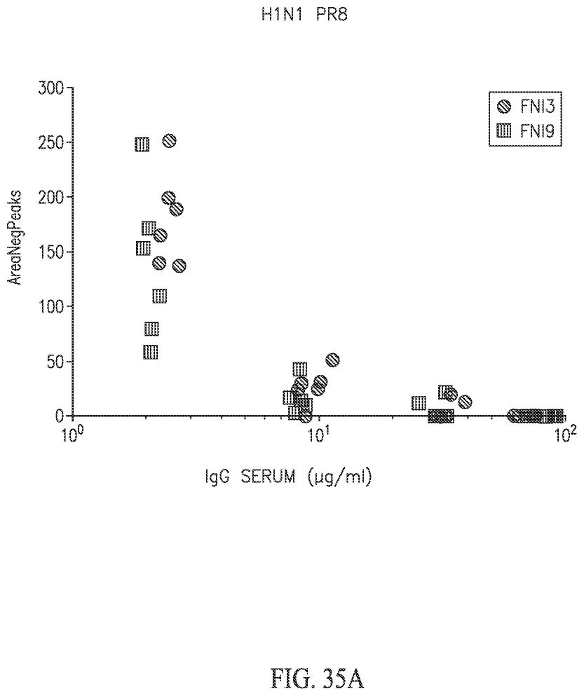

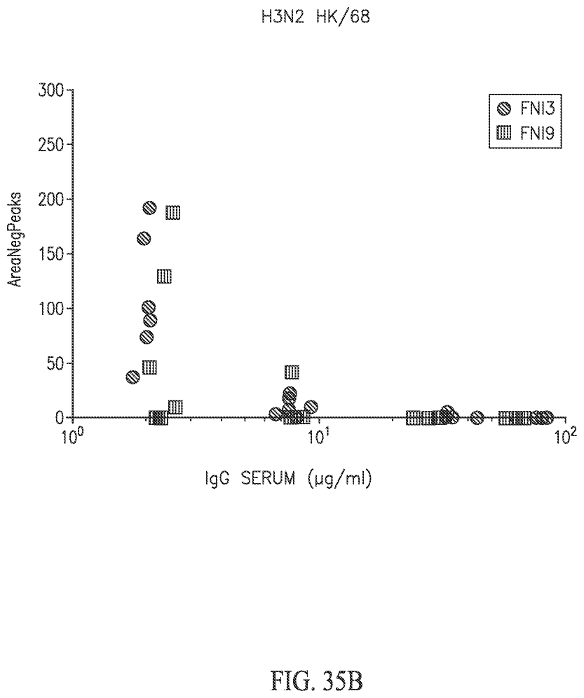

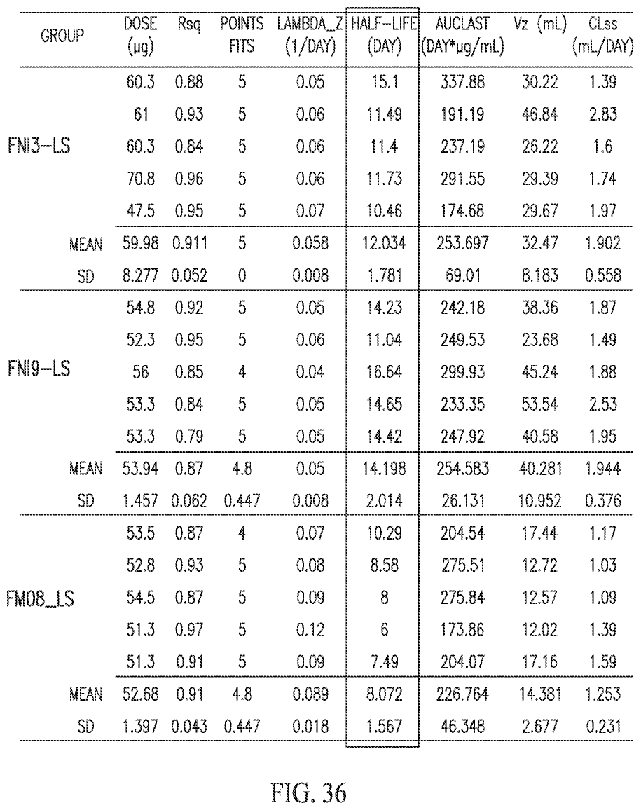

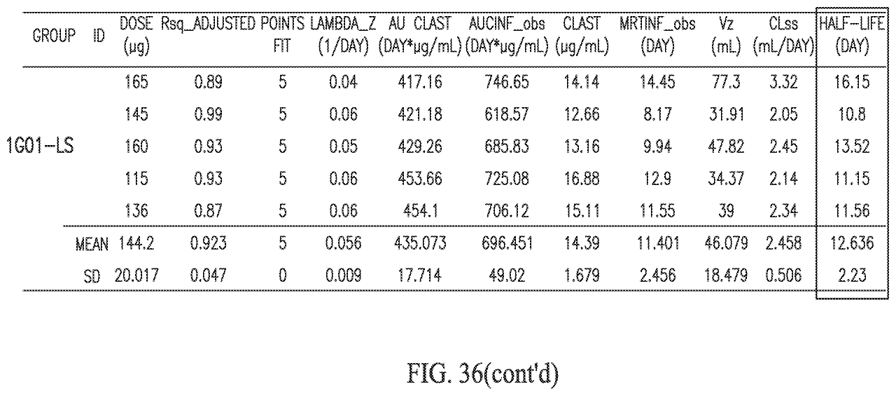

Prophylactic activity of FNI3 and FNI9 was evaluated in a murine BALB/c model of IAV infection. Briefly, BALB/c mice, 7-8 weeks of age, were administered (i.v.) FNI3 (“mAb-03” in FIG. 28A), FNI9 (“mAb-09” in FIG. 28A), or vehicle control one day prior to intranasal infection at LD90 (90%/o of a lethal dose) with H1N1 subtype A/Puerto Rico/8/34 or H3N2 subtype A/Hong Kong/1/68 (FIGS. 28A and 28B). Antibody was administered (i.v.) at 0.2. 0.6, 2, or 6 mg/kg. Baseline serum was collected at the start of infection, and both body weight and mortality were evaluated on each of Days 2-14 post-infection (FIG. 28B). Body weight measurements over fifteen days are shown in FIGS. 29A-29D (A/Puerto Rico/8/34 FNI3 test group), 30A-30D (A/Puerto Rico/8/34 FNI9 test group), 31A-31D (A/Hong Kong/1/68 FNI3 test group) and 32A-32D (A/Hong Kong/1/68 FNI9 test group). Overall mortality was also measured (FIG. 33A, A/Puerto Rico/8/34-infected mice; FIG. 33B, A/Hong Kong/1/68-infected mice). FIGS. 34A and 34B show body weight loss reported as area-under-the-curve in mice infected with A/Puerto Rico/8/34 (FIG. 34A) or A/Hong Kong/8/68 (FIG. 34B). Negative area-under-the-curve peaks compared with IgG in serum from area-under-the-curve analyses of body weight loss in BALB/c mice infected with A/Puerto Rico/8/34 (FIG. 35A) or A/Hong Kong/8/68 (FIG. 35B) are also shown. Pharmacokinetics of FNI3 (“FNI3-LS”), FNI9 (“FNI9-LS”) and comparator antibodies FM08_LS and 1G01 (“1G01-LS”) in tg32 mice is shown in FIG. 36.

### Example 4

### Pharmacokinetic Study

Pharmacokinetic analysis of Fc variants (M428L/N434S mutations) of FNI3 (“FNI3-LS”), FNI9 (“FNI9-LS”), and comparator antibodies FM08_LS and 1G01-LS was peformed in in tg32 mice, and half-life was performed, with results summarized in FIG. 36. Plasma concentration of the antibodies was determined in vitro using an ELISA assay. Goat anti-human IgG antibody (Southern Biotechnology: 2040-01) was diluted to 10 μg/ml in PBS and 25 μl was added to the wells of a 96-well flat bottom 1/2-area ELISA plate for coating over night at 4° C. After coating, the plates were washed twice with 0.5×PBS supplemented with 0.05% Tween20 (wash solution) using an automated ELISA washer. Then, plates were blocked with 100 μl/well of PBS supplemented with 1% BSA (blocking solution) for 1 h at room temperature (RT) and then washed twice. Samples were then diluted 1:2 stepwise in duplicates for a total of 8 dilutions. Standards for each antibody to be tested were prepared similarly via diluting the antibodies to 0.5 μg/ml. Standards were then diluted 1:3 stepwise in blocking solution in duplicates for a total of 8 dilutions. Twenty-five μl of the prepared samples or standards were added to Goat anti human IgG-coated wells and incubated for 1 h at RT. After four washes, 25 μl of polyclonal anti-IgG-alkaline phosphatase conjugated antibodies (Southern Biotechnology: 2040-04) diluted in blocking solution 1:500 were added per well for detection and incubated at RT for 1 h. After four washes, plates were developed by adding 80 μl/well of substrate solution (1 tablet of p-NitroPhenyl Phosphate (Sigma-Aldrich: N2765-100TAB) in 20 ml bicarbonate buffer). After 30 min incubation at RT, the absorbance was measured at 405 nm using a spectrophotometer.

To determine the concentration of the antibodies in mouse plasma, OD values from ELISA data were plotted vs. concentration in Gen5 software (BioTek). A non-linear curve fit was applied using a variable slope model, four parameters, and the equation: Y=(A−D)/(1+(X/C)\hat{ }B)+D). The OD values of the sample dilutions that fell within the predictable assay range of the standard curve % as determined in setup experiment by quality control samples in the upper, medium, or lower range of the curve ¾ were interpolated to quantify the samples. Plasma concentration of the antibodies were then determined considering the final dilution of the sample. If more than one value of the sample dilutions fell within the linear range of the standard curve, an average of these values was used. Pharmacokinetics (PK) data were analyzed by using WINNONLIN NONCOMPARTMENTAL ANALYSIS PROGRAM (8.1.0.3530 Core Version, Phoenix software, Certara) with the following settings: Model: Plasma Data, i.v. Bolus Administration; Number of non-missing observations: 8; Steady state interval Tau: 1.00; Dose time: 0.00; Dose amount: 5.00 mg/kg; Calculation method: Linear Trapezoidal with Linear Interpolation; Weighting for lambda_z calculations: Uniform weighting; Lambda_z method: Find best fit for lambda_z, Log regression. Graphing and statistical analyses (linear regression or outlier analysis) were performed using Prism 7.0 software (GraphPad, La Jolla, CA, USA).

### Example 5

### Generation of FNI3 and FNI9 Variant Antibodies

Variants of FNI3 and FNI9 were generated by mutating amino acids in the variable regions. See Tables 1 and 2.

### Example 6

### Additional Studies

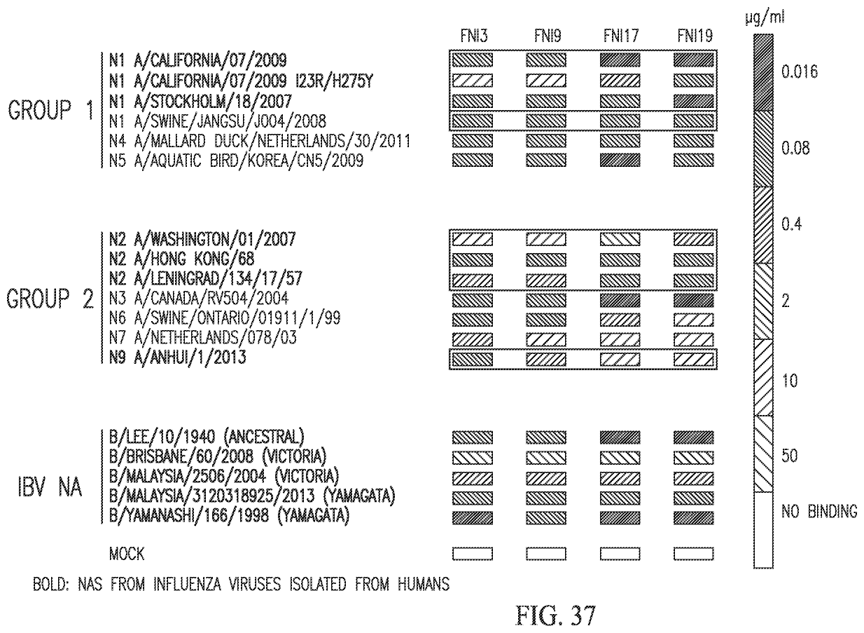

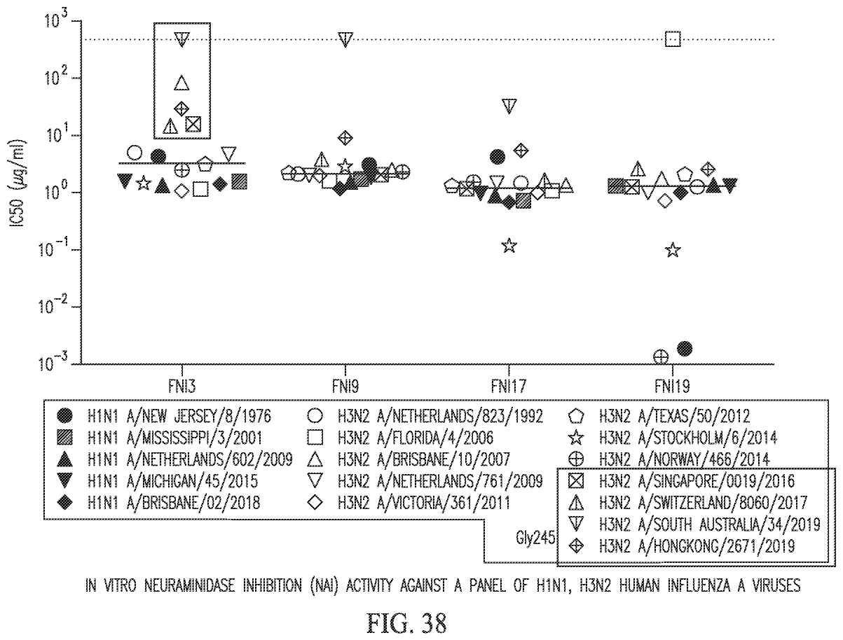

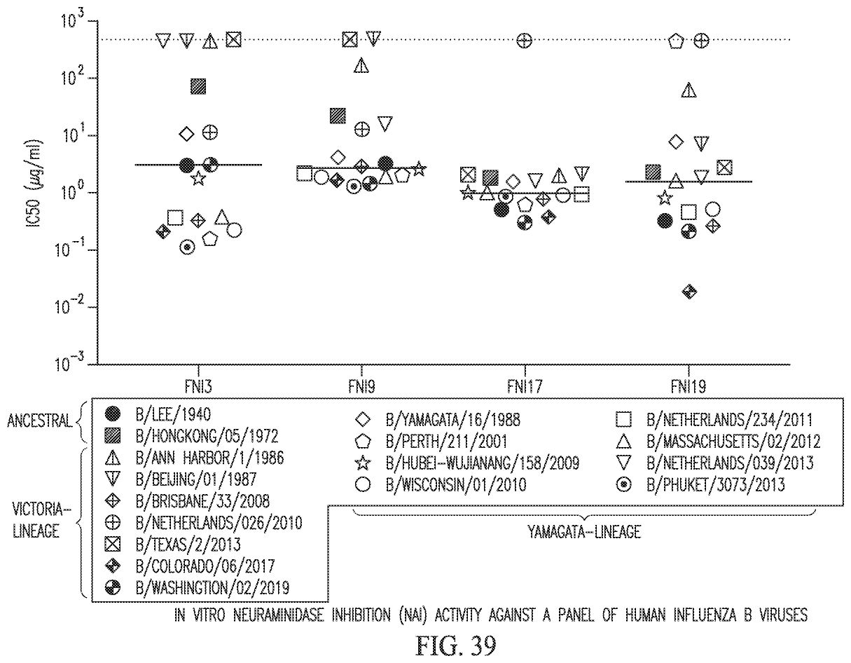

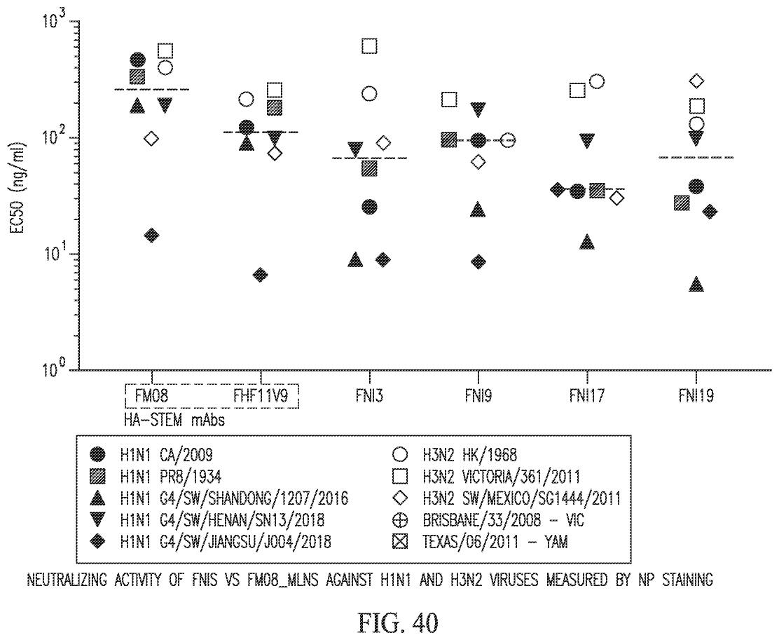

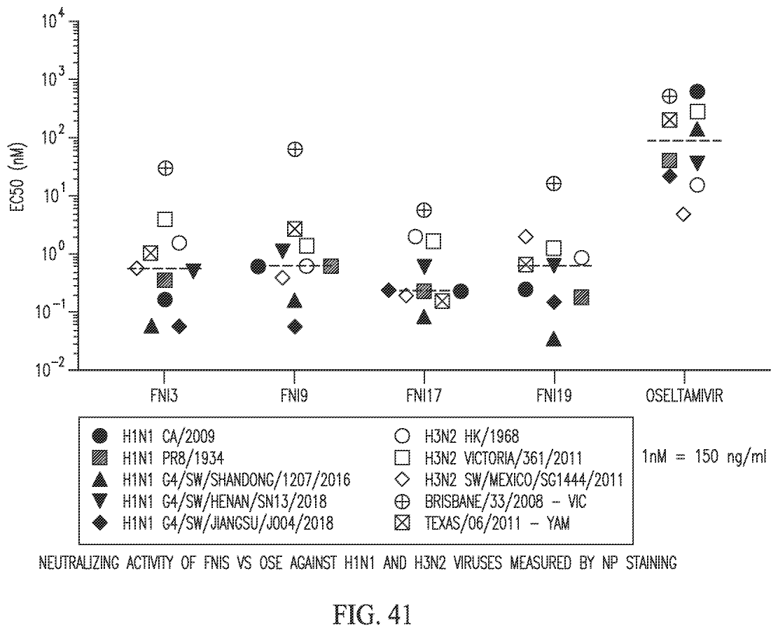

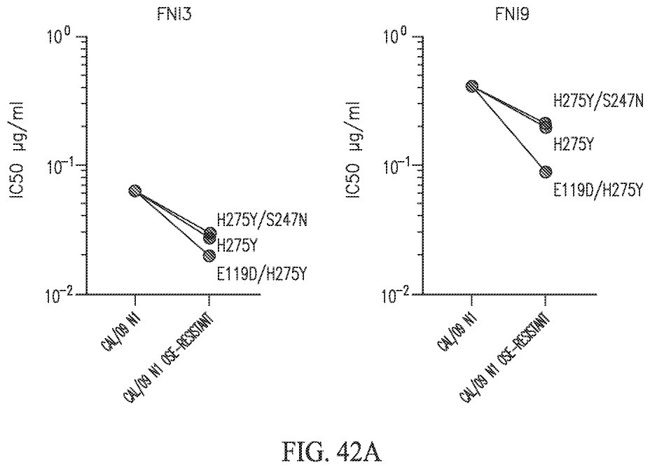

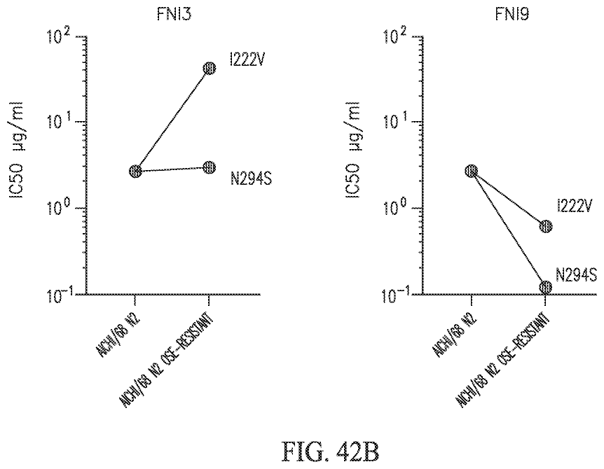

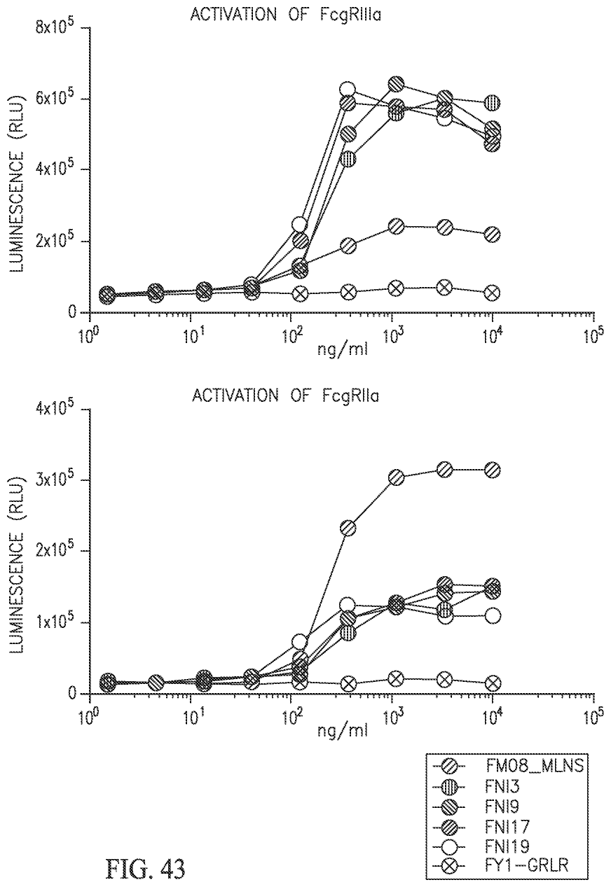

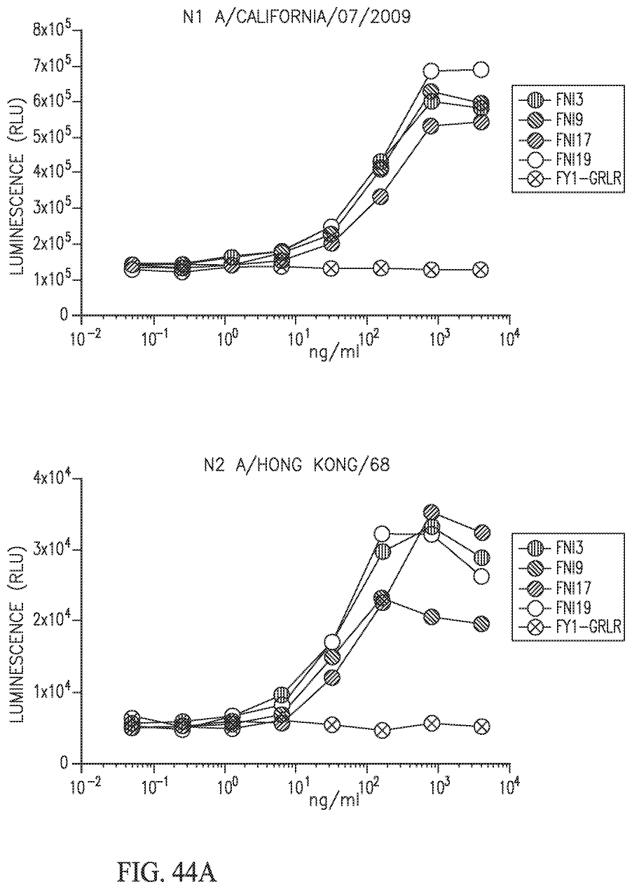

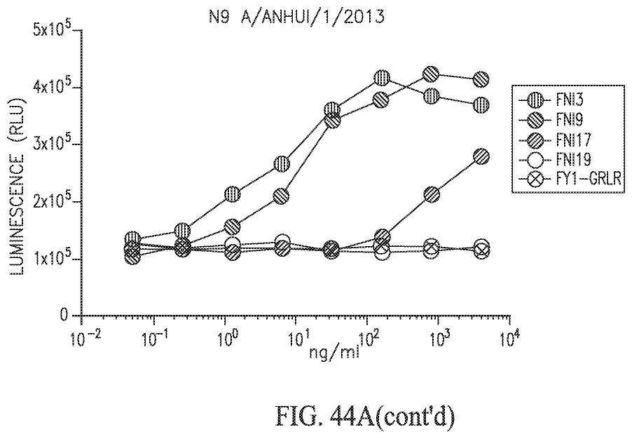

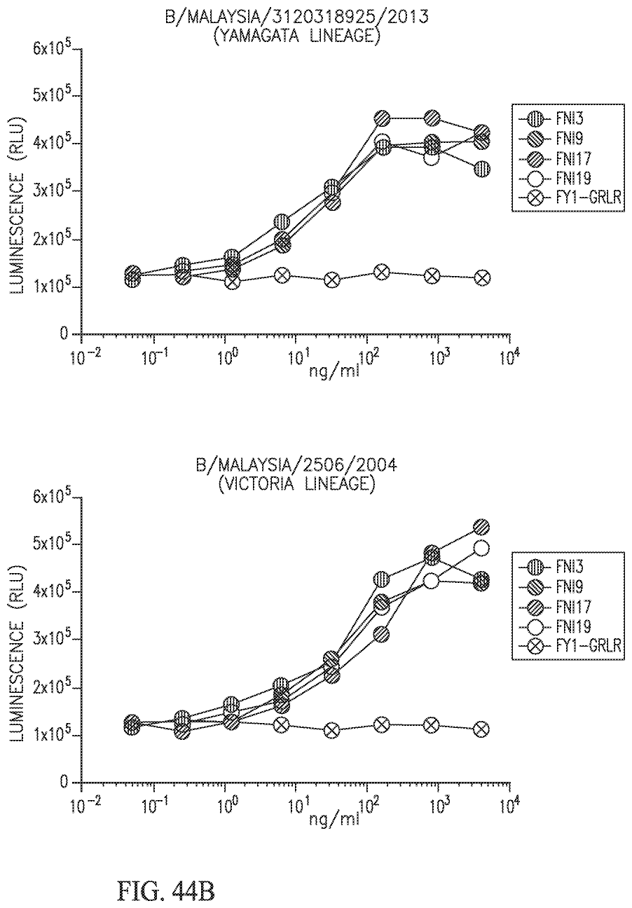

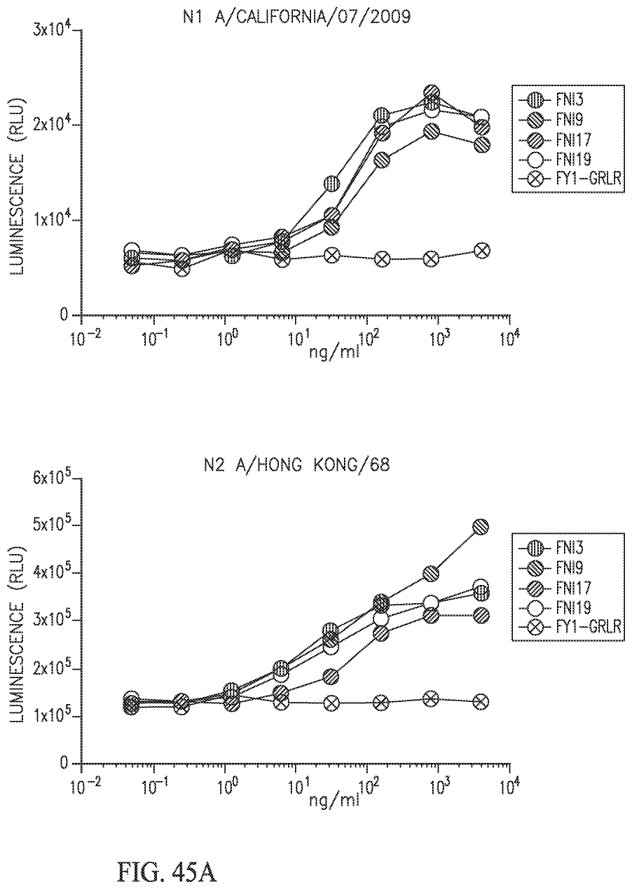

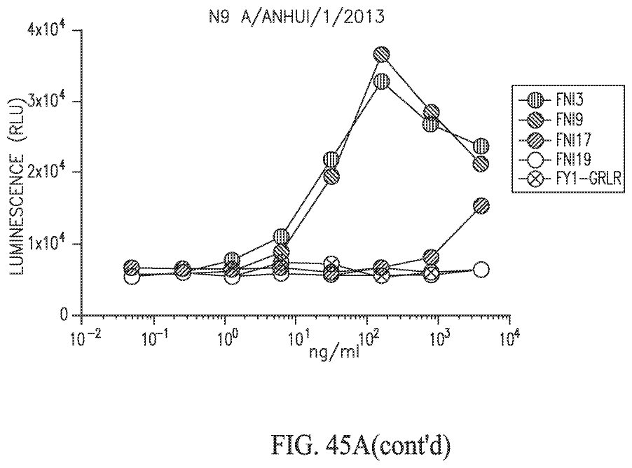

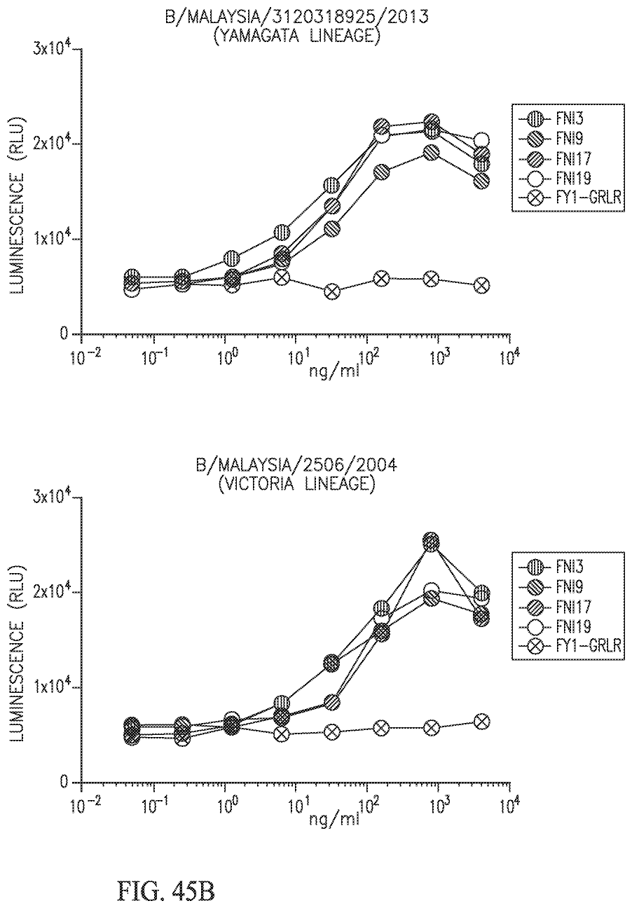

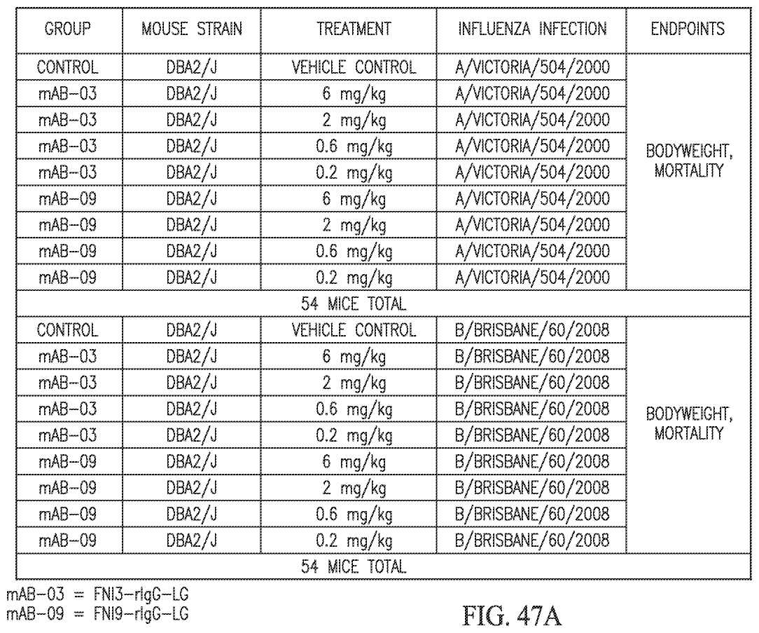

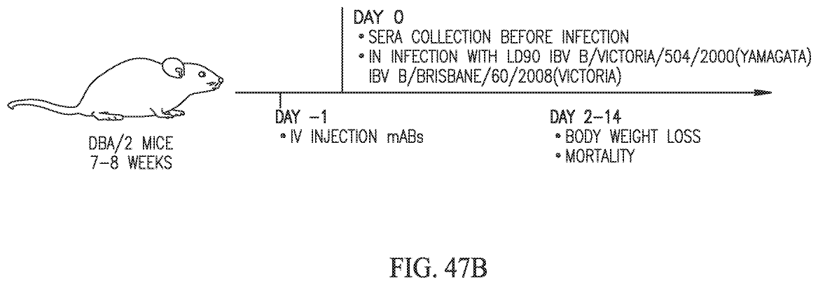

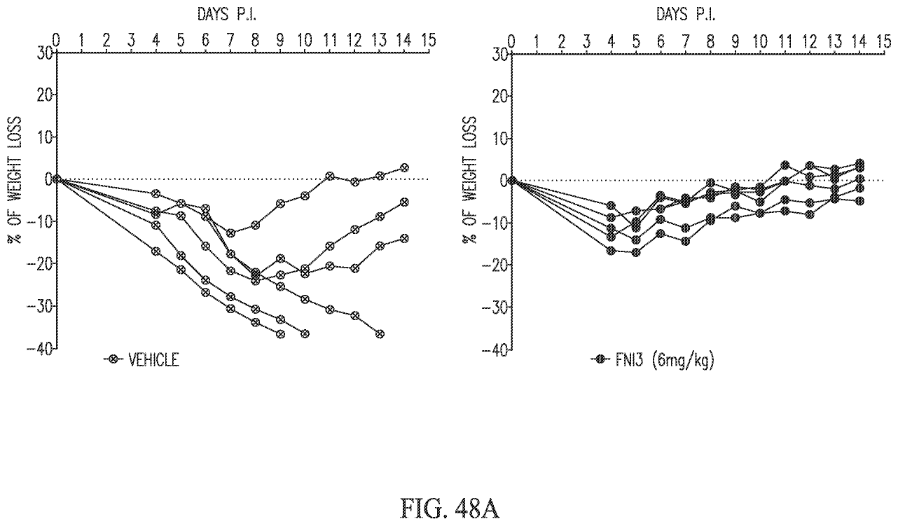

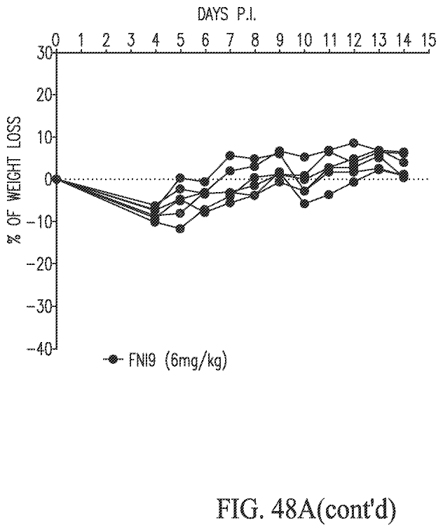

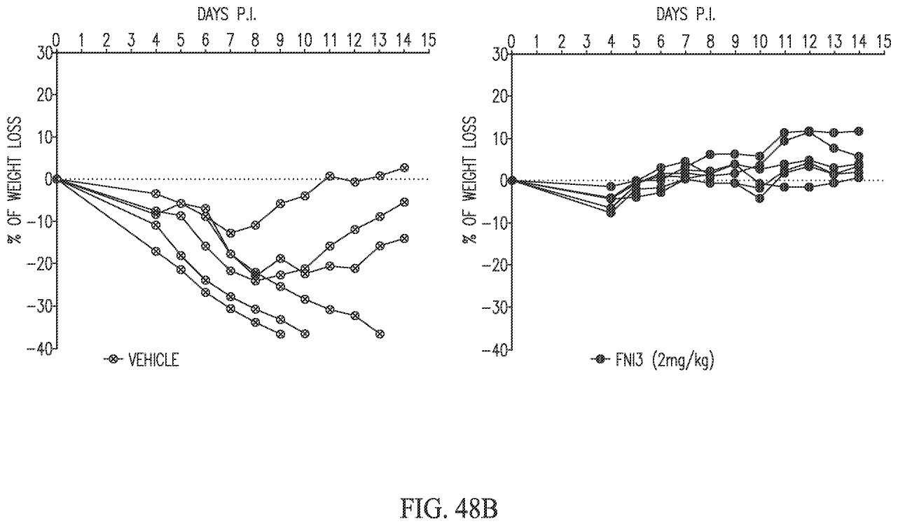

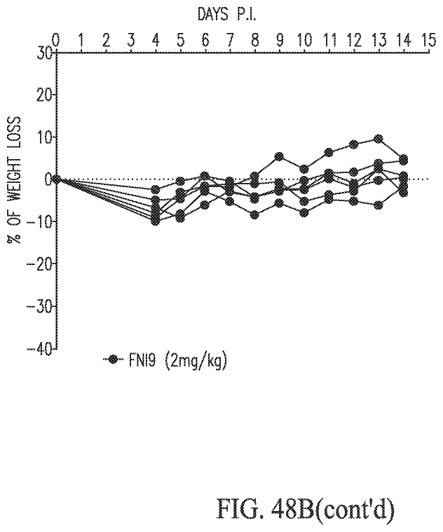

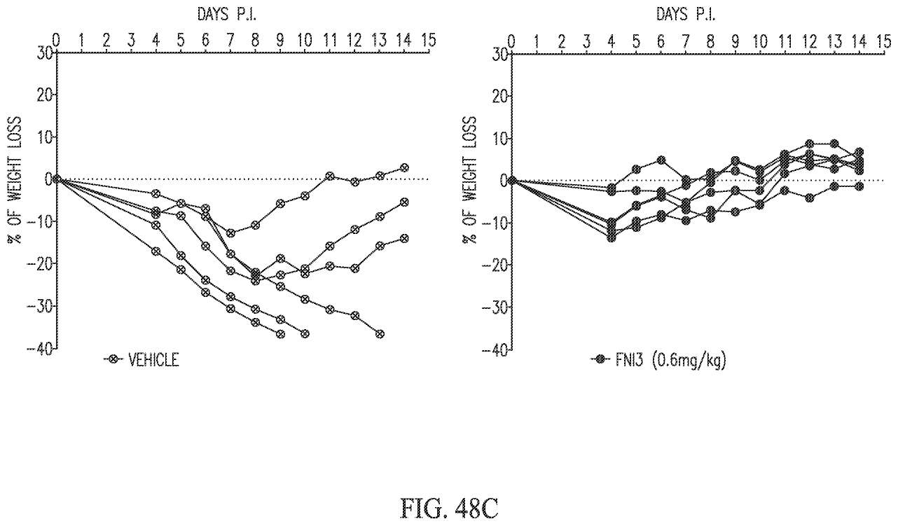

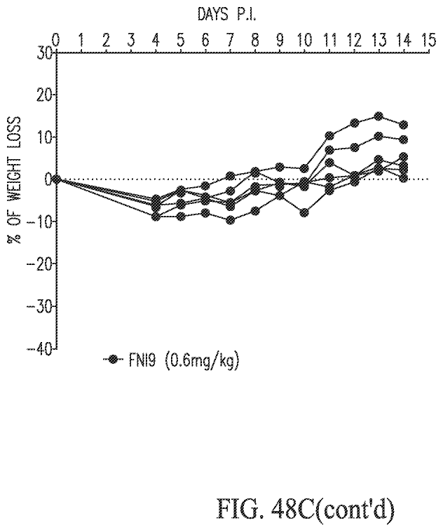

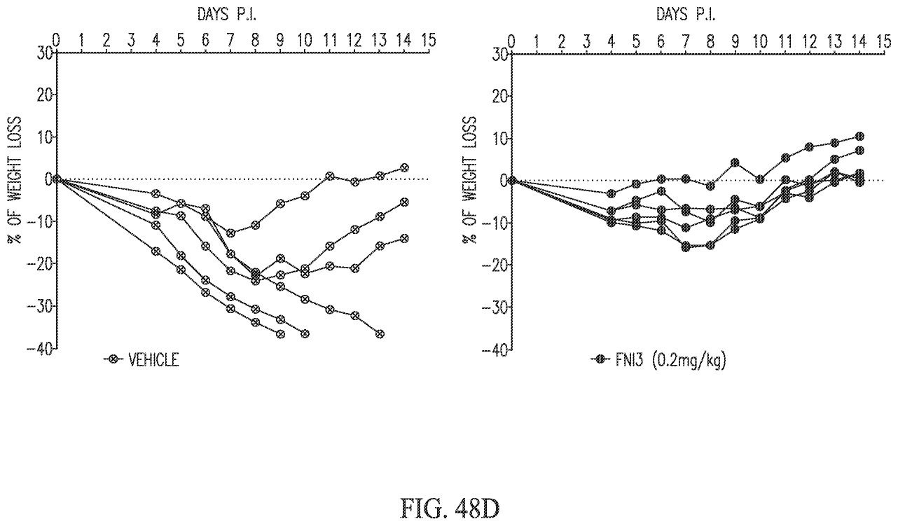

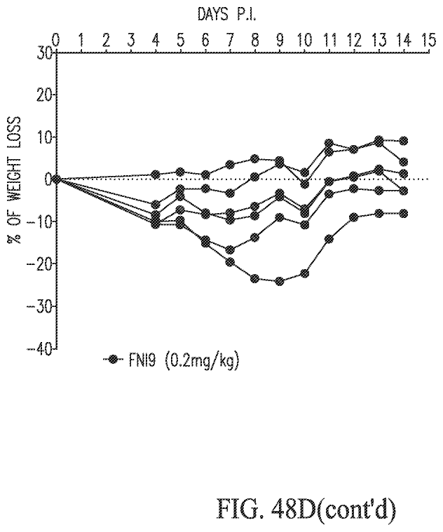

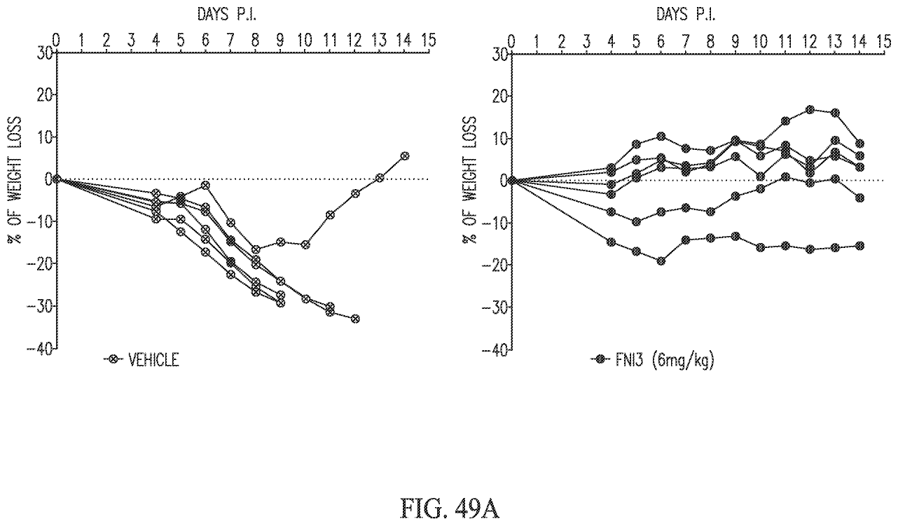

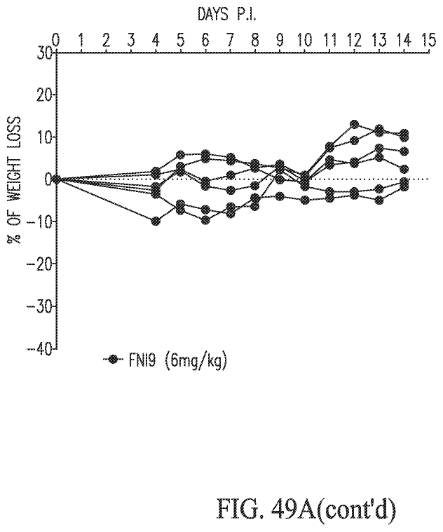

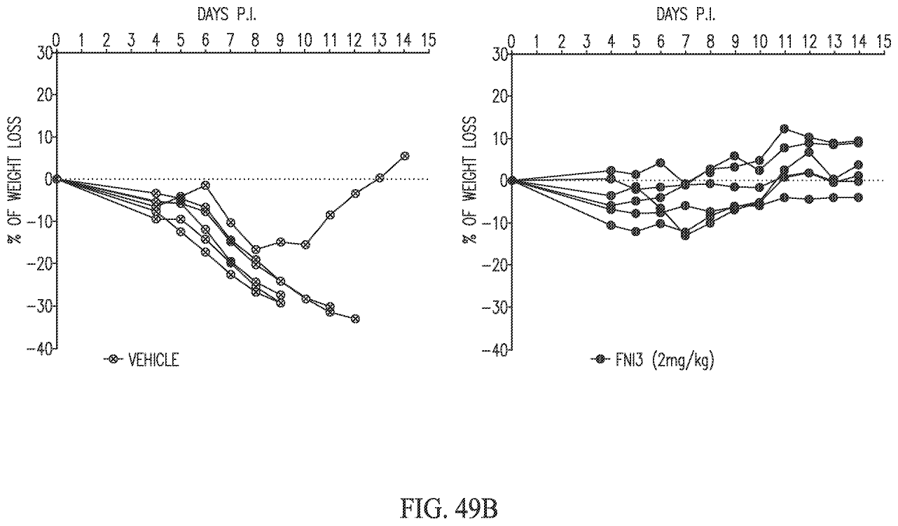

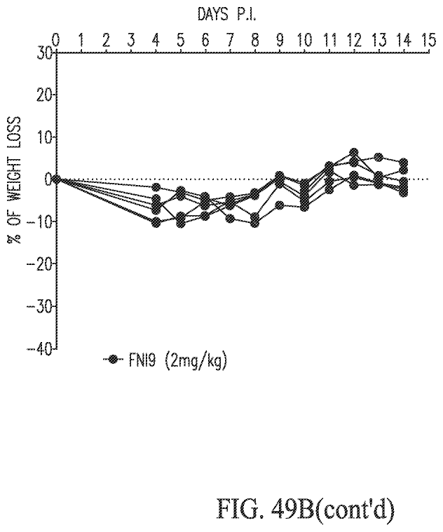

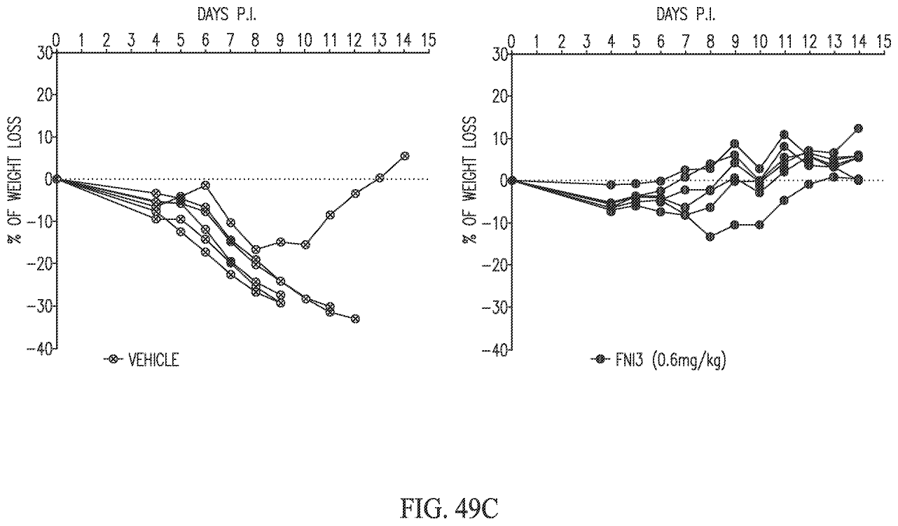

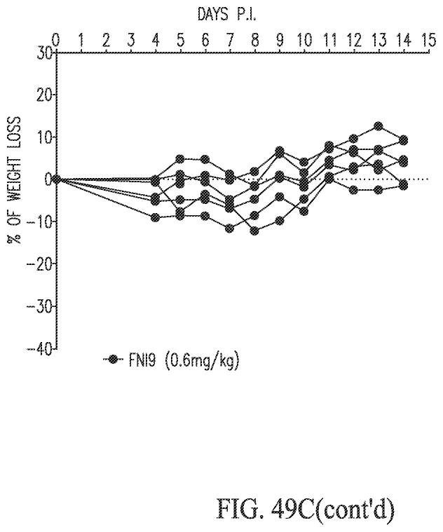

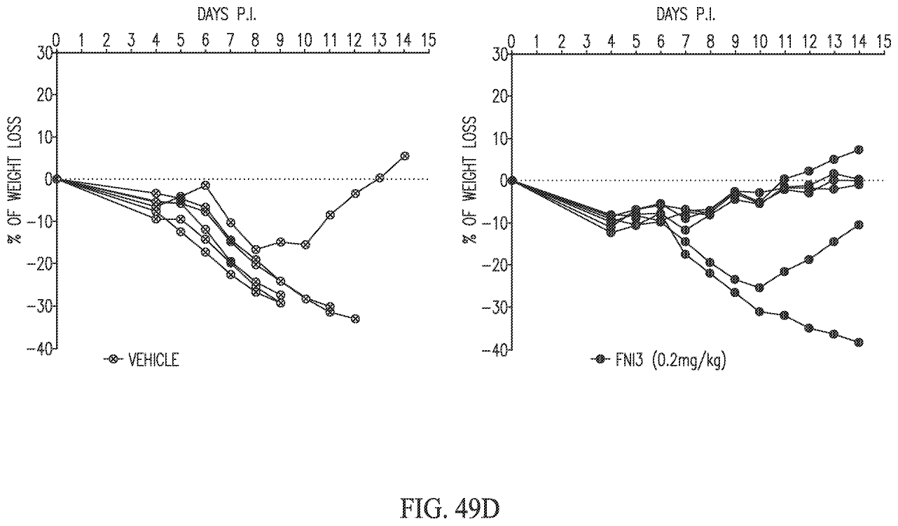

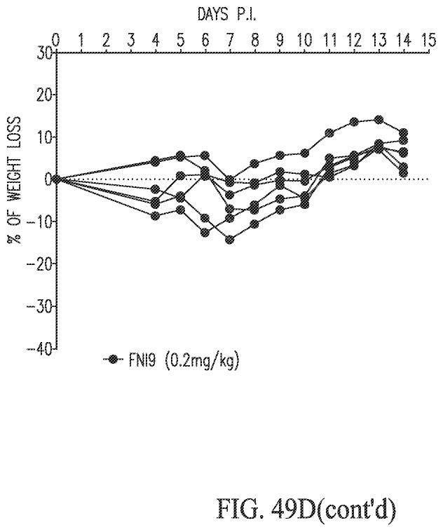

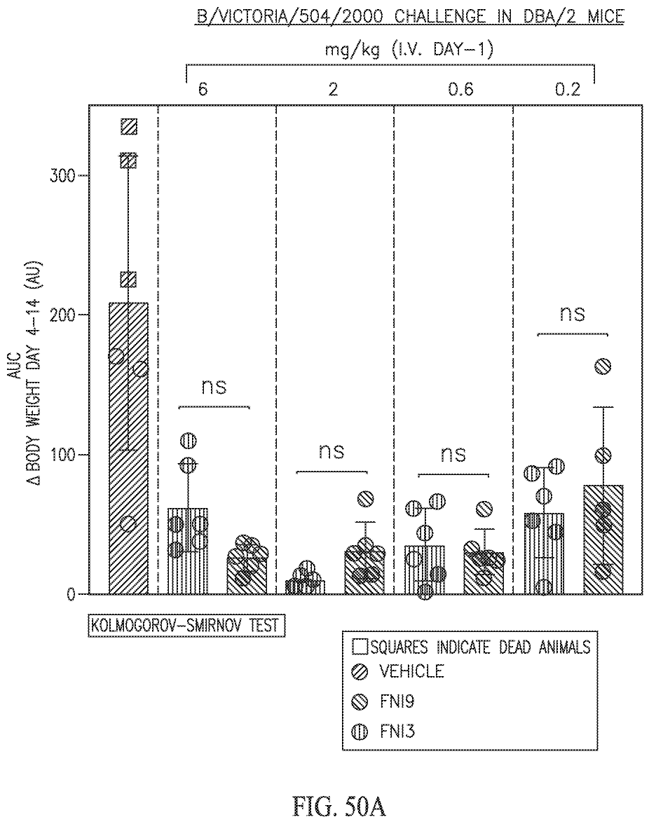

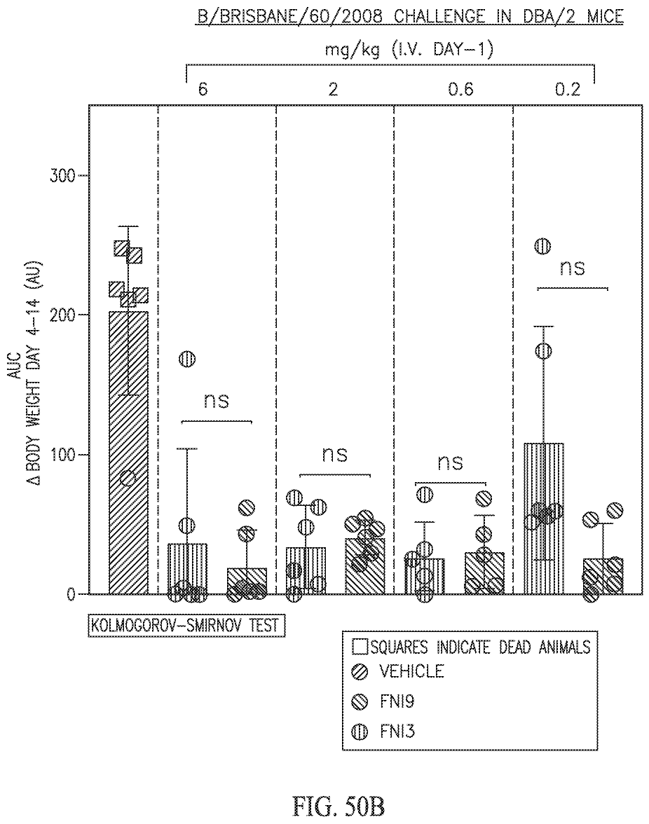

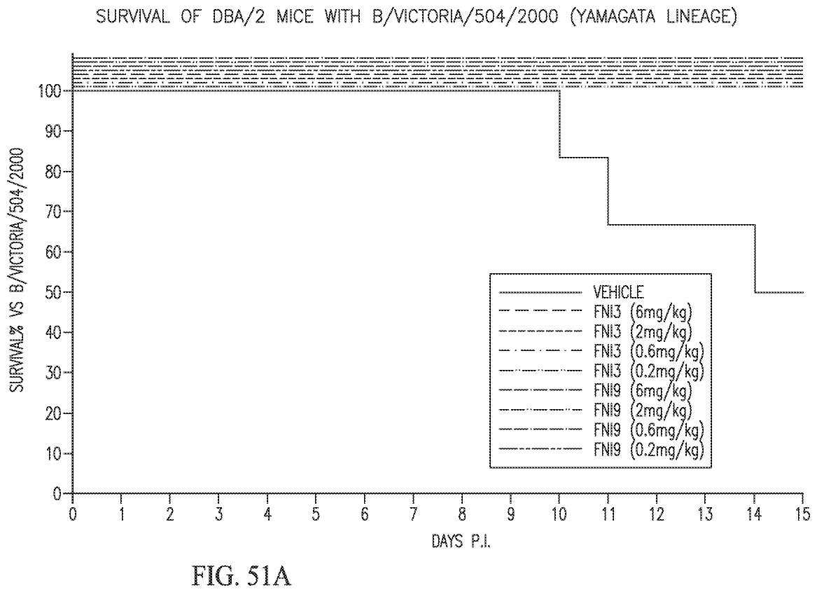

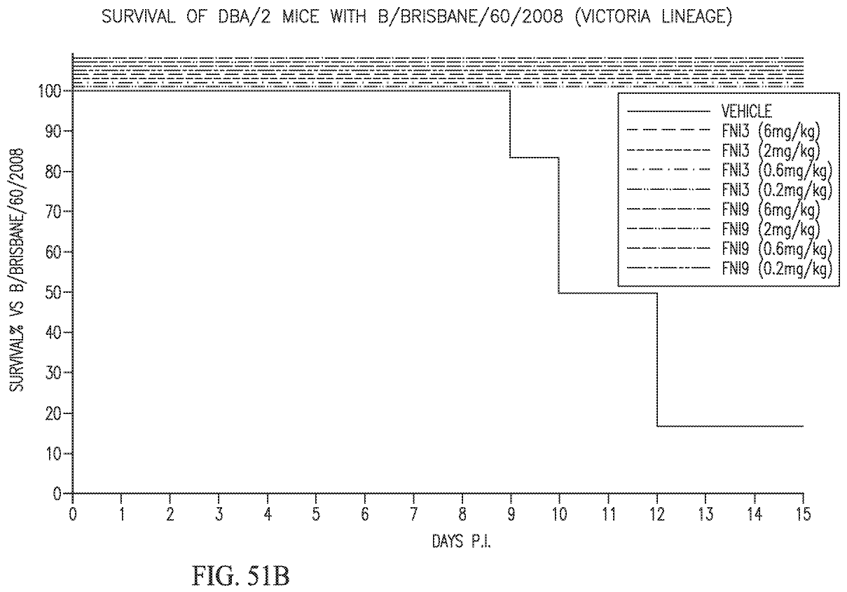

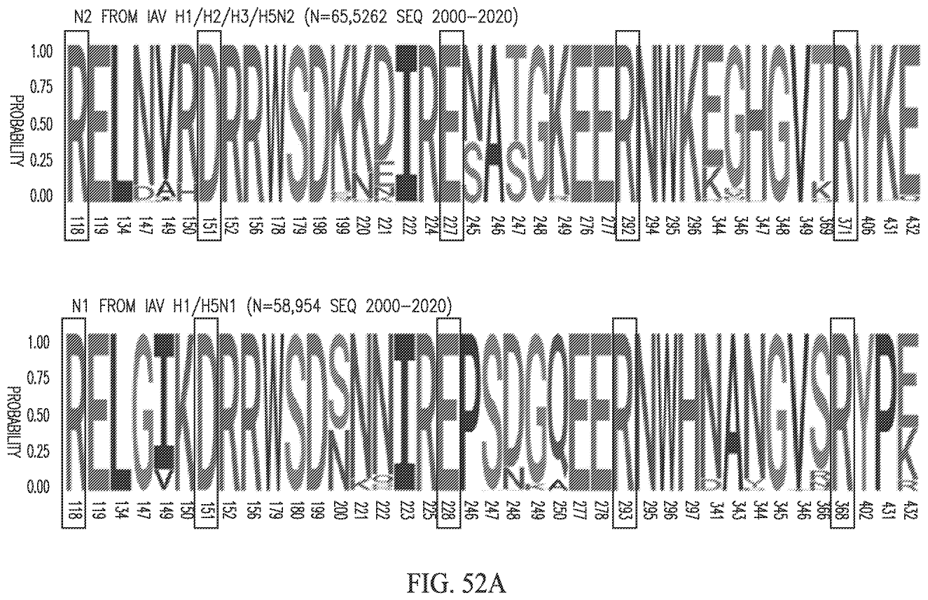

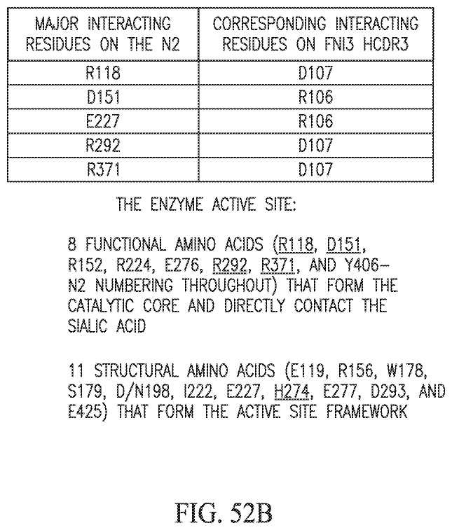

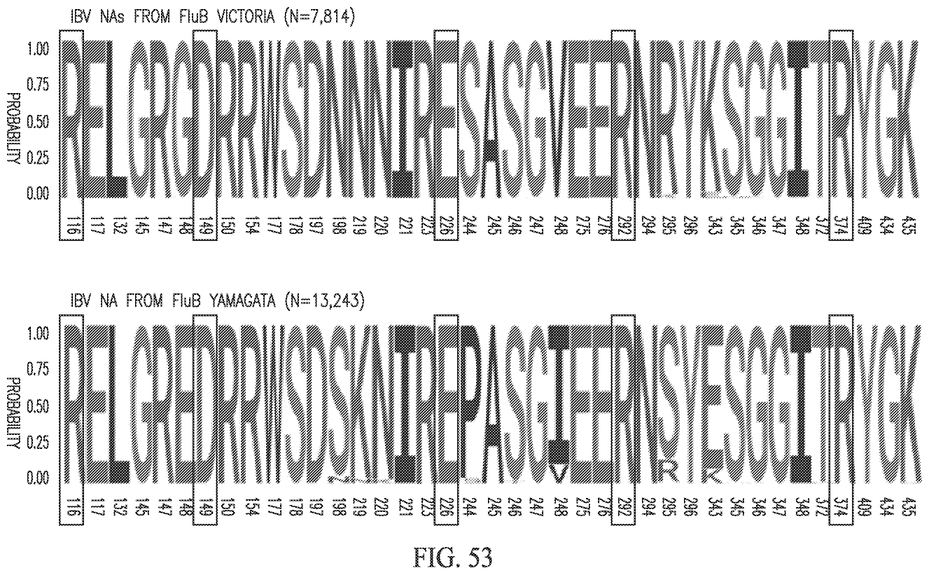

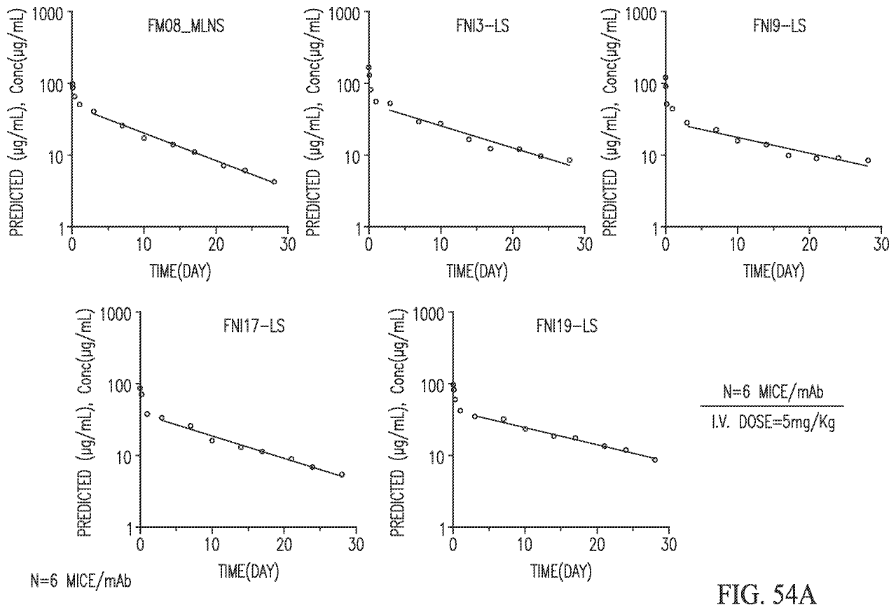

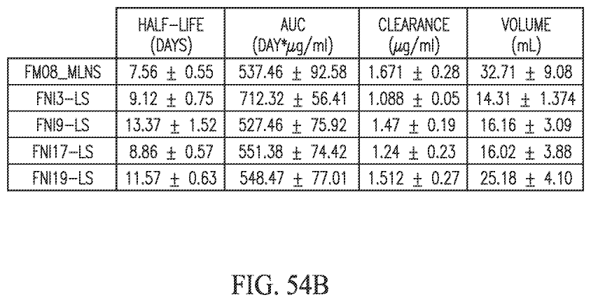

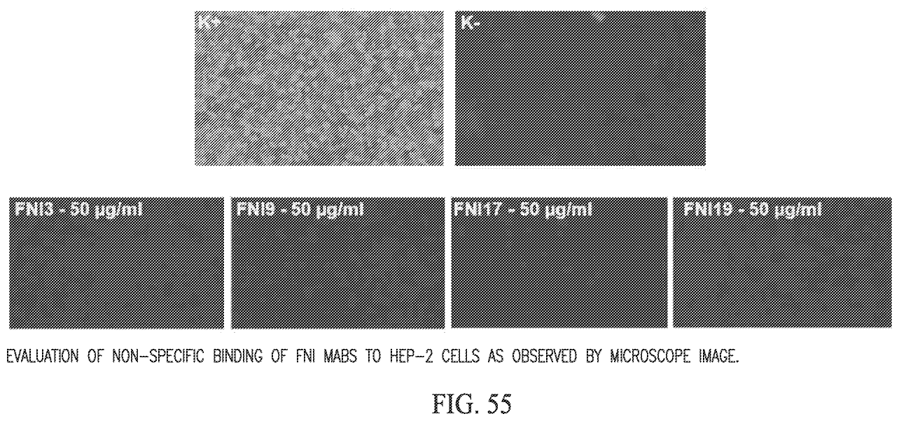

FNI antibodies were evaluated for binding and NAI activity against a panel of IAV NAs and IBV NAs (FIG. 37). FNI17 and FNI19 bound NA from human IAV circulating strains (e.g. N1 from A/California/07/2009 or N2 from A/Washington/01/2007) at a lower concentration than FNI3 and FNI9 (see data highlighted by rectangle in FIG. 37). FNI3 and FNI9 displayed higher cross-reactivity toward NAs from zoonotic strains (e.g. N9 from A/Anhui/1/2013, see data highlighted by rectangle in FIG. 37). All FNI antibodies bound to N1 from A/Swine/Jiangsu/J004/2018 (see data highlighted by rectangle, second from top in FIG. 37) which has been characterized as having pandemic potential (Sun et al. Proc Natl Acad Sci USA. 2020). The FNI sequence variants were analyzed for function. FNI antibodies were tested in further neutralization and NAI studies against IAVs and viruses bearing OSE-resistant mutations. FNI antibodies were tested for activation of FcγRs following incubation with IAV and BV NAs. Epitope conservation studies and in vitro resistance selection studies were performed. In vivo prophylaxis studies of FNI3 and FNI9 against IAVs and against B/Victoria/504/2000 and B/Brisbane/60/2008 were performed in Balb/c and DBA/2 mice, respectively. In vivo pharmacokinetics of FNI antibodies bearing MLNS Fc mutations was tested in SCID Tg32 mice. Data from the above-mentioned studies are shown in FIGS. 37-55.

Further studies (cryo-EM, resistance vs. H1N1 A/California/07/2009, PK in NHP, efficacy) are performed using FNI9, FNI7, and FNI19.

### Example 7

### Binding Studies Using FNI mAbs

Binding interactions between anti-NA antibodies and NA were evaluated by crystal structure studies and docking analysis. FNI3 docking on N2 NA is shown in FIG. 56A. An overlay of FNI3, FNI17, and FNI19 antibodies docking with NA is shown in FIG. 56B. The codes indicated in FIG. 56B correspond with the ribbon structures of FNI3, FNI17, and FNI19. CDRH3, which interacts with NA, is highlighted by a rectangle in FIG. 56C, which shows VH amino acid sequence alignments of FNI3, FNI9, FNI17, and FNI19 with unmutated common ancestor, “UCA”. No major differences in the angle of approach were observed between NA and FNI3, FNI9, FNI17, and FNI19 antibodies.

The crystal structure of FNI17 in complex with N2 NA, including residues of light chain CDRs (L-1, L-2, L-3) and heavy chain CDRs (H-1, H-2, H-3) is shown in FIG. 57A. CDRH3 residues D107 and R106 of FNI17 are inserted within the NA enzymatic pocket, mimicking the sialic acid receptor. The sequence location of D107 and R106 is shown in the rectangle of FIG. 57B.

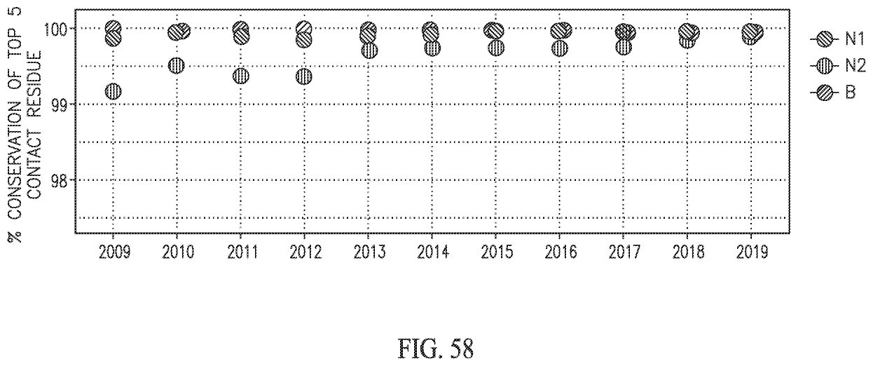

Conservation of the top five interacting residues within the FNI NA epitope in group I IAVs, group II IAVs, and IBVs from 2009 to 2019 are shown in FIG. 58.

### Example 8

### In Vitro Potency: Comparison of FNI Antibodies with FM08 and Oseltamivir

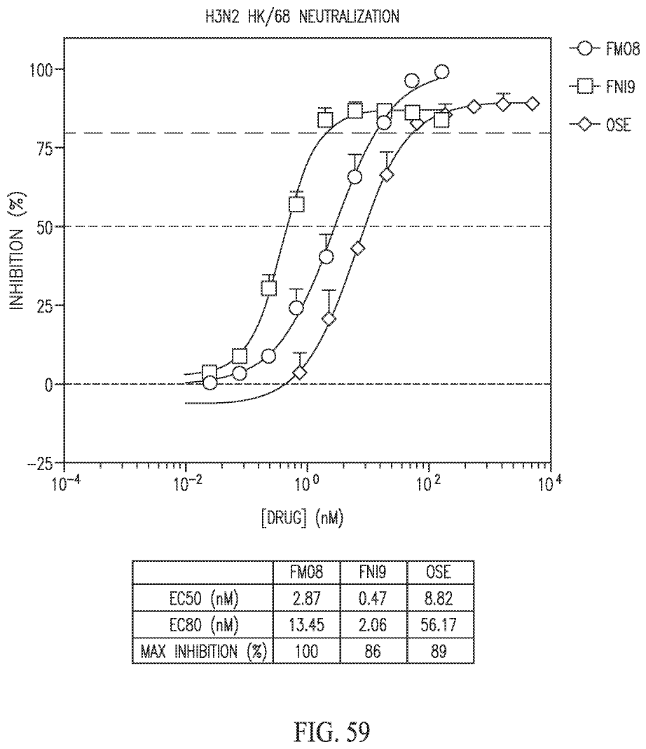

In vitro potency of FNI antibodies was evaluated in comparison with potency of OSE and FM08. In vitro neutralizing activity of FNI9, OSE, and a comparator antibody “FM08”, measured by nucleoprotein (NP) staining against H3N2 A/Hong Kong/8/68 virus, is shown in FIG. 59.

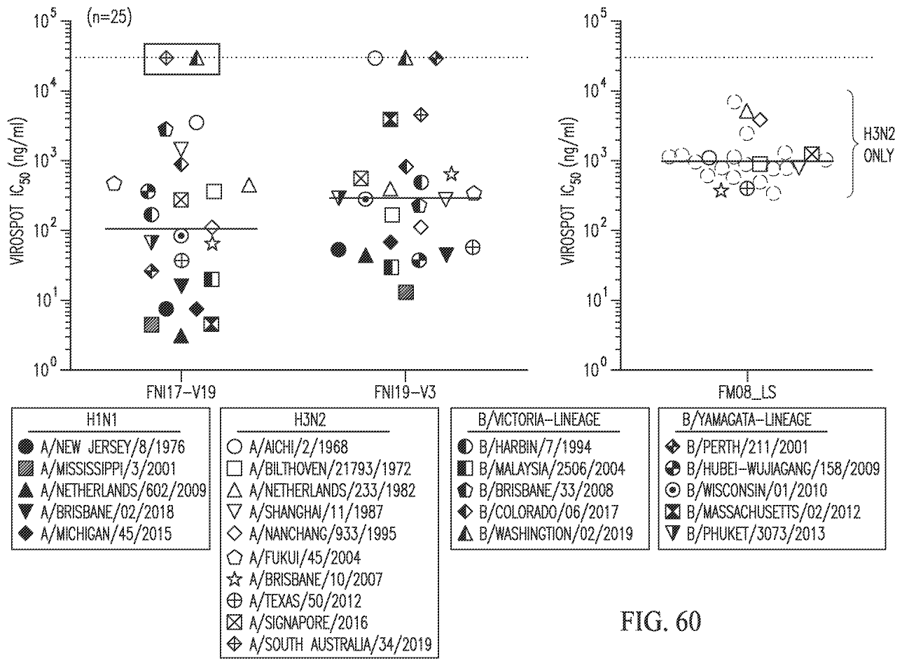

In vitro inhibition of sialidase activity by FNI17 variant FNI17-v19 (VH: SEQ ID NO.:199; VL: SEQ ID NO.: 201), FNI19 variant FNI19-v3 (VH: SEQ ID NO.:203; VL: SEQ ID NO.: 205), and FM08-LS of group I (H1N1) IAV, group II (H3N2) IAV, Victoria-lineage IBV, and Yamagata-lineage IBV NAs, as measured by ViroSpot microneutralization assay, is shown in FIG. 60. The ViroSpot microneutralization assay is a tool for the detection and phenotypic characterization of influenza viruses. In brief, the technique involves microtiter-format virus culture combined with automated detection of immunostained virally-infected cells (Baalen et al., Vaccine. 35:46, 2017).

### Example 9

### In Vivo Potency: Comparison of FNI Antibodies with FM08 and Oseltamivir

In vivo potency of FNI antibodies was evaluated in comparison with potency of OSE and FM08.

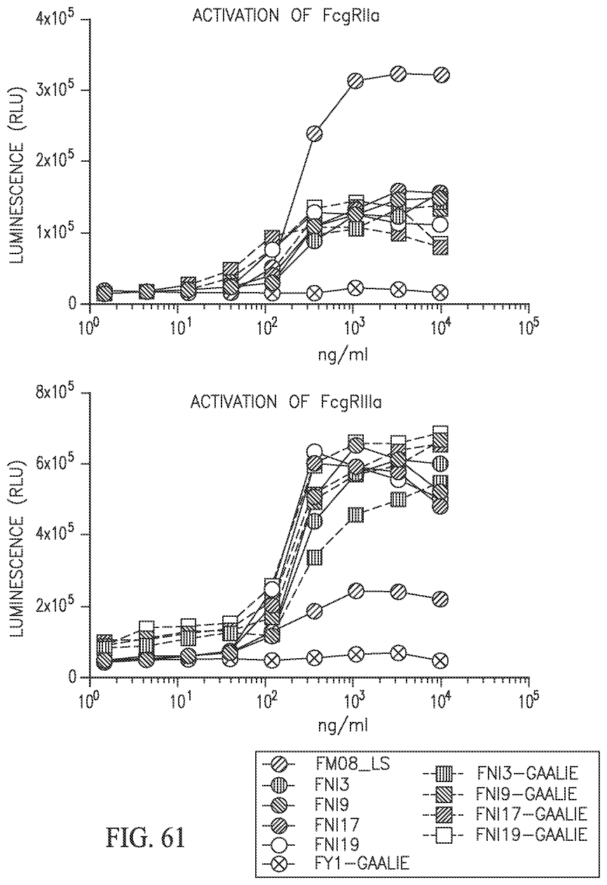

Antibody activation of FcγRIIIa and FcγRIIa by “GAALIE” variant antibodies (G236A/A330L/I332E variants) was tested, as shown in FIG. 61. Activation of FcγRIIIa (F158 allele) and FcγRIIa (H131 allele) was measured using an NFAT-mediated Luciferase reporter in engineered Jurkat cells. Activation was assessed following incubation with A549 cells infected with H1N1 influenza strain A/Puerto Rico/8/34 at a multiplicity of infection (MOI) of 6. FNI3, FNI9, FNI17, and FNI19 were tested, along with FNI3, FNI9, FNI17, and FNI19 antibodies bearing GAALIE mutations (suffix “-GAALIE”). A comparator antibody “FM08_LS” and a negative control antibody (FY1-GRLR) were also tested.

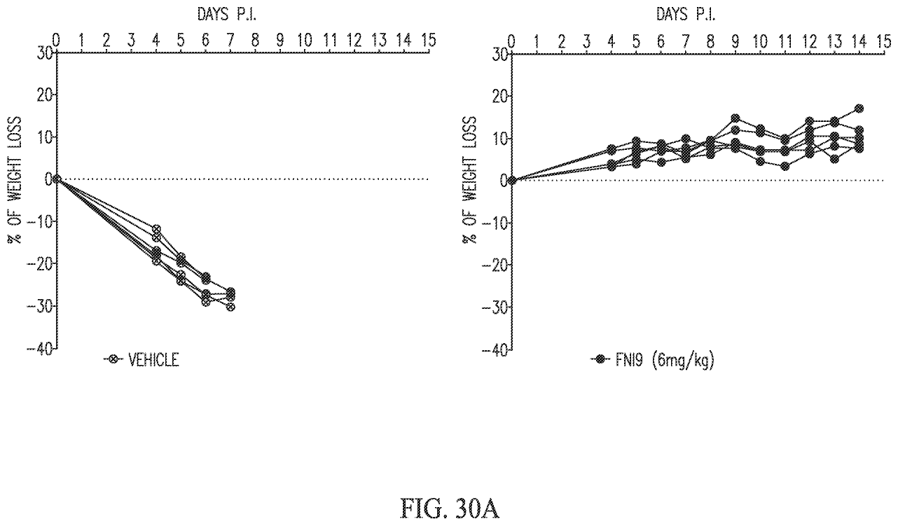

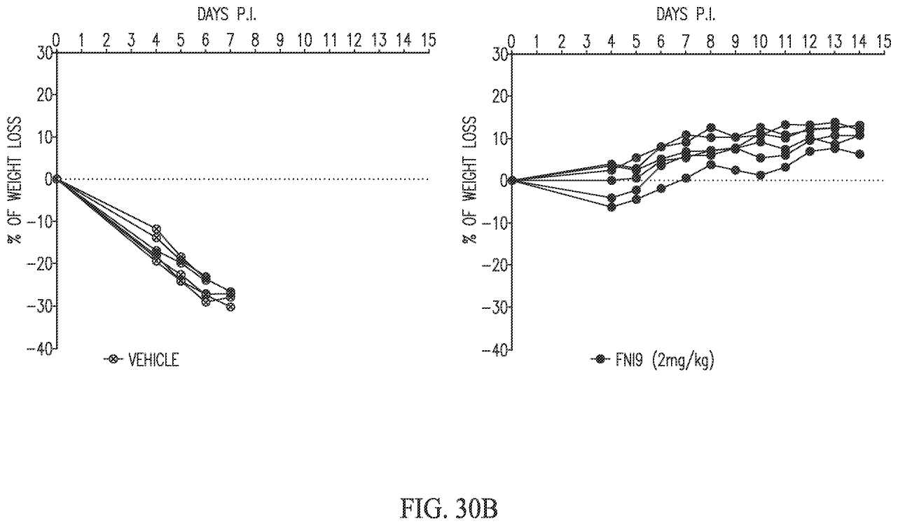

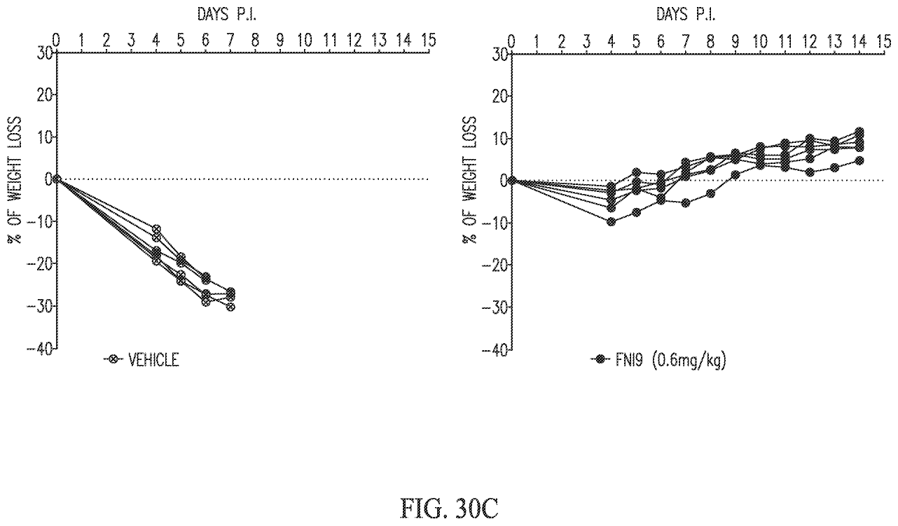

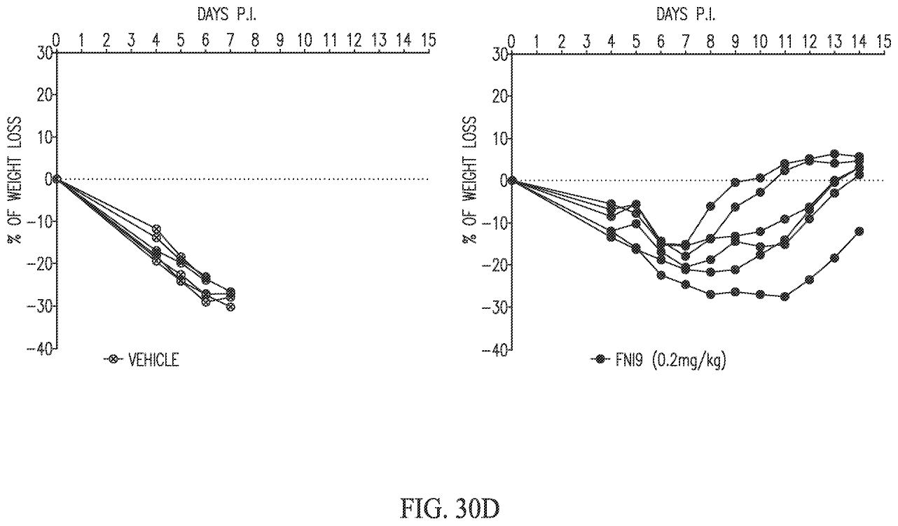

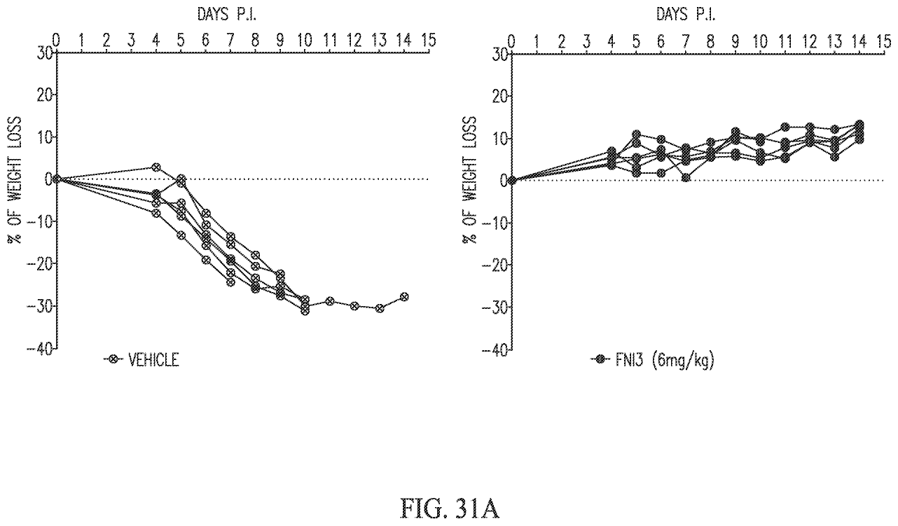

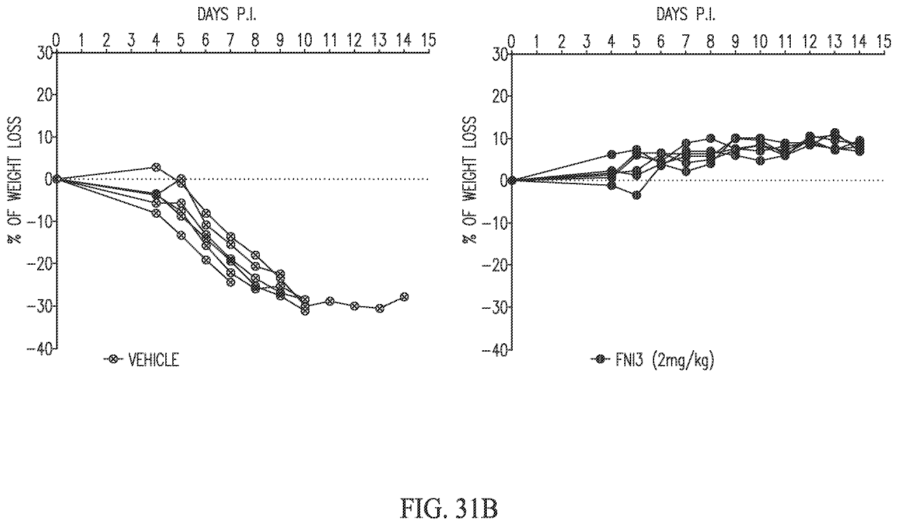

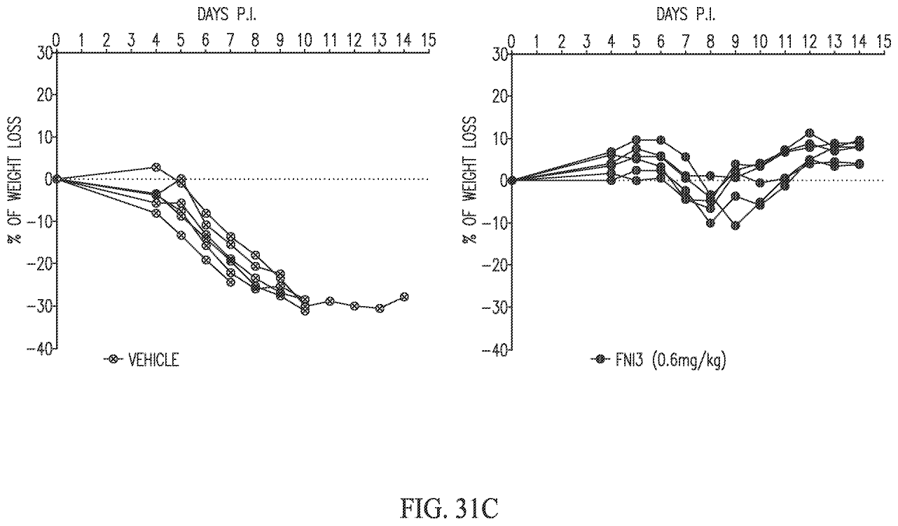

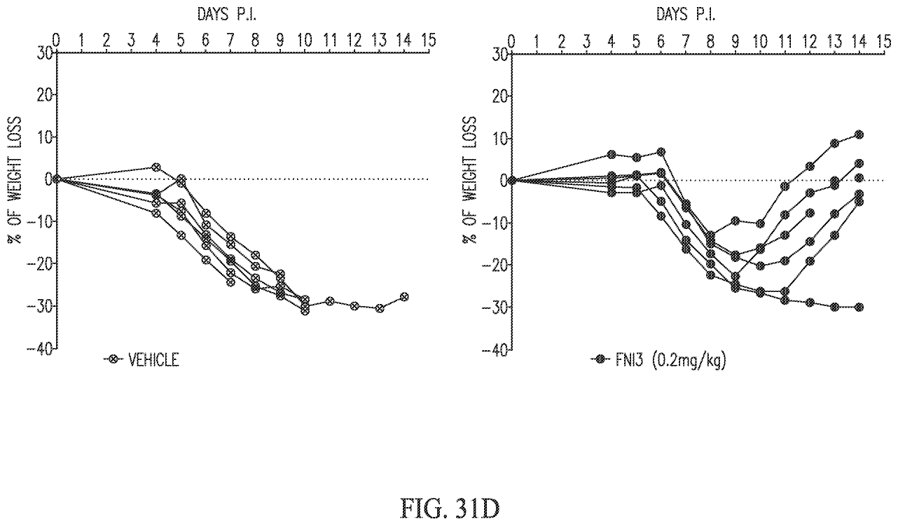

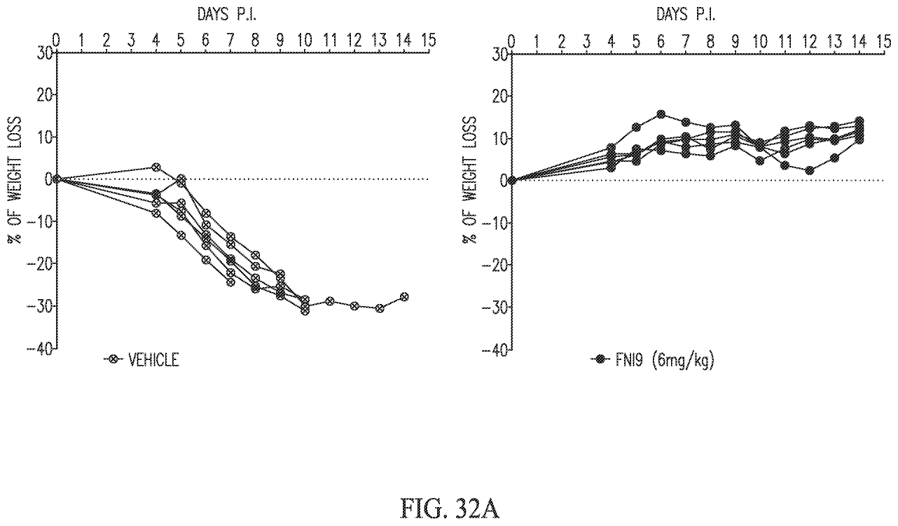

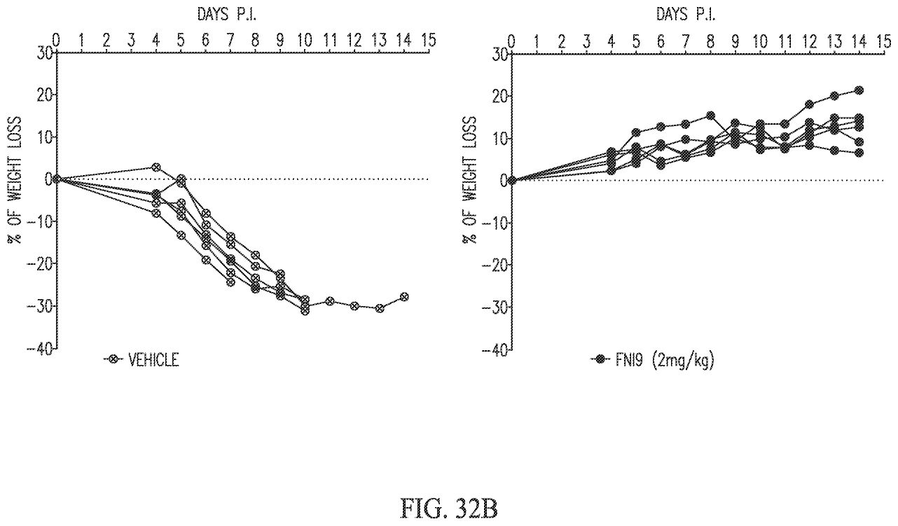

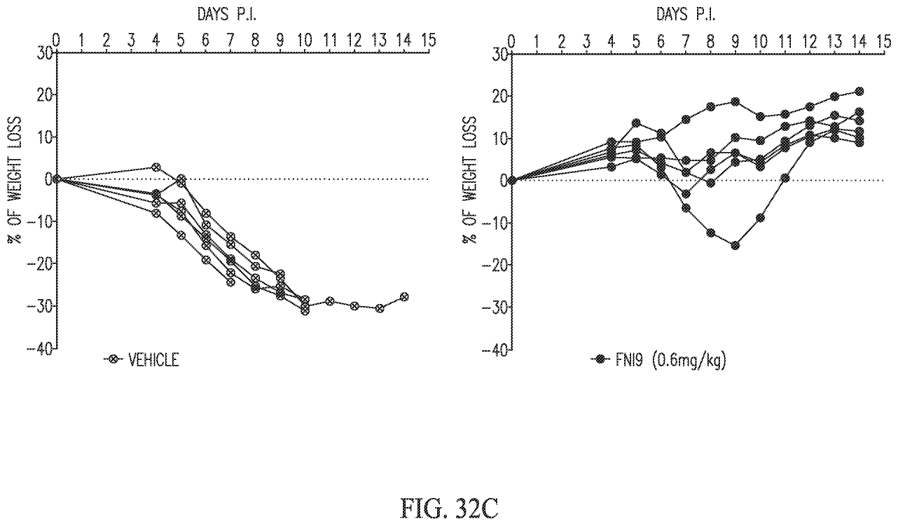

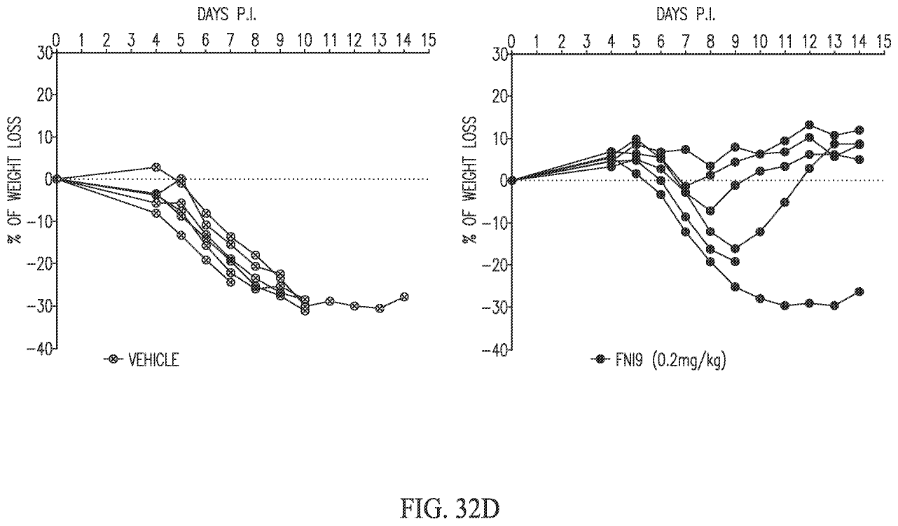

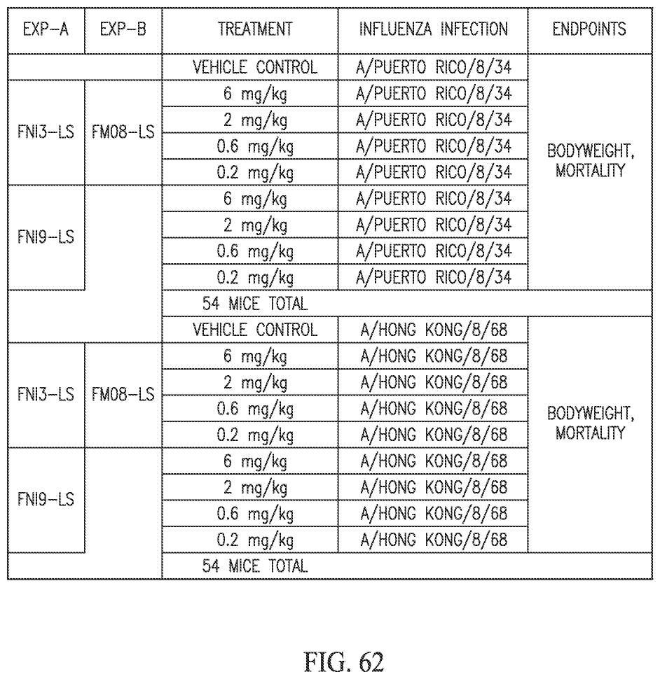

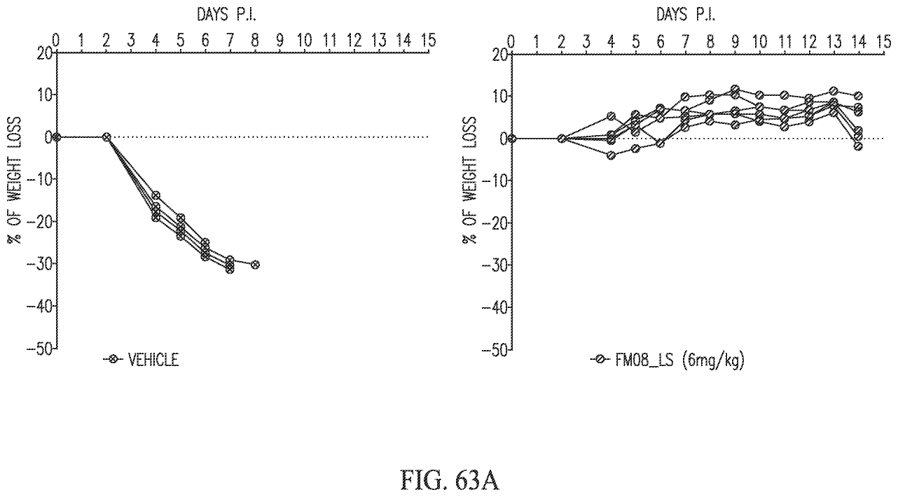

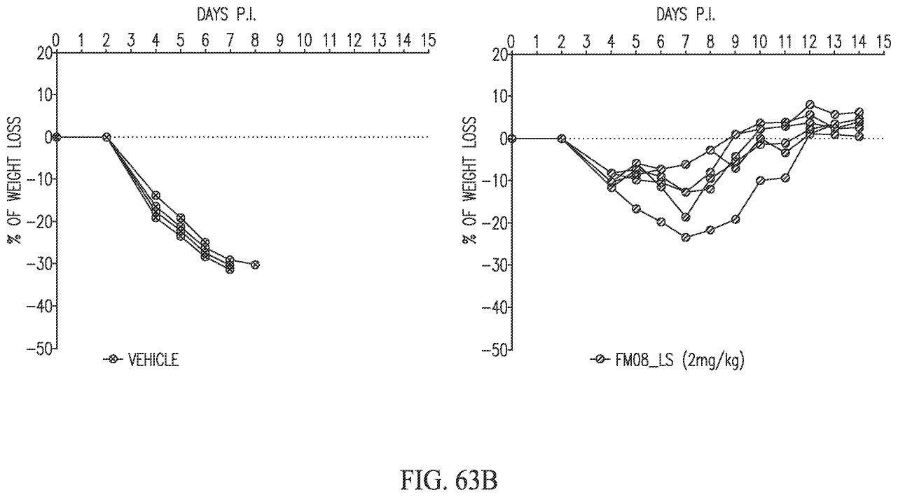

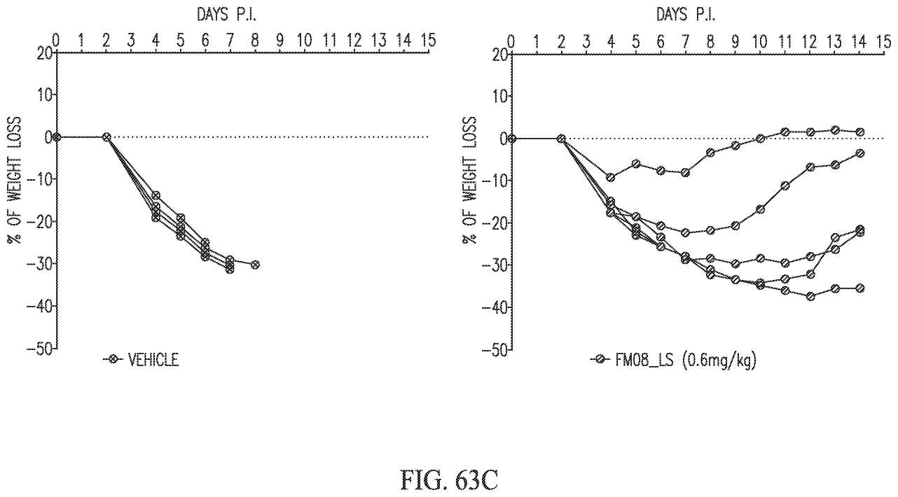

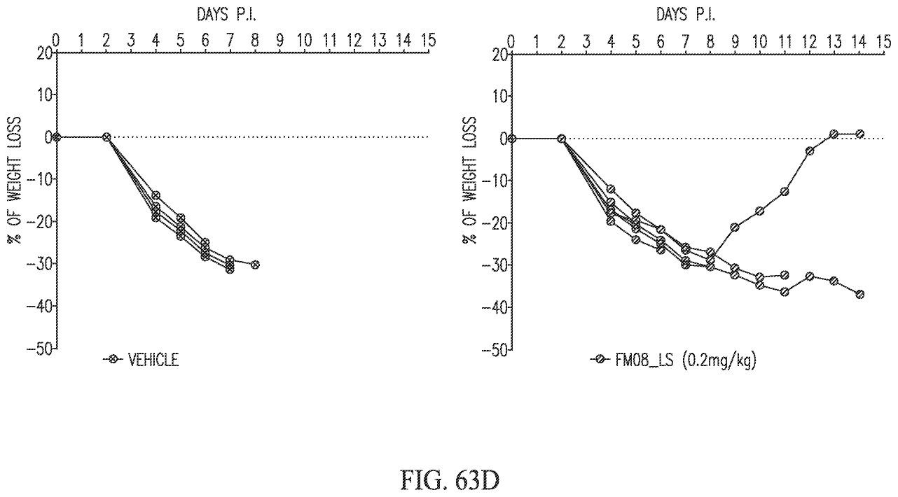

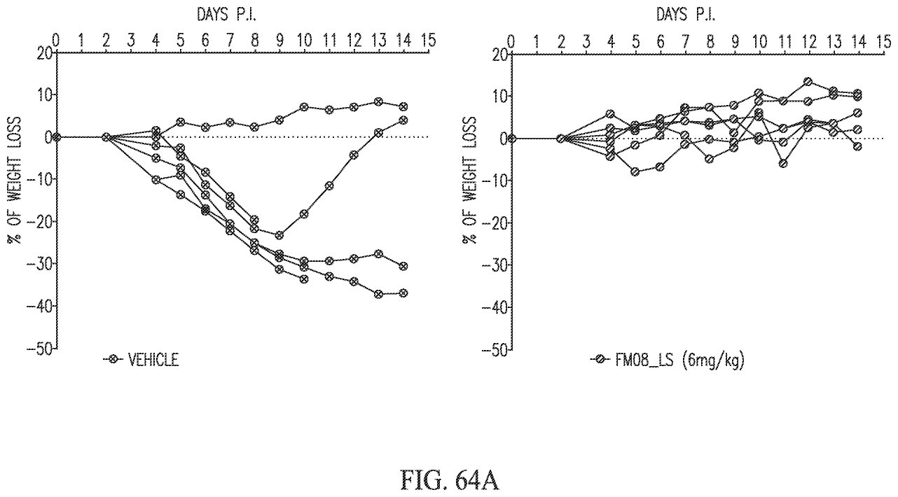

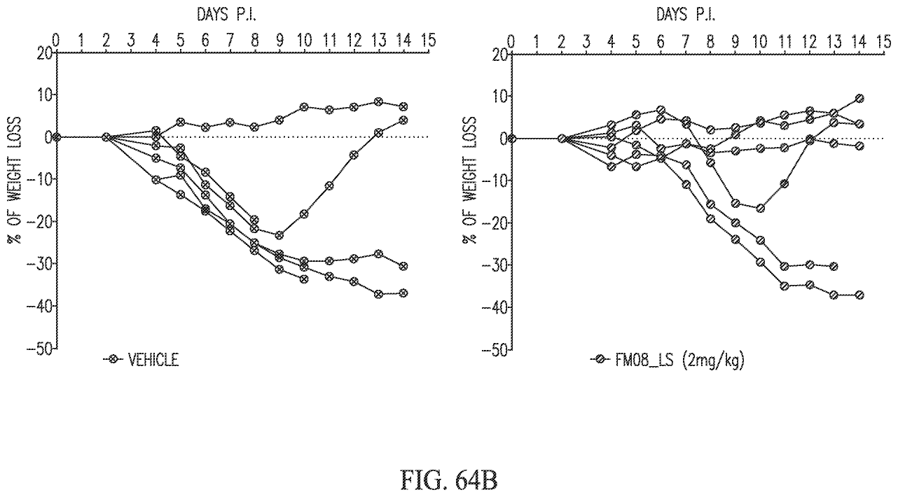

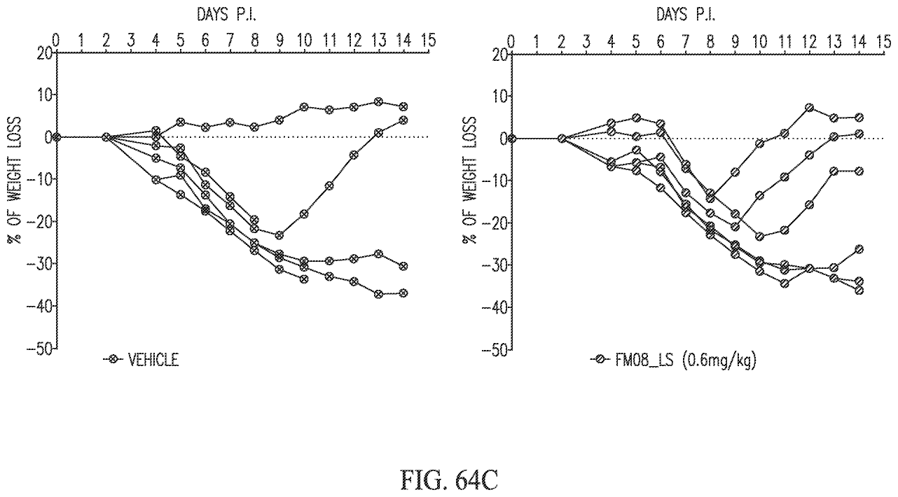

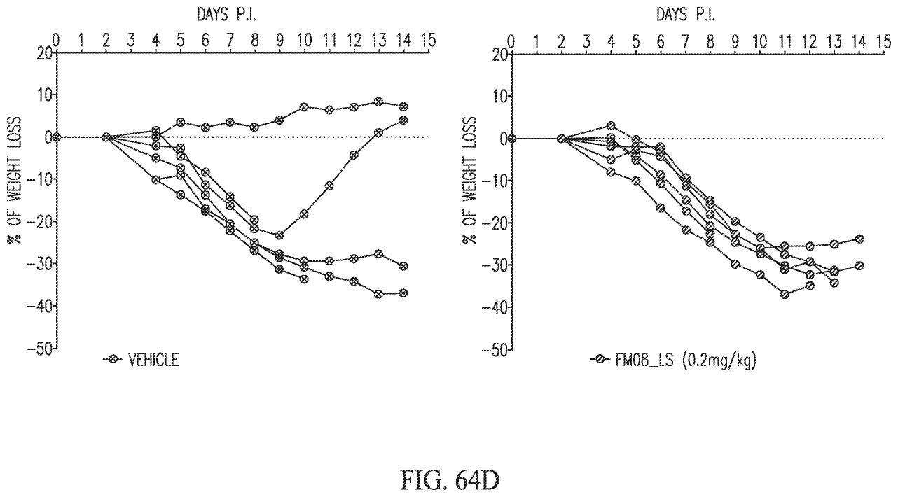

An inter-experiment in vivo study was designed to compare prophylactic activity of FM08_LS with FNI3 and FNI9 in BALB/c mice infected with H1N1 IAV A/Puerto Rico/8/34 or H3N2 IAV A/Hong Kong/8/68 (FIG. 62). Antibody was administered at 6 mg/kg, 2 mg/kg, 0.6 mg/kg, or 0.2 mg/kg, one day prior to infection with a LD90 (90% lethal dose) of A/Puerto Rico/8/34 or H3N2 IAV A/Hong Kong/8/68. The timeline, data collection, and endpoints of the study are the same as those seen in FIG. 28B. In Experiment A (“Exp-A”) BALB/c mice were infected with A/Puerto Rico/8/34 following pre-treatment with FNI3 (FIGS. 29A-29D) or FNI9 (FIGS. 30A-30D). In another arm of Experiment A, BALB/c mice were infected with A/Hong Kong/8/68 following pre-treatment with FNI3 (FIGS. 31A-31D) or FNI9 (FIGS. 32A-32D). In Experiment B (“Exp-B”) BALB/c mice were infected with A/Puerto Rico/8/34 (FIGS. 63A-63D) or A/Hong Kong/8/68 (FIGS. 64A-64D) following pre-treatment with FM08_LS.

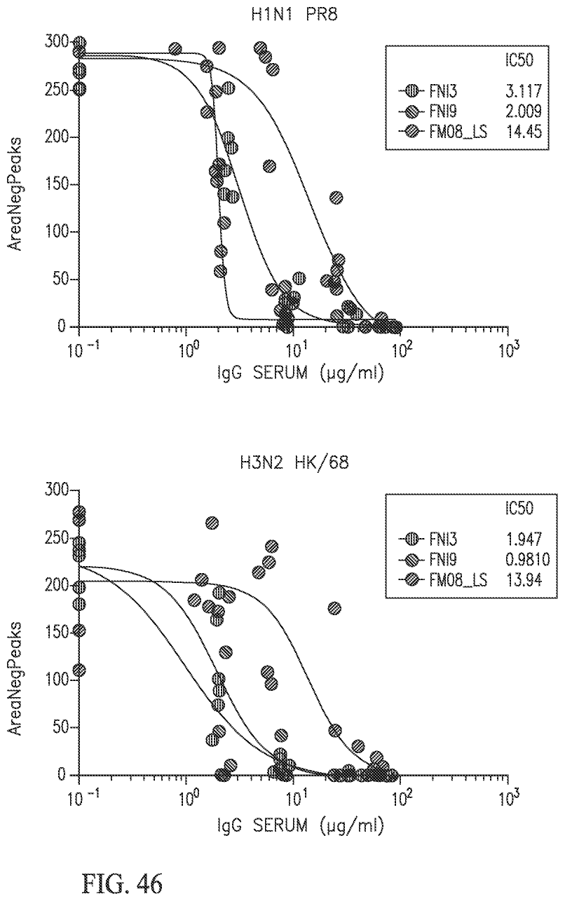

Body weight measurements over fifteen days are shown in FIGS. 29A-29D (A/Puerto Rico/8/34 FNI3 test group), 30A-30D (A/Puerto Rico/8/34 FNI9 test group), 31 A-31D (A/Hong Kong/l/68 FNI3 test group), 32A-32D (A/Hong Kong/1/68 FNI9 test group), 63A-63D (A/Puerto Rico/8/34 FM08_LS test group), and 64A-64D (A/Puerto Rico/8/34 FM08_LS test group). Negative area-under-the-curve peak values compared with IgG in serum from area-under-the-curve analysis of body weight loss in BALB/c mice infected with A/Puerto Rico/8/34 (H1N1) or A/Hong Kong/8/68 (H3N2) following treatment with FNI3 or, FNI9, or FM08_LS are shown in FIG. 46.

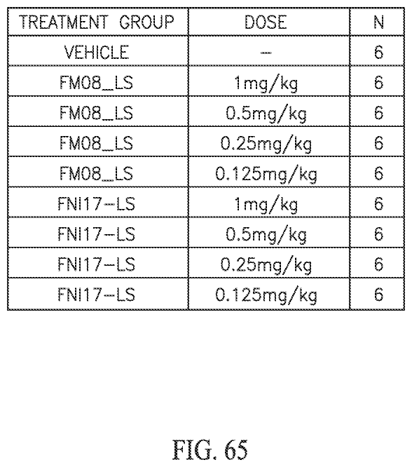

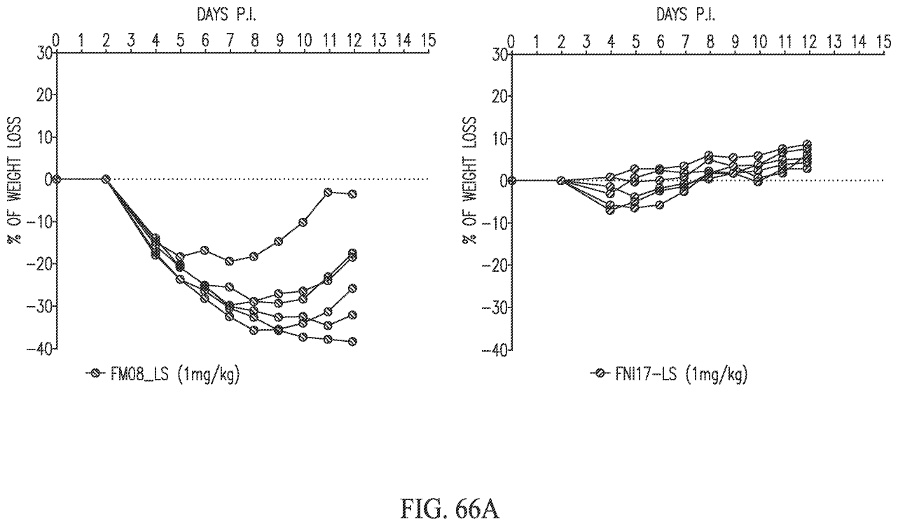

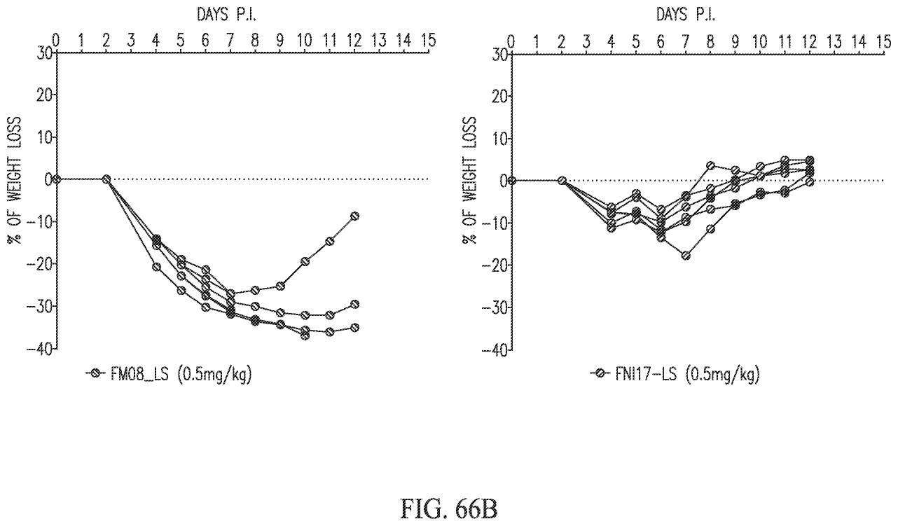

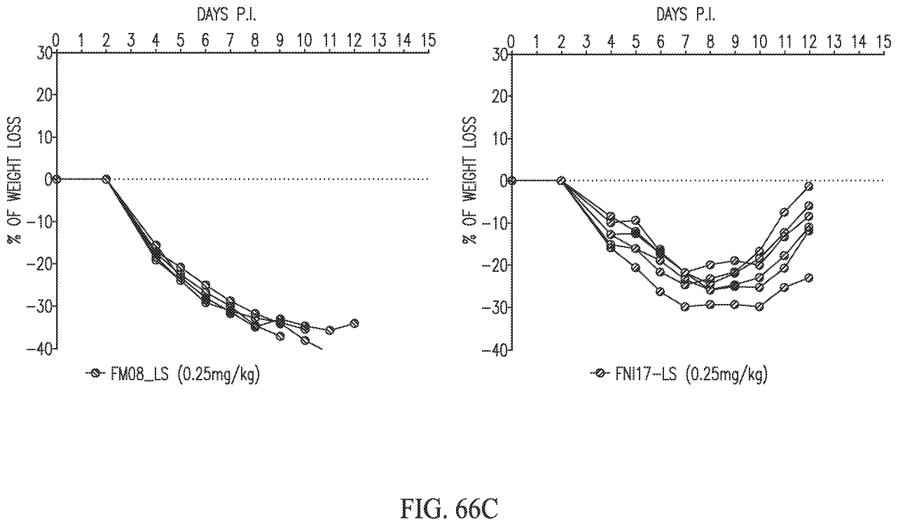

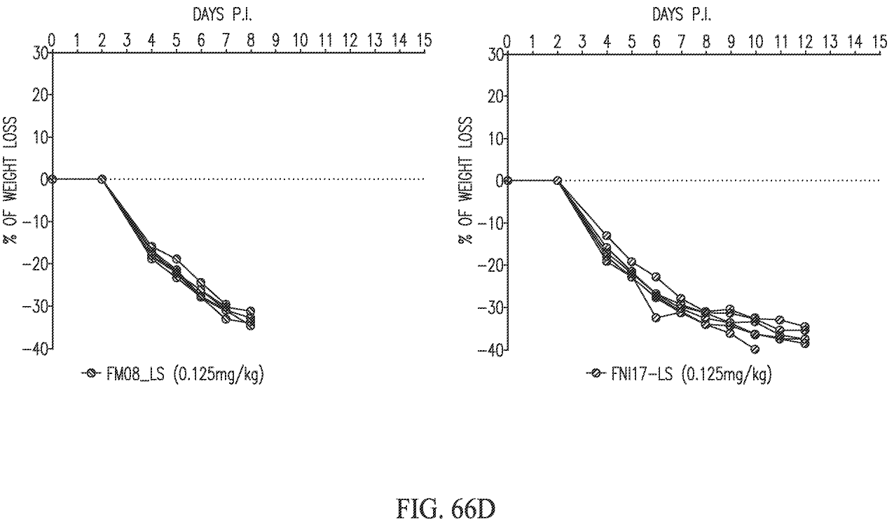

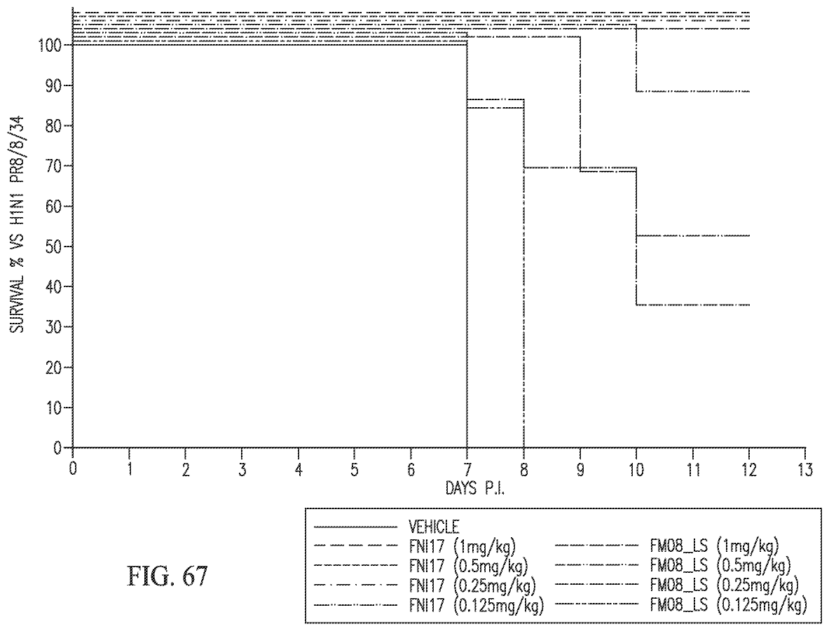

An in vivo study was designed to compare prophylactic activity of FM08_LS with FNI17 in BALB/c mice infected with H1N1 IAV A/Puerto Rico/8/34 (FIG. 65). Antibody was administered at 1 mg/kg (FIG. 66A), 0.5 mg/kg (FIG. 66B), 0.25 mg/kg (FIG. 66C), or 0.125 mg/kg (FIG. 66D), one day prior to infection with a LD90 (90% lethal dose) of A/Puerto Rico/8/34. Body weight measurements over twelve days are shown in FIGS. 66A-66D and survival over twelve days is shown in FIG. 67.

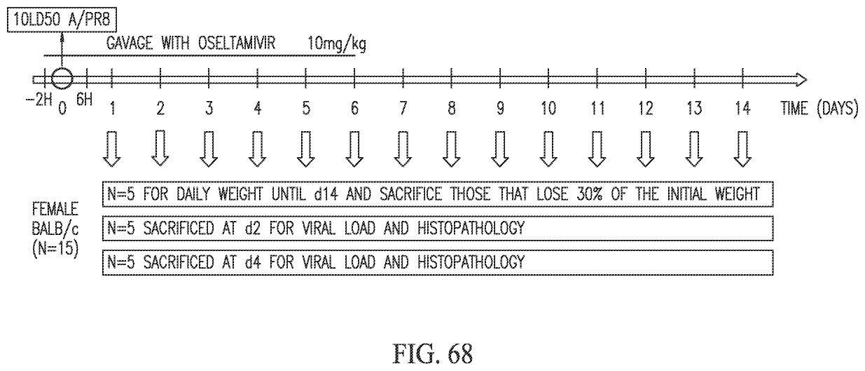

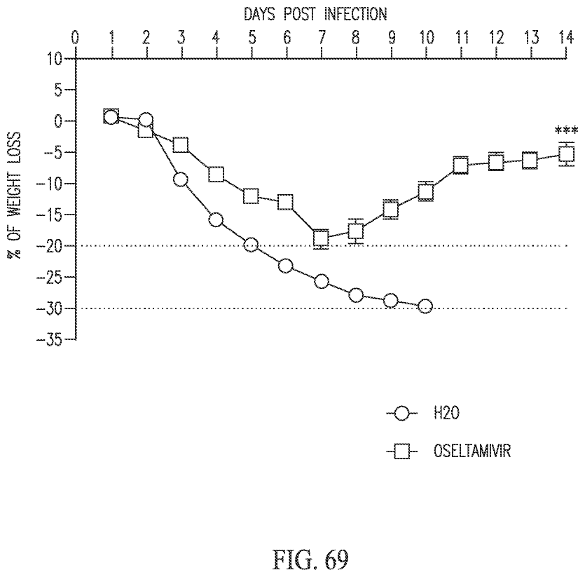

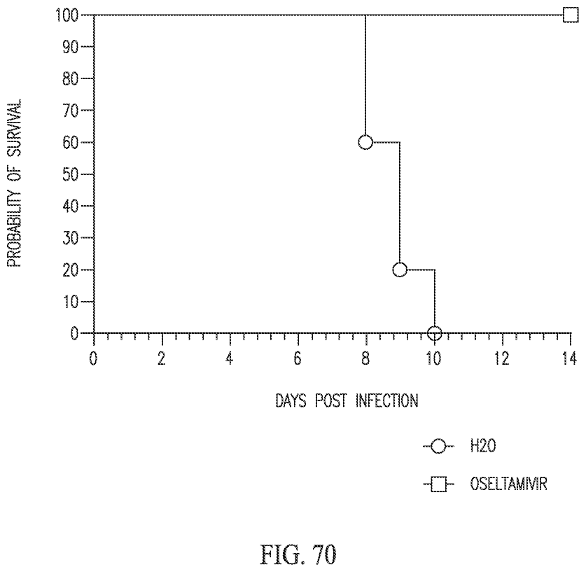

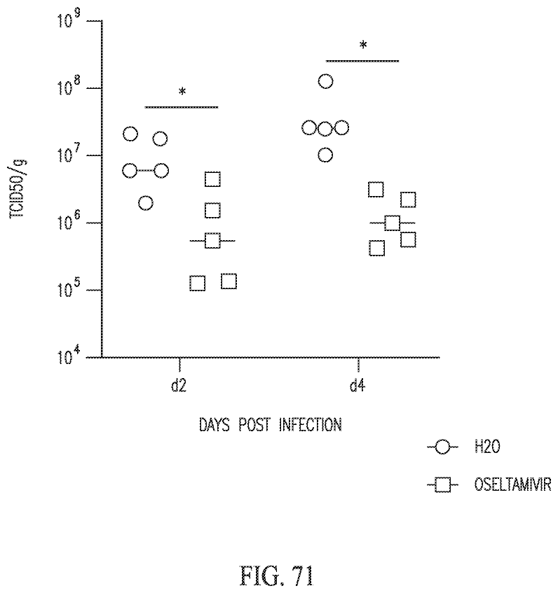

An in vivo study was designed to evaluate biological potency of oseltamivir (OSE) in female BALB/c mice infected with IAV A/Puerto Rico/8/34 (FIG. 68). OSE was administered at 10 mg/kg by oral gavage on Day 0 beginning at two hours prior to infection with 10-fold LD50 (50% lethal dose) of A/Puerto Rico/8/34. OSE was administered at the same dose at 6 hours post-infection and then twice daily until day 6 post-infection. Body weight measurements over fourteen days are shown in FIG. 69 and survival over fourteen days is shown in FIG. 70. Viral titers in lung homogenates from OSE-treated mice were measured from samples obtained at two and four days post-infection (FIG. 71).

### Example 10

### Generation and Characterization of FNI3, FNI9, FNI17, and FNI19 Variant Antibodies

Varaible domain sequence variants were generated from FNI3, FNI9, FNI17, and FNI19 and characterized for binding and neutralization. A total of thirty-two (32) variant antibodies were generated, in which twenty-six (26) variants contained a reversion of VH and/or VL framework amino acid(s) to germline sequence, three (3) FNI17 variants contained a reversion of VH framework regions to germline sequence and a W97A/L/Y mutation in VL, and three (3) FNI17 variants contained a wild-type VH and a W97A/L/Y mutation in VL. A total of 11 variants were generated from FNI3, variants from FNI9, 11 variants from FNI17, and 5 variants from FNI19. FIGS. 72A-72B show acid sequences of FNI3, FNI9, FNI17, and FNI19 VH (FIG. 72A) and VK (FIG. 72B) aligned to unmutated common ancestor, “UCA”.

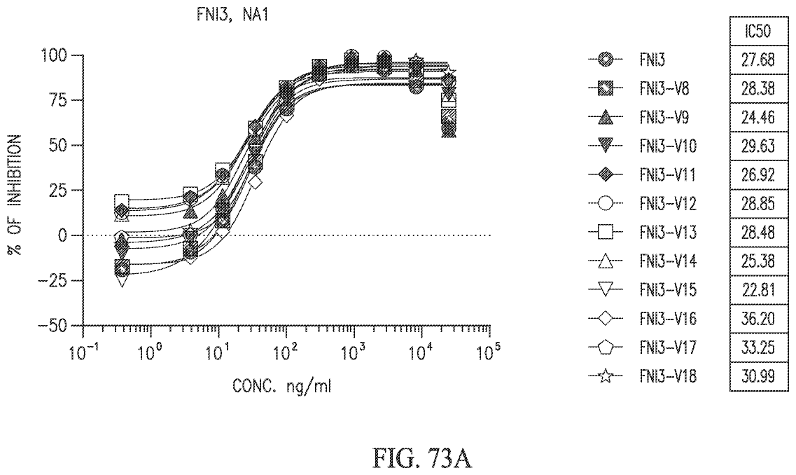

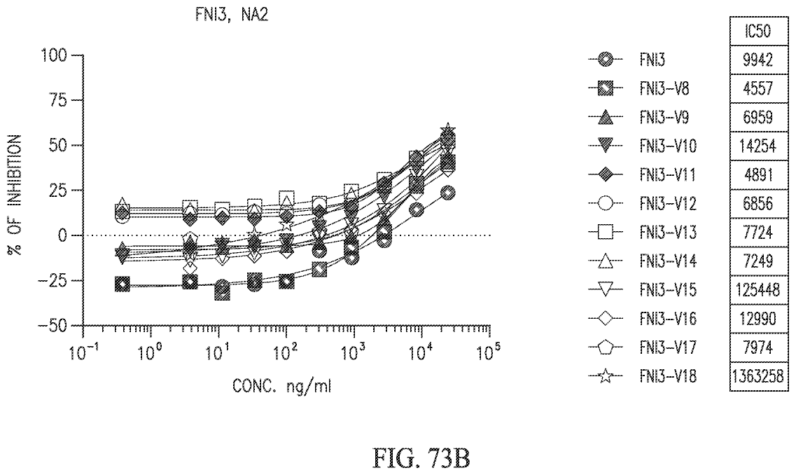

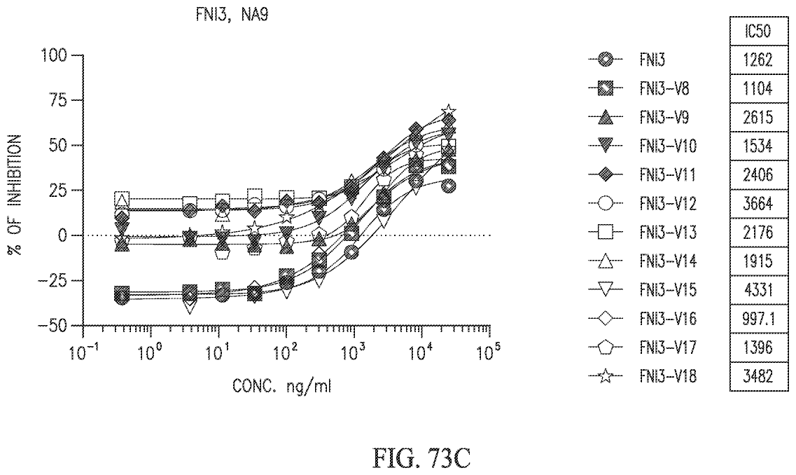

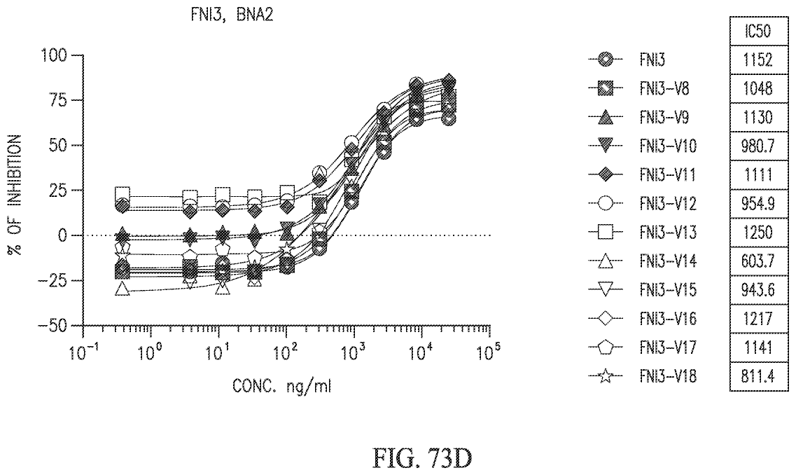

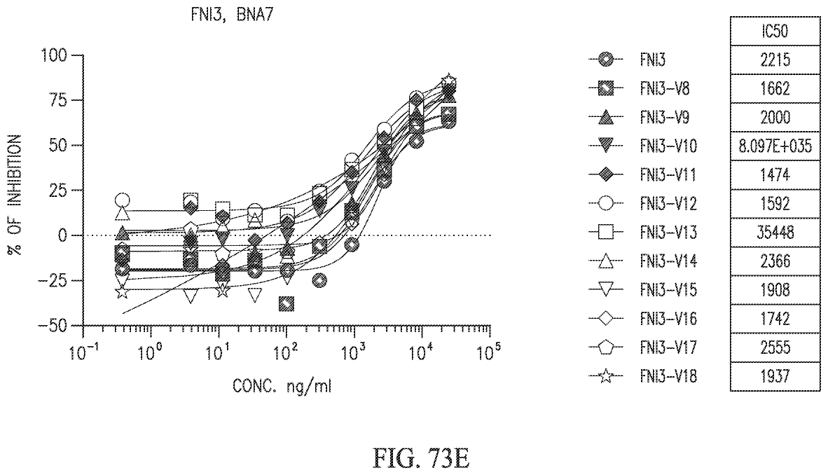

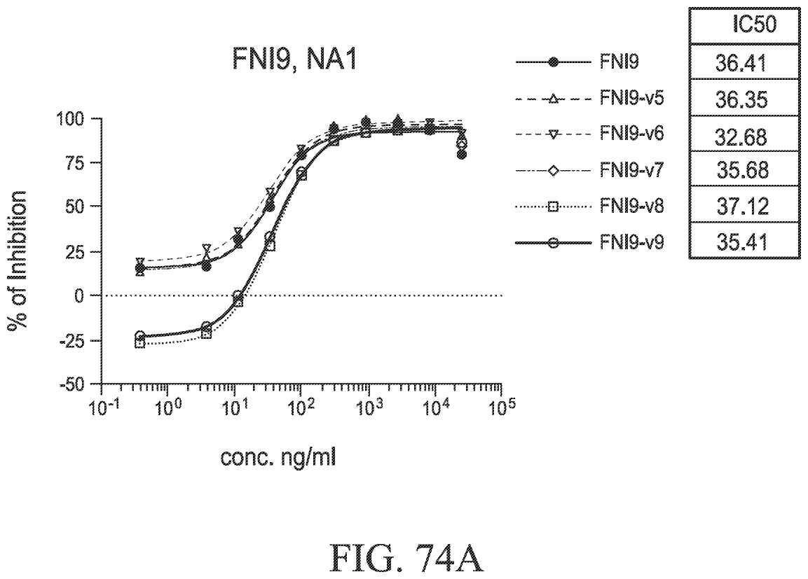

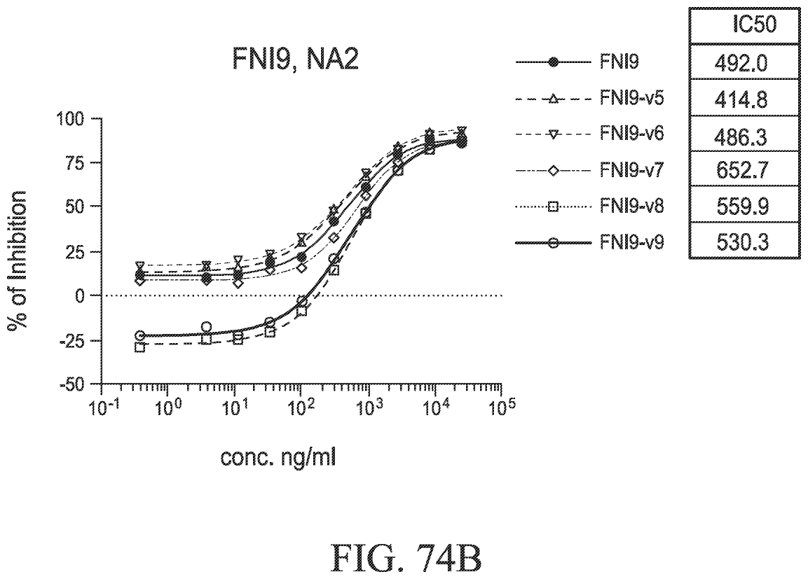

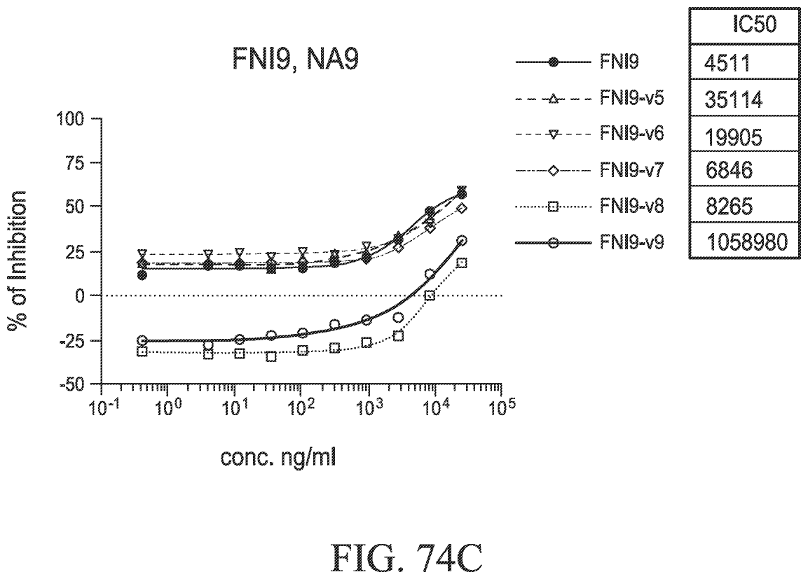

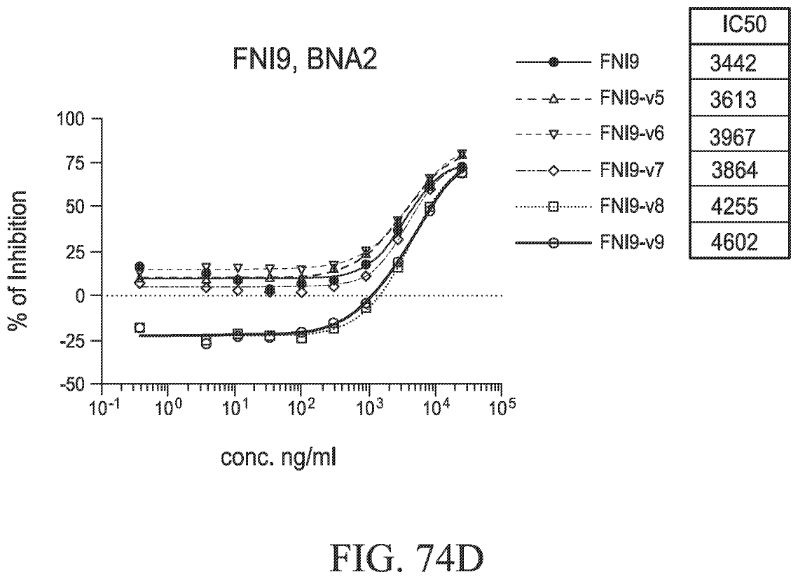

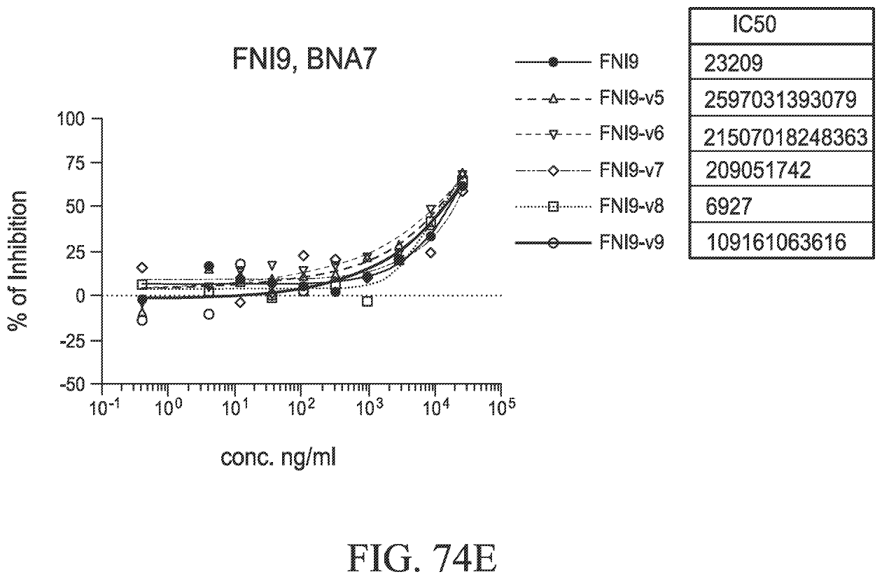

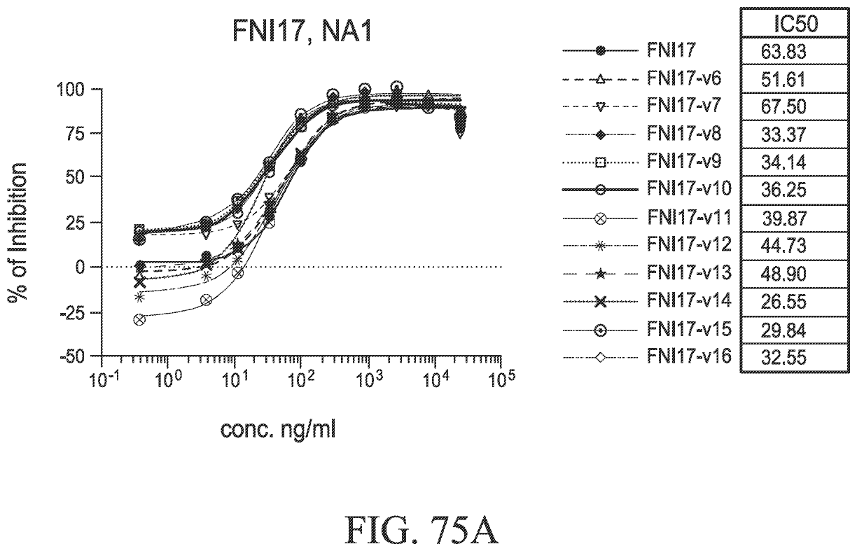

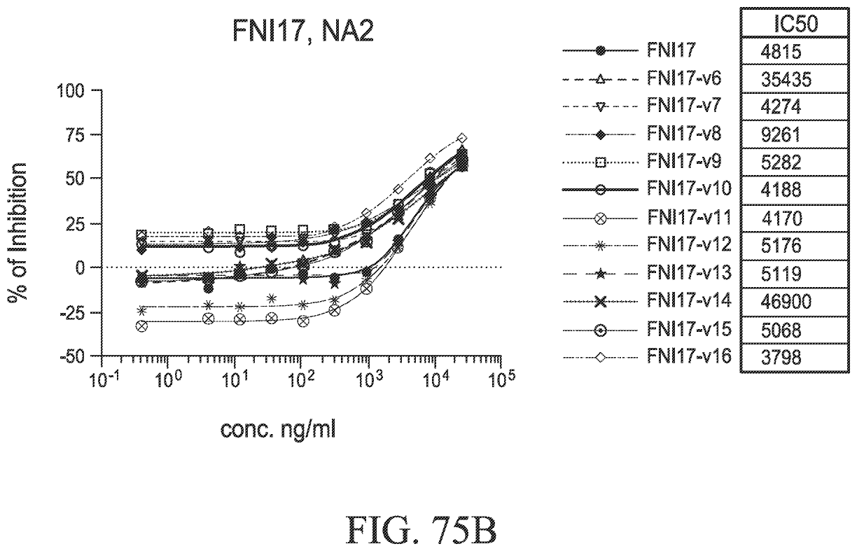

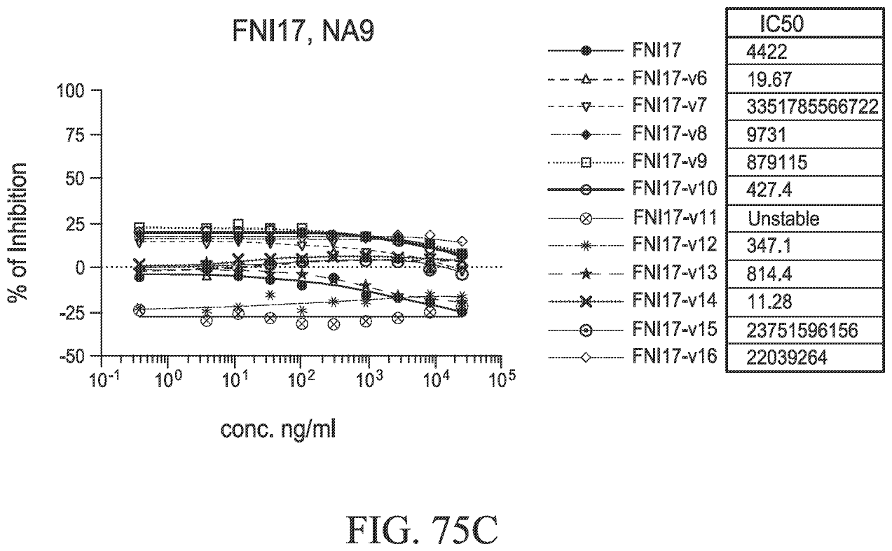

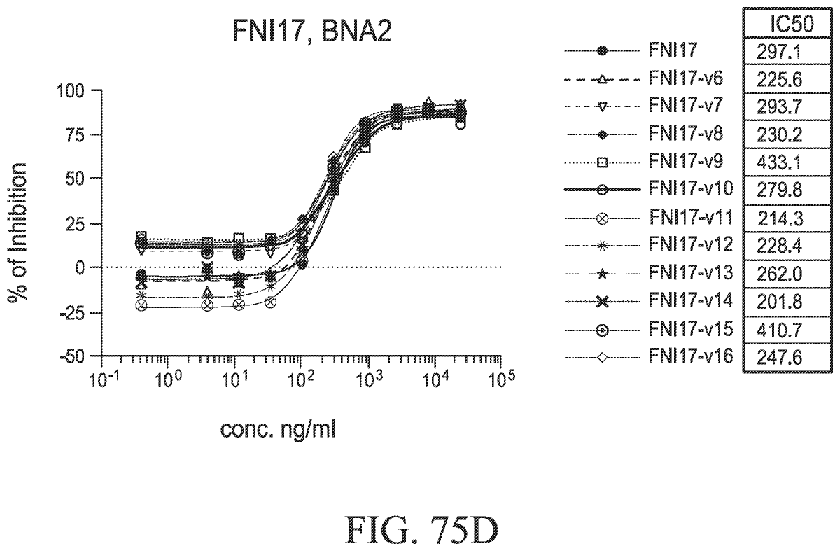

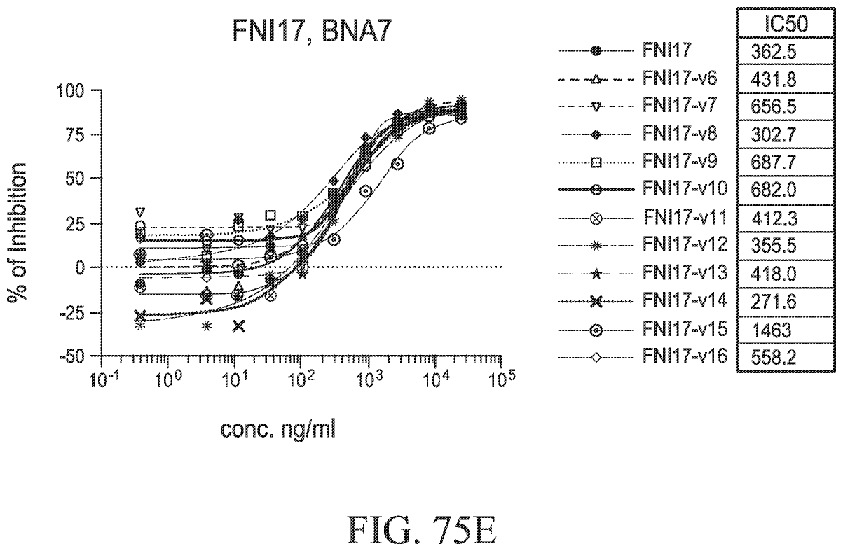

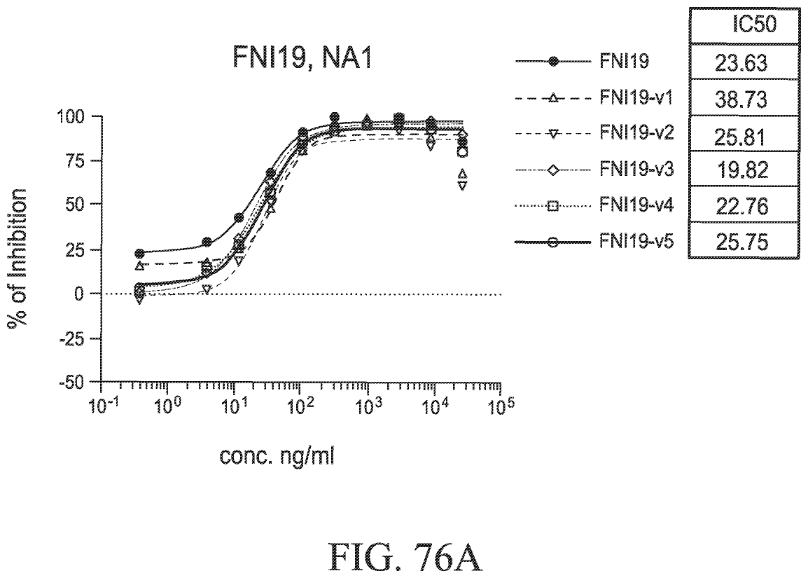

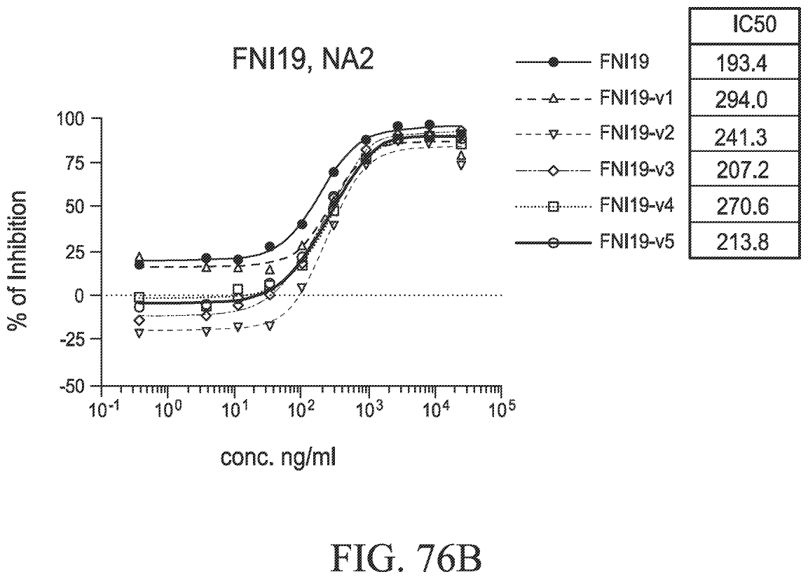

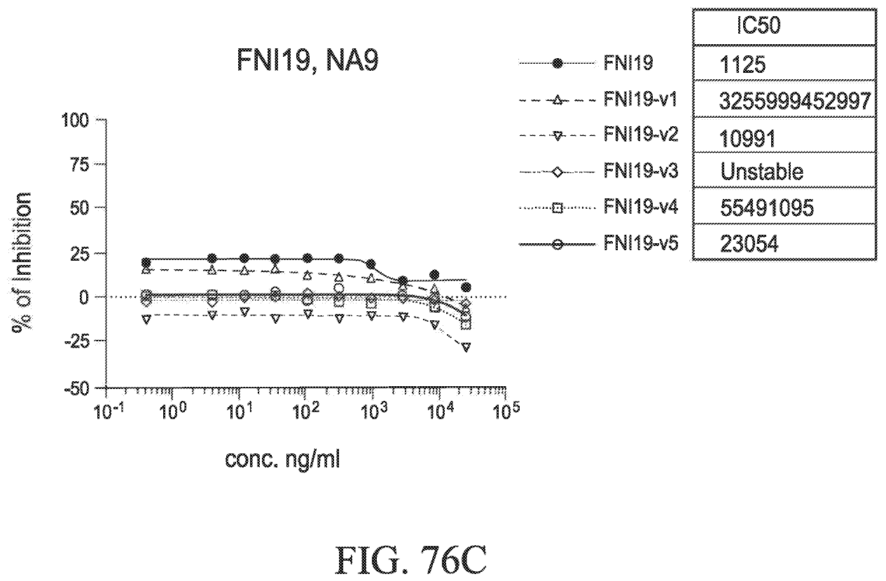

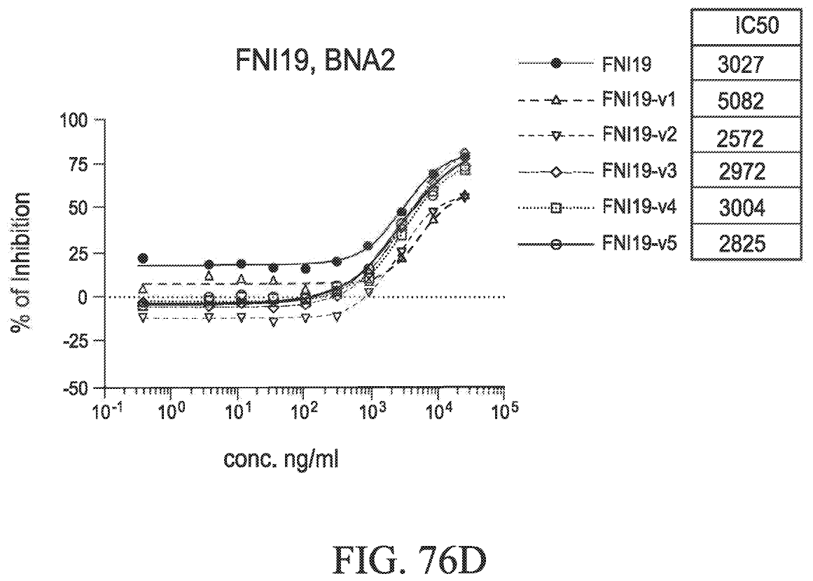

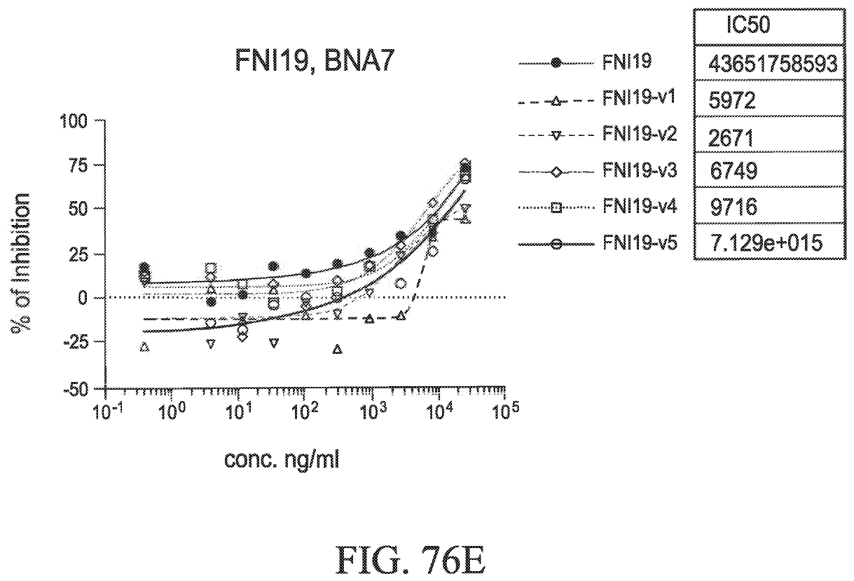

In vitro inhibition of sialidase activity against IAV NAs (NA1 from H5N1 A/Vietnam/1203/2004; NA2 from H3N2 A/Tanzania/205/2010; NA9 from H7N9 A/Hong Kong/56/2015) and IBV NAs (BNA7 from B/Malaysia/2506/2004; BNA2 from B/Perth/211/2011) by the developability variants was measured. Inhibition activity is shown in FIGS. 73A-73E (FNI3 and variants FNI3-v8 through FNI3-v18; see Table 2 for amino acid and nucleic acid sequences), FIGS. 74A-74E (FNI9 and variants FNI9-v5 through FNI9-v9; see Table 2 for amino acid and nucleic acid sequences), FIGS. 75A-75E (FNI17 and variants FNI17-v6 through FNI17-v16; see Table 2 for amino acid and nucleic acid sequences), and FIGS. 76A-76E (FNI19 and variants FNI19-v1 through FNI19-v5; see Table 2 for amino acid and nucleic acid sequences).

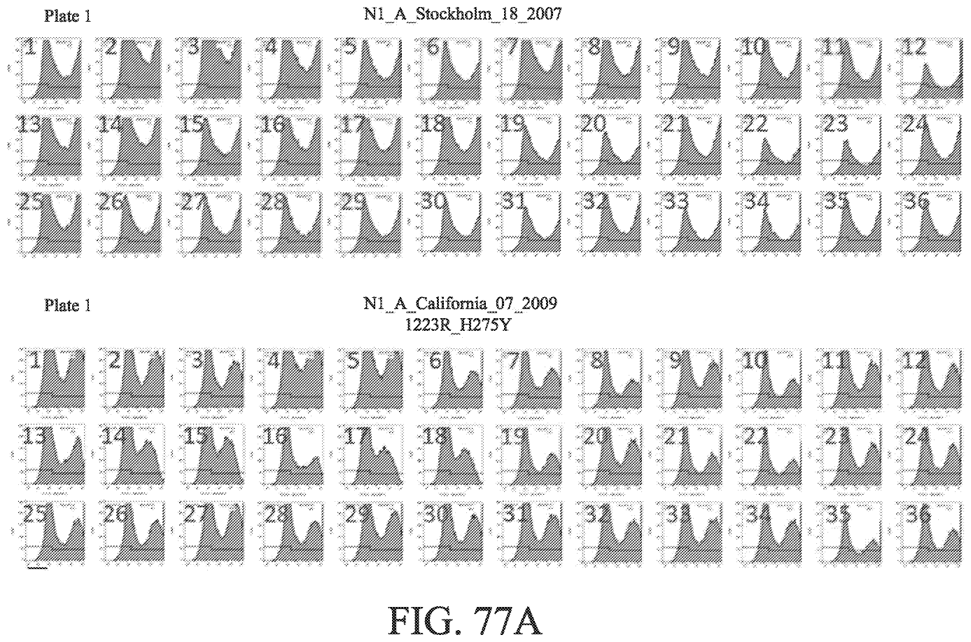

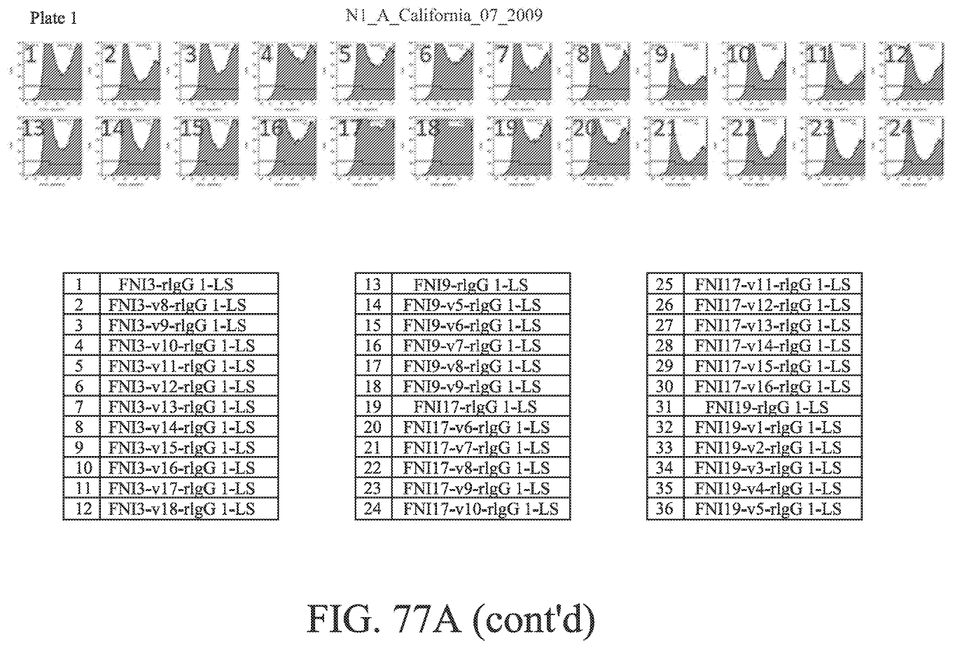

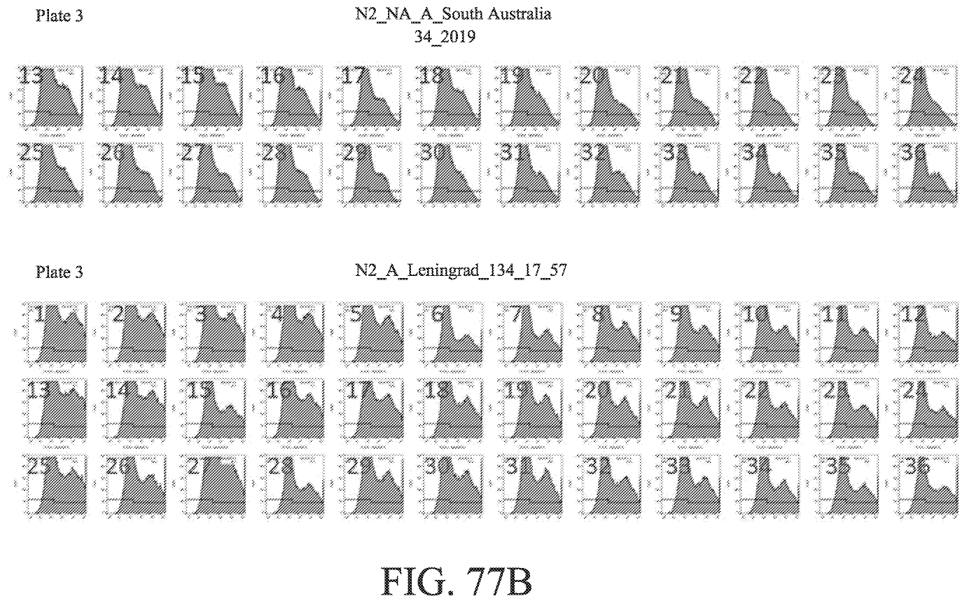

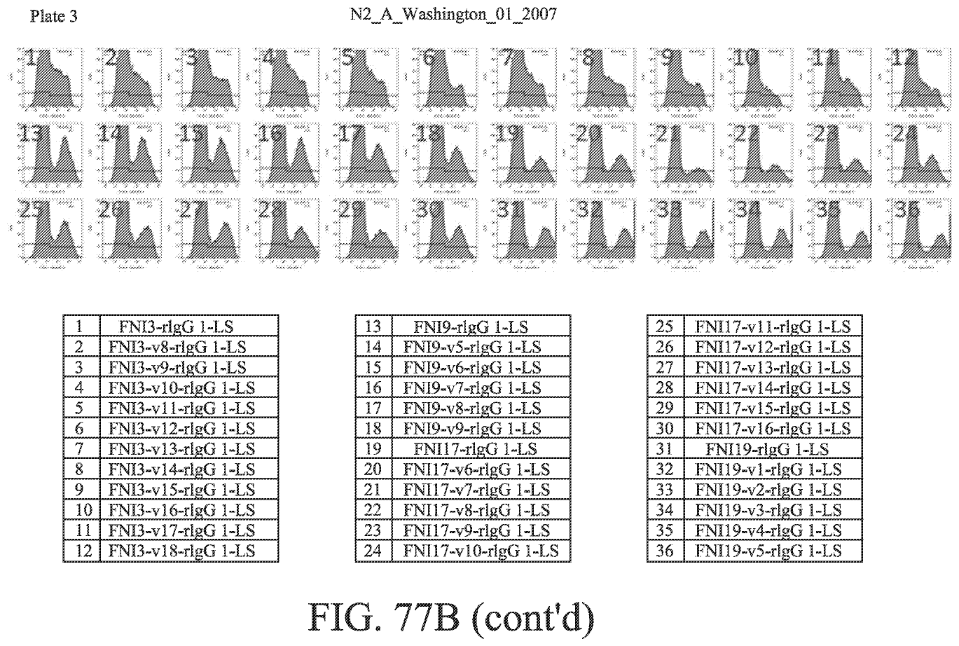

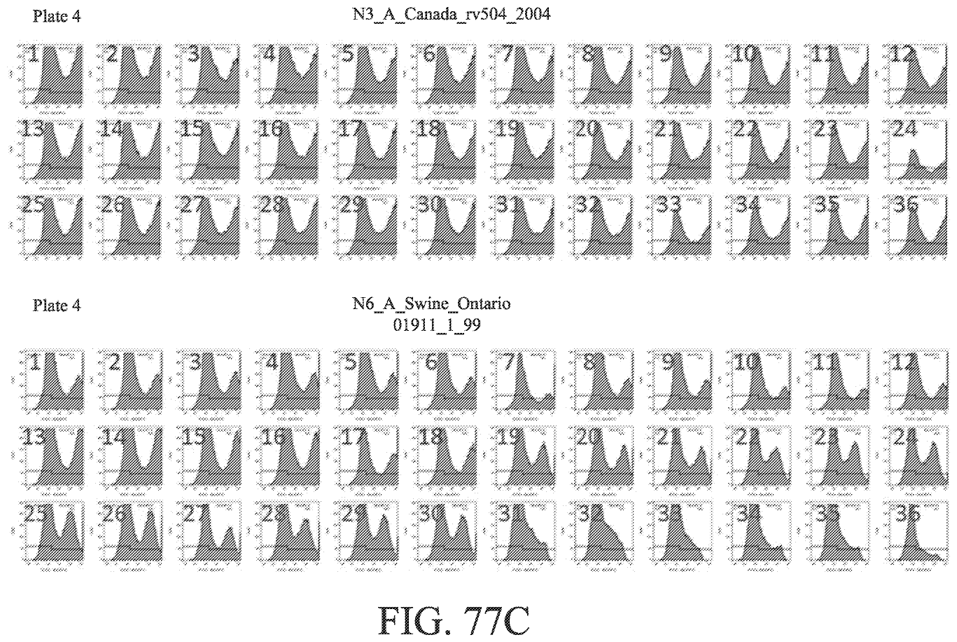

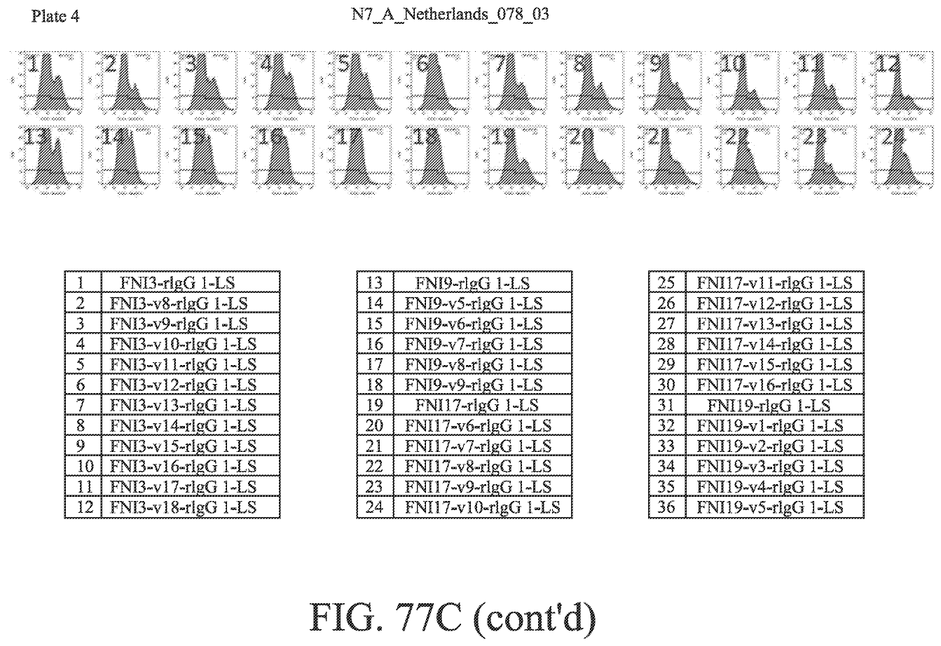

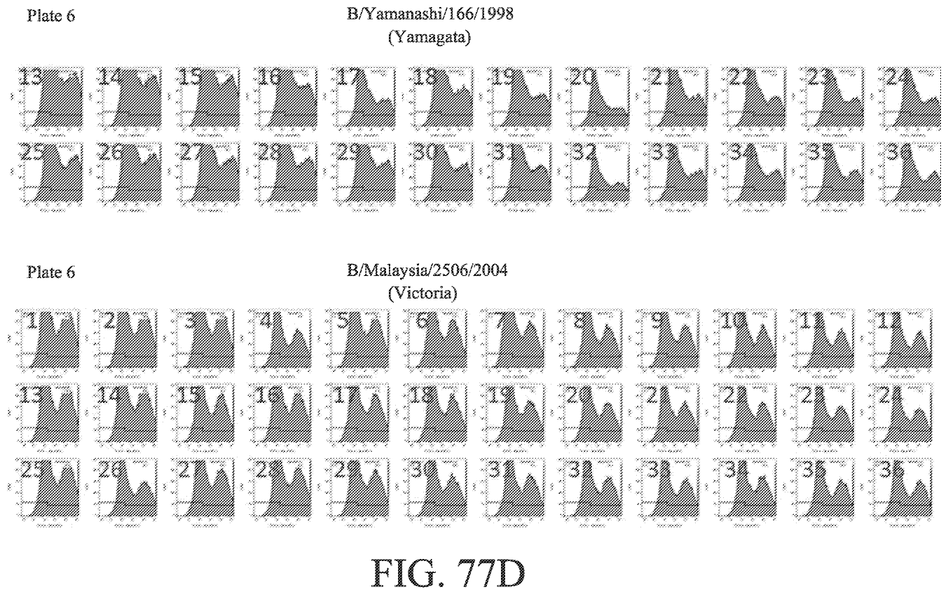

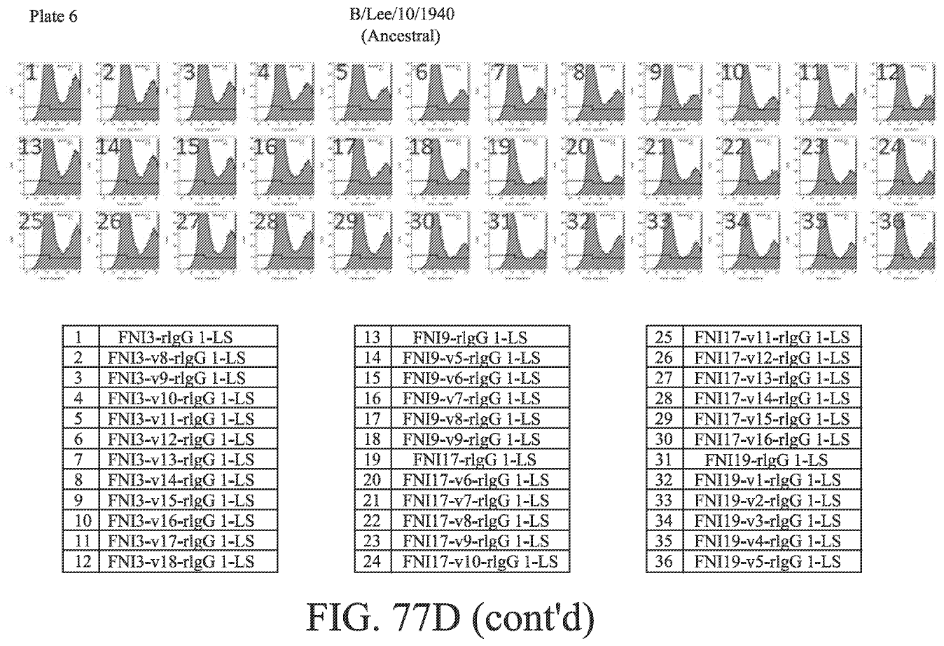

Binding of all thirty-two (32) variants to IAV NAs and IBV NAs was evaluated by FACS to exclude potential loss of breadth due to reversion to germline of mAb framework regions. Binding was measured against N1 from A/Stockholm/18/2007, A/California/07/2009, and A/California/07/2009 I23R/H275Y (FIG. 77A); N2 from A/South Australia/34/2019, A/Leningrad/134/17/57, and A/Washington/01/2007 (FIG. 77B); N3 from A/Canada/rv504/2004 (FIG. 77C); N6 from A/swine/Ontario/01911/1/99 (FIG. 77C); N7 from A/Netherlands/078/03 (FIG. 77C); IBV NA from B/Yamanashi/166/1998 (Yamagata), B/Malaysia/2506/2004 (Victoria), and B/Lee/10/1940 (Ancestral) (FIG. 77D).

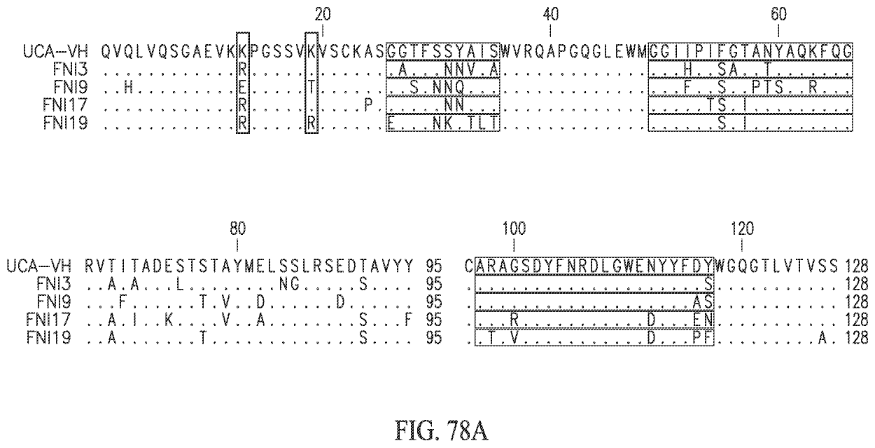

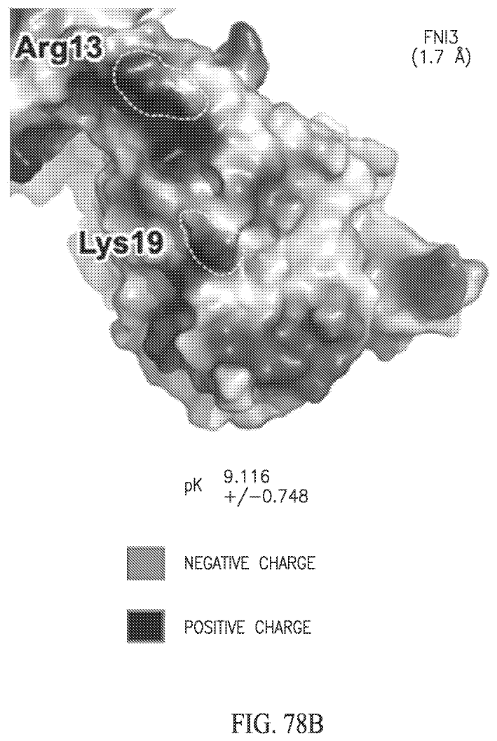

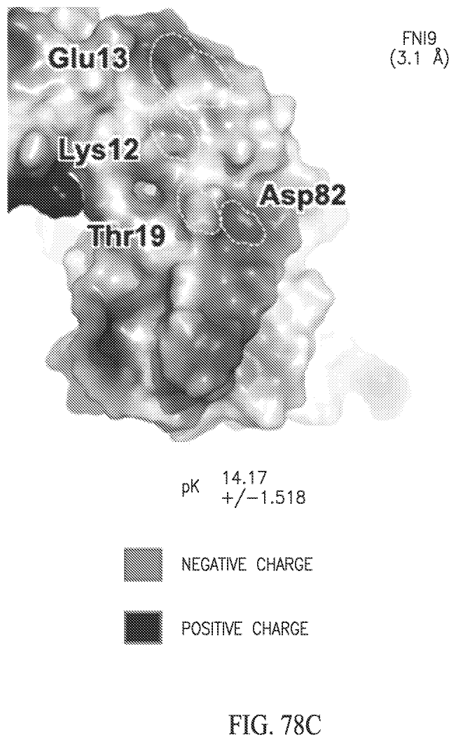

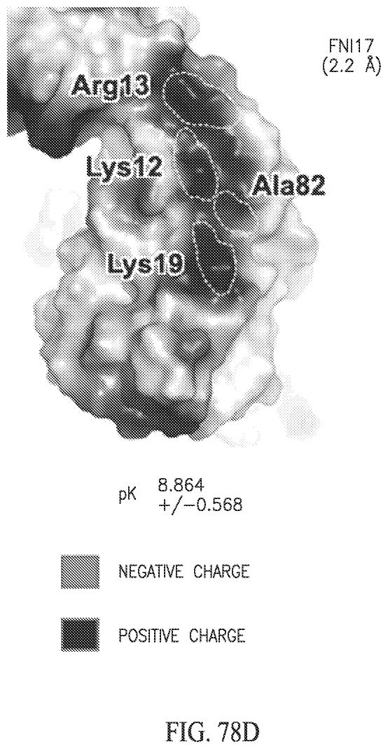

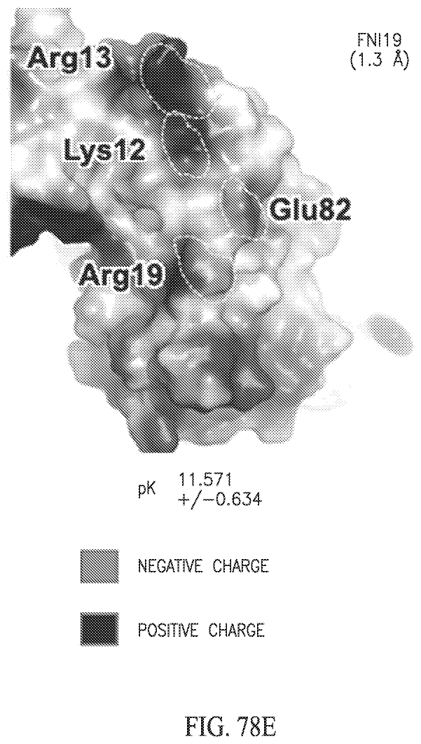

Surface charge and pharmacokinetic (pK) values were determined for FNI3, FNI9, FNI17, and FNI19. FIG. 78A shows an alignment of FNI3, FNI9, FNI17, and FNI19 VH amino acid sequence with that of the unmutated common ancestor, “UCA”, wherein the vertical rectangles indicate positively charged Lys12 and Lys19 residues in the UCA sequence and corresponding residues at the same position in germ-line reverted FNI3, FNI9, FNI17, and FNI19. Overall surface charge maps generated using PyMOL (The PyMOL Molecular Graphics System, Version 1.2r3pre, Schrödinger, LLC) are shown for FNI3 (FIG. 78B), FNI9 (FIG. 78C), FNI17 (FIG. 78D), and FNI19 (FIG. 78E), along with pK values and resolution (reported in A). Decreases in overall positive charge on the surface of the antibody may serve to reduce sequestration of the antibody by pinocytosis on the cell surface. FNI9 presented a more negative surface charge and a correspondingly improved pK value in comparison to FNI3, FNI17, and FNI19.

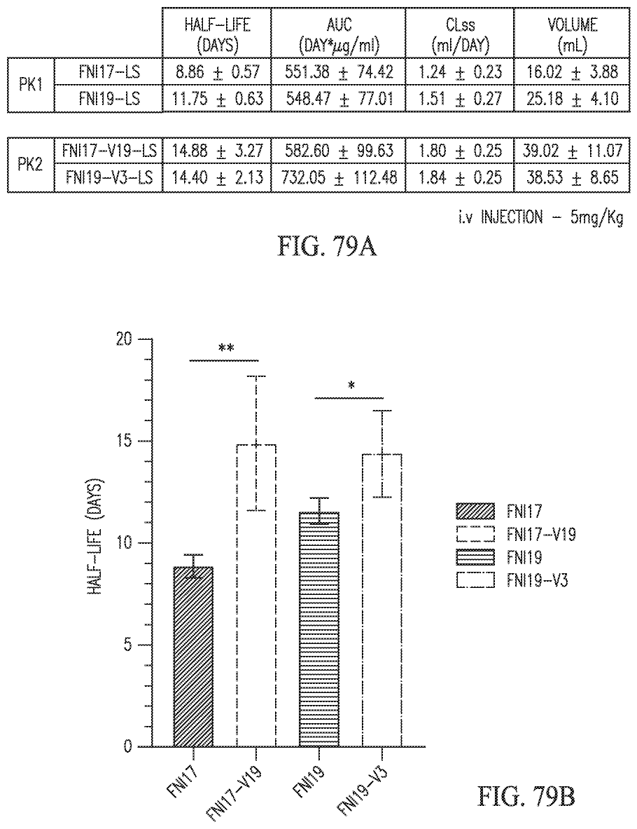

Two variants (expressed as rIgG1 with MLNS muations in Fc) were selected for pharmacokinetic evaluation: FNI17-v19-LS (VH: SEQ ID NO.:199; VL: SEQ ID NO.: 201) and FNI19-v3-LS (VH: SEQ ID NO.:203; VL: SEQ ID NO.: 205). FNI17-v19 was generated by further engineering FNI17-v13 to incorporate somatic mutations within the framework 1 (FR1) region of the heavy chain (R/E and K/T) to reduce the positive charge and decrease pinocytosis thus increasing the half-life. Inter-experiment pharmacokinetic analyses were performed between FNI17-LS and FNI19-LS (“PK1”), and FNI19-v3-LS and FNI17-v19 (“PK2”). Tg32 mice were intravenously injected with 5 mg/kg antibody. Half-life (FIG. 79A) as well as area-under-the-curve (AUC), steady state clearance (CLss), and total volume analyzed (Volume) (FIG. 79B) were determined.

### Example 11

### In Vivo Potency: Comparison of FNI17-V19 Antibody with Oseltamivir

In vivo potency of FNI17-v19 was evaluated in comparison with that of OSE. An in vivo study was designed to evaluate prophylactic activity of FNI17-v19-rIgG1-LS compared with oseltamivir (OSE) in BALB/c mice infected with IAVs and IBVs, as shown in FIG. 80. Treatment groups were administered 9 mg/kg, 3 mg/kg, 0.9 mg/kg, or 0.3 mg/kg of FNI17-v19-rIgG1-LS 24 hours prior to infection at LD90 (90% lethal dose). FNI17-v19-rIgG1-GRLR was also tested at 9 mg/kg and 0.3 mg/kg for mice administered IAV viruses (H1N1 A/Puerto Rico/8/34 or H3N2 A/Hong Kong/8/68). The GRLR mutation abrogates binding by FcgRs and complement thus abrogating activation of effector functions. Twenty-four hours after antibody administration, mice were infected at LD90 (90% lethal dose) with IAVs, H1N1 A/Puerto Rico/8/34 or H3N2 A/Hong Kong/8/68, or IBVs, B/Victoria/504/2000 (Yamagata) or B/Brisbane/60/2008 (Victoria). OSE was orally administered daily at 10 mg/kg from 2 hours before infection to 3 days post-infection to mimic dosing regimens used for human treatment in a prophylactic setting.

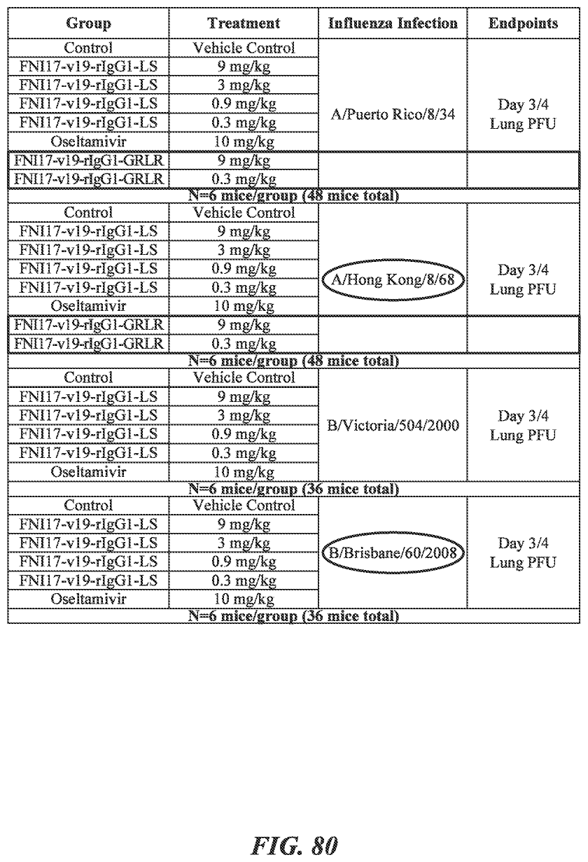

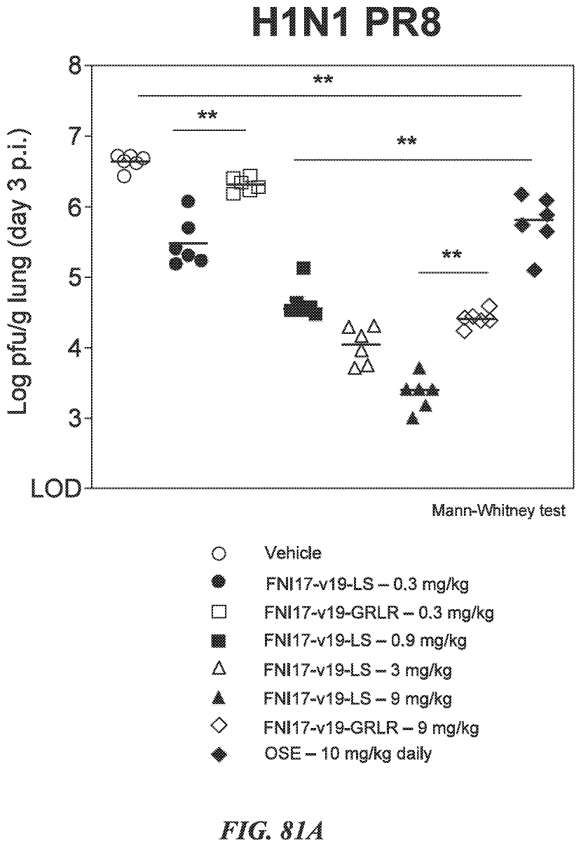

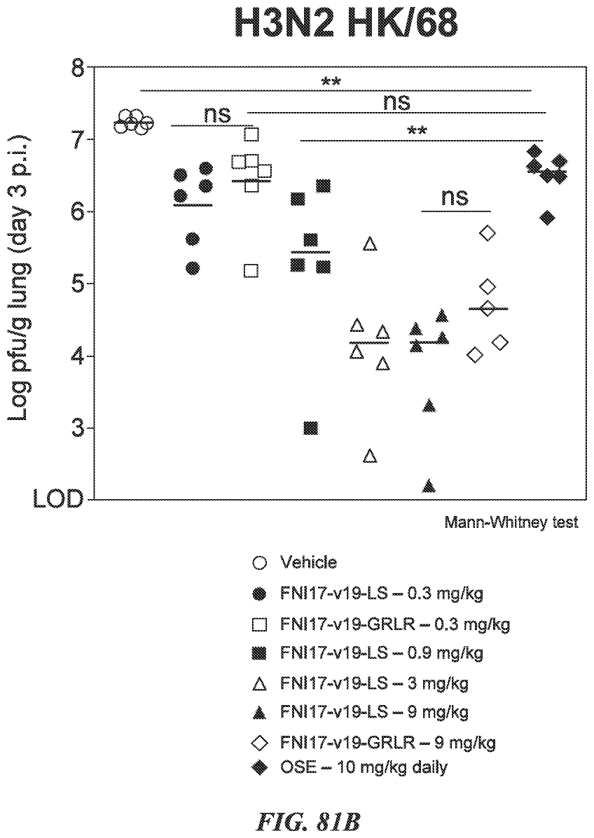

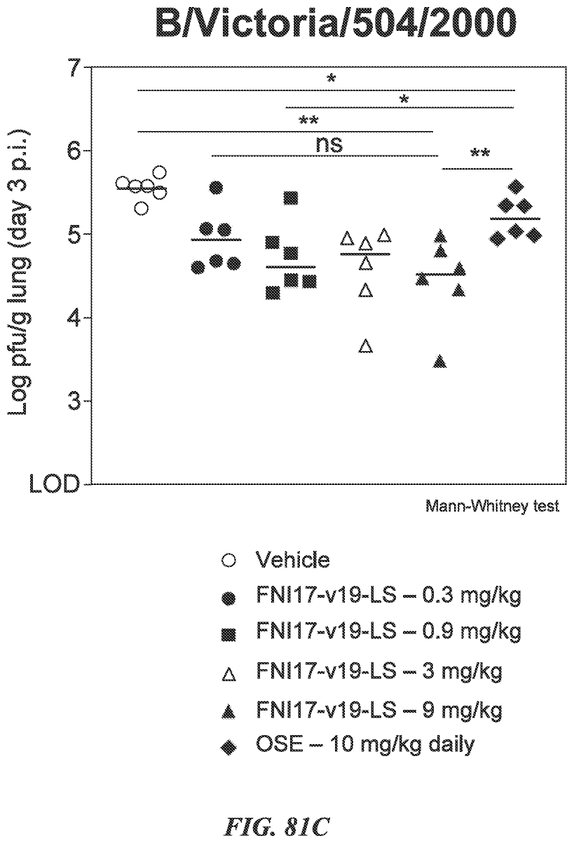

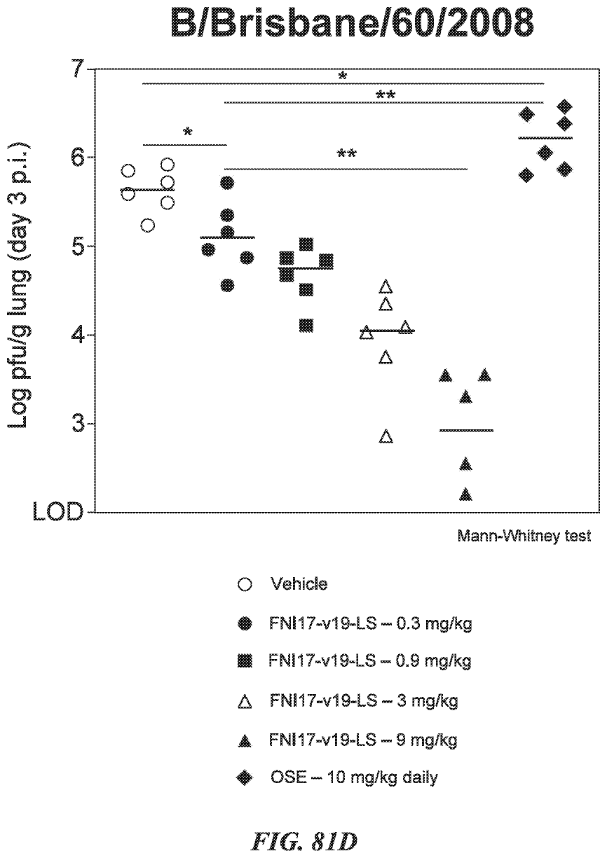

Viral titer in the lungs was evaluated in mice from the in vivo model described in FIG. 80. At day 3 post-infection, mice were euthanized, lungs were collected, and lung viral titres were measured using plaque assay following infection with H1N1 A/Puerto Rico/8/34 (FIG. 81A), H3N2 A/Hong Kong/8/68 (FIG. 81B), B/Victoria/504/2000 (Yamagata; FIG. 81C), or B/Brisbane/60/2008 (Victoria; FIG. 81D). Administration of OSE resulted in a 1 log reduction in viral titres in comparison to the vehicle with all the virus tested with exception of the B/Brisbane/60/2008. A single administration of FNI17-v19-rIgG1-LS at 0.3 mg/kg outperformed the prophylactic activity of oseltamivir with all tested viruses, further, reduction in viral lung titre by FNI17-v19-rIgG1-LS was dose-dependent. Administration of the GRLR version of the mAb (FNI17-v19-rIgG1-GRLR) resulted in a lower level of protection in comparison to the parental antibody. The decrease in prophylactic activity associated to the abrogation of the effector functions appeared consistent and independent of the dose used. It was observed that the difference in reduction of PFU between the FNI17-v19-rIgG1-LS and FNI17-v19-rIgG1-GRLR mAbs was the same when comparing these mAbs at the doses of 9 mg/kg and 0.3 mg/kg.

### Example 12

### In Vivo Potency: Prophylactic Activity of FNI17-V19 Antibody in Humanized FcγR Mice

In vivo potency of FNI17-v19 was evaluated in an FcγR-humanized mouse model.

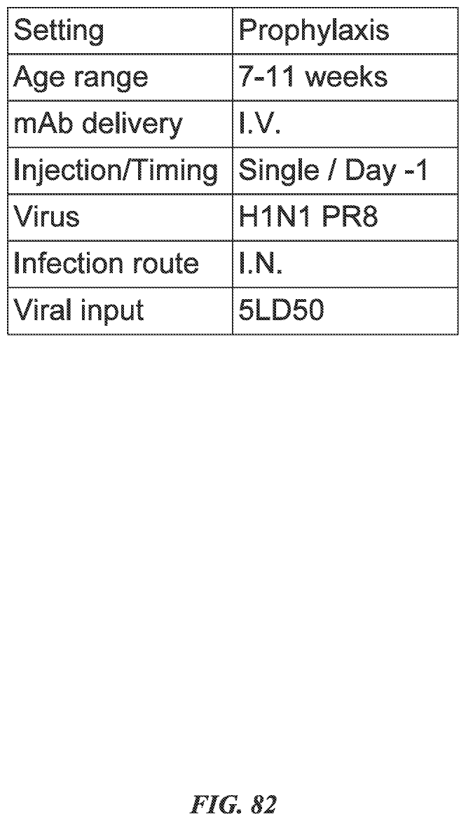

The design of an in vivo study to evaluate prophylactic activity of FNI17-v19 in humanized FcγR mice infected with H1N1A/Puerto Rico/8/34 is shown in FIG. 82. Mice were pre-administered FNI17-v19 mAb at 0.9 mg/kg, 0.3 mg/kg, or 0.09 mg/kg, 24 hours prior to intranasal infection at 5LD50 (five times 50% lethal dose) of H1N1 A/Puerto Rico/8/34. Animals were then monitored for body weight loss and mortality over the course of 14 days. Mice losing more than 30% body weight were euthanized.

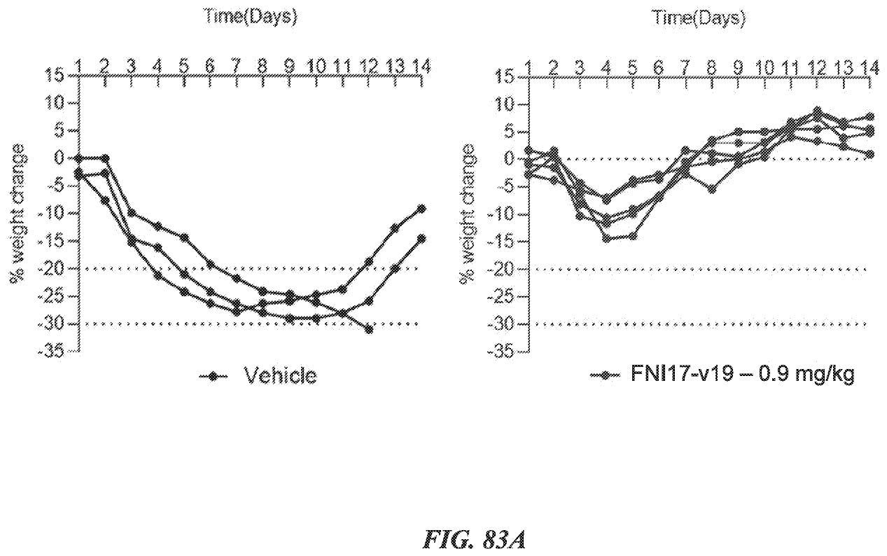

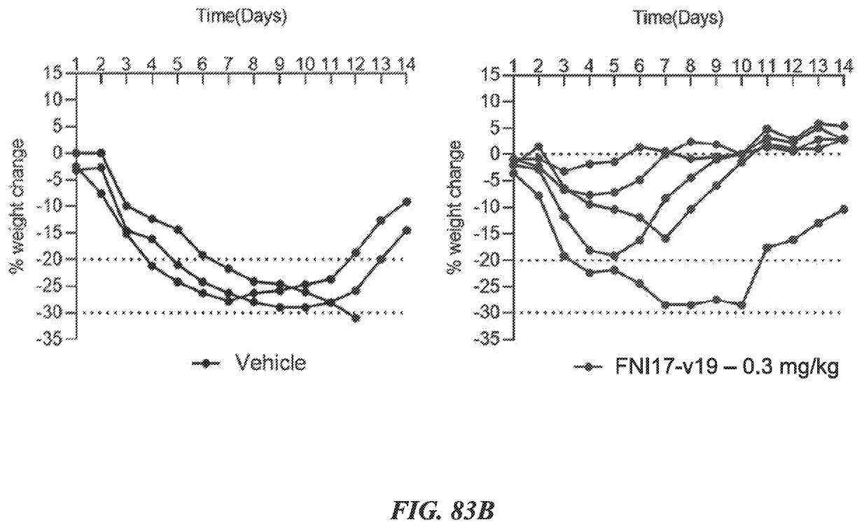

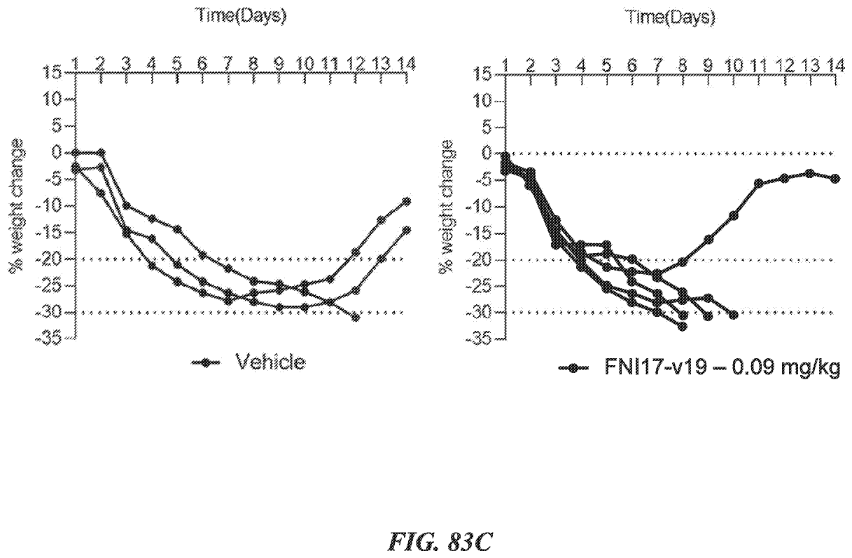

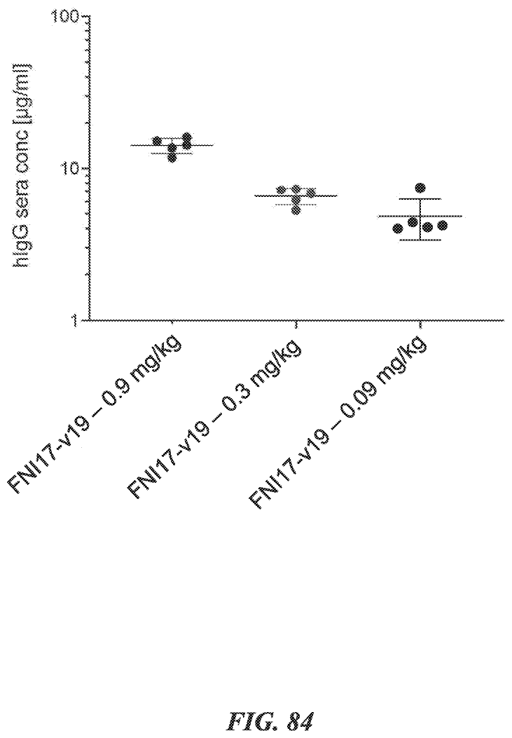

FIG. 84 shows pre-infection concentration of human IgG in sera from humanized FcγR mice pre-treated with FNI17-v19 from the study outlined in FIG. 82. Sera was collected from mice 2 hours prior to infection with 5LD50 H1N1 A/Puerto Rico/8/34. Body weight over fourteen days is shown in FIGS. 83A-83C.

Animals administered FNI17-v19 displayed limited to moderate body weight loss (and no mortality) down to 0.3 mg/kg. Human IgG quantification in the sera collected from the animals 2 hours before infection showed that mice receiving different doses of the mAb have similar human IgG concentrations, thus excluding potential problems associated to the administration of the antibody.

### Example 13

### Further Studies

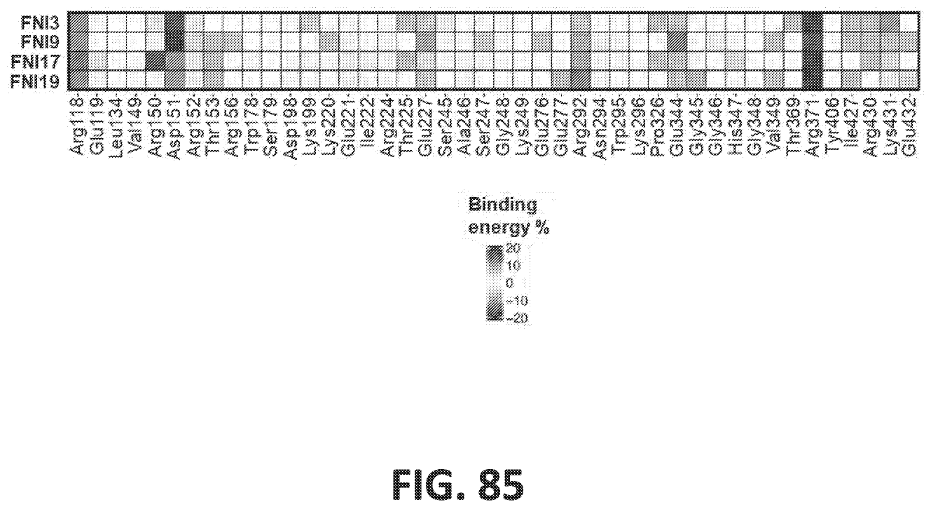

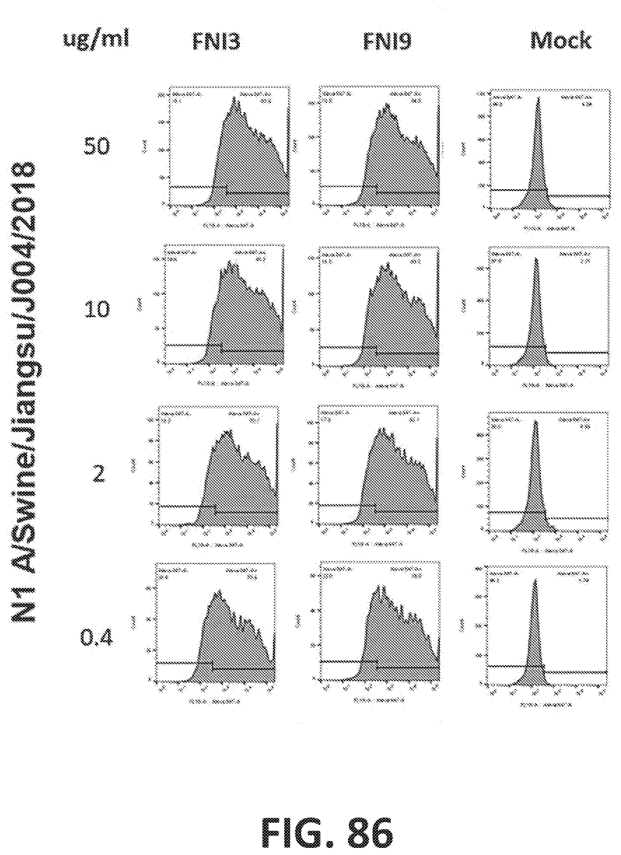

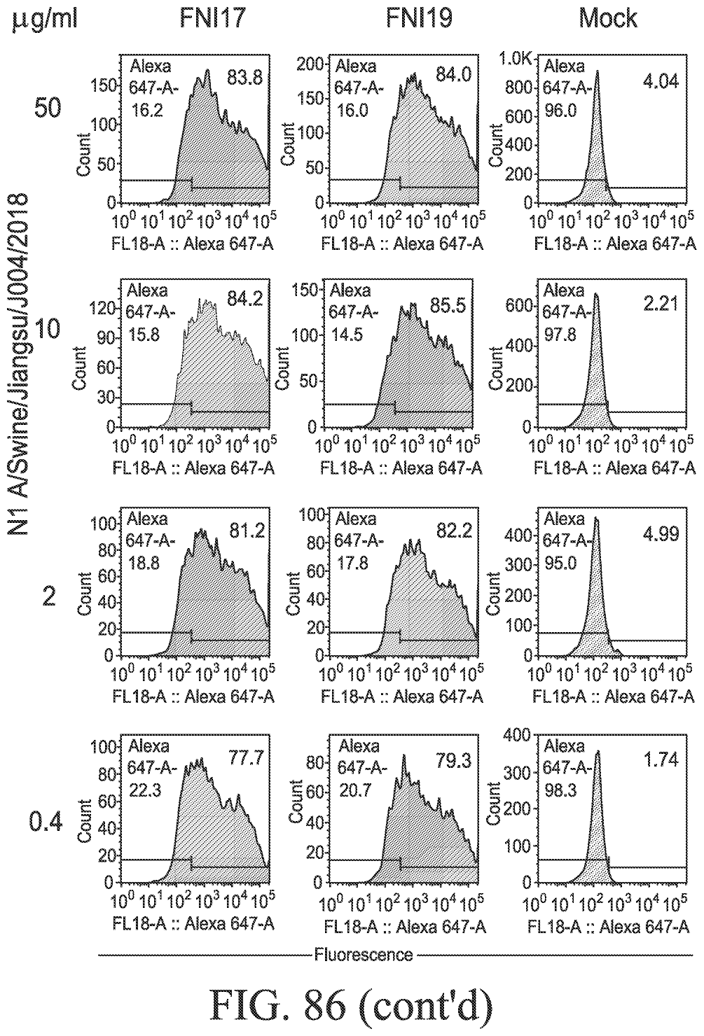

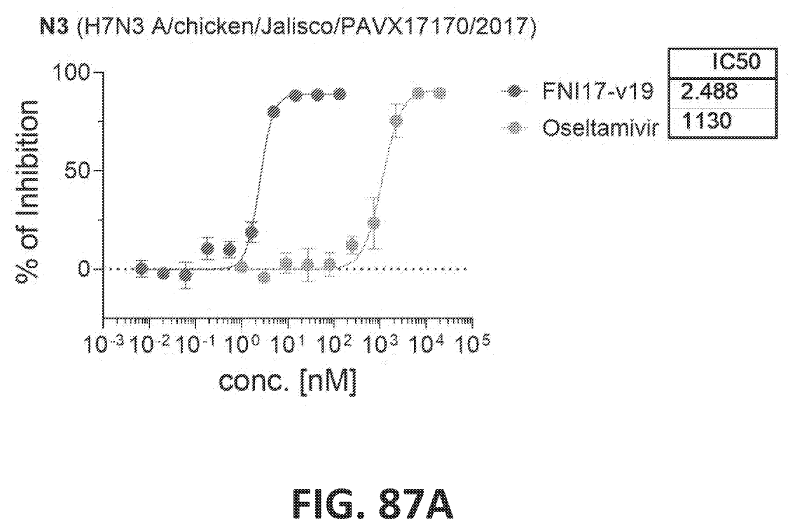

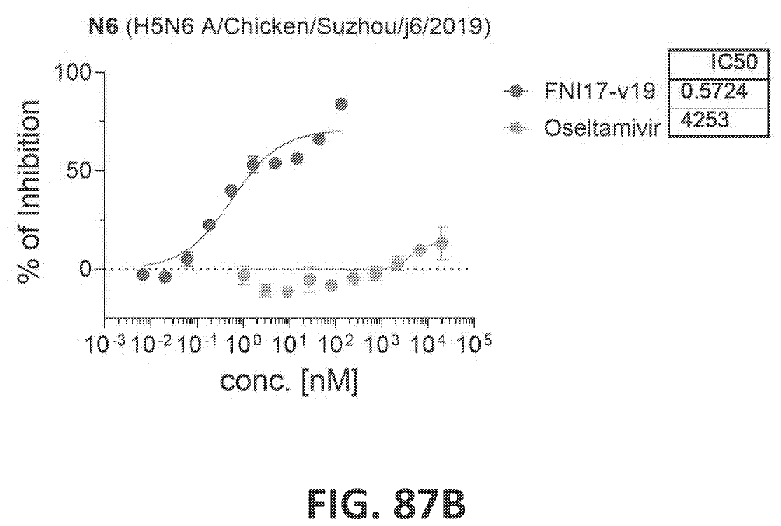

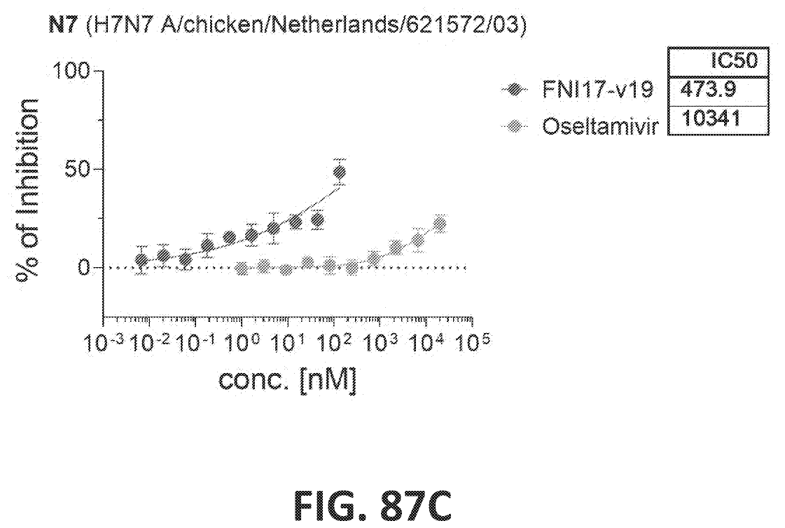

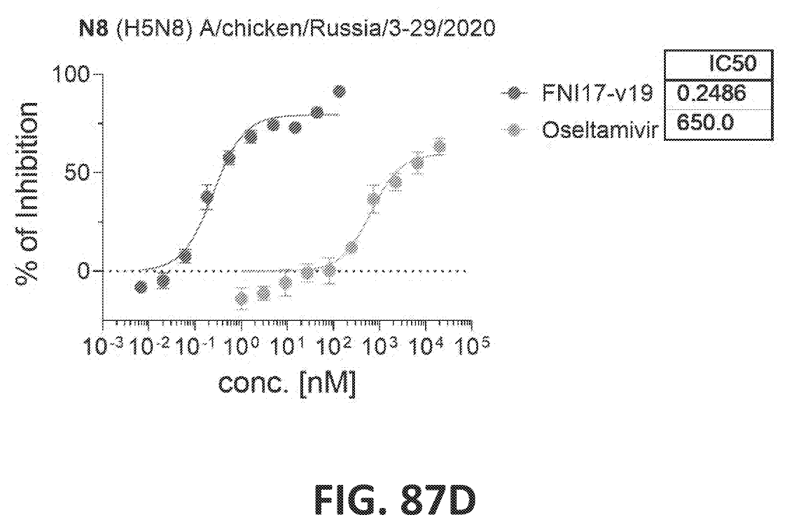

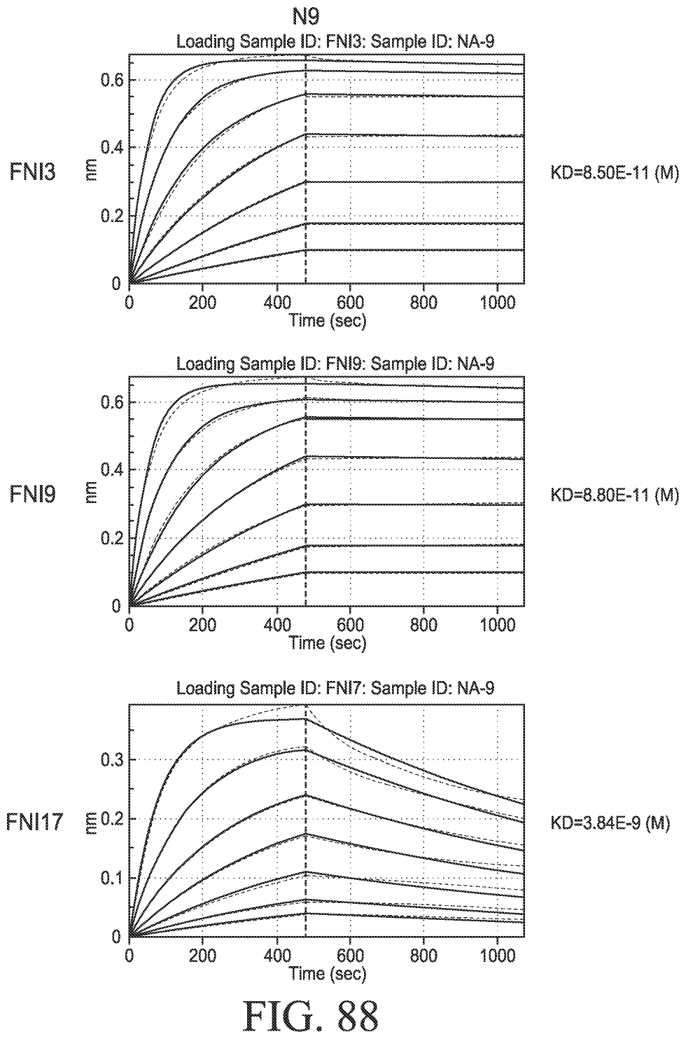

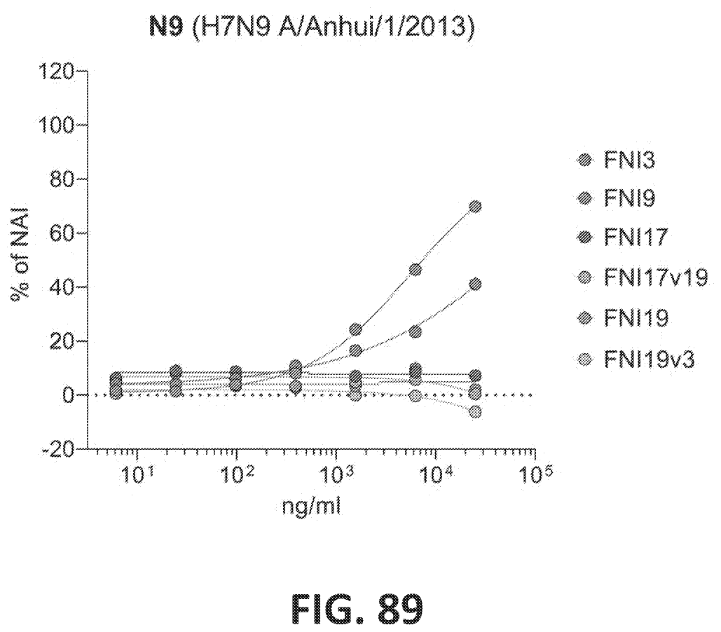

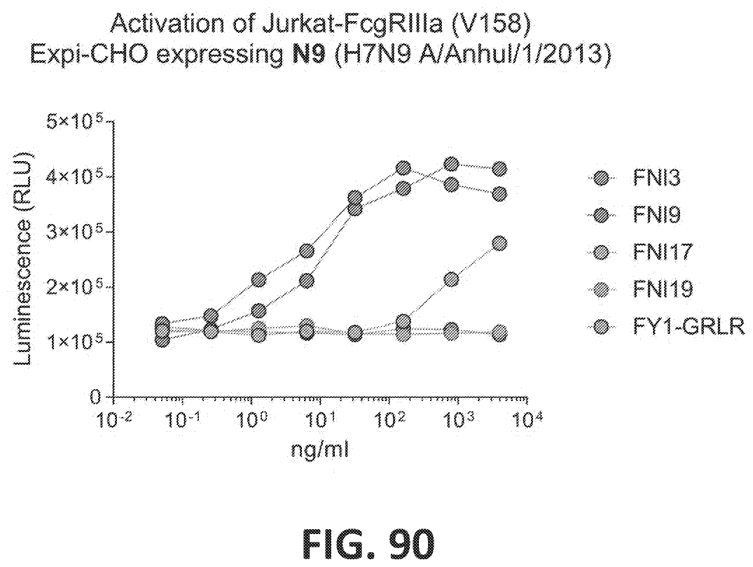

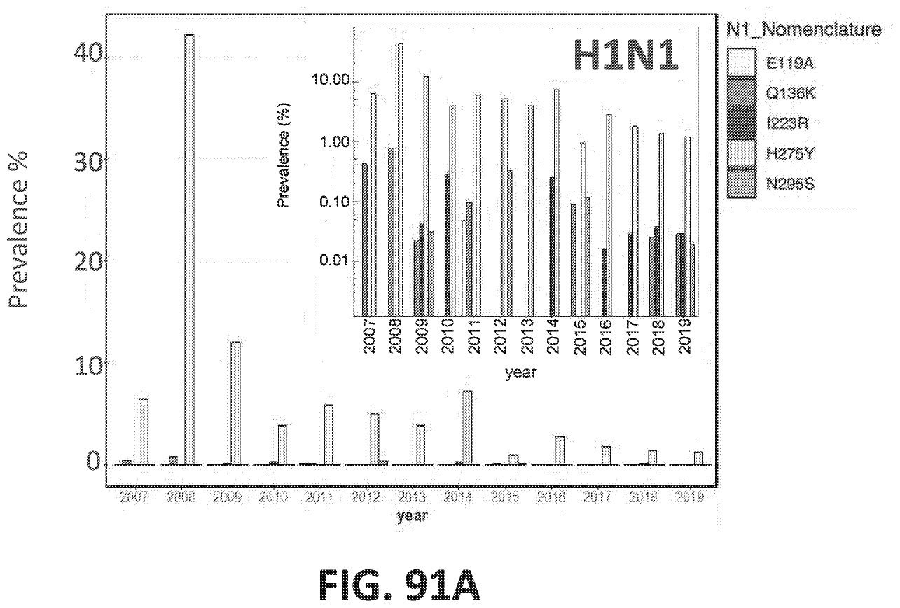

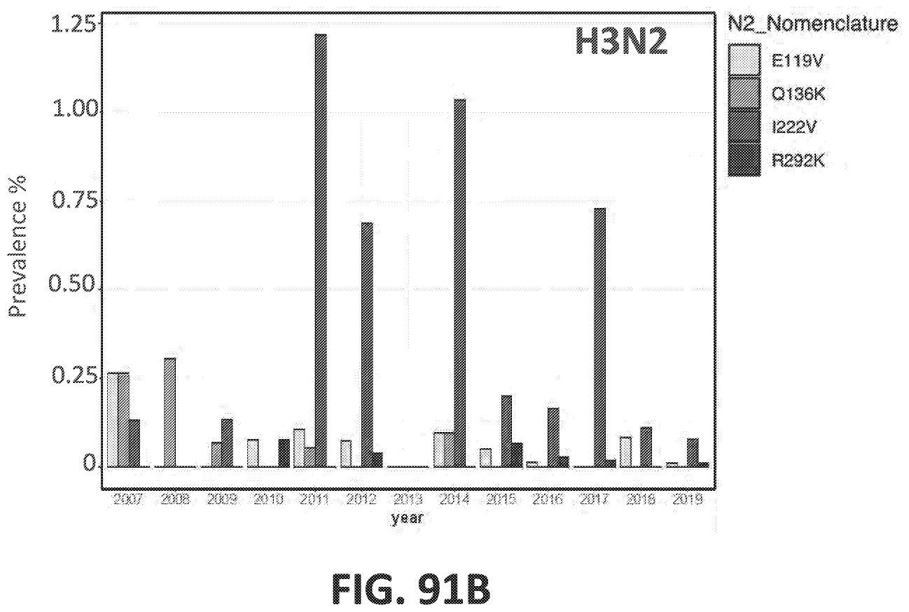

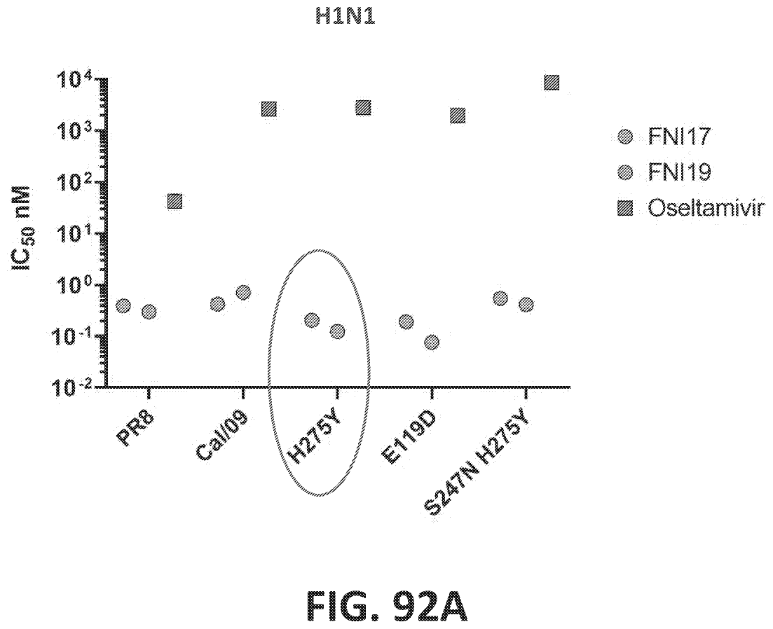

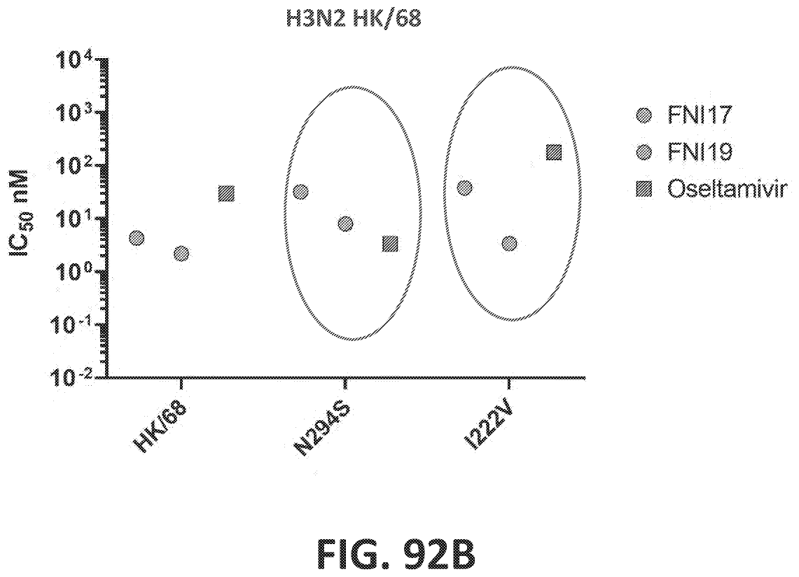

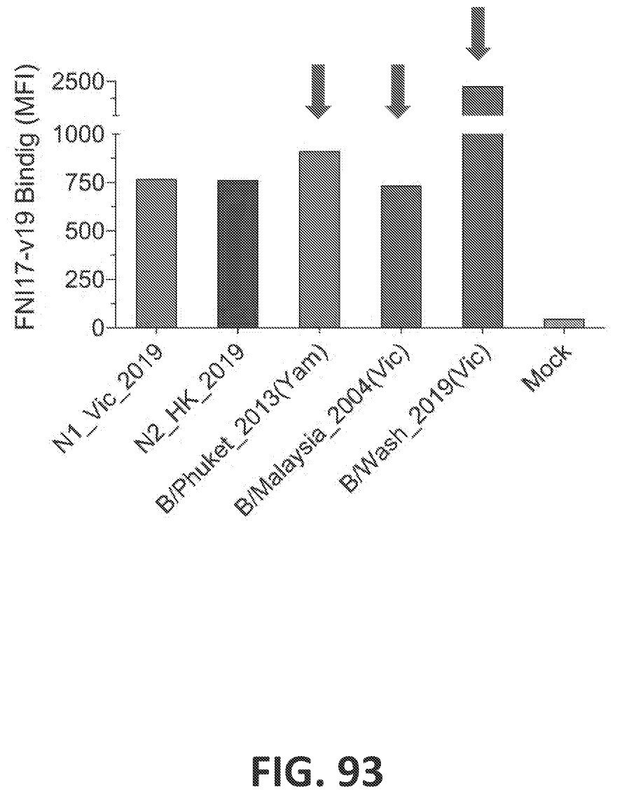

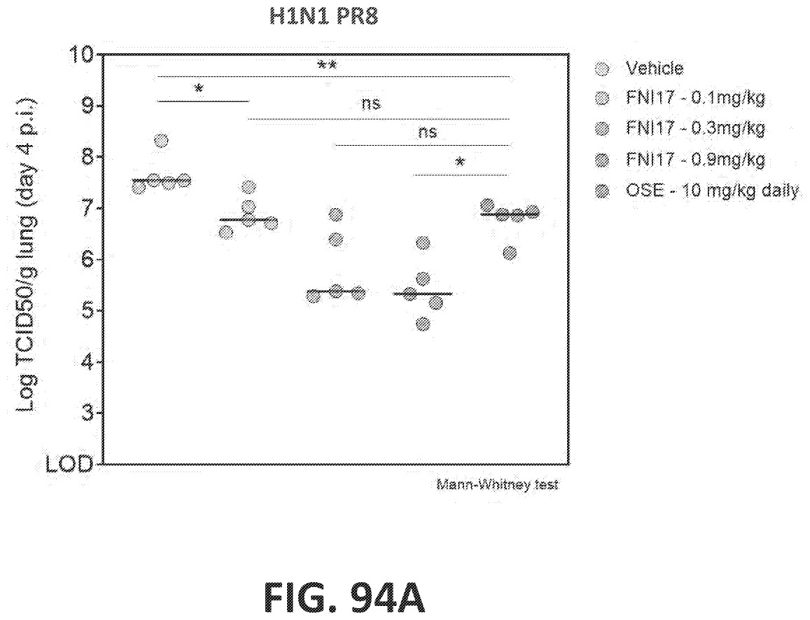

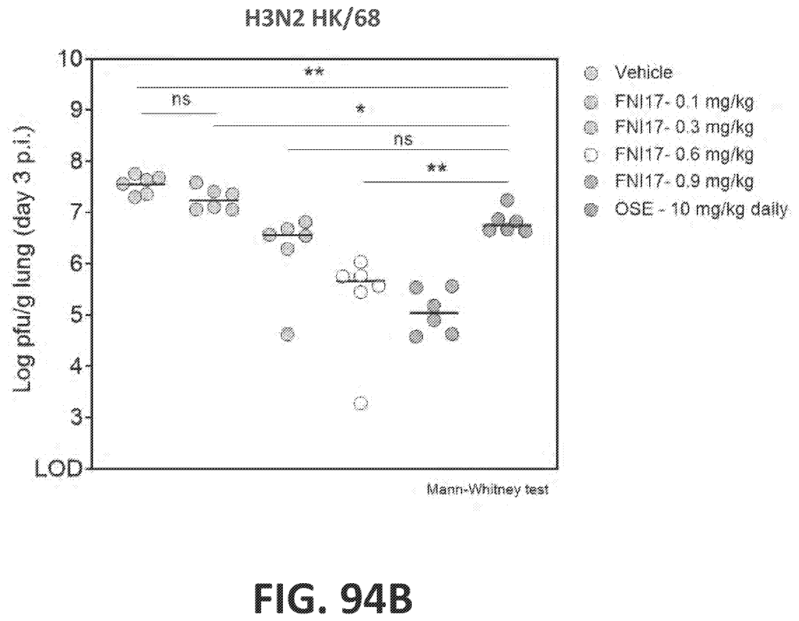

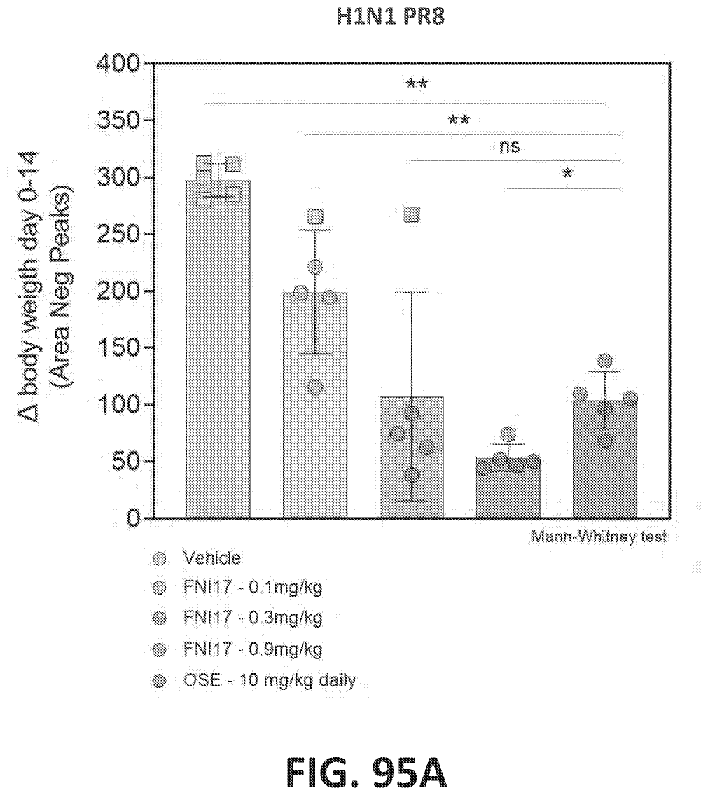

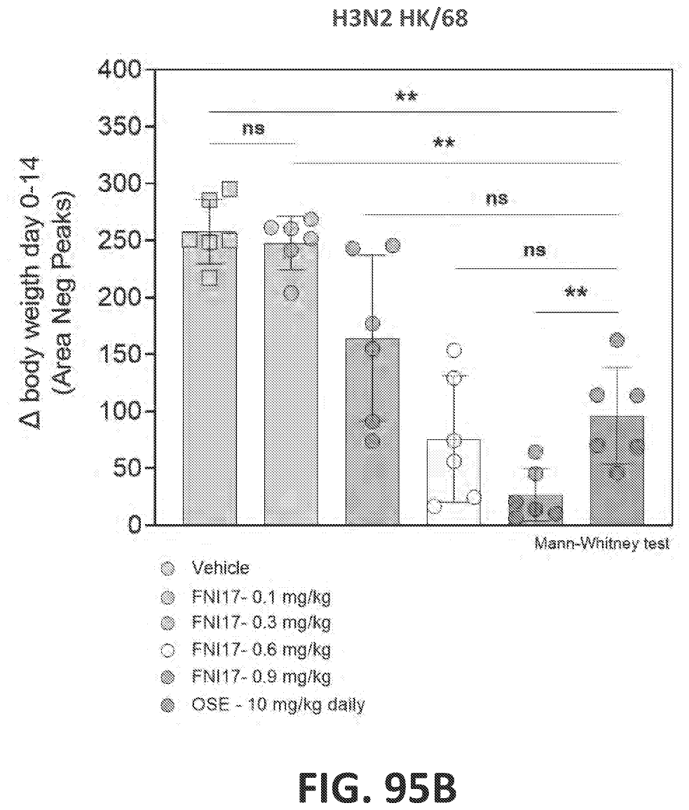

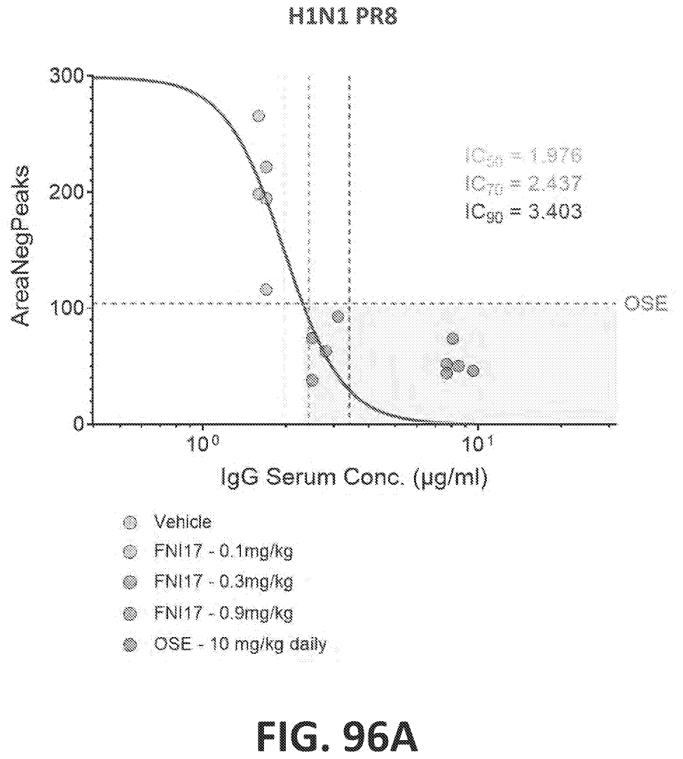

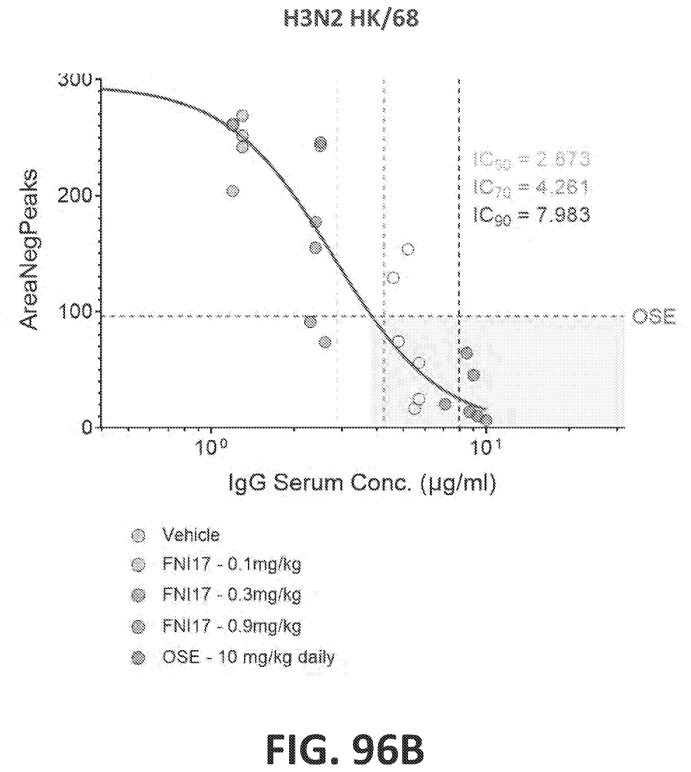

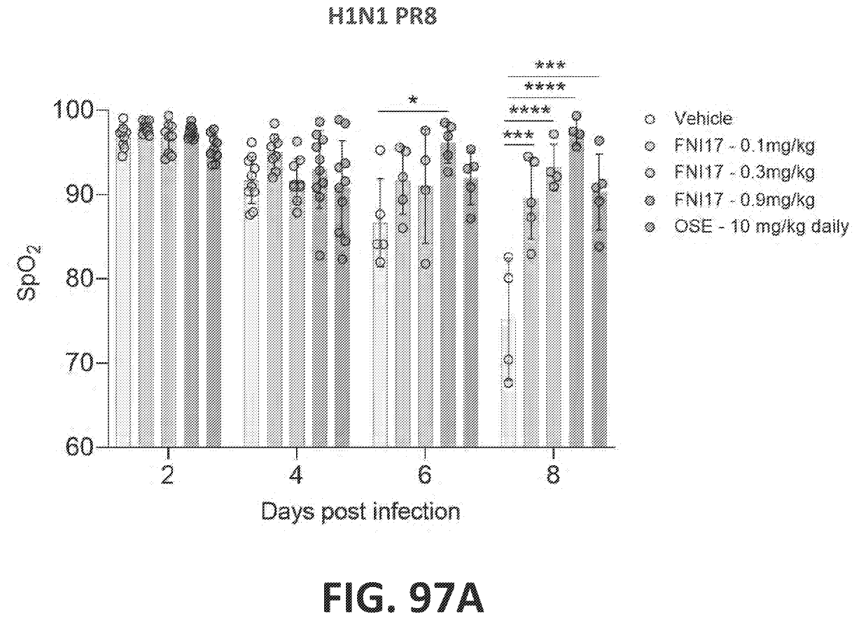

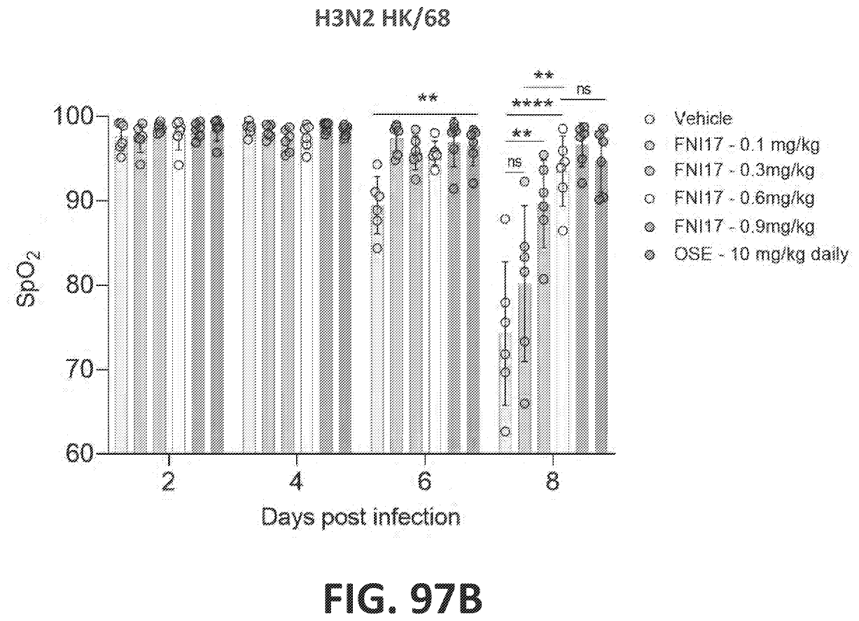

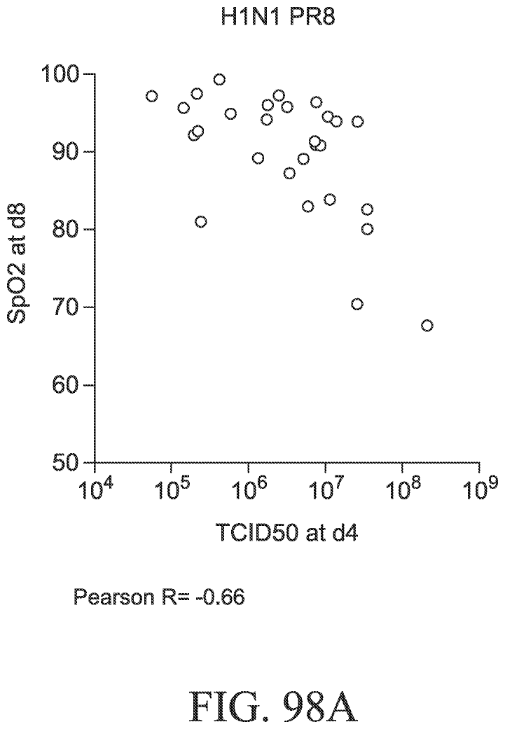

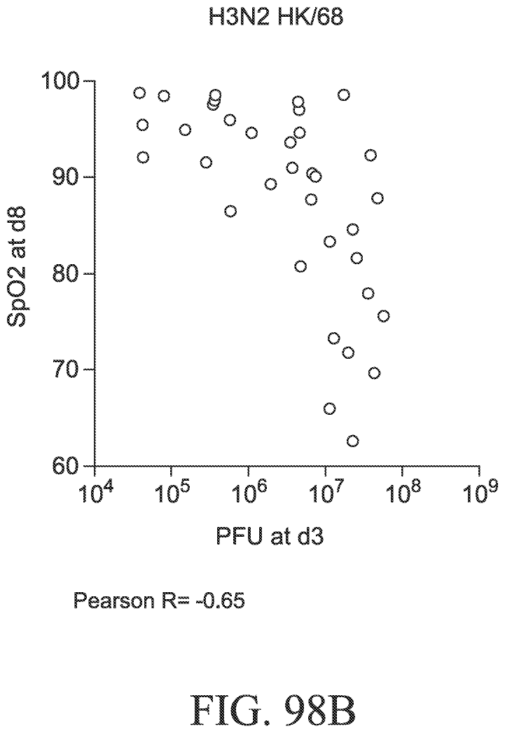

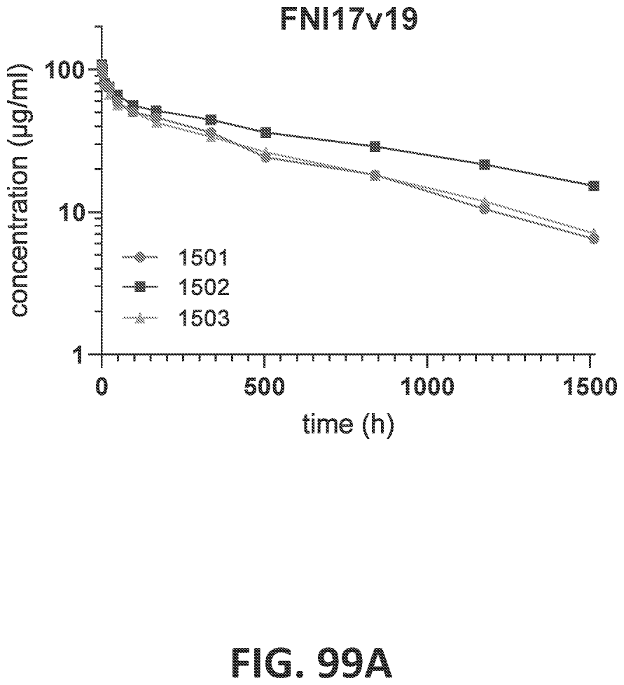

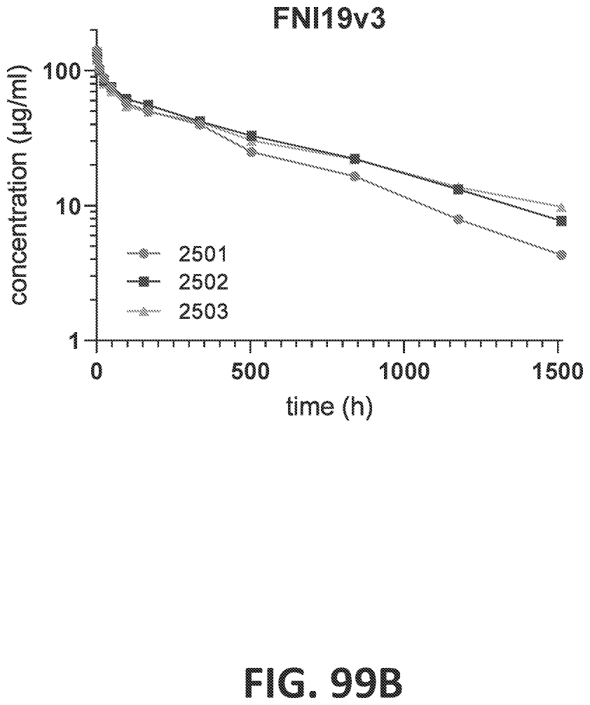

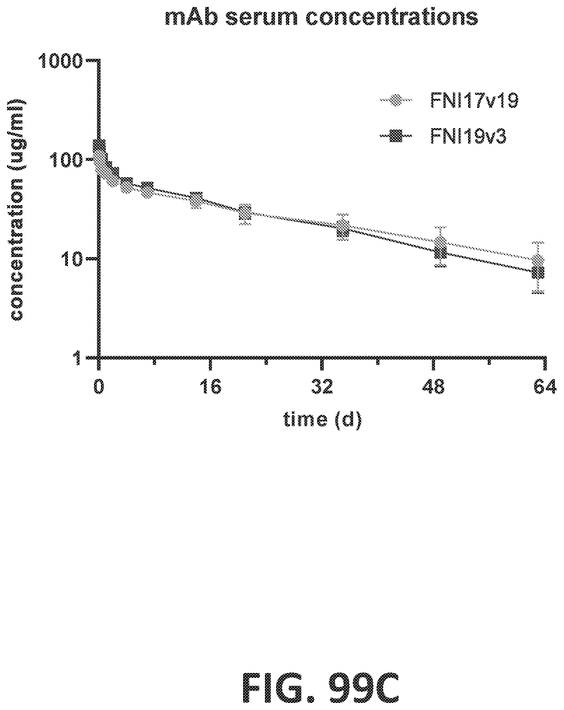

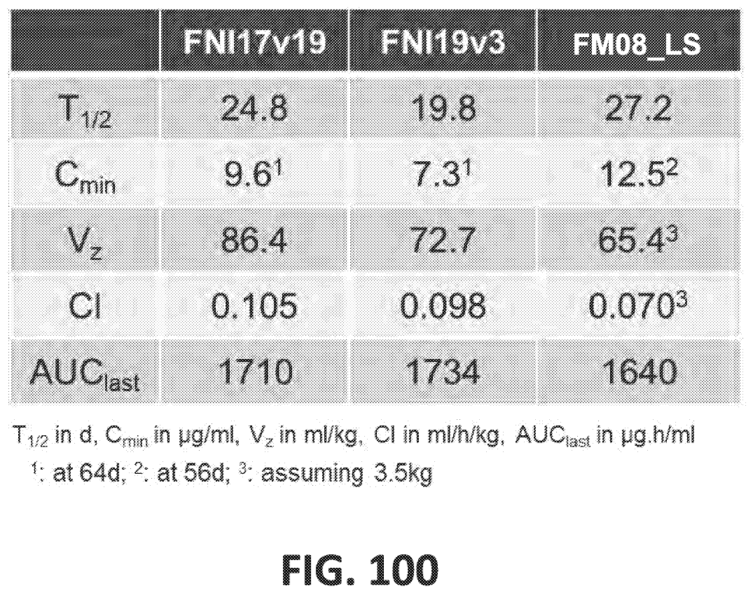

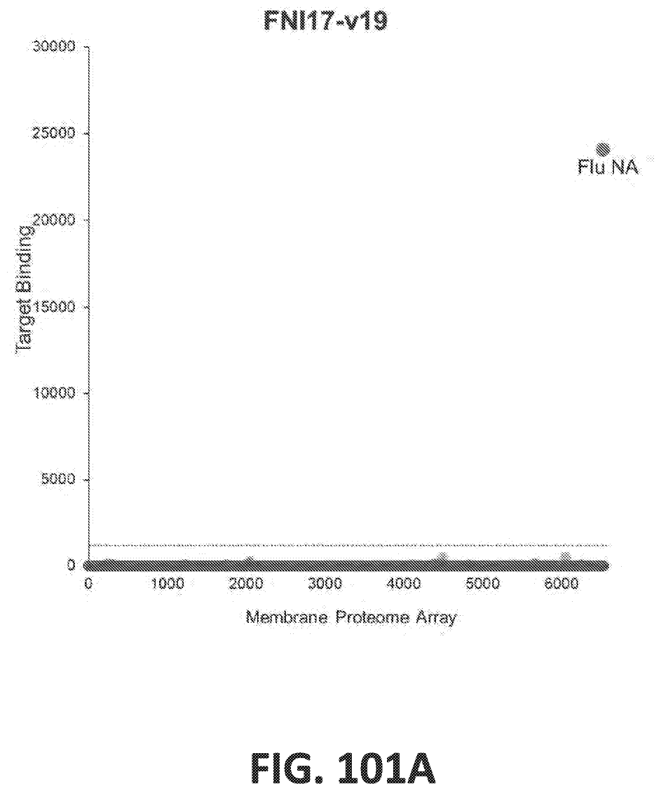

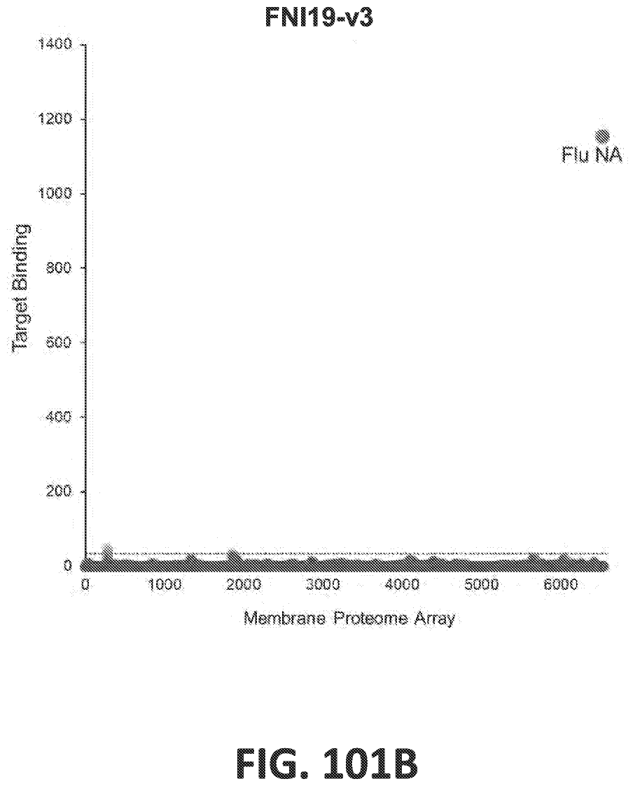

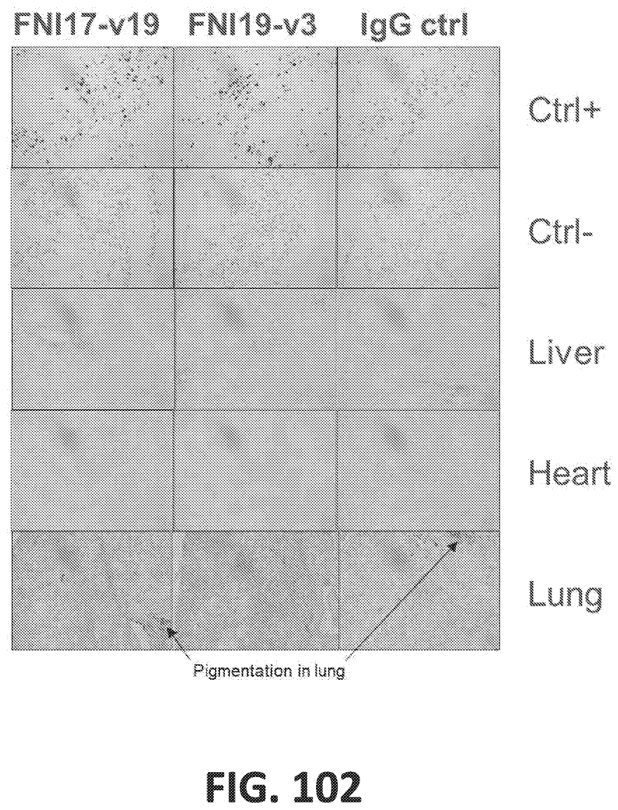

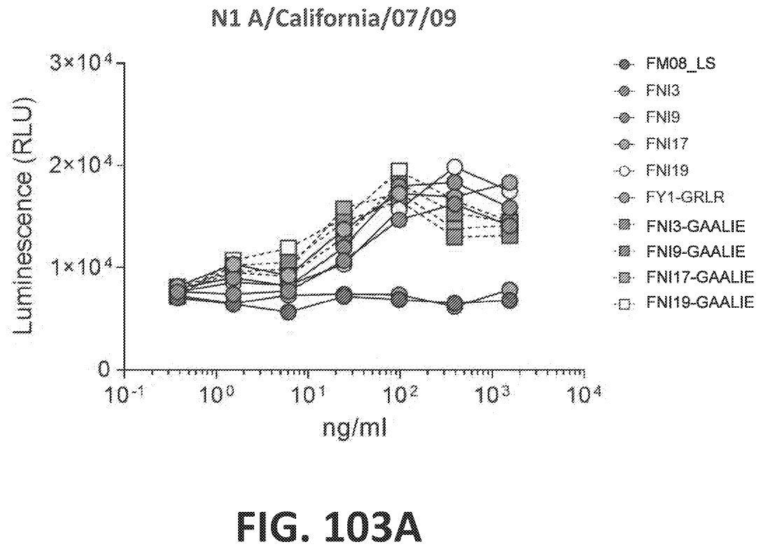

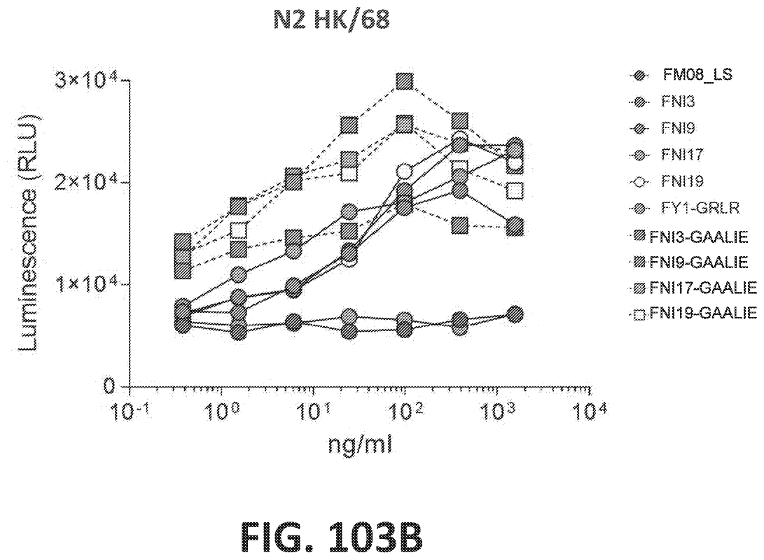

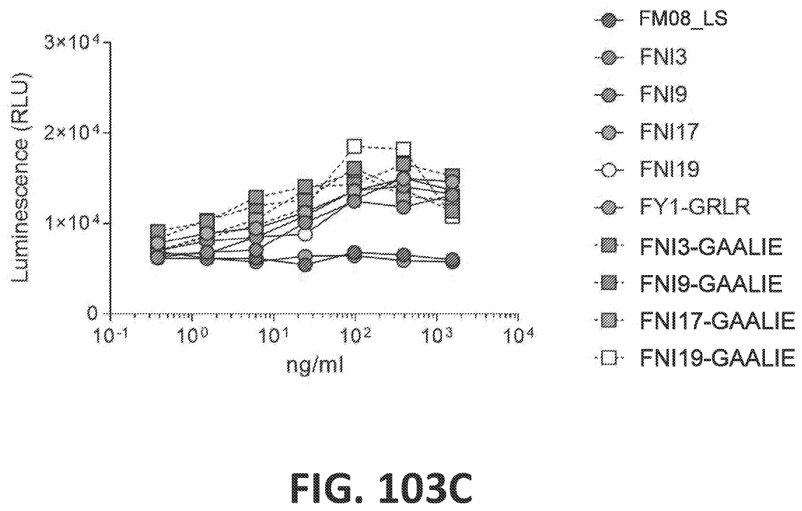

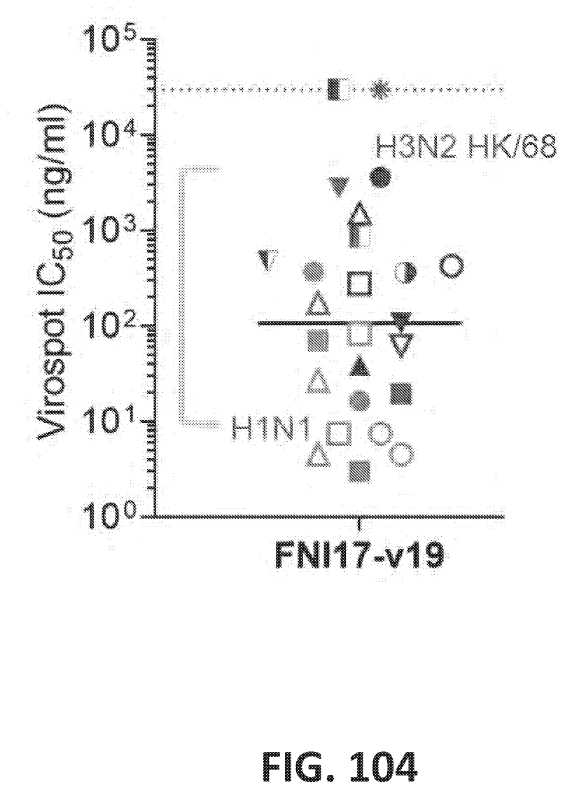

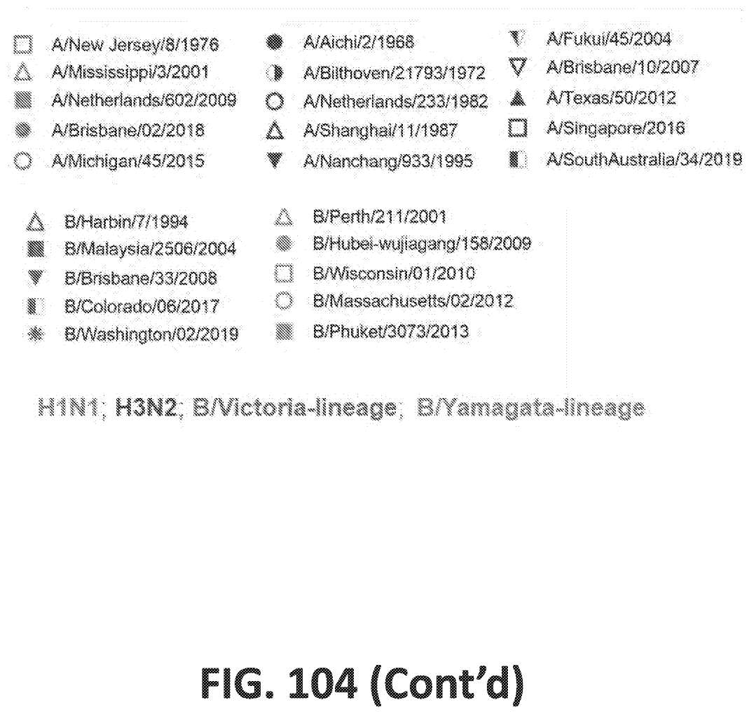

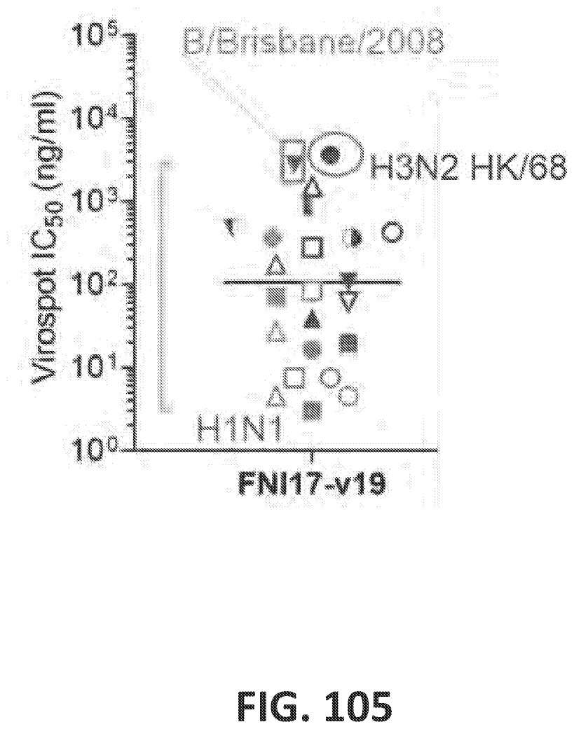

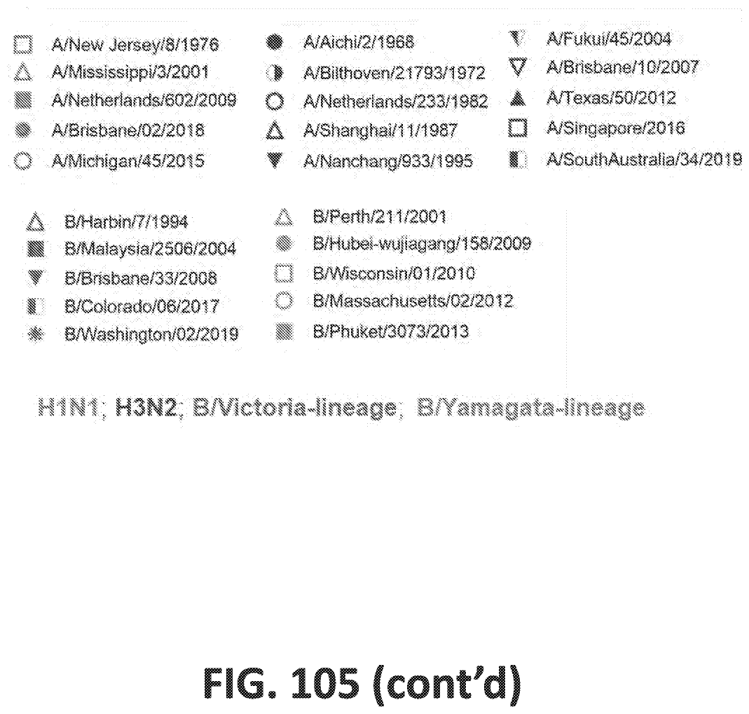

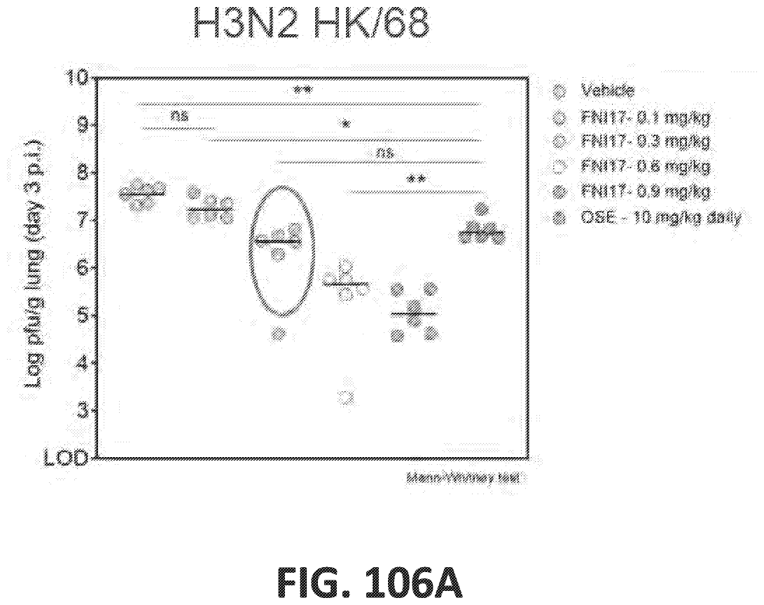

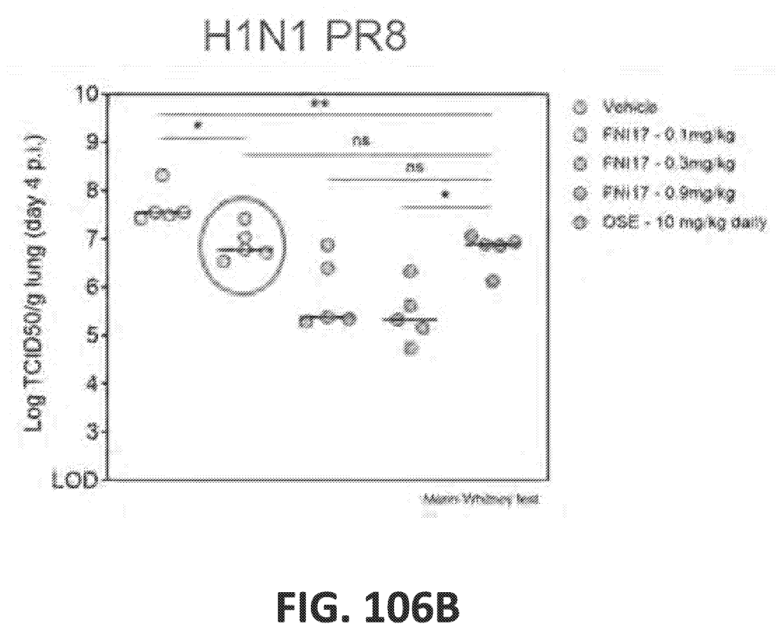

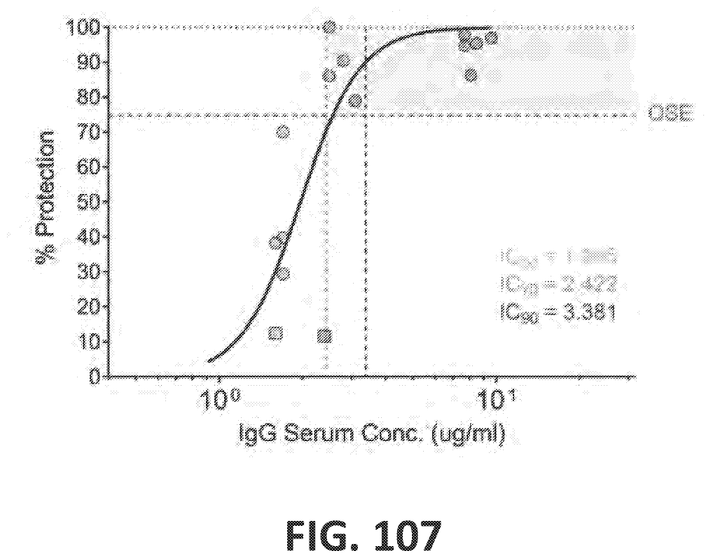

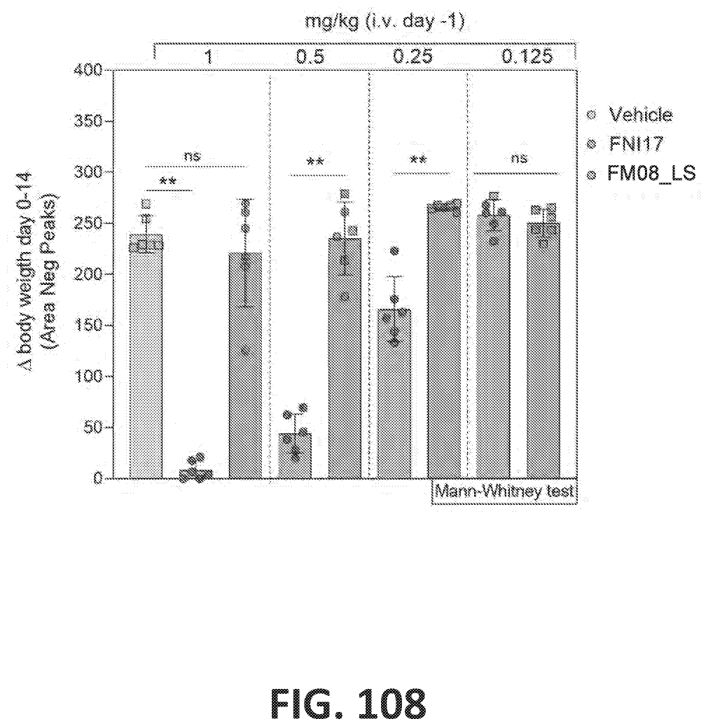

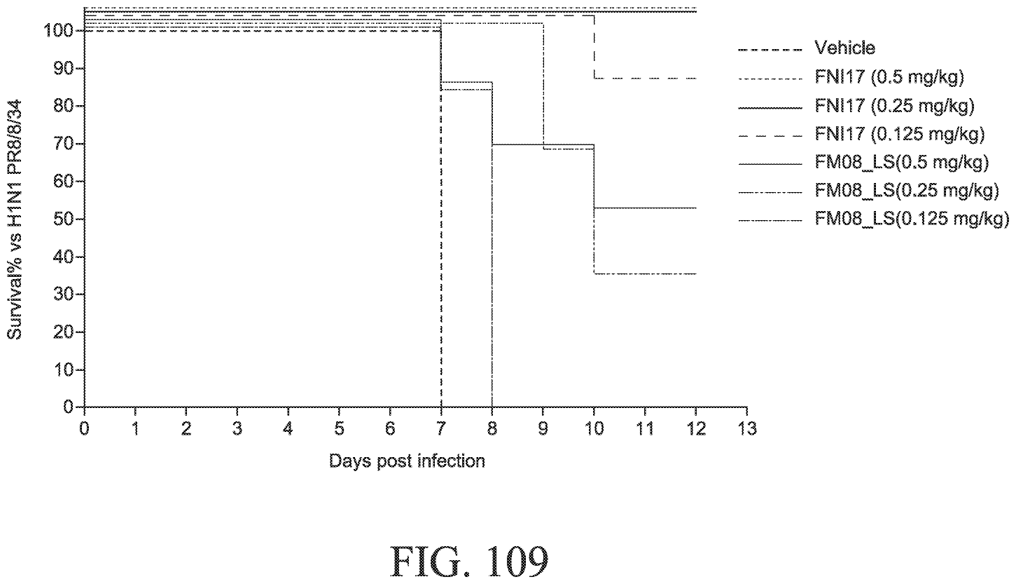

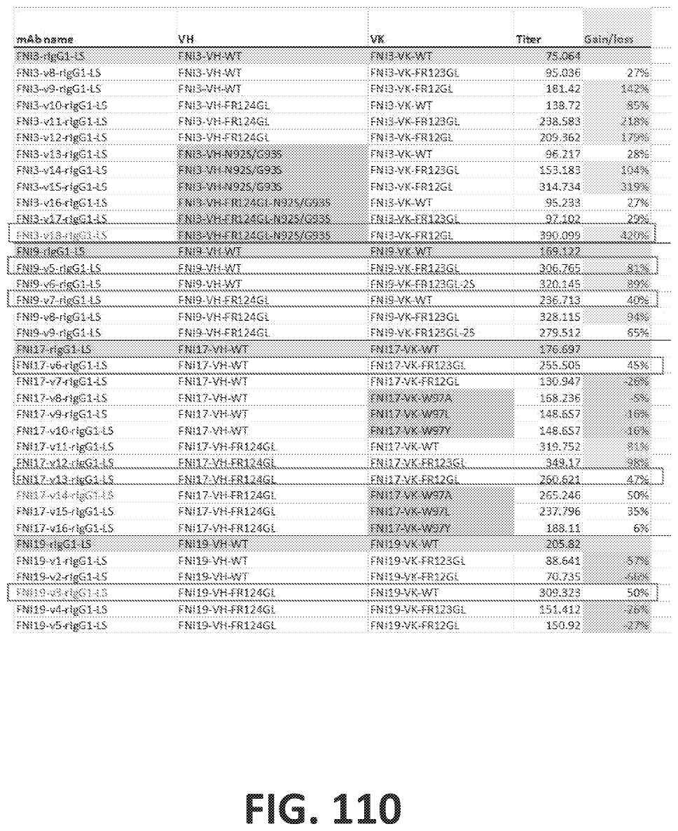

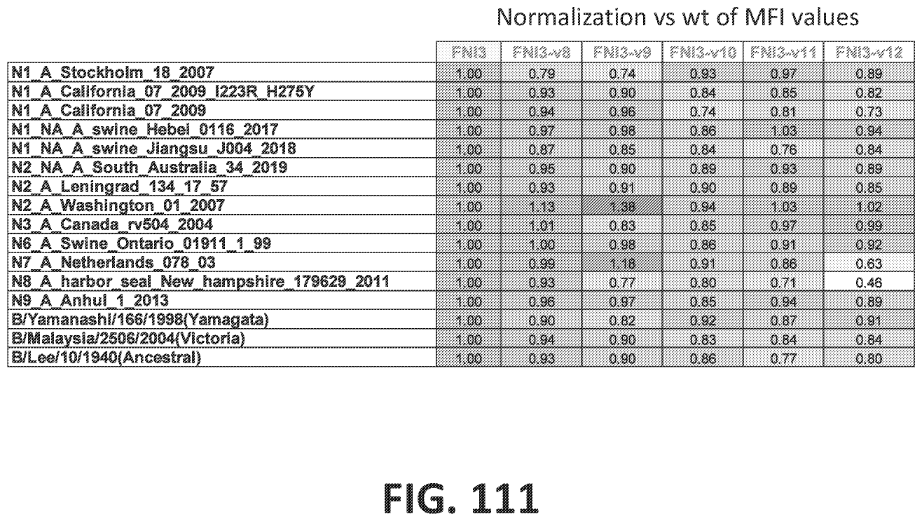

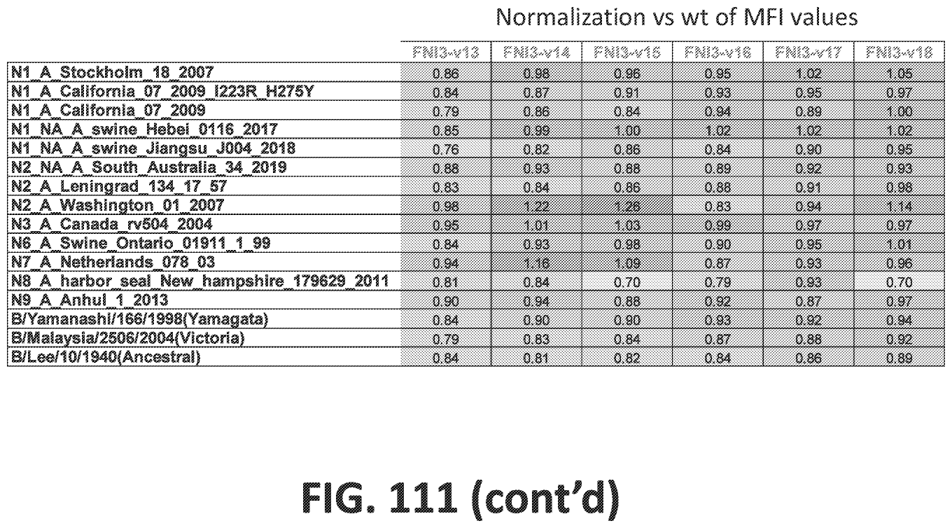

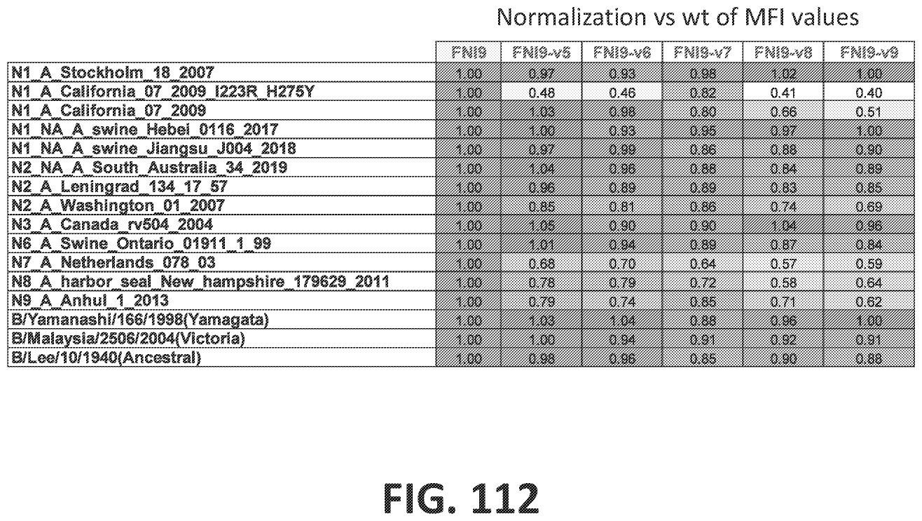

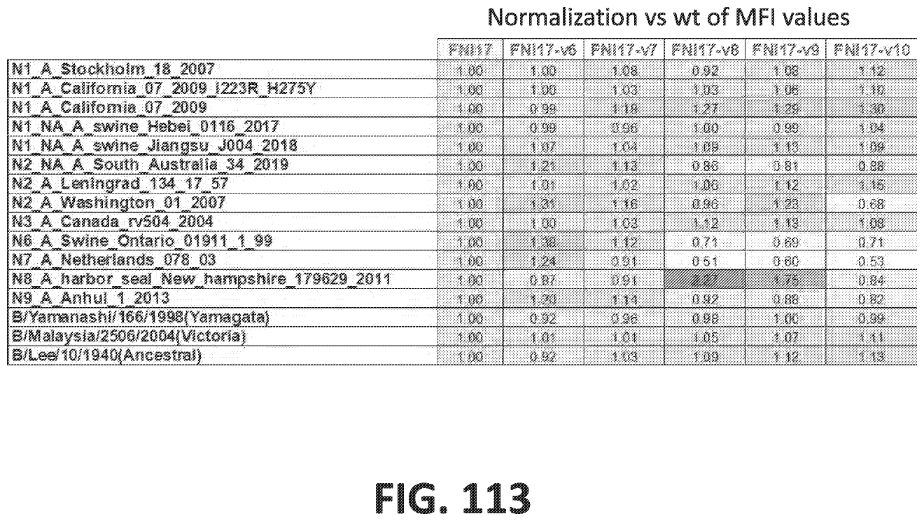

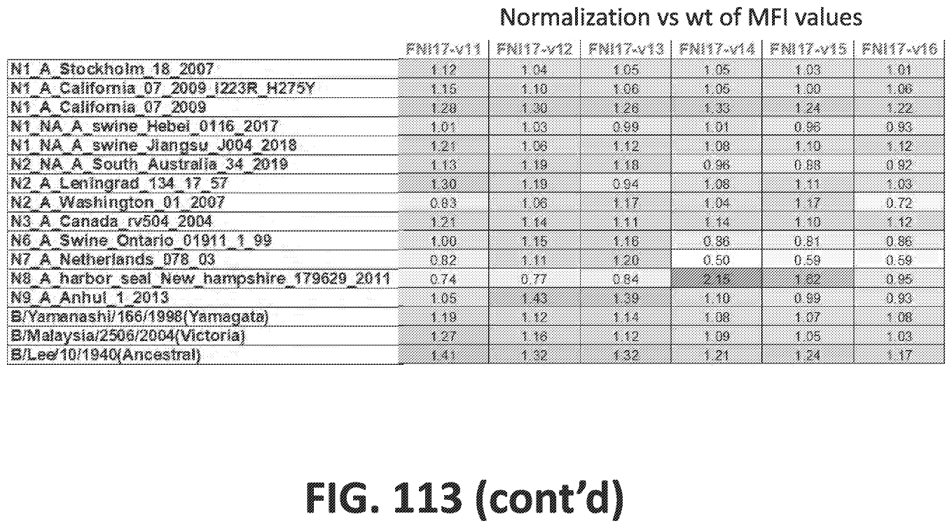

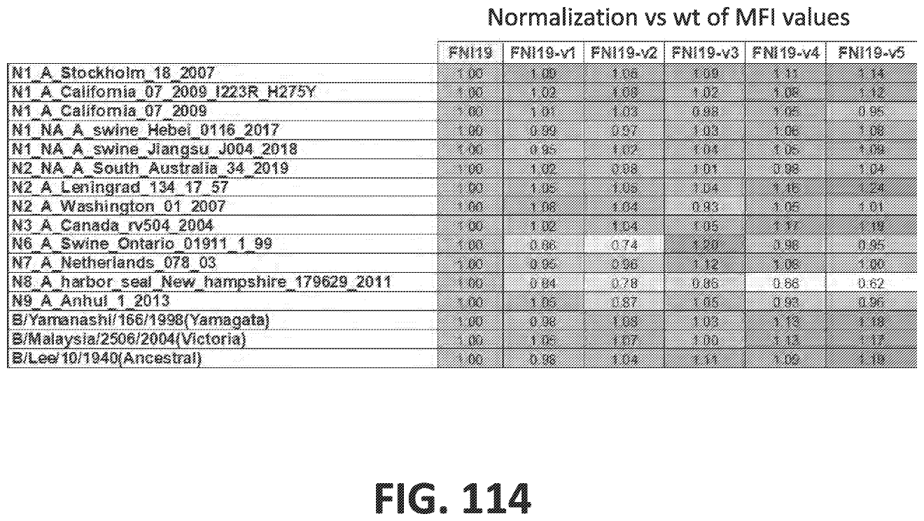

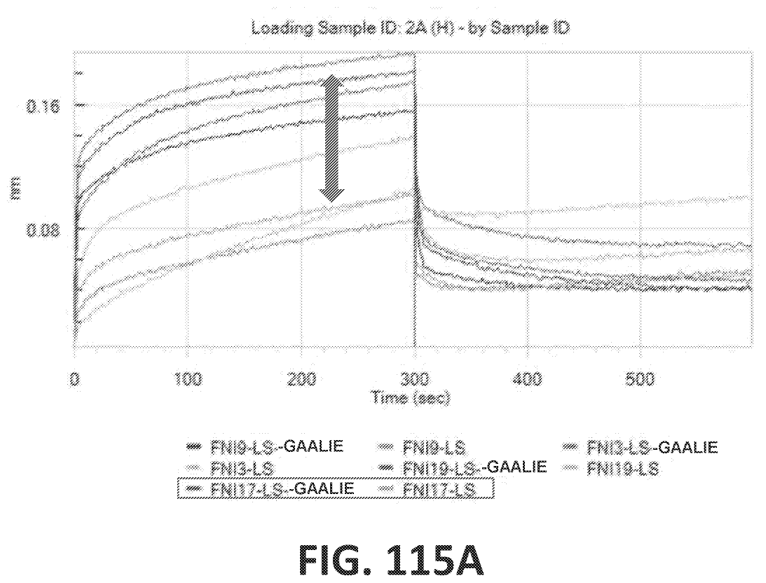

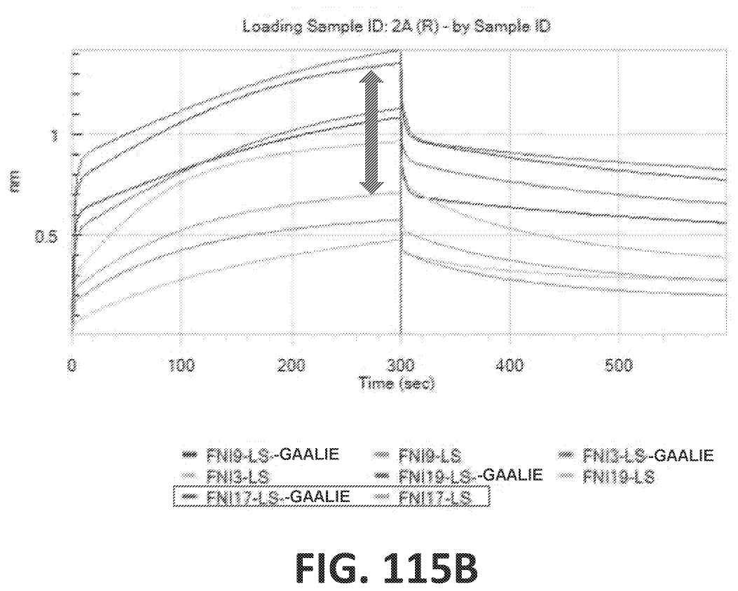

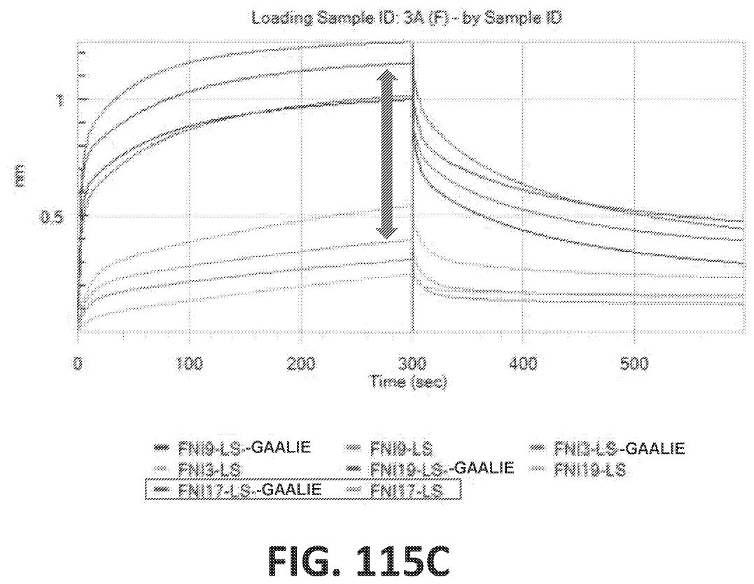

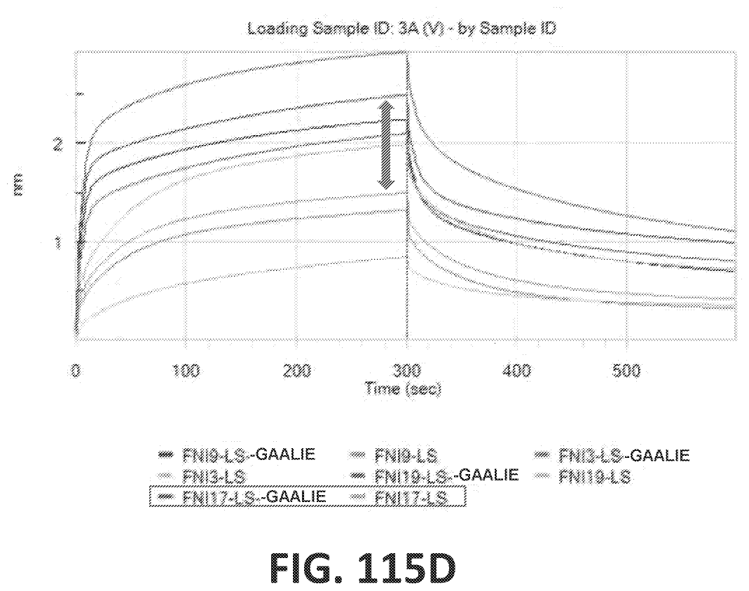

Additional studies were performed, as described and shown in FIGS. 80-115D.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/117,448, filed Nov. 23, 2020; U.S. Provisional Application No. 63/123,424, filed Dec. 9, 2020. U.S. Provisional Application No. 63/197,160, filed Jun. 4, 2021 and U.S. Provisional Application No. 63/261,463, filed Sep. 21, 2021 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antibody, or an antigen-binding fragment thereof, that is capable of binding to a neuraminidase (NA) from:
(i) an influenza A virus (IAV), wherein the IAV comprises a Group 1 IAV, a Group 2 IAV, or both; and
(ii) an influenza B virus (IBV).

2.-32. (canceled)

33. The antibody or antigen-binding fragment of claim 1, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein:
(i) optionally, the CDRH1 comprises or consists of the amino acid sequence set forth in any one of SEQ ID NOs.: 147, 3, 15, 27, 39, 51, 63, 75, 87, 99, 111, 123, 135, 159, and 231, or a functional variant thereof comprising one, two, or three acid substitutions, one or more of which substitutions is optionally a conservative substitution and/or is a substitution to a germline-encoded amino acid;
(ii) optionally, the CDRH2 comprises or consists of the amino acid sequence set forth in any one of SEQ ID NOs.: 148, 4, 16, 28, 40, 52, 64, 76, 88, 100, 112, 124, 136, 160, and 232, or a functional variant thereof comprising one, two, or three amino acid substitutions, one or more of which substitutions is optionally a conservative substitution and/or is a substitution to a germline-encoded amino acid;
(iii) the CDRH3 comprises or consists of the amino acid sequence set forth in any one of SEQ ID NOs.: 149, 5, 17, 29, 172, 41, 53, 65, 77, 89, 184, 101, 113, 125, 137, 161, and 233, or a functional variant thereof comprising one, two, or three amino acid substitutions, one or more of which substitutions is optionally a conservative substitution and/or is a substitution to a germline-encoded amino acid;
(iv) optionally, the CDRL1 comprises or consists of the amino acid sequence set forth in any one of SEQ ID NOs.: 153, 9, 21, 33, 45, 57, 69, 81, 93, 105, 117, 129, 141, 165, and 234, or a functional variant thereof comprising one, two, or three amino acid substitutions, one or more of which substitutions is optionally a conservative substitution and/or is a substitution to a germline-encoded amino acid;
(v) optionally, the CDRL2 comprises or consists of the amino acid sequence set forth in any one of SEQ ID NOs.: 154, 10, 22, 34, 46, 58, 70, 82, 94, 106, 118, 130, 142, 166, and 235, or a functional variant thereof comprising one, two, or three amino acid substitutions, one or more of which substitutions is optionally a conservative substitution and/or is a substitution to a germline-encoded amino acid; and/or
(vi) optionally, the CDRL3 comprises or consists of the amino acid sequence set forth in any one of SEQ ID NOs.: 155, 11, 23, 35, 175, 178, 181, 47, 59, 71, 83, 95, 187, 193, 107, 119, 131, 143, 190, 167, and 236, or a functional variant thereof comprising having one, two, or three amino acid substitutions, one or more of which substitutions is optionally a conservative substitution and/or is a substitution to a germline-encoded amino acid.

34. (canceled)

35. The antibody or antigen-binding fragment of claim 1, wherein:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 199, 2, 14, 26, 171, 38, 50, 62, 74, 86, 183, 98, 110, 122, 134, 146, 158, 203, 207, 216, and 228, wherein sequence variation is optionally limited to one or more framework regions and/or sequence variation comprises comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence set forth in any one of SEQ ID NOs.: 201, 8, 20, 32, 44, 56, 68, 80, 92, 104, 116, 128, 140, 152, 174, 177, 180, 186, 189, 192, 164, 205, 209, 217, and 230, wherein sequence variation is optionally limited to one or more framework regions and/or sequence variation comprises one or more substitution to a germline-encoded amino acid.

36.-44. (canceled)

45. A polypeptide comprising an amino acid sequence according to SEQ ID NO.:219, wherein the polypeptide is capable of binding to an influenza virus neuraminidase (NA).

46. The polypeptide of claim 45, wherein the polypeptide comprises an antibody heavy chain variable domain (VH), or a fragment thereof, and the amino acid sequence according to SEQ ID NO.:219 is comprised in the VH or fragment thereof.

47. The polypeptide of claim 45, wherein the amino acid sequence according to SEQ ID NO.:219 comprises any one of SEQ ID NOs.: 149, 5, 17, 29, 172, 41, 53, 65, 77, 89, 184, 101, 113, 125, 137, and 161.

48. The polypeptide of claim 46, wherein the VH further comprises:
(i) an amino acid sequence sequence according to SEQ ID NO.:220; and/or
(ii) an amino acid sequence according to SEQ ID NO.:221.

49. The polypeptide of claim 46, further comprising an antibody light chain variable domain (VL), wherein, optionally, the VL comprises:
(i) an amino acid sequence according to SEQ ID NO.:222;
(ii) an amino acid sequence according to SEQ ID NO.:223; and/or
(iii) an amino acid sequence according to SEQ ID NO.:224.

50. The polypeptide of claim 46, wherein the VH comprises or consists of an amino acid sequence having at least 90%, at least 92%, at least 95%, at least 97%, or at least 99% identity to the amino acid sequence of any one of SEQ ID NOs.: 199, 2, 14, 26, 171, 38, 50, 62, 74, 86, 183, 98, 110, 122, 134, 146, 158, 203, 207, 216, and 228.

51. The polypeptide of claim 49, wherein the VL comprises or consists of an amino acid sequence having at least 90%, at least 92%, at least 95%, at least 97%, or at least 99% identity to the amino acid sequence of any one of SEQ ID NOs.: 201, 8, 20, 32, 44, 56, 68, 80, 92, 104, 116, 128, 140, 152, 174, 177, 180, 186, 189, 192, 164, 205, 209, 217, and 230.

52. The polypeptide of claim 45, wherein the polypeptide comprises an antibody or an antigen-binding fragment thereof.

53. An antibody or an antigen-binding fragment thereof, comprising a heavy chain variable domain (VH) amino acid sequence and a light chain variable domain (VL) amino acid sequence, wherein the VH comprises or consists of an amino acid sequence having at least 90%, at least 92%, at least 95%, at least 97%, or at least 99% identity to the amino acid sequence of any one of SEQ ID NOs.: 199, 2, 14, 26, 171, 38, 50, 62, 74, 86, 183, 98, 110, 122, 134, 146, 158, 203, 207, 216, and 228, and wherein the VL comprises or consists of an amino acid sequence having at least 90%, at least 92%, at least 95%, at least 97%, or at least 99% identity to the amino acid sequence of any one of SEQ ID NOs.: 201, 8, 20, 32, 44, 56, 68, 80, 92, 104, 116, 128, 140, 152, 174, 177, 180, 186, 189, 192, 164, 205, 209, 217, and 230,
wherein the antibody or antigen-binding fragment thereof is capable of binding to a neuraminidase (NA) from: (i) an influenza A virus (IAV), wherein the IAV comprises a Group 1 IAV, a Group 2 IAV, or both; and/or (ii) an influenza B virus (IBV).

54.-62. (canceled)

63. The antibody or antigen-binding fragment of claim 1, which is a IgG, IgA, IgM, IgE, or IgD isotype.

64. The antibody or antigen-binding fragment of claim 1, which is an IgG isotype selected from IgG1, IgG2, IgG3, and IgG4.

65. The antibody or antigen-binding fragment of claim 1, wherein the antibody, or the antigen-binding fragment, comprises a human antibody, a monoclonal antibody, a purified antibody, a single chain antibody, a Fab, a Fab′, a F(ab′)2, or Fv.

66. The antibody or antigen-binding fragment of claim 1, wherein the antibody or antigen-binding fragment is a multi-specific antibody or antigen-binding fragment.

67.-68. (canceled)

69. The antibody or antigen-binding fragment of claim 1, wherein the antibody or antigen-binding fragment comprises an (e.g., IgG1) Fc polypeptide or a fragment thereof.

70. The antibody or antigen-binding fragment of claim 69, wherein the Fc polypeptide or fragment thereof comprises:
(i) a mutation that increases binding affinity to a human FcRn (e.g., as measured using surface plasmon resonance (SPR) (e.g., Biacore, e.g., T200 instrument, using manufacturer's protocols)), as compared to a reference Fc polypeptide that does not comprise the mutation; and/or
(ii) a mutation that increases binding affinity to a human FcγR (e.g., as measured using surface plasmon resonance (SPR) (e.g., Biacore, e.g., T200 instrument, using manufacturer's protocols)) as compared to a reference Fc polypeptide that does not comprise the mutation.

71. The antibody or antigen-binding fragment of claim 70, wherein the mutation that increases binding affinity to a human FcRn comprises: M428L; N434S; N434H; N434A; N434S; M252Y; S254T; T256E; T250Q; P257I; Q311I; D376V; T307A; E380A; or any combination thereof.

72.-73. (canceled)

74. The antibody or antigen-binding fragment of claim 70, wherein the mutation that enhances binding to a FcγR comprises S239D; I332E; A330L; G236A; or any combination thereof.

75.-80. (canceled)

81. An isolated polynucleotide encoding the antibody or antigen-binding fragment of claim 1, or encoding a VH, a heavy chain, a VL, and/or a light chain of the antibody or the antigen-binding fragment.

82. An isolated polynucleotide encoding the polypeptide of claim 45.

83.-90. (canceled)

91. A recombinant vector comprising the polynucleotide of claim 81.

92. A host cell comprising the polynucleotide of claim 81, wherein the polynucleotide is optionally heterologous to the host cell and/or wherein the host cell is capable of expressing the encoded antibody or antigen-binding fragment or polypeptide.

93. (canceled)

94. A composition comprising the antibody or antigen-binding fragment of claim 1,
and a pharmaceutically acceptable excipient, carrier, or diluent.

95. (canceled)

96. A composition comprising the polynucleotide of claim 81 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

97. A method of making an antibody or antigen-binding fragment of claim 1, comprising culturing a host cell comprising a polynucleotide encoding the antibody or antigen-binding fragment for a time and under conditions sufficient for the host cell or human B cell, respectively, to express the antibody or antigen-binding fragment.

98. (canceled)

99. A method of treating or preventing an IAV infection and/or an IBV infection in a subject, the method comprising administering to the subject an effective amount of the antibody or antigen-binding fragment of claim 1.

100.-114. (canceled)
